(19) United States
(12) Patent Application Publication
(10) Pub. No.: US 2014/0309137 A1
(43) Pub. Date: Oct. 16, 2014

(54) GENE EXPRESSION PROFILES ASSOCIATED WITH LEAN PHENOTYPE AND USES THEREOF

(71) Applicant: NESTEC SA, Vevey (CH)

(72) Inventors: Yuanlong Pan, Chesterfield, MO (US); Stephen S. Hannah, Chesterfield, MO (US); Rondo P. Middleton, Creve Coeur, MO (US)

(21) Appl. No.: 14/314,088

(22) Filed: Jun. 25, 2014

Related U.S. Application Data
(63) Continuation of application No. 12/737,745, filed on Mar. 18, 2011.
(60) Provisional application No. 61/190,369, filed on Aug. 28, 2008.

(51) Int. Cl. C12Q 1/68

(52) U.S. Cl. CPC C12Q 1/6881
USPC 506/9; 435/6.11

(57) ABSTRACT

Gene expression profiles associated with improved or maintained lean body mass or reduced body fat are disclosed. The gene expression profiles were determined in adipose, liver, and muscle tissue of animals subjected to lean-promoting regimens such as consumption of a high protein diet, ingestion of conjugated linolenic acid, and/or increased exercise. Methods of using such profiles for the identification of pharmaceutical substances, nutraceutical substances, dietary substances, or treatment regimens that modulate or contribute to desired phenotypes in animals are also disclosed

### CROSS REFERENCE TO RELATED APPLICATIONS

This application is a continuation of U.S. patent application Ser. No. 12/737,745 filed on Mar. 18, 2011, which is the National Stage of International Application No. PCT/US2009/04582 filed on Aug. 10, 2009, which claims the benefit of U.S. Provisional Application Ser. No. 61/190,369 filed Aug. 28, 2008, the disclosure of which is incorporated herein by reference.

### BACKGROUND OF THE INVENTION

1. Field of the Invention

The invention relates generally to nutritional or pharmaceutical modulation of body composition and particularly to gene expression profiles associated with improved or maintained lean body mass or reduced body fat and the use of such profiles for the identification of pharmaceutical, nutraceutical, or dietary substances that modulate or contribute to desired phenotypes in animals.

2. Description of the Related Art

Body weight is primarily a function of lean body mass and fat mass in an individual. Lean body mass is the weight of bones, muscles, organs, body water, and all other non-fat constituents of the body. Fat mass is the weight of the body's storage lipids. Disproportionate or excessive fat mass is a hallmark for an individual being overweight or obese.

Excess fat mass and obesity are recognized as a worldwide health problem among humans. The World Health Organization estimates that there are over 1 billion overweight adults, with just under one third of them classified as obese. In addition, obesity is also increasingly recognized as a problem for animals, particularly for companion animals such as dogs and cats. According to the Centers for Disease Control (CDC), obesity is closely associated with at least a risk of other health problems, including hypertension, dyslipidemia, Type II diabetes, heart disease, stroke, sleep apnea, and certain cancers such as breast, endometrial, and colon cancers. Risk factors for an individual becoming obese include genetics, emotions/stress, overeating, and a sedentary lifestyle.

Enhancing lean body mass can enhance the body's basal metabolic rate. Enhancing metabolic rate can facilitate the loss of excess fat mass when dietary caloric intake is insufficient to meet the body's energy needs or can reduce the accumulation of fat mass when dietary caloric intake exceeds the body's maintenance energy requirement. Various approaches to increase lean body mass have been described. One such approach is dietary supplementation with conjugated linoleic acid (CLA). CLA is a term used to describe isomers of octadecadienoic acid that are found in many foods such as dairy products (Terpstra A H M (2004) Am. J. Clin. Nutr. 79:352-61). CLA has been shown to reduce fat mass in mice and humans and has been implicated in an increase in lean body mass (Bhattacharya A et al. (2005) J. Nutr. 135:1124-30; Gaullier J-M et al. (2004) Am. J. Clin. Nutr. 79:1118-25; Blankson H et al. (2000) J. Nutr. 130:2943-8; and, Park Y et al. (1997) Lipids 32:853-8). Another approach to increase lean body mass is consumption of a high protein diet. Studies suggest that diets with higher protein content, coupled with reduced carbohydrate consumption and/or regular exercise, can enhance the loss of fat mass and reduce the loss of lean body mass (Layman D K et al. (2005) J. Nutr. 135:1903-10; Layman D K et al. (2004) J. Nutr. 134:968 S-73S; Marsset-Baglieri A et al. (2004) J. Nutr. 134:2646-52; and, Due A et al. (2004) Int. J. Obes. Relat. Metab. Disord. 28:1283-90). A third approach to increase lean body mass and decrease fat mass is regular exercise (Bhattacharya A et al. (2005) J. Nutr. 135:1124-30; Layman D K et al. (2005) J. Nutr. 135:1903-10; and, Tsai A C et al. (2003) J. Nutr. Biochem. 14:541-9).

Various studies have evaluated different aspects of the genetics of lean body mass and obesity. Association studies have revealed links between bodyweight, overweight, and obesity and polymorphisms in various genes (Chagnon Y C et al. (2003) Obesity Res. 11:313-67). Similarly, association studies have identified genes relating to body fat mass, percentage of body fat, and skin folds, body fat distribution (waist-to-hip ratio, waist circumference, etc.), resting energy expenditure, and adipocyte lipolysis (Chagnon Y C et al. (2003) Obesity Res. 11:313-67). In addition, studies have evaluated associations between candidate genes and changes in body weight, including genes associated with spontaneous weight gain over time, genes associated with endurance training-induced changes in fat mass, and genes associated with weight loss during a three year lifestyle change (Chagnon Y C et al. (2003) Obesity Res. 11:313-67). A listing of all such genes can be found in the prior art (Chagnon Y C et al. (2003) Obesity Res. 11:313-67). Studies have also analyzed the genetic aspects of lean body mass. Lean body mass studies have linked polymorphisms in Type 1 Deiodinase with higher lean body mass and muscle strength (Peeters R P et al. (2005) J. Clin. Endocrinol. 90:256-63), and polymorphisms in the glucocorticoid receptor with higher muscle mass and muscle strength (van Rossum E F C et al. (2004) J. Clin. Endocrinol. 89:4004-9).

Although lean body mass and fat mass are directly related, few studies have attempted to explore the genetic mechanisms that mediate a higher proportion of one type relative to the other. A detailed knowledge of these mechanisms would provide a better understanding of the conditions that favor a high level of lean body mass and/or reduced body fat and would provide a better understanding of how to promote a lean phenotype in an animal. Because a higher proportion of lean body mass, especially relative to fat mass, has positive implications for improved health and decreased risk for obesity-related ailments, it is desirable for an individual to increase the ratio of lean body mass to fat mass, either by increasing lean body mass and/or by reducing body fat.

### SUMMARY OF THE INVENTION

It is, therefore, an object of the invention to provide one or more genes or gene segments that are differentially expressed in animals exhibiting a lean phenotype resulting from one or more lean phenotype-promoting treatments comprising (1) administration of CLA, (2) consumption of a high protein diet, and (3) increased exercise.

It is another object of the invention to provide a combination comprising a plurality of polynucleotides that are differentially expressed in animals exhibiting a lean phenotype resulting from one or more lean phenotype-promoting treatments comprising (1) administration of CLA, (2) consumption of a high protein diet, and (3) increased exercise.

It is another object of the invention to provide compositions of two or more polynucleotide or polypeptide probes suitable for detecting the expression of genes differentially expressed in animals exhibiting a lean phenotype resulting from one or more lean phenotype-promoting treatments comprising (1) administration of CLA, (2) consumption of a high protein diet, and (3) increased exercise, and devices such as substrate arrays containing the probes.

It is a further object of the invention to provide methods for detecting differential expression of one or more genes differentially expressed in animals exhibiting a lean phenotype resulting from one or more lean phenotype-promoting treatments comprising (1) administration of CLA, (2) consumption of a high protein diet, and (3) increased exercise, as compared with normal or untreated animals.

It is another object of the invention to provide a method for measuring the effect of a test substance (e.g., lean body mass promoting nutrients or bioactives) on the expression profile of one or more genes differentially expressed in animals exhibiting a lean phenotype resulting from one or more lean phenotype-promoting treatments comprising (1) administration of CLA, (2) consumption of a high protein diet, and (3) increased exercise, as compared with normal or untreated animals.

One or more of these other objects are achieved using novel combinations of polynucleotides or polypeptides representing genes and gene segments that are differentially expressed in animals exhibiting a lean phenotype resulting from one or more lean phenotype-promoting treatments comprising (1) administration of CLA, (2) consumption of a high protein diet, and (3) increased exercise. The polynucleotides are used to produce compositions, probes, devices based on the probes, and methods for determining the status of polynucleotides differentially expressed in animals exhibiting a lean phenotype as compared to normal or untreated animals, which are useful for achieving the above-identified objects, e.g., prognosing and diagnosing conditions relating to the phenotype and for screening substances to determine if they are likely to be useful for promoting the phenotype. Such substances, once identified, may be used to promote the phenotype. Various kits comprising combinations of probes, devices utilizing the probes, and substances are also provided, as are various computer programs for manipulating information, and communication media for communicating information pertaining to the differentially expressed genes and methods of their use.

Other and further objects, features, and advantages of the invention will be readily apparent to those skilled in the art.

### DETAILED DESCRIPTION OF THE INVENTION

### Definitions

All percentages expressed herein are by weight of the composition on a dry matter basis unless specifically stated otherwise. The skilled artisan will appreciate that the term “dry matter basis” means that an ingredient's concentration in a composition is measured after any free moisture in the composition is removed.

As used throughout, ranges are used herein as shorthand, so as to avoid having to set out at length and describe each and every value within the range. Any appropriate value within the range can be selected, where appropriate, as the upper value, lower value, or the terminus of the range. It is understood that any and all whole or partial integers between any ranges or intervals set forth herein are included herein.

As used herein and in the appended claims, the singular form of a word includes the plural, and vice versa, unless the context clearly dictates otherwise. Thus, the references “a,” “an,” and “the” are generally inclusive of the plurals of the respective terms. For example, reference to “an animal”, “a method”, or “a substance” includes a plurality of such “animals”, “methods”, or “substances”. Similarly, the words “comprise”, “comprises”, and “comprising” are to be interpreted inclusively rather than exclusively.

The term “animal” means a human or other animal, including avian, bovine, canine, equine, feline, hicrine, murine, ovine, and porcine animals, that has adipose tissue. When the term is used in the context of comparing test subjects, the animals that are compared are animals of the same species and possibly of the same race or breed. A “companion animal” is any domesticated animal, and includes, without limitation, cats, dogs, rabbits, guinea pigs, ferrets, hamsters, mice, gerbils, horses, cows, goats, sheep, donkeys, pigs, and the like. Preferably, the animal is a human or a companion animal such as a canine or feline.

The term “antibody” means any immunoglobulin that binds to a specific antigen, including IgG, IgM, IgA, IgD, and IgE antibodies. The term includes polyclonal, monoclonal, monovalent, humanized, heteroconjugate, antibody compositions with polyepitopic specificity, chimeric, bispecific antibodies, diabodies, single-chain antibodies, and antibody fragments such as Fab, Fab′, F(ab′)2, and Fv, or other antigen-binding fragments.

The term “array” means an ordered arrangement of at least two probes on a substrate. At least one of the probes is a control or standard and at least one of the probes is a diagnostic probe. The arrangement of from about two to about 40,000 probes on a substrate assures that the size and signal intensity of each labeled complex formed between a probe and a sample polynucleotide or polypeptide is individually distinguishable.

The term “binding complex” refers to a complex formed when a polypeptide in a sample specifically binds (as defined herein) to a binding partner, such as an antibody or functional fragment thereof.

The term “dietary supplement” means a product that is intended to be ingested in addition to the normal diet of an animal. Dietary supplements may be in any form—e.g., solid, lid, gel, tablets, capsules, powder, and the like. Preferably they are provided in convenient dosage forms. In some embodiments they are provided in bulk consumer packages such as bulk powders or liquids. In other embodiments, supplements are provided in bulk quantities to be included in other food items such as snacks, treats, supplement bars, beverages and the like.

The term “differential expression” or “differentially expressed” means increased or unregulated gene expression or means decreased or downregulated gene expression as detected by the absence, presence, or at least statistically significant in the amount of transcribed messenger RNA or translated protein in a sample.

The term “food” or “food composition” means a composition that is intended for consumption by an animal, including a human, and provides nutrition thereto. A “food product formulated for human consumption” is any composition specifically intended for ingestion by a human being. “Pet foods” are compositions intended for consumption by pets, preferably by companion animals. A “complete and nutritionally balanced pet food,” is one that contains all known required nutrients for the intended recipient or consumer of the food, in appropriate amounts and proportions, based for example on recommendations of recognized authorities in the field of companion animal nutrition. Such foods are therefore capable of serving as a sole source of dietary intake to maintain life or promote production, without the addition of supplemental nutritional sources. Nutritionally balanced pet food compositions are widely known and widely used in the art.

The term “fragment” means (1) an oligonucleotide or polynucleotide sequence that is a portion of a complete sequence and that has the same or similar activity for a particular use as the complete polynucleotide sequence or (2) a peptide or polypeptide sequence that is a portion of a complete sequence and that has the same or similar activity for a particular use as the complete polypeptide sequence. Such fragments can comprise any number of nucleotides or amino acids deemed suitable for a particular use. Generally, oligonucleotide or polynucleotide fragments contain at least about 10, 50, 100, or 1000 nucleotides and polypeptide fragments contain at least about 4, 10, 20, or 50 consecutive amino acids from the complete sequence. The term encompasses polynucleotides and polypeptides variants of the fragments.

The term “gene” or “genes” means a complete or partial segment of DNA involved in producing a polypeptide, including regions preceding and following the coding region (leader and trailer) and intervening sequences (introns) between individual coding segments (exons). The term encompasses any DNA sequence that hybridizes to the complement of gene coding sequences.

The term “gene product” means the product of transcription of a gene, such as mRNA or derivatives thereof (e.g., cDNA), or translation of a gene transcript. The term “gene product” generally refers to the translation product, which is a protein. The term “gene product” may be used interchangeably with the term “protein” herein.

The term “high protein diet” refers to a diet comprising foods or dietary supplements that result in an animal's intake of protein on a regular basis being at least about 10% higher than a comparable control animal. In certain embodiments, the animal's protein intake may be 20, 30, 40, 50, 60, 70, 80, 90 or 100% (i.e., two-fold in the latter case) higher than that of a comparable control animal. In other embodiments, the animal's protein intake may be three- or four-fold or more higher than that of a comparable control animal. Typically, a high protein diet is formulated to comprise the same calorie intake as a regular diet. Often, but not always, this is accomplished by lowering the carbohydrate content of the diet. Alternatively or in addition, the fat content of the diet may be lowered. In particular embodiments, the protein content of a high protein diet may comprise at least about 25% of the total calories as protein. In other embodiments, the protein content of a high protein diet may comprise at least about 30%, 35%, 40%, 45%, 50%, 55%, 60%, 65%, 70%, 75% or 80% of the total calories as protein.

The term “homolog” means (1) a polynucleotide, including polynucleotides from the same or different animal species, having greater than 30%, 50%, 70%, or 90% sequence similarity to a reference polynucleotide, and having the same or substantially the same properties and performing the same or substantially the same function as the reference polynucleotide, or having the capability of specifically hybridizing to a reference polynucleotide under stringent conditions or (2) a polypeptide, including polypeptides from the same or different animal species, having greater than 30%, 50%, 70%, or 90% sequence similarity to a reference polypeptide and having the same or substantially the same properties and performing the same or substantially the same function as the reference polypeptide, or having the capability of specifically binding to a reference polypeptide. When referring to fragments of full length coding sequences, the function of those fragments may simply be to encode a selected portion of a polypeptide of a certain sequence, or to be of suitably similar sequence to hybridize to another polynucleotide fragment encoding that polypeptide. When referring to fragments of polypeptides, the function of those fragments may simply be to form an epitope suitable for generation of an antibody. Sequence similarity of two polypeptide sequences or of two polynucleotide sequences is determined using methods known to skilled artisans, e.g., the algorithm of Karlin and Altschul (Proc. Natl. Acad. Sci. USA 87:2264-2268 (1990)). Such an algorithm is incorporated into the NBLAST and XBLAST programs of Altschul et al. (J. Mol. Biol. 215:403-410 (1990)). To obtain gapped alignments for comparison purposes, Gapped Blast can be utilized as described in Altschul et al. (Nucl. Acids Res. 25: 3389-3402 (1997)). When utilizing BLAST and Gapped BLAST programs, the default parameters of the respective programs (e.g., XBLAST and NBLAST) are used. See http://ww.ncbi.nlm.nih.gov.

The term “hybridization complex” means a complex that is formed between sample polynucleotides when the purines of one polynucleotide hydrogen bond with the pyrimidines of the complementary polynucleotide, e.g., 5′-A-G-T-C-3′ base pairs with 3′-T-C-A-G-5′. The degree of complementarily and the use of nucleotide analogs affect the efficiency and stringency of hybridization reactions.

The term “increased exercise” refers to an increase in physical activity of least about 10% higher than that of a comparable control animal in the same period of time. In certain embodiments, the animal's physical activity may be 20, 30, 40, 50, 60, 70, 80, 90 or 100% (i.e., two-fold in the latter case) higher than that of a comparable control animal. In other embodiments, the animal's activity may be three- or four-fold or more higher than that of a comparable control animal. An animal's physical activity may be measured by a variety of techniques well known to the person of skill in the art.

The term “individual” when referring to an animal means an individual animal of any species or kind.

The term “lean phenotype” refers to any molecular, biochemical, physiologic, cellular, systemic, and physical effects observed in an animal resulting from the differential expression of genes that occurs when the animal exercises, consumes a specialized diet regimen such as a high protein diet, and/or is administered a compound, composition, or dietary supplement to modulate the expression of genes associated with increasing or maintaining lean body mass, and/or reducing body fat. An exemplary compound of this type is CLA. The term “lean phenotype” also includes the “transition to lean phenotype,” which refers to any molecular, biochemical, physiologic, cellular, systemic, and physical effects observed in an animal resulting from the differential expression of genes that occurs when the animal is undergoing the change from normal (as defined below) to lean phenotype.

The term “administration” means to administer a substance, a diet, or a test treatment (such as increased physical exercise) to an animal. Administration periods include terms consistent with the particular substance, diet, or treatment and the animal. “Long term administration” generally refers to periods in excess of one week. Periods of longer than two or three weeks, or one, two, three, or four months are contemplated. Also included are more extended periods that include longer than 5, 6, 7, 8, 9, or 10 months. Periods in excess of 11 months or 1 year are also included. Long term use extending over 1, 2, 3 years or more is also contemplated herein. In the case of certain animals, it is envisioned that the animal would be administered substances or treatment regimens identified by the present methods on a regular basis. “Regular basis” refers to at least monthly administration. More frequent administration, such as weekly or two or three times weekly is included. Also included are regimens that comprise at least once, twice, three times or more daily administration. Any dosing frequency, regardless of whether expressly exemplified herein, is considered useful. The skilled artisan will appreciate that dosing frequency will be a function of the substance that is being administered, and some compositions may require more or less frequent administration to maintain a desired biochemical, physiological or gene expression effects, namely effects including one or more of food intake, satiety, lipid metabolism, and fat utilization, and the gene expression profile associated therewith. The term “extended regular basis” refers to long term administration of a substance on a regular basis.

“Normal” as used in relation to animals manifesting a lean phenotype, refers to the absence of molecular, biochemical, physiologic, cellular, systemic, and physical effects resulting from the differential expression of genes associated with a lean phenotype.

The term “oral administration” means that an animal ingests, or a human is directed to feed, or does feed, the animal one or more of the substances described herein. The term “ingestion” is used herein interchangeably with the term “oral administration.” The term “consumption” is also used herein to refer to ingestion of a substance, particularly a food composition, on an extended regular basis. When a human is directed to orally administer or feed the substance, such direction may be that which instructs and/or informs the human that use of the substance may and/or will provide the referenced benefit. Such direction may be oral direction (e.g., through oral instruction from, for example, a physician, veterinarian, or other health professional, or radio or television media (i.e., advertisement), or written direction (e.g., through written direction from, for example, a physician, veterinarian, or other health professional (e.g., prescriptions), sales professional or organization (e.g., through, for example, marketing brochures, pamphlets, or other instructive paraphernalia), written media (e.g., internet, electronic mail, or other computer-related media), and/or packaging associated with the substance.

The term “polynucleotide” or “oligonucleotide” means a polymer of nucleotides. The term encompasses DNA and RNA (including cDNA and mRNA) molecules, either single or double stranded and, if single stranded, its complementary sequence in either linear or circular form. The term also encompasses fragments, variants, homologs, and alleles, as appropriate for the sequences, which have the same or substantially the same properties and perform the same or substantially the same function as the original sequence. In particular, the term encompasses homologs from different species, e.g., a mouse and a dog or cat. The sequences may be fully complementary (no mismatches) when aligned or may have up to about a 30% sequence mismatch. Preferably, for polynucleotides, the chain contains from about 50 to 10,000 nucleotides, more preferably from about 150 to 3,500 nucleotides. Preferably, for oligonucleotides, the chain contains from about 2 to 100 nucleotides, more preferably from about 6 to 30 nucleotides. The exact size of a polynucleotide or oligonucleotide will depend on various factors and on the particular application and use of the polynucleotide or oligonucleotide. The term includes nucleotide polymers that are synthesized and that are isolated and purified from natural sources. The term “polynucleotide” is inclusive of “oligonucleotide.”

The term “polypeptide,” “peptide,” or “protein” means a polymer of amino acids. The term encompasses naturally occurring and non-naturally occurring (synthetic) polymers and polymers in which artificial chemical mimetics are substituted for one or more amino acids. The term also encompasses fragments, variants, and homologs that have the same or substantially the same properties and perform the same or substantially the same function as the original sequence. The term encompass polymers of any length, preferably polymers containing from about 2 to 1000 amino acids, more preferably from about 5 to 500 amino acids. The term includes amino acid polymers that are synthesized and that are isolated and purified from natural sources.

The term “probe” means (1) an oligonucleotide or polynucleotide, either RNA or DNA, whether occurring naturally as in a purified restriction enzyme digest or produced synthetically, that is capable of annealing with or specifically hybridizing to a polynucleotide with sequences complementary to the probe or (2) a compound or substance, including a peptide or polypeptide, capable of specifically binding a particular protein or protein fragment to the substantial exclusion of other proteins or protein fragments. An oligonucleotide or polynucleotide probe may be either single or double stranded. The exact length of the probe will depend upon many factors, including temperature, source, and use. For example, for diagnostic applications, depending on the complexity of the target sequence, an oligonucleotide probe typically contains about 10 to 100, 15 to 50, or 15 to 25 nucleotides. In certain diagnostic applications, a polynucleotide probe contains about 100-1000, 300-600, nucleotides, preferably about 300 nucleotides. The probes herein are selected to be “substantially” complementary to different strands of a particular target sequence. This means that the probes must be sufficiently complementary to specifically hybridize or anneal with their respective target sequences under a set of predetermined conditions. Therefore, the probe sequence need not reflect the exact complementary sequence of the target. For example, a noncomplementary nucleotide fragment may be attached to the 5′ or 3′ end of the probe, with the remainder of the probe sequence being complementary to the target sequence. Alternatively, noncomplementary bases or longer sequences can be interspersed into the probe provided that the probe sequence has sufficient complementarity with the sequence of the target polynucleotide to specifically anneal to the target polynucleotide. A peptide or polypeptide probe may be any molecule to which the protein or peptide specifically binds, including DNA (for DNA binding proteins), antibodies, cell membrane receptors, peptides, cofactors, lectins, sugars, polysaccharides, cells, cell membranes, organelles and organellar membranes.

The term “sample” means any animal tissue or fluid containing, e.g., polynucleotides, polypeptides, antibodies, metabolites, and the like, including cells and other tissue containing DNA and RNA. Examples include adipose, blood, cartilage, connective, epithelial, lymphoid, muscle, nervous, sputum, and the like. A sample may be solid or liquid and may be DNA, RNA, cDNA, bodily fluids such as blood or urine, cells, cell preparations or soluble fractions or media aliquots thereof, chromosomes, organdies, and the like.

The term “single package” means that the components of a kit are physically associated in or with one or more containers and considered a unit for manufacture, distribution, sale, or use. Containers include, but are not limited to, bags, boxes, bottles, shrink wrap packages, stapled or otherwise affixed components, or combinations thereof. A single package may be containers of individual food compositions physically associated such that they are considered a unit for manufacture, distribution, sale, or use.

The term “specifically bind” means a special and precise interaction between two molecules which is dependent upon their structure, particularly their molecular side groups. For example, the intercalation of a regulatory protein into the major groove of a DNA molecule, the hydrogen bonding along the backbone between two single stranded nucleic acids, or the binding between an epitope of a protein and an agonist, antagonist, or antibody.

The term “specifically hybridize” means an association between two single stranded polynucleotides of sufficiently complementary sequence to permit such hybridization under predetermined conditions generally used in the art (sometimes termed “substantially complementary”). For example, the term may refer to hybridization of a polynucleotide probe with a substantially complementary sequence contained within a single stranded DNA or RNA molecule according to an aspect of the invention, to the substantial exclusion of hybridization of the polynucleotide probe with single stranded polynucleotides of non-complementary sequence.

The term “standard” means (1) a control sample that contains tissue from an animal administered a control or reference substance, or no substance, as compared with a sample that contains tissue from an animal administered a test substance, for example, to determine if the test substance causes differential gene expression, as appropriate for the context of its use.

The term “stringent conditions” means (1) hybridization in 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C., (2) hybridization in 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C.; with washes at 42° C. in 0.2×SSC and 0.1% SDS or washes with 0.015 M NaCl, 0.0015 M sodium citrate, 0.1% Na2SO4 at 50° C. or similar procedures employing similar low ionic strength and high temperature washing agents and similar denaturing agents.

The term “variant” means (1) a polynucleotide sequence containing any substitution, variation, modification, replacement, deletion, or addition of one or more nucleotides from or to a polynucleotide sequence and that has the same or substantially the same properties and performs the same or substantially the same function as the original sequence and (2) a polypeptide sequence containing any substitution, variation, modification, replacement, deletion, or addition of one or more amino acids from or to a polypeptide sequence and that has the same or substantially the same properties and performs the same or substantially the same function as the original sequence. The term therefore includes single nucleotide polymorphisms (SNPs) and allelic variants and includes conservative and non-conservative amino acid substitutions in polypeptides. The term also encompasses chemical derivatization of a polynucleotide or polypeptide and substitution of nucleotides or amino acids with nucleotides or amino acids that do not occur naturally, as appropriate.

The term “virtual package” means that the components of a kit are associated by directions on one or more physical or virtual kit components instructing the user how to obtain the other components, e.g., in a bag containing one component and directions instructing the user to go to a website, contact a recorded message, view a visual message, or contact a caregiver or instructor to obtain instructions on how to use the kit.

The combinations, compositions, devices, methods, probes, and other advances disclosed herein are not limited to particular methodology, protocols, and reagents described herein because, as the skilled artisan will appreciate, they may vary. Further, the terminology used herein is for the purpose of describing particular embodiments only, and is not intended to and does not limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the invention, the preferred compositions, methods, articles of manufacture, or other means or materials are described herein.

All patents, patent applications, publications, and other references cited or referred to herein are incorporated herein by reference to the extent allowed by controlling law. The discussion of those references is intended merely to summarize the assertions made therein. No admission is made that any such patents, patent applications, publications or references, or any portion thereof, is relevant, material, or prior art. The right to challenge the accuracy and pertinence of any assertion of such patents, patent applications, publications, and other references as relevant, material, or prior art is specifically reserved.

### The Invention

The invention is based in part on the clear demonstration that treatments known to promote a lean phenotype as defined herein are associated with significant changes in the gene expression profiles in three different tissues of animals subjected to those treatments. The association was determined by comparing expression of the genes in normal tissue, namely muscle, liver and adipose tissue, with tissue from animals manifesting a lean phenotype (LP) as a result of one or more LP-promoting treatments, namely (1) administration of CLA, (2) consumption of a high protein diet, and/or (3) increased exercise.

In one embodiment, several hundred genes were found to be differentially expressed as a result of all three treatments (sometimes referred to herein as “three treatments”); i.e., CLA supplementation, a high protein diet, or increased exercise caused the differential expression of this subset of genes, the encoded proteins of which are listed in Table 6 (Example 3) herein. The proteins set forth in Table 6 are divided into groups based upon different criteria. First, the proteins are listed by tissue in Table 6, representing genes differentially expressed in adipose tissue (Table 6A), liver (Table 6B) and muscle (quadriceps) (Table 6C). Table 6 also lists subsets of proteins representing genes differentially expressed in two or more of the three tissue types, namely (1) adipose and liver (Table 6D), (2) adipose and muscle (Table 6E), (3) liver and muscle (Table 6F), and (4) all three tissues (Table 60). Second, the proteins are divided into groups based upon the function or physiological role of the encoded protein, as well as the tissue in which the differential expression occurs. Those groupings are set forth in Table 7 (adipose), Table 8 (liver) and Table 9 (muscle). Those functions include: (1) in adipose tissue, cholesterol biosynthetic pathway, statin pathway, adipogenesis, apoptosis, cell motility, mitochondrial fatty acid betaoxidation, fatty acid biosynthesis, fatty acid metabolism, glycolysis, regulation of cell proliferation, inflammation, immunity and stress response (including the subcategories of Rn T-cell receptor, Rn B-cell receptor, leukocyte transendothelial migration, tight junction, adherens junction, antigen processing, response to unfolded proteins, response to wounding, response to external stimulus, inflammatory response, immune response, T-cell activation and Rn IL-4), multicellular organismal development, and regulation of apoptosis; (2) in liver, PPAR signaling pathway, and fatty acid metabolism; and (3) in muscle, lipid metabolism.

In another embodiment, the high protein diet treatment, analyzed singly, was found to cause differential expression of several thousand genes. The proteins encoded by these genes are listed in Table 10 (Example 4) herein. As with the “three treatments” subset, the proteins set forth in Table 10 are divided into groups based upon different criteria. First, the proteins are listed by tissue in Table 10, representing genes differentially expressed in adipose tissue (Table 10A), liver (Table 10B) and muscle (quadriceps) (Table 10C). Table 10 also lists subsets of proteins representing genes differentially expressed in two or more of the three tissue types, namely (1) adipose and liver (Table 10D), (2) adipose and muscle (Table 10E), (3) liver and muscle (Table 10F), and (4) all three tissues (Table 100). Second, the proteins are divided into groups based upon the function or physiological role of the encoded protein, as well as the tissue in which the differential expression occurs. Those groupings are set forth in Table 11 (adipose), Table 12 (liver) and Table 13 (muscle). Those functions include: (1) in adipose tissue, immune response, inflammatory response, response to stress, chemotaxis, response to unfolded protein, defense response, cell activation, lymphocyte activation, locomotory behavior, lipid metabolic process, lipid biosynthetic process, steroid biosynthetic process, cholesterol metabolic process, steroid metabolic process, glycolysis, glucose metabolic process, organ development, muscle development, positive regulation of cell proliferation, angiogenesis, blood vessel morphogenesis, anti-apoptosis, muscle contraction, phosphate transport, protein complex assembly, calcium-mediated signaling, regulation of GTPase activity, protein amino acid glycosylation, regulation of cell shape, Rattus norvegicus (Rn) B cell receptor NetPath 12 (Johns Hopkins University and the Institute of Bioinformatics (www.netpath.org/)), Rn T-cell receptor NetPath 11, Rn IL-4 NetPath 16, Rn IL-7 NetPath 19, Rn eicosanoid synthesis, Rn insulin signaling, Rn cholesterol biosynthesis, Rn fatty acid synthesis BiGCaT (University of Maastricht (www.bigcat.unimass.n1/index.html)), Rn Krebs-TCA cycle, Rn mitochondrial fatty acid betaoxidation, Rn striated muscle contraction, leukocyte transendothelial migration, T cell receptor signaling pathway, tight junction, B cell receptor signaling pathway, complement and coagulation cascades, Fc epsilon RI signaling pathway, toll-like receptor signaling pathway, PPAR signaling pathway, biosynthesis of steroids, glycolysis/gluconeogenesis, arachidonic acid metabolism, pyruvate metabolism, and riboflavin metabolism; (2) in liver, lipid metabolic process, fatty acid metabolic process, lipid biosynthetic process, fatty acid biosynthetic process, steroid metabolic process, proteolysis, carbohydrate metabolic process, glucose metabolic process, gluconeogenesis, amino acid metabolic process, amine metabolic process, nitrogen compound metabolic process, one-carbon compound metabolic process, xenobiotic metabolic process, sodium ion transport, multicellular organismal development, regulation of cell growth, myelination, regulation of progression through cell cycle, antigen processing and presentation, Rn adipogenesis, Rn fatty acid synthesis BiGCaT, Rn glycolysis and gluconeogenesis, Rn nuclear receptors in lipid metabolism and toxicity, Rn fatty acid beta oxidation 1 BiGCaT, Rn fatty acid omega oxidation BiGCaT, PPAR signaling pathway, metabolism of xenobiotics by cytochrome P450, fatty acid metabolism, alanine and aspartate metabolism, arginine and proline metabolism, pyruvate metabolism, glutamate metabolism, nitrogen metabolism, cysteine metabolism, and tyrosine metabolism; and (3) in muscle, immune response, defense response, inflammatory response, and Rn circadian exercise.

Thus, a number of genes have been identified that are differentially expressed in animals manifesting a lean phenotype, resulting from LP-promoting treatments including a high protein diet, administration of CLA and/or increased exercise. Polynucleotides and fragments thereof that form these genes, as well as their encoded proteins and fragments, can be used, for example, in diagnostic or prognostic assays to measure a shift to a lean phenotype, or assays useful for screening test substances for their effectiveness to promote or support a lean phenotype.

In certain embodiments, expression of at least one differentially expressed gene is measured. In preferred embodiments, expression of two or more differentially expressed genes is measured, providing a gene expression pattern or gene expression profile. More preferably, measurement of a multiplicity of differentially expressed genes is performed, providing additional information for a gene expression pattern or profile.

In various embodiments of the invention, changes in gene expression may be measured in one or both of two ways: (1) measuring transcription through detection of mRNA produced by a particular gene; and (2) measuring translation through detection of protein produced by a particular transcript.

Decreased or increased expression can be measured at the RNA level using any of the methods well known in the art for the quantitation of polynucleotides, such as, for example, PCR (including, without limitation, RT-PCR and qPCR), RNase protection, Northern blotting, microarray, macroarray, and other hybridization methods. The genes that are assayed or interrogated according to the invention are typically in the form of mRNA or reverse transcribed mRNA. The genes may be cloned and/or amplified. The cloning itself does not appear to bias the representation of genes within a population. However, it may be preferable to use polyA+ RNA as a source, as it can be used with fewer processing steps.

Thus, in one aspect, the invention provides a combination comprising a plurality of polynucleotides or proteins expressed therefrom that are differentially expressed in animals exhibiting a lean phenotype resulting from one or more lean phenotype-promoting treatments comprising (1) administration of CLA, (2) consumption of a high protein diet, and (3) increased exercise, wherein the polynucleotides are selected from genes encoding proteins listed in Table 6 or Table 10, or fragments thereof.

In one embodiment, the polynucleotides are differentially expressed in each of the lean phenotype promoting treatments comprising (1) administration of CLA, (2) consumption of a high protein diet, and (3) increased exercise, and the polynucleotides are selected from genes encoding proteins listed in Table 6, or fragments thereof. In more specific embodiments, the polynucleotides are selected from genes encoding proteins listed in tissue-specific subsets of Table 6 selected from Table 6A, Table 6B, Table 6C, Table 6D, Table 6E, Table 6F, and Table 6G, or fragments thereof. In other embodiments, the polynucleotides are differentially expressed in adipose tissue and encode proteins involved in functions selected from those recited hereinabove and set forth in Table 7. In other embodiments, the polynucleotides are differentially expressed in liver and encode proteins involved in the functions recited hereinabove and set forth in Table 8. In yet another embodiment, the polynucleotides are differentially expressed in muscle and encode proteins involved in lipid metabolism, as set forth in Table 9.

In another embodiment, the polynucleotides are differentially expressed in the lean phenotype promoting treatment comprising consumption of a high protein diet, and the polynucleotides are selected from genes encoding proteins listed in Table 10, or fragments thereof. In more specific embodiments, the polynucleotides are selected from genes encoding proteins listed in tissue-specific subsets of Table 10 selected from: Table 10A, Table 10B, Table 10C, Table 10D, Table 10E, Table 10F, and Table 100, or fragments thereof. In other embodiments, the polynucleotides are differentially expressed in adipose tissue and encode proteins involved in functions selected from those recited above, and listed in Table 11. In other embodiments, the polynucleotides are differentially expressed in liver and encode proteins involved in functions selected from those recited above and set forth in Table 12. In yet another embodiment, the polynucleotides are differentially expressed in muscle and encode proteins involved in functions selected from those recited above and listed in Table 13.

In one embodiment, the combination comprises two or more polynucleotides or proteins expressed from the polynucleotides. Preferably, the combination comprises a plurality of polynucleotides or proteins expressed from polynucleotides, generally about 5, 10, 15, 20, 25, 30, 35, 40, 45, 50, 60, 70, 80, 90, 100 or more polynucleotides or proteins, or fragments thereof, as appropriate for a particular Group and use. When the combination comprises one or more fragments, the fragments can be of any size that retains the properties and function of the original polynucleotide or protein, preferably from about 30%, 60%, or 90% of the original.

The polynucleotides and proteins can be from any animal, preferably canines and felines, most preferable canines. Homologs of the polynucleotides and proteins from different animal species are obtainable by standard information mining and molecular methods well known to the skilled artisan. For example, the name, or description of function of a gene or protein may be entered into one of several publicly available databases, which will generate a list of sources providing information about that gene from different species, including sequence information. One such database is the “Information Hyperlinked over Proteins (iHOP) database, which is accessible on the internet via the url: ihop-net.org. Alternatively, a public database accession number of a known gene or protein may be utilized to access sequence information for that gene or protein and to search for homologs or orthologs in other species using a sequence comparison search. For example, the GenBank accession number of a gene or protein from mouse may be entered into the National Institutes of Health's National Center for Biotechnology Information (NCBI) database, thereby accessing DNA or polypeptide sequences for that mouse gene. Using the same database, a BLAST search may be performed on the mouse DNA or protein sequence, or fragments thereof of sufficient length to define the gene or protein, to identify sequences of sufficient homology from other species, e.g., a canine. Accession numbers of the sequences from the other species of interest may then be entered into the database to obtain information pertaining to those full-length nucleotide or protein sequences, as well as other descriptive information.

In another aspect, the invention provides a composition comprising two or more probes for detecting differential gene expression in animals exhibiting a lean phenotype resulting from one or more lean phenotype-promoting treatments comprising (1) administration of CLA, (2) consumption of a high protein diet, and (3) increased exercise, wherein the probes comprise (a) polynucleotides that specifically hybridize to two or more genes encoding proteins listed in Table 6 or Table 10, or fragments thereof, or (b) polypeptide binding agents that specifically bind to two or more polypeptides selected from proteins listed in Table 6 (genes differentially expressed in all three treatments) or Table 10 (genes differentially expressed in high protein diet treatment), or fragments thereof.

In certain embodiments, the probes specifically hybridize to genes encoding proteins listed in listed in tissue-specific subsets of Table 6 selected from Table 6A, Table 6B, Table 6C, Table 6D, Table 6E, Table 6F, and Table 6G, or fragments thereof, or specifically bind to polypeptides comprising proteins listed in listed in tissue-specific subsets of Table 6 selected from Table 6A, Table 6B, Table 6C, Table 6D, Table 6E, Table 6F, and Table 6G, or fragments thereof. In other embodiments, the probes specifically hybridize to, or specifically bind to, polynucleotides encoding proteins, or polypeptides comprising proteins having certain functions or biochemical roles, as listed in Table 7, Table 8 or Table 9.

In other embodiments, the probes specifically hybridize to genes encoding proteins listed in tissue-specific subsets of Table 10 selected from Table 10A, Table 10B, Table 10C, Table 10D, Table 10E, Table 10F, and Table 100, or fragments thereof, or specifically bind to polypeptides comprising proteins listed in listed in tissue-specific subsets of Table 10 selected from Table 10A, Table 10B, Table 10C, Table 10D, Table 10E, Table 10F, and Table 10G, or fragments thereof. In other embodiments, the probes specifically hybridize to, or specifically bind to, polynucleotides encoding proteins, or polypeptides comprising proteins having certain functions or biochemical roles, as listed in Table 11, Table 12 or Table 13.

Preferably, the composition comprises a plurality of probes, generally about 5, 10, 15, 20, 25, 30, 35, 40, 45, 50, 60, 70, 80, 90, 100, 200, 500 or more probes for detecting the polynucleotides or proteins, or fragments thereof, as appropriate for a particular Group and use. It will be understood by the skilled artisan that multiple different probes for a single target gene or protein may be utilized, in order to refine the sensitivity or accuracy of an assay utilizing the probes. For example, several oligonucleotide probes, specifically hybridizing to different sequences on a target polynucleotide, may be employed. Likewise, several antibodies, immunologically specific for different epitopes on a target protein, may be utilized.

One or more oligonucleotide or polynucleotide probes for interrogating a sample may be prepared using the sequence information for any of the genes listed herein, from any species, preferably canine or feline. The probes should be of sufficient length to specifically hybridize substantially exclusively with appropriate complementary genes or transcripts. In certain embodiments, the oligonucleotide probes will be at least about 10, 12, 14, 16, 18, 20 or 25 nucleotides in length. In some embodiments, longer probes of at least about 30, 40, 50, 60, 70, 80, 90 or 100 nucleotides are desirable, and probes longer than about 100 nucleotides may be suitable in some embodiments. The probes may comprise full length sequences encoding functional proteins. The nucleic acid probes are made or obtained using methods known to skilled artisans, e.g., in vitro synthesis from nucleotides, isolation and purification from natural sources, or enzymatic cleavage of the polynucleotides of the invention.

Hybridization complexes comprising nucleic acid probes hybridized to a polynucleotide of the invention may be detected by a variety of methods known in the art. In certain embodiments of the invention, immobilized nucleic acid probes may be used for the rapid and specific detection of polynucleotides and their expression patterns. Typically, a nucleic acid probe is linked to a solid support and a target polynucleotide (e.g., a gene, a transcription product, an amplicon, or, most commonly, an amplified mixture) is hybridized to the probe. Either the probe, or the target, or both, can be labeled, typically with a fluorophore or other tag, such as streptavidin. Where the target is labeled, hybridization may be detected by detecting bound fluorescence. Where the probe is labeled, hybridization is typically detected by quenching of the label. Where both the probe and the target are labeled, detection of hybridization is typically performed by monitoring a color shift resulting from proximity of the two bound labels. A variety of labeling strategies, labels, and the like, particularly for fluorescent based applications, are known in the art.

In another embodiment, the probes comprise polypeptide binding agents that specifically bind to polypeptides produced by expression of one or more of the polypeptides listed herein, or fragments thereof. Such protein binding probes may be prepared using the sequence information available for any of the proteins identified in Table 6 and Table 10, or fragments thereof.

Assay techniques that can be used to determine levels of a protein in a sample are also well known to those of skill in the art. Such assay methods include radioimmunoassays, competitive-binding assays, Western blot analysis and ELISA assays. In the assay methods utilizing antibodies, both polyclonal and monoclonal antibodies are suitable for use in the invention. Such antibodies may be immunologically specific for a particular protein, or an epitope of the protein, or a protein fragment, as would be well understood by those of skill in the art. Methods of making polyclonal and monoclonal antibodies immunologically specific for a protein or peptide are also well known in the art.

Preferred embodiments of the invention may utilize antibodies for the detection and quantification of proteins produced by expression of the genes described herein. Though proteins may be detected by immunoprecipitation, affinity separation, Western blot analysis and the like, a preferred method utilizes ELISA-type methodology wherein the antibody is immobilized on a solid support and a target protein or peptide is exposed to the immobilized antibody. Either the probe, or the target, or both, can be labeled. A variety of labeling strategies, labels, and the like, are known in the art.

In another aspect, the invention provides a device comprising a solid support to which is affixed an array comprising a plurality of probes for detecting differential gene expression in animals exhibiting a lean phenotype resulting from one or more lean phenotype-promoting treatments comprising (1) administration of CLA, (2) consumption of a high protein diet, and (3) increased exercise. In particularly preferred embodiments of the invention, expression patterns or profiles of a plurality of genes differentially expressed in lean versus normal phenotypes as defined herein, are observed utilizing arrays of probes for detecting target polynucleotides or proteins. The device may be used to detect differential expression of genes encoding the gene products set forth in Table 6 or table 10, or in subsets thereof, namely tissue-specific subsets as listed in Table 6A-G and Table 10A-G, or functional subsets as set forth in Tables 7, 8 and 9 (for three-treatment analysis) and Tables 11, 12 and 13 (for high protein diet analysis). In a preferred embodiment, the device is uses to detect differential expression of genes from canines or felines.

In one embodiment, arrays of oligonucleotide or polynucleotide probes may be utilized, whereas another embodiment may utilize arrays of antibodies or other proteins that bind specifically to the differentially expressed gene products. Such arrays may be custom made according to known methods, such as, for example, in-situ synthesis on a solid support or attachment of pre-synthesized probes to a solid support via micro-printing techniques. In preferred embodiments, arrays of nucleic acid or protein-binding probes are custom made to specifically detect transcripts or proteins produced by two or more of the differentially expressed genes or gene fragments described herein.

In a further aspect, the invention provides a method for detecting differential expression of one or more genes differentially expressed in animals exhibiting a lean phenotype resulting from one or more lean phenotype-promoting treatments comprising (1) administration of CLA, (2) consumption of a high protein diet, and (3) increased exercise, as compared with normal or untreated animals. The method generally comprises: (a) providing probes comprising (i) polynucleotides that specifically hybridize to two or more genes encoding proteins listed in Table 6 or Table 10, or fragments thereof; or (ii) polypeptide binding agents that specifically bind to two or more polypeptides selected from proteins listed in Table 6 or Table 10, or fragments thereof, (b) adding the probes to a sample comprising mRNA or proteins from an animal exhibiting the lean phenotype, in a manner enabling hybridization or binding of the probes to the mRNA or proteins in the sample, thereby forming hybridization or binding complexes in the sample (c) optionally, adding the probes to another sample comprising mRNA or proteins from a normal animal, in a manner enabling hybridization or binding of the probes to the mRNA or proteins in the second sample, thereby forming hybridization or binding complexes in the other sample; (d) detecting the hybridization complexes in the sample or samples; and (e) comparing the hybridization or binding complexes from the first sample with the hybridization or binding complexes from a standard or, optionally, from the other sample, wherein at least one difference between the amount of hybridization or binding in the sample as compared with the standard or the optional other sample indicates differential expression of the one or more genes differentially expressed in animals exhibiting the lean phenotype, as compared to animals that do not exhibit the phenotype.

The method may be used to detect differential expression of genes encoding the gene products set forth in Table 6 or table 10, or in subsets thereof, namely tissue-specific subsets as listed in Table 6A-G and Table 10A-G, or functional subsets as set forth in Tables 7, 8 and 9 (for three-treatment analysis) and Tables 11, 12 and 13 (for high protein diet analysis). In a preferred embodiment, the device is uses to detect differential expression of genes from canines or felines. In particular embodiments, the probes are bound to a substrate, preferably in an array.

Step (c) and part of steps (d) and (e) are optional and are used if a relatively contemporaneous comparison of two or more test systems is to be conducted. However, in a preferred embodiment, the standard used for comparison is based upon data previously obtained using the method.

These probes are exposed to a sample to form hybridization or binding complexes that are detected and compared with those of a standard. The differences between the hybridization or binding complexes from the sample and standard indicate differential expression of polynucleotides and therefore genes differentially expressed in the lean phenotype versus normal phenotype in the sample. In a preferred embodiment, probes are made to specifically detect polynucleotides or fragments thereof produced by one or more of the genes or gene fragments identified by the invention. Methods for detecting hybridization complexes are known to skilled artisans.

In one embodiment, the method further comprises exposing the animal or sample to a test substance before performing the assay. Then, the comparison is indicative of whether the test substance altered the expression of genes differentially expressed in treated versus untreated animals.

The assays described herein for the detection of lean phenotype-associated transcription and translation products are useful in methods for identifying a lean phenotype in an animal, or the absence of a lean phenotype in the animal. Such methods may be useful for implementing, facilitating, or guiding a weight-loss regimen (especially for loss of fat mass, and particular for loss of fat while preserving or improving lean body mass) or physical fitness regimen, or for diagnostic purposes to identify animals at risk for obesity or obesity-associated health risks or diseases. Such methods comprise obtaining a sample of cells or tissue from an animal manifesting a lean phenotype as defined herein, or an overweight or obese animal. Such cells or tissues can include, without limitation, adipose, muscle, or liver tissues. The cell or tissue sample is then analyzed for modulated expression of one or more genes associated with a lean phenotype, via detection of mRNA or protein, or for a particular lean phenotype-associated gene expression profile using a gene- or protein-array as described herein. The results of the analysis will reveal whether the animal is manifesting a lean phenotype or transitioning to a lean phenotype. The methods can also provide information regarding the efficacy of an animal's weight loss or physical fitness regimen, or to monitor an animal's relative risk for obesity or obesity-associated health risks or diseases over time. In these situations, the method is carried out at intervals during the animal's weight loss or physical fitness regimen, or life generally, wherein a change in expression or pattern of expression of a target gene associated with a lean phenotype is indicative of the animal's progress, improvement, or continued risk.

In another aspect, the invention provides a method of determining if a test substance is likely to be useful in promoting a lean phenotype when administered to an animal. The method typically comprises (a) determining a first gene expression profile by measuring the transcription or translation products of two or more polynucleotides selected from genes encoding proteins listed in Table 6 or Table 10, or fragments thereof, in a test system in the absence of the test substance; (b) determining a second gene expression profile by measuring the transcription or translation products of two or more polynucleotides selected from genes encoding proteins listed in Table 6 or Table 10, or fragments thereof, in a test system in the presence of the test substance; and (c) comparing the first gene expression profile with the second gene expression profile, wherein a change in the second gene expression profile as compared with the first gene expression profile indicates that the test substance is likely to be useful in promoting a lean phenotype when administered to an animal.

In certain embodiments, the method may further include the step of comparing at least the second gene expression profile with a reference or standard gene expression profile obtained by measuring the transcription or translation products of two or more polynucleotides selected from genes encoding proteins listed in Table 6 or Table 10, or fragments thereof, in a test system in the presence of a reference substance known to promote a lean phenotype when administered to animals. Such a substance may be, for example, CLA.

In one embodiment, the test system comprises a population of cultured cells. A nucleic acid construct comprising a lean phenotype-associated gene according to the invention is introduced into cultured host cells. The host cells can be mammalian cell lines, such as but are not limited, to NIH3T3, CHO, HFLA, and COS, although non-mammalian cells such as yeast, bacteria and insect cells can also be used. The coding sequences of the genes are operably linked to appropriate regulatory expression elements suitable for the particular host cell to be utilized. The nucleic acid constructs can be introduced into the host cells according to any acceptable means in the art, including but not limited to, transfection, transformation, calcium phosphate precipitation, electroporation and lipofection. Such techniques are well known and routine in the art. Transformed cells can be also used to identify compounds that modulate expression of the lean phenotype-associated genes.

Gene expression assays can be carried out using a gene construct comprising the promoter of a selected lean phenotype-associated gene operably linked to a reporter gene. The reporter construct may be introduced into a suitable cultured cell, including, without limitation, the standard host cell lines described above, or cells freshly isolated from an animal such as adipose, muscle, or liver cells. The assay is performed by monitoring expression of the reporter gene in the presence or absence of a test substance such as a test compound.

In a preferred embodiment, the test system comprises animals. Typically, a test substance is administered to an animal and the gene expression profile of the animal is analyzed to determine the effect of the test substance on transcription or the translation of the genes or gene products of the invention. Gene expression can be analyzed in situ or ex vivo to determine the effect of the test substance. In another embodiment, a test substance is administered to an animal and the activity of a protein expressed from a gene is analyzed in situ or ex vivo according to any means suitable in the art to determine the effect of the test substance on the activity of the proteins of interest. In addition, where a test substance is administered to an animal, the physiological, systemic, and physical effects of the compound, as well as potential toxicity of the compound can also be evaluated. Test substances can be administered to the animals for periods appropriate for the test substance, the animal, and the objective, including long term administration, administration on a regular basis, and on an extended regular basis. Administration can be via any suitable route, including, but are not limited to, oral, rectal, nasal, topical, intradermal, subcutaneous, intravenous, intramuscular, and intraparenteral modes of administration. Oral administration is preferred, most preferably oral administration as a food component.

Test substances can be any substance that may have an effect on polynucleotides or genes differentially expressed in animals exhibiting a lean phenotype. Suitable test substances include, but are not limited to, amino acids; proteins, peptides, polypeptides, nucleic acids, oligonucleotides, polynucleotides, small molecules, macromolecules, vitamins, minerals, simple sugars; complex sugars; polysaccharides; carbohydrates; medium-chain triglycerides (MCTs); triacylglycerides (TAGS); n-3 (omega-3) fatty acids including DHA, EPA, ALA; n-6 (omega-6) fatty acids including LA, γ-linolenic acid (GLA) and ARA; SA, conjugated linoleic acid (CLA); choline sources such as lecithin; fat-soluble vitamins including vitamin A and precursors thereof such as carotenoids (e.g., (β-carotene), vitamin D sources such as vitamin D2 (ergocalciferol) and vitamin D3 (cholecalciferol), vitamin E sources such as tocopherols (e.g., α-tocopherol) and tocotrienols, and vitamin K sources such as vitamin K1 (phylloquinone) and vitamin K2 (menadione); water-soluble vitamins including B vitamins such as riboflavin, niacin (including nicotinamide and nicotinic acid), pyridoxine, pantothenic acid, folic acid, biotin and cobalamin; and vitamin C (ascorbic acid); antioxidants, including some of the vitamins listed above, especially vitamins E and C; also bioflavonoids such as catechin, quercetin and theaflavin; quinones such as ubiquinone; carotenoids such as lycopene and lycoxanthin; resveratrol; and α-lipoic acid; L-carnitine; D-limonene; glucosamine; S-adenosylmethionine; and chitosan. In a preferred embodiment, test substances are nutrients that may be added to food or consumed as a supplement. Substances identified by the foregoing method are also contemplated as part of the invention.

In a further aspect, the invention provides a computer system comprising a database containing information identifying expression levels of one or more polynucleotides that are differentially expressed in animals administered a substance that affects one or more of food intake, satiety, lipid metabolism, and fat utilization, wherein the polynucleotides are selected from genes encoding proteins listed in Table 6 or Table 10, or fragments thereof, and a user interface that enables a user to access or manipulate the information in the database. The system comprises a database containing information identifying the expression level of one or more polynucleotides selected from genes encoding proteins listed in Table 6 or Table 10 and/or polypeptides that specifically bind to the proteins listed in Table 6 or Table 10, and a user interface to interact with the database, particularly to input, manipulate, and review the information for different animals or categories of animals. In one embodiment, the database further contains information identifying the activity level of one or more polypeptides listed in Table 6 or Table 10. In another, the database further comprises sequence information for one or more of the polynucleotides or polypeptides as listed in Table 6 or Table 10, preferably from a variety of species. In other embodiments, the database contains additional information pertaining to the description of the genes in one or more animal species. The computer system is any electronic device capable of containing and manipulating the data and interacting with a user, e.g., a typical computer or an analytical instrument designed to facilitate using the invention and outputting the results relating to the status of an animal.

In another aspect, the invention provides kits comprising a container containing a collection of two or more probes for detecting differential gene expression of the lean phenotype resulting from one or more lean phenotype-promoting treatments comprising (1) administration of CLA, (2) consumption of a high protein diet, and (3) increased exercise. The kits comprise in separate containers in a single package or in separate containers in a virtual package, as appropriate for the use and kit component, two or more probes for detecting differential gene expression in animals exhibiting a lean phenotype resulting from one or more lean phenotype-promoting treatments comprising (1) administration of CLA, (2) consumption of a high protein diet, and (3) increased exercise, wherein the probes comprise: (a) polynucleotides that specifically hybridize to two or more genes encoding proteins listed in Table 6 or Table 10, or fragments thereof, or (b) polypeptide binding agents that specifically bind to two or more polypeptides selected from proteins listed in Table 6 or Table 10, or fragments thereof; and further comprises at least one of (1) instructions for how to use the probes in a gene expression assay for detecting differential gene expression in animals exhibiting a lean phenotype resulting from one or more lean phenotype-promoting treatments comprising administration of CLA, consumption of a high protein diet, and/or increased exercise, (2) reagents and equipment for using the probes, and (3) a composition known to induce the lean phenotype. Preferably, the probes are affixed to a solid support at known locations. Suitable reference substances known to induce the lean phenotype include CLA, for example.

When the kit comprises a virtual package, the kit is limited to instructions in a virtual environment in combination with one or more physical kit components. In one embodiment, the kit contains probes and/or other physical components and the instructions for using the probes and other components are available via the internes. The kit may contain additional items such as a device for mixing samples, probes, and reagents and device for using the kit, e.g., test tubes or mixing utensils.

In another aspect, the invention provides a means for communicating information about or instructions for one or more of the compositions and methods described herein. Such means typically comprise documents, digital storage media, optical storage media, audio presentations, visual displays or the like, containing the information or instructions. For example, the communication means may be a displayed web site, a kiosk, brochure, product label, package insert, advertisement, handout, public announcement audiotape, videotape, DVD, CD, computer-readable chip, computer-readable card, computer-readable disk, computer memory, or any combination thereof. Useful information includes one or more of (1) methods for promoting the health and wellness of animals and (2) contact information for the animal's caregivers to use if they have a question about the invention and its use. Useful instructions include techniques for using the probes, instructions for performing a gene expression assay, and administration amounts and frequency for the substances. The communication means is useful for instructing on the benefits of using the invention.

### EXAMPLES

Various aspects of the invention can be further illustrated by the following examples. It will be understood that these examples are provided merely for purposes of illustration and do not limit the scope of the invention disclosed herein unless otherwise specifically indicated.

### Example 1

The experimental results set forth in this example demonstrate that animals can be induced to transition to a lean phenotype in three ways: (1) dietary supplementation with CLA, (2) high protein diet, and (3) increased exercise.

### Materials and Methods

Four-week old male Sprague Dawley rats were allowed to acclimate for two weeks on a control diet comprising AIN-93M (American Institute of Nutrition purified diet formula for maintenance of mature rodents). Rats were divided into four groups of 12. Group 1 was fed the control diet supplemented with CLA (Table 1). Group 2 was fed a modified diet increased in protein and decreased in carbohydrate (Table 1). Group 3 was fed the control diet, and was given the opportunity to engage in supplemental exercise. Specifically, a running wheel was placed in the cage of each rat in group 3, and daily use of the wheel was monitored by recording each turn of the wheel using a sensor connected to a computer. Group 4 was fed the control diet (Table 1). The energy content of the control and test diets is shown in Table 2.

TABLE 1
Control and Test Diet Composition

| | Modified | | |
|---|---|---|---|
| | AIN-93M | High Protein | 3% CLA |
| | (Control) | Diet | Diet |
| | (g/kg diet) | (g/kg diet) | (g/kg diet) |
| Casein (greater than 85% protein) | 200 | 500 | 200 |
| Cornstarch | 417 | 302 | 417 |
| Dextrinized cornstarch | 132 | 0 | 132 |
| (90-94% tetrasaccharides) | | | |
| Sucrose | 100 | 50 | 100 |
| Soybean oil (no additives) | 50 | 50 | 20 |
| Fiber | 50 | 50 | 50 |
| Mineral Mix (AIN-93G-MX) | 35 | 35 | 35 |
| Vitamin Mix (AIN-93G-VX) | 10 | 10 | 10 |
| L-cystine | 3 | 0 | 3 |
| Choline bitartrate (41.1% choline) | 2.5 | 2.5 | 2.5 |
| Tert-butylhydroquinone | 0.014 | 0.014 | 0.014 |
| Conjugated linoleic acid (CLA) | 0 | 0 | 30 |

TABLE 2
Energy Density of Control and Test Diets

| | Control | High Protein | 3% CLA |
|---|---|---|---|
| Total energy (kcal) | 3859.94 | 3859.94 | 3859.94 |
| % as protein | 17.6 | 44 | 17.6 |
| % as carbohydrate | 70.7 | 44.3 | 70.7 |
| % as fat | 11.7 | 11.7 | 11.7 |

Measurements were taken at 7 and 60 days after initiation of the feeding or exercise protocols. At the completion of the 60-day feeding protocol, all animals were sacrificed and analyses were performed.

### Results

Body composition analyses were performed on the test animals. Compared with the control group, none of the test regimens (CLA supplementation, exercise or high dietary protein) had any substantial effect on (1) food intake or final body weight, (2) stripped carcass weight, (3) total organ weight, digestive tract weight, heart weight, kidney weight, liver weight, lung weight or spleen weight (with the exception of the high protein regimen, which resulted in increased kidney and spleen weight), (4) protein and fat content of the carcass, or (5) protein and fat content of the organs (except that high protein regimen increased the protein content and decreased the fat content of the organs).

By comparison, as can be seen from Table 3, each of the three treatments decreased total fat pad weight and retroperitoneal fat pad weight, as compared with the Control group. Thus, the data showed that CLA supplementation, increased exercise or high protein diet reduced body fat, resulting in the lean body mass (LBM) phenotype.

TABLE 3
Body Fat Analysis Following 60 Days of Treatment

| | Control | CLA | Exercise | High Protein |
|---|---|---|---|---|
| Total fat pad (g) | 44.97 | 31.80 | 35.99 | 33.26 |
| Retroperitoneal fat (g) | 16.74 | 10.07 | 12.85 | 12.32 |

Biochemical analyses of blood were performed on the test animals; results are shown in Table 4.

TABLE 4
Blood Analysis at Day 7 and Day 60

| | Control | | CLA | | Exercise | | High Protein | |
|---|---|---|---|---|---|---|---|---|
| | Day 7 | Day 60 | Day 7 | Day 60 | Day 7 | Day 60 | Day 7 | Day 60 |
| Fasting blood | 10.65 | 12.11 | 10.53 | 11.08 | 10.93 | 11.39 | 11.09 | 10.95 |
| glucose | | | | | | | | |
| (mmol/L) | | | | | | | | |
| Fasting blood | 102.42 | 148.94 | 111.65 | 120.77 | 100.86 | 155.44 | 151.02 | 182.09 |
| glucagon | | | | | | | | |
| (pg/ml) | | | | | | | | |
| Fasting blood | 210.09 | 239.92 | 233.58 | 172.66 | 176.58 | 199.33 | 227.08 | 183.50 |
| insulin | | | | | | | | |
| (pmol/L) | | | | | | | | |

After seven days of the respective treatments, it was found that the increased exercise treatment reduced blood insulin as compared with the CLA supplementation and the high protein diet. The high protein diet treatment increased blood glucagon level compared with the control, the CLA supplementation and the exercise treatment. Blood glucose was not affected by any treatment after 7 days. After 60 days of the respective treatments, it was found that the CLA supplementation and the high protein diet reduced blood glucose level compared with the control diet and the exercise treatment. The CLA supplementation and high protein diet also reduced blood insulin compared with the control diet and the exercise treatment. Blood glucagon was highest in the high protein diet group as compared with the other groups. These data show that CLA supplementation, exercise or the high protein diet altered the test animals' biochemical profiles in a manner consistent with the LBM phenotype.

### Example 2

This example sets forth the initial analysis of gene expression profiles in liver, muscle and/or adipose of the control and test animals comprising three treatments that promote a LBM phenotype, as described in the previous example at day 60.

### Materials and Methods

Messenger RNA was prepared in accordance with standard techniques applicable to the various tissues. Affymetrix GeneChip® Rat Genome 230 2.0 Arrays were interrogated with mRNA from adipose (subcutaneous), liver and quadricep muscle from 6 rats from each of the four groups: (1) control, (2) CLA supplementation, (3) high protein diet, and (4) increased exercise regimen (total 72 samples, utilizing 72 GeneChip® arrays).

The RMA (Robust Multi-chip Analysis) procedure was used to normalize; correlation matrix and PCA were used to assess the quality control. Two-way Analysis of Variance (ANOVA) for repeated measures was used for a first step analysis (gene by gene). Then as a second step, the GEA (Global Error Assessment) procedure was applied, the robust statistic then replaced the error term of the ANOVA calculated in the first step. Genes were selected based on p<0.01 for overall treatment effect within tissue and then p<0.01 for a specific treatment versus control within that tissue.

### Results

The aforementioned processes identified several hundred genes that were differentially expressed between the control group and at least one of the three treatments, in at least one of the three tissues that were analyzed. Table 5 below shows a breakdown of results from an analysis of (1) the high protein diet treatment alone and (2) a combination of all three treatments, i.e., genes found to be differentially expressed in each of the three treatments as compared with the control group.

TABLE 5
Numbers of Genes Differentially Expressed in Treated Versus Control
Animals in Three Tissues and Combinations of Three Tissues

| | | | | Adipose + | Adipose + | Liver + | Adipose + |
|---|---|---|---|---|---|---|---|
| Treatment | Adipose | Liver | Quadricep | Liver | Quad | Quad | Liver + Quad |
| High protein | 2041 | 607 | 274 | 128 | 78 | 36 | 23 |
| treatment only | | | | | | | |
| Combination | 774 | 105 | 50 | 14 | 10 | 3 | 3 |
| of all three | | | | | | | |
| treatments* | | | | | | | |

*Combination of all three treatments: number of genes differentially expressed in each of the three treatments, i.e., CLA supplementation and high protein diet and increased exercise.

### Example 3

This example sets forth the further analysis of gene expression profiles in liver, muscle and/or adipose of the control and test animals comprising three treatments that promote a LBM phenotype, as described in Example 1 at day 60 and initially analyzed in Example 2.

Table 6 sets forth database identifiers and the names of the genes found to be differentially expressed in each of the three treatments.

TABLE 6
Genes Differentially Expressed in Three Tissues Individually, or in Two or More Tissues, or in
all Three Tissues as a Result of CLA Supplementation, High Protein Diet and Increased Exercise

| | | |
|---|---|---|
| Public ID | Gene Symbol | Gene Title |

A. Adipose

| | | |
|---|---|---|
| AA800587 | Gpx2 | glutathione peroxidase 2 |
| AA801220 | — | Transcribed locus |
| AA817708 | LOC310190 | similar to hypothetical protein FLJ11127 |
| AA817742 | Ms4a1_predicted | membrane-spanning 4-domains, subfamily A, member 1 |
| | | (predicted) |
| AA818807 | Emp2 | epithelial membrane protein 2 |
| AA818954 | — | Transcribed locus |
| AA848820 | Hpgd | hydroxyprostaglandin dehydrogenase 15 (NAD) |
| AA848821 | LOC684681 | similar to Histone H1.2 (H1 VAR.1) (H1c) |
| AA848944 | — | Transcribed locus |
| AA850195 | Pank1_predicted | pantothenate kinase 1 (predicted) |
| AA850618 | Sorl1 | sortilin-related receptor, LDLR class A repeats-containing |
| AA850773 | — | — |
| AA851046 | — | — |
| AA851313 | — | — |
| AA851345 | — | Transcribed locus |
| AA859029 | — | Transcribed locus |
| AA859235 | Cdca7 | cell division cycle associated 7 |
| AA859277 | — | Transcribed locus |
| AA859508 | RGD1564315_predicted | similar to RIKEN cDNA 9330161F08 (predicted) |
| AA859652 | — | Transcribed locus |
| AA859982 | Dhx36_predicted | DEAH (Asp-Glu-Ala-His) box polypeptide 36 (predicted) |
| AA875132 | — | Transcribed locus |
| AA875609 | Smarca2 | SWI/SNF related, matrix associated, actin dependent |
| | | regulator of chromatin, subfamily a, member 2 |
| AA875647 | — | Transcribed locus |
| AA891634 | — | Transcribed locus, weakly similar to XP_343521.3 |
| | | PREDICTED: similar to Discs large homolog 5 (Placenta |
| | | and prostate DLG) (Discs large protein P-dlg) |
| | | [Rattus norvegicus] |
| AA891826 | Tacstd2 | tumor-associated calcium signal transducer 2 |
| AA892778 | — | Transcribed locus |
| AA893195 | — | Transcribed locus |
| AA894233 | Amid_predicted | apoptosis-inducing factor (AIF)-like mitochondrion- |
| | | associated inducer of death (predicted) |
| AA894262 | 1-Sep | septin 1 |
| AA894336 | Ly96 | lymphocyte antigen 96 |
| AA899923 | Eml4_predicted | echinoderm microtubule associated protein like 4 |
| | | (predicted) |
| AA900322 | — | Transcribed locus |
| AA900477 | Vav2_predicted | Vav2 oncogene (predicted) |
| AA901088 | RGD1561302_predicted | similar to Hypothetical protein A030013D21 (predicted) |
| AA901290 | — | Transcribed locus |
| AA924459 | RGD1560293_predicted | similar to RIKEN cDNA 1200013B08 (predicted) |
| AA925583 | Blnk | B-cell linker |
| AA925804 | Pmpcb | peptidase (mitochondrial processing) beta |
| AA925924 | Crlf1_predicted | cytokine receptor-like factor 1 (predicted) |
| AA926072 | — | Transcribed locus |
| AA926082 | LOC500046 | similar to hypothetical protein FLJ21986 |
| AA943147 | Herc6 | potential ubiquitin ligase |
| AA943808 | — | Transcribed locus |
| AA944073 | Rpl41 | ribosomal protein L41 |
| AA944162 | Olfml2a_predicted | olfactomedin-like 2A (predicted) |
| AA944574 | — | Transcribed locus |
| AA945727 | Card11_predicted | caspase recruitment domain family, member 11 |
| | | (predicted) |
| AA946353 | Satb1 | Special AT-rich sequence binding protein 1 |
| AA955354 | Stat4 | signal transducer and activator of transcription 4 |
| AA955771 | — | — |
| AA957260 | Rrm2 | ribonucleotide reductase M2 |
| AA957585 | Ehd3 | EH-domain containing 3 |
| AA964146 | LOC498662 | similar to RIKEN cDNA 2610019F03 |
| AA964219 | Lipg | lipase, endothelial |
| AA964492 | — | Transcribed locus |
| AA965084 | Tnn_predicted | tenascin N (predicted) |
| AA965250 | LOC314964 | similar to PHD finger protein 20-like 1 isoform 1 |
| AA996557 | LOC678701 /// | similar to immunoglobulin heavy chain 6 (Igh-6) /// |
| | RGD1359202 | hypothetical protein LOC678701 |
| AA996717 | RGD1307583_predicted | LOC361111 (predicted) |
| AA996804 | — | Transcribed locus |
| AA996885 | Ccl19_predicted | chemokine (C-C motif) ligand 19 (predicted) |
| AA996927 | LOC690795 | similar to H2A histone family, member J |
| AA997873 | — | Transcribed locus |
| AA998001 | — | Transcribed locus |
| AA998516 | Ccna2 | cyclin A2 |
| AA998540 | LOC313934 | similar to Intersectin-2 (SH3 domain-containing protein |
| | | 1B) (SH3P18) (SH3P18-like WASP-associated protein) |
| AA998903 | — | CDNA clone IMAGE: 7374368 |
| AA998997 | — | Transcribed locus |
| AB012232 | Nfib | nuclear factor I/B |
| AB035507 | Mcam | melanoma cell adhesion molecule |
| AB039823 | LOC259244 /// | alpha-2u globulin PGCL3 /// |
| | LOC259245 /// | alpha-2u globulin PGCL5 /// |
| | LOC259246 /// | alpha-2u globulin PGCL1 /// |
| | LOC298109 /// | alpha-2u globulin PGCL4 /// |
| | LOC298111 /// | major urinary protein 5 /// |
| | LOC298116 /// | alpha-2u globulin PGCL2 /// |
| | LOC366380 /// | alpha2u globulin /// |
| | LOC500473 /// | alpha-2u-globulin (L type) /// |
| | LOC502954 /// | major urinary protein 4 /// |
| | LOC681426 /// | alpha 2U globulin /// |
| | LOC682731 /// | similar to alpha-2u globulin PGCL2 /// |
| | LOC685482 /// | similar to alpha-2u-globulin (L type) /// |
| | LOC685577 /// | similar to alpha-2u globulin PGCL4 isoform 1 /// |
| | LOC688457 /// | similar to major urinary protein 5 /// |
| | LOC688547 /// | similar to alpha2u globulin /// |
| | Mup4 /// | similar to Major urinary protein precursor (MUP) |
| | Mup5 /// | (Alpha-2u-globulin) (Alpha(2)-euglobulin) (Allergen |
| | Obp3 | Rat n 1) (Rat n I) /// similar to major urinary protein 4 |
| AB039826 | LOC259245 /// | alpha-2u globulin PGCL5 /// major urinary protein 5 |
| | Mup5 | |
| AB039828 | LOC259244 /// | alpha-2u globulin PGCL3 /// |
| | LOC259245 /// | alpha-2u globulin PGCL5 /// |
| | LOC259246 /// | alpha-2u globulin PGCL1 /// |
| | LOC298109 /// | alpha-2u globulin PGCL4 /// |
| | LOC298111 /// | major urinary protein 5 /// |
| | LOC298116 /// | alpha-2u globulin PGCL2 /// |
| | LOC366380 /// | alpha2u globulin /// |
| | LOC500473 /// | alpha-2u-globulin (L type) /// |
| | LOC502954 /// | major urinary protein 4 /// |
| | LOC681426 /// | alpha 2U globulin /// |
| | LOC682731 /// | similar to alpha-2u globulin PGCL2 /// |
| | LOC685482 /// | similar to alpha-2u-globulin (L type) /// |
| | LOC685577 /// | similar to alpha-2u globulin PGCL4 isoform 1 /// |
| | LOC688457 /// | similar to major urinary protein 5 /// |
| | LOC688547 /// | similar to alpha2u globulin /// |
| | Mup4 /// | similar to Major urinary protein precursor (MUP) |
| | Mup5 /// | (Alpha-2u-globulin) (Alpha(2)-euglobulin) (Allergen |
| | Obp3 | Rat n 1) (Rat n I) /// similar to major urinary protein 4 |
| AF002251 | Rassf5 | Ras association (RalGDS/AF-6) domain family 5 |
| AF035963 | Havcr1 | kidney injury molecule 1 |
| AF068860 | Defb1 | defensin beta 1 |
| AF228917 | Zdhhc2 | zinc finger, DHHC domain containing 2 |
| AF255614 | Magi3 | membrane associated guanylate kinase, WW and PDZ |
| | | domain containing 3 |
| AF283276 | Gabbr1 | gamma-aminobutyric acid (GABA) B receptor 1 |
| AF411318 | Mt1a | metallothionein 1a |
| AI008369 | Coro7 | coronin 7 |
| AI009791 | — | Transcribed locus |
| AI009823 | Sectm1b | secreted and transmembrane 1B |
| AI009944 | Txk | TXK tyrosine kinase |
| AI010107 | — | Transcribed locus |
| AI010237 | — | Transcribed locus |
| AI010476 | Rac2 | RAS-related C3 botulinum substrate 2 |
| AI011101 | RGD1560320_predicted | similar to brain specific protein 4 (predicted) |
| AI011713 | — | CDNA clone IMAGE: 7320582 |
| AI012081 | Rhoh | ras homolog gene family, member H |
| AI012247 | — | Transcribed locus, strongly similar to XP_892770.1 |
| | | PREDICTED: similar to Tyrosine-protein kinase FLK |
| | | [Mus musculus] |
| AI012250 | — | Transcribed locus |
| AI012520 | — | — |
| AI012590 | — | Transcribed locus |
| AI012884 | — | Transcribed locus |
| AI029631 | Igl@ | Immunoglobulin light chain, lambda gene cluster |
| AI029798 | — | Transcribed locus |
| AI029975 | — | Transcribed locus, strongly similar to NP_038482.3 ATP- |
| | | binding cassette 1, sub-family A, member 1 |
| | | [Mus musculus] |
| AI030203 | Irx3_predicted | Iroquois related homeobox 3 (Drosophila) (predicted) |
| AI030853 | Calml3 | calmodulin-like 3 |
| AI043752 | — | Transcribed locus |
| AI043753 | — | Transcribed locus |
| AI044494 | — | Transcribed locus |
| AI044500 | Ctdspl_predicted | CTD (carboxy-terminal domain, RNA polymerase II, |
| | | polypeptide A) small phosphatase-like (predicted) |
| AI044622 | MGC125004 | similar to T cell receptor interacting molecule |
| AI044631 | Cd3g | CD3 antigen, gamma polypeptide |
| AI044632 | — | — |
| AI044784 | — | Transcribed locus |
| AI045116 | — | Transcribed locus |
| AI045155 | Ccr6 | chemokine (C-C motif) receptor 6 |
| AI045311 | Bin2a | beta-galactosidase-like protein |
| AI045321 | — | Transcribed locus |
| AI045904 | — | — |
| AI045965 | RGD1311456_predicted | similar to RIKEN cDNA B230380D07 (predicted) |
| AI058292 | Sox7_predicted | SRY-box containing gene 7 (predicted) |
| AI058307 | Phemx | pan hematopoietic expression |
| AI059204 | Cpne8_predicted | copine VIII (predicted) |
| AI059295 | RGD1561653_predicted | similar to HECT domain containing 1 (predicted) |
| AI059468 | LOC308990 | hypothetical protein LOC308990 |
| AI059486 | Dennd2d_predicted | DENN/MADD domain containing 2D (predicted) |
| AI060043 | — | Transcribed locus |
| AI070558 | — | — |
| AI070991 | — | Transcribed locus |
| AI071227 | RGD1563603_predicted | similar to DNA repair protein RAD51 homolog 1 |
| | | (predicted) |
| AI071569 | Il7r_predicted | interleukin 7 receptor (predicted) |
| AI071674 | — | Transcribed locus |
| AI071686 | — | Transcribed locus |
| AI072042 | — | — |
| AI072252 | RGD1565169_predicted | similar to apolipoprotein B48 receptor (predicted) |
| AI072663 | — | Transcribed locus |
| AI072892 | Frzb | frizzled-related protein |
| AI073219 | — | Transcribed locus |
| AI101659 | — | — |
| AI101952 | MGC125215 | multiple coiled-coil GABABR1-binding protein |
| AI102190 | — | Transcribed locus |
| AI102482 | RGD1561521_predicted | similar to 1110014F24Rik protein (predicted) |
| AI103939 | LOC363091 | similar to hypothetical protein FLJ30973 |
| AI104533 | Ttn | Titin |
| AI105042 | Cabc1 | chaperone, ABC1 activity of bc1 complex like (S. pombe) |
| AI136525 | Degs2 | degenerative spermatocyte homolog 2 (Drosophila), lipid |
| | | desaturase |
| AI136555 | Cipar1 | castration induced prostatic apoptosis-related protein 1 |
| AI137113 | Tmed5 | transmembrane emp24 protein transport domain |
| | | containing 5 |
| AI137137 | Lck | lymphocyte protein tyrosine kinase |
| AI137640 | Cldn1 | claudin 1 |
| AI137791 | — | Transcribed locus |
| AI144946 | Dynlrb2_predicted | dynein light chain roadblock-type 2 (predicted) |
| AI144948 | — | Transcribed locus |
| AI145398 | — | Transcribed locus |
| AI146108 | — | Transcribed locus |
| AI169601 | Tnfrsf14 | tumor necrosis factor receptor superfamily, member 14 |
| | | (herpesvirus entry mediator) |
| AI170002 | LOC689134 | similar to Protein transport protein SEC61 gamma subunit |
| AI170541 | — | Transcribed locus |
| AI170987 | — | Transcribed locus |
| AI171093 | Prkcq | protein kinase C, theta |
| AI172110 | — | — |
| AI175668 | — | — |
| AI175728 | — | — |
| AI176129 | LOC686611 | similar to Protein FAM60A (Tera protein) |
| AI176327 | RGD1562408_predicted | similar to SH2 domain protein 1A (Signaling lymphocyte |
| | | activation molecule-associated protein) (predicted) |
| AI176755 | Ptpn22_predicted | protein tyrosine phosphatase, non-receptor type 22 |
| | | (lymphoid) (predicted) |
| AI177373 | Cr2_predicted | Complement receptor 2 (predicted) |
| AI177589 | Sorl1 | sortilin-related receptor, LDLR class A repeats-containing |
| AI177645 | RGD1310168_predicted | similar to cDNA sequence BC032204 (predicted) |
| AI177743 | — | Transcribed locus, strongly similar to XP_573486.1 |
| | | PREDICTED: hypothetical protein XP_573486 |
| | | [Rattus norvegicus] |
| AI177934 | LOC474169 | pre-eosinophil-associated ribonuclease-2 |
| AI178243 | Donson | downstream neighbor of SON |
| AI178384 | Klhl6_predicted | kelch-like 6 (Drosophila) (predicted) |
| AI178618 | — | — |
| AI178693 | RGD1305754_predicted | similar to 3110080A02Rik protein (predicted) |
| AI178784 | RGD1310174_predicted | hypothetical LOC298504 (predicted) |
| AI178808 | Il2rg | interleukin 2 receptor, gamma (severe combined |
| | | immunodeficiency) |
| AI179227 | Cybasc3 | cytochrome b, ascorbate dependent 3 |
| AI179665 | RGD1305755 | Similar to RIKEN cDNA 5033406L14 |
| AI179988 | Enc1 | ectodermal-neural cortex 1 |
| AI180352 | Herc1_predicted | hect (homologous to the E6-AP (UBE3A) carboxyl |
| | | terminus) domain and RCC1 (CHC1)-like domain (RLD) |
| | | 1 (predicted) |
| AI180370 | RGD1304592_predicted | similar to KIAA0528 protein (predicted) |
| AI227638 | Satb1 | special AT-rich sequence binding protein 1 |
| AI227800 | Kifap3_predicted | kinesin-associated protein 3 (predicted) |
| AI230591 | RGD1565540_predicted | similar to ctla-2-beta protein (141 AA) (predicted) |
| AI231826 | — | Transcribed locus |
| AI233243 | Rhot2 | ras homolog gene family, member T2 |
| AI233902 | — | Transcribed locus |
| AI234943 | Sf3b1 | Splicing factor 3b, subunit 1 |
| AI235414 | — | — |
| AI235468 | Dst_predicted | dystonin (predicted) |
| AI235528 | — | Transcribed locus |
| AI236141 | LOC686590 | similar to IQ motif and Sec7 domain 1 |
| AI237227 | — | Transcribed locus |
| AI237640 | LOC683446 /// | similar to killer activatory receptor-like protein p91D |
| | RGD1562625_predicted | (predicted) /// similar to paired-Ig-like receptor A11 |
| AI406660 | — | — |
| AI406854 | — | Transcribed locus |
| AI407163 | — | Transcribed locus |
| AI407239 | — | Transcribed locus |
| AI408164 | RGD1306176 | similar to FCRL |
| AI408343 | RGD1562552_predicted | similar to hypothetical protein LOC340061 (predicted) |
| AI408425 | Tlr7_predicted | toll-like receptor 7 (predicted) |
| AI408839 | Rwdd2_predicted | RWD domain containing 2 (predicted) |
| AI408948 | Ca2 | carbonic anhydrase 2 |
| AI409142 | LOC679811 | similar to RIKEN cDNA D930015E06 |
| AI409262 | Rhebl1 | Ras homolog enriched in brain like 1 |
| AI409635 | — | Transcribed locus |
| AI409904 | — | Transcribed locus |
| AI410068 | Fcer2a | Fc receptor, IgE, low affinity II, alpha polypeptide |
| AI411185 | — | Transcribed locus |
| AI411326 | LOC303057 /// Slu7 | similar to step II splicing factor SLU7; DNA segment, Chr |
| | | 11, ERATO Doi 730, expressed; DNA segment, Chr 3, |
| | | Brigham & Womens Genetics 0878 expressed /// step II |
| | | splicing factor SLU7 (S. cerevisiae) |
| AI411413 | LOC685778 /// | pyruvate dehydrogenase E1 alpha 1 /// pyruvate |
| | Pdha1 | dehydrogenase E1 alpha 1 pseudogene |
| AI411425 | Esd | esterase D/formylglutathione hydrolase |
| AI411563 | Fgd2_predicted | FYVE, RhoGEF and PH domain containing 2 (predicted) |
| AI411747 | Art2b | ADP-ribosyltransferase 2b |
| AI412322 | Acat2 | acetyl-Coenzyme A acetyltransferase 2 |
| AI412781 | LOC680281 /// | similar to RIKEN cDNA 4930555G01 (predicted) /// |
| | LOC685769 /// | similar to Discs large homolog 5 (Placenta and prostate |
| | LOC690576 /// | DLG) (Discs large protein P-dlg) /// hypothetical protein |
| | LOC691414 /// | LOC691414 |
| | RGD1563862_predicted | |
| AI412866 | RGD1306100_predicted | similar to RRP22 (predicted) |
| AI454224 | — | Transcribed locus |
| AI501187 | Dsg1c_predicted /// | desmoglein 1 gamma (predicted) /// similar to |
| | LOC679010 | Desmoglein-1 alpha precursor (Dsg1-alpha) (Desmoglein- |
| | | 1) (Desmosomal glycoprotein I) (DG1) (DGI) |
| AI502114 | Abca1 | ATP-binding cassette, sub-family A (ABC1), member 1 |
| AI502349 | — | Transcribed locus |
| AI502459 | — | — |
| AI502837 | — | Transcribed locus |
| AI535113 | — | Transcribed locus |
| AI547876 | — | Transcribed locus |
| AI547937 | — | Transcribed locus |
| AI548897 | Soat1 | Sterol O-acyltransferase 1 |
| AI549199 | Ptger4 | Prostaglandin E receptor 4 (subtype EP4) |
| AI549209 | — | Transcribed locus |
| AI549335 | Dock11 | dedicator of cytokinesis 11 |
| AI549450 | Cd209b | CD209b antigen |
| AI555257 | RGD1562305_predicted | similar to suprabasal-specific protein suprabasin |
| | | (predicted) |
| AI555447 | RGD1565975_predicted | RGD1565975 (predicted) |
| AI555526 | — | Transcribed locus |
| AI555855 | — | Transcribed locus |
| AI556056 | Cr2_predicted | complement receptor 2 (predicted) |
| AI556235 | RGD1310061 | similar to hypothetical protein FLJ10154 |
| AI556638 | — | — |
| AI556957 | Aff4_predicted | AF4/FMR2 family, member 4 (predicted) |
| AI575442 | Ryr1 | ryanodine receptor 1, skeletal muscle |
| AI576002 | — | Transcribed locus |
| AI576184 | — | Transcribed locus |
| AI576518 | — | Transcribed locus |
| AI577567 | Rasal2_predicted | RAS protein activator like 2 (predicted) |
| AI578087 | Tm4sf1_predicted | transmembrane 4 superfamily member 1 (predicted) |
| AI578566 | Ubn1_predicted | ubinuclein 1 (predicted) |
| AI599133 | LOC363060 | similar to RIKEN cDNA 1600029D21 |
| AI599423 | Gadd45g | growth arrest and DNA-damage-inducible 45 gamma |
| AI599463 | Dguok_predicted | deoxyguanosine kinase (predicted) |
| AI638986 | Neb_predicted | nebulin (predicted) |
| AI639108 | — | — |
| AI639117 | Cfb | complement factor B |
| AI706673 | Wasl | Wiskott-Aldrich syndrome-like (human) |
| AI710604 | — | Transcribed locus |
| AI713204 | Mgll | Monoglyceride lipase |
| AI714002 | Mki67_predicted | antigen identified by monoclonal antibody Ki-67 |
| | | (predicted) |
| AI715999 | LOC688864 | similar to transmembrane protein 61 |
| AI716456 | Tiam1 | T-cell lymphoma invasion and metastasis 1 |
| AI716987 | Dsg1c_predicted /// | desmoglein 1 gamma (predicted) /// similar to |
| | LOC679010 | Desmoglein-1 alpha precursor (Dsg1-alpha) (Desmoglein- |
| | | 1) (Desmosomal glycoprotein I) (DG1) (DGI) |
| AI717644 | RGD1563764_predicted | similar to PHD finger protein 14 isoform 1 (predicted) |
| AI948410 | — | — |
| AJ243304 | Trdn | Triadin |
| AW141642 | RGD1306067 | similar to chromosome 20 open reading frame 6 |
| AW142773 | — | Transcribed locus |
| AW142978 | Traf3ip3 | TRAF3 interacting protein 3 |
| AW144132 | — | Transcribed locus |
| AW144509 | Dsp | Desmoplakin |
| AW144823 | Sorl1 | sortilin-related receptor, LDLR class A repeats-containing |
| AW251280 | — | — |
| AW251860 | Tcf7_predicted | transcription factor 7, T-cell specific (predicted) |
| AW433901 | Celsr1 | cadherin EGF LAG seven-pass G-type receptor 1 |
| AW433947 | Tnfrsf5 | tumor necrosis factor receptor superfamily, member 5 |
| AW434166 | Nicn1 | nicolin 1 |
| AW434972 | Rbm25_predicted /// | RNA binding motif protein 25 (predicted) /// similar to |
| | RGD1565486_predicted | RNA binding motif protein 25 (predicted) |
| AW522357 | — | Transcribed locus |
| AW523955 | — | Transcribed locus |
| AW524359 | — | Transcribed locus |
| AW526072 | — | Transcribed locus |
| AW526268 | RGD1561817_predicted | Similar to Traf2 and NCK interacting kinase, splice |
| | | variant 4 (predicted) |
| AW526631 | Sorl1 | sortilin-related receptor, LDLR class A repeats-containing |
| AW526982 | Tlr2 | toll-like receptor 2 |
| AW527270 | RGD1309360 | hypothetical LOC294715 |
| AW527313 | Rdx | Radixin |
| AW528106 | — | Transcribed locus |
| AW528448 | — | Transcribed locus |
| AW529736 | Wfdc5_predicted | WAP four-disulfide core domain 5 (predicted) |
| AW530769 | Fdft1 | farnesyl diphosphate farnesyl transferase 1 |
| AW530812 | RGD1311224_predicted | similar to fatty acid desaturase 2; linoleoyl-CoA |
| | | desaturase (delta-6-desaturase)-like 2; delta-6 fatty acid |
| | | desaturase (predicted) |
| AW531135 | — | Transcribed locus |
| AW532114 | Rasgrp2_predicted | RAS guanyl releasing protein 2 (calcium and DAG- |
| | | regulated) (predicted) |
| AW532165 | Sema3d | sema domain, immunoglobulin domain (Ig), short basic |
| | | domain, secreted, (semaphorin) 3D |
| AW532179 | LOC500449 | similar to SHP2-interacting transmembrane adaptor |
| | | protein |
| AW533234 | LOC688915 | similar to cardiomyopathy associated 5 |
| AW533569 | Clca2_predicted | chloride channel, calcium activated, family member 2 |
| | | (predicted) |
| AW534046 | — | — |
| AW534218 | RGD1310722_predicted | similar to RIKEN cDNA D130059P03 gene (predicted) |
| AW534561 | — | Transcribed locus |
| AW534965 | RGD1560358_predicted | similar to cell division cycle and apoptosis regulator 1 |
| | | (predicted) |
| AW915049 | — | Transcribed locus |
| AW915948 | Cd3e_predicted | CD3 antigen, epsilon polypeptide (predicted) |
| AW916093 | — | Transcribed locus |
| AW917933 | — | Transcribed locus |
| AW918480 | — | Transcribed locus |
| AW918614 | Spnb2 | spectrin beta 2 |
| AW919180 | Pygm | muscle glycogen phosphorylase |
| AW919569 | MGC156825 | similar to MIR-interacting saposin-like protein precursor |
| | | (Transmembrane protein 4) (Putative secreted protein |
| | | ZSIG9) |
| AW920026 | LOC364393 /// | PHD finger protein 11 /// similar to RIKEN cDNA |
| | LOC684778 /// | 4933417L10 /// hypothetical protein LOC684778 |
| | Phf11 | |
| AW920881 | Actr2 /// | ARP2 actin-related protein 2 homolog (yeast) /// similar to |
| | LOC301861 | ARP2 actin-related protein 2 homolog (yeast) (predicted) |
| BE096652 | Ly6g6c | lymphocyte antigen 6 complex, locus G6C |
| BE096750 | RGD1305928_predicted | hypothetical LOC300207 (predicted) |
| BE096812 | — | Transcribed locus |
| BE097933 | Mitf | microphthalmia-associated transcription factor |
| BE098153 | Slc24a3 | solute carrier family 24 (sodium/potassium/calcium |
| | | exchanger), member 3 |
| BE098726 | — | Transcribed locus |
| BE098732 | Uhrf1 | ubiquitin-like, containing PHD and RING finger domains, 1 |
| BE099050 | Nfib | nuclear factor I/B |
| BE099838 | — | Non-coding RNA expressed in the brain, repeat sequence, |
| | | clone 12 |
| BE100543 | Ncor1 | nuclear receptor co-repressor 1 |
| BE100811 | Epb4.1l4a_predicted | erythrocyte protein band 4.1-like 4a (predicted) |
| BE102340 | — | — |
| BE102861 | — | Transcribed locus |
| BE104676 | — | Transcribed locus, strongly similar to NP_001029332.1 |
| | | hypothetical protein LOC619476 [Rattus norvegicus] |
| BE104961 | Cenpj_predicted | centromere protein J (predicted) |
| BE105050 | — | Transcribed locus |
| BE105498 | — | Transcribed locus |
| BE106199 | Rora_predicted | RAR-related orphan receptor alpha (predicted) |
| BE106791 | — | Transcribed locus |
| BE106891 | — | Transcribed locus |
| BE107024 | — | Transcribed locus |
| BE107282 | Vof16 | ischemia related factor vof-16 |
| BE107351 | — | Transcribed locus |
| BE108131 | Wdfy1 | WD repeat and FYVE domain containing 1 |
| BE108135 | — | Transcribed locus |
| BE108165 | Cep1_predicted | centrosomal protein 1 (predicted) |
| BE108294 | — | Transcribed locus |
| BE108409 | — | Transcribed locus |
| BE108587 | Nisch | Nischarin |
| BE108716 | — | Transcribed locus |
| BE108758 | — | Transcribed locus |
| BE108849 | — | Transcribed locus |
| BE108905 | Nap1l1 | Nucleosome assembly protein 1-like 1 |
| BE109260 | — | Transcribed locus |
| BE109322 | Plk4_predicted | polo-like kinase 4 (Drosophila) (predicted) |
| BE109926 | Pik3cd_predicted | phosphatidylinositol 3-kinase catalytic delta polypeptide |
| | | (predicted) |
| BE110332 | RGD1307829_predicted /// | similar to chromosome 14 open reading frame 6 |
| | Rnase11 | (predicted) /// ribonuclease 11 |
| BE110369 | Arhgef18_predicted | rho/rac guanine nucleotide exchange factor (GEF) 18 |
| | | (predicted) |
| BE110674 | LOC686259 /// | hypothetical protein LOC686259 /// hypothetical protein |
| | LOC690243 | LOC690243 |
| BE111706 | Marcks | myristoylated alanine rich protein kinase C substrate |
| BE112261 | Ctdspl_predicted | CTD (carboxy-terminal domain, RNA polymerase II, |
| | | polypeptide A) small phosphatase-like (predicted) |
| BE112895 | Pea15 | phosphoprotein enriched in astrocytes 15 |
| BE112927 | Cyfip2_predicted | cytoplasmic FMR1 interacting protein 2 (predicted) |
| BE113247 | — | — |
| BE113270 | Igfbp5 | insulin-like growth factor binding protein 5 |
| BE113961 | — | Transcribed locus |
| BE115061 | Lef1 | Lymphoid enhancer binding factor 1 |
| BE115155 | Rhpn2_predicted | rhophilin, Rho GTPase binding protein 2 (predicted) |
| BE115432 | Utrn | Utrophin |
| BE115521 | RGD1310433_predicted | similar to mKIAA1757 protein (predicted) |
| BE115641 | — | — |
| BE116021 | Slc5a3 | solute carrier family 5 (inositol transporters), member 3 |
| BE116111 | — | Transcribed locus |
| BE116127 | — | — |
| BE116152 | Elovl6 | ELOVL family member 6, elongation of long chain fatty |
| | | acids (yeast) |
| BE116698 | RGD1564964_predicted | similar to WD repeat domain 11 protein (predicted) |
| BE116855 | — | CDNA clone IMAGE: 7384525 |
| BE117044 | Cd8a | CD8 antigen, alpha chain |
| BE117361 | LOC681423 /// | Nipped-B homolog (Drosophila) /// similar to delangin |
| | NIPBL | isoform A |
| BE117736 | Tgfb2 | Transforming growth factor, beta 2 |
| BE118096 | — | Transcribed locus |
| BE118580 | — | Transcribed locus |
| BE118901 | Gata3 | GATA binding protein 3 |
| BE119167 | — | Transcribed locus |
| BE120370 | — | Transcribed locus |
| BE120660 | — | Transcribed locus |
| BE120816 | — | Transcribed locus |
| BE120823 | Smc4l1 | SMC4 structural maintenance of chromosomes 4-like 1 |
| | | (yeast) |
| BE120852 | — | Transcribed locus |
| BE126475 | Prrx1 | paired related homeobox 1 |
| BE328934 | RGD1310958_predicted | similar to RIKEN cDNA C130090K23 (predicted) |
| BE328951 | Cldn4 | claudin 4 |
| BE329244 | — | — |
| BF281337 | Krt2-8 | keratin complex 2, basic, gene 8 |
| BF282187 | — | Transcribed locus |
| BF283053 | Atp6v1c2 | ATPase, H+ transporting, V1 subunit C, isoform 2 |
| BF283556 | Plxnc1_predicted | plexin C1 (predicted) |
| BF283610 | — | Transcribed locus |
| BF283642 | LOC363060 | similar to RIKEN cDNA 1600029D21 |
| BF283924 | Lamp3 | lysosomal-associated membrane protein 3 |
| BF284262 | Irf8 | Interferon regulatory factor 8 |
| BF285731 | — | Transcribed locus |
| BF287629 | Il27ra_predicted | interleukin 27 receptor, alpha (predicted) |
| BF288000 | — | — |
| BF288109 | — | Transcribed locus |
| BF288130 | Ptprc | protein tyrosine phosphatase, receptor type, C |
| BF288177 | Csnk2a1 | casein kinase II, alpha 1 polypeptide |
| BF288361 | — | — |
| BF288533 | — | Transcribed locus |
| BF289150 | — | Transcribed locus |
| BF290113 | RGD1311584 | similar to stem cell adaptor protein STAP-1 |
| BF290181 | — | — |
| BF290410 | — | Transcribed locus |
| BF290784 | — | — |
| BF386742 | — | Transcribed locus |
| BF387238 | RGD1311117_predicted | similar to KIAA1409 protein (predicted) |
| BF387865 | — | Transcribed locus |
| BF388585 | Znf292 | zinc finger protein 292 |
| BF389793 | Crabp1 | cellular retinoic acid binding protein 1 |
| BF390257 | RGD1309863 | similar to hypothetical protein DKFZp434K1421 |
| BF391127 | Arid4a_predicted | AT rich interactive domain 4A (Rbp1 like) (predicted) |
| BF393607 | LOC689147 | hypothetical protein LOC689147 |
| BF394095 | — | Transcribed locus |
| BF395171 | Ehd4 | EH-domain containing 4 |
| BF396210 | Trim59_predicted | tripartite motif-containing 59 (predicted) |
| BF396282 | Kif15 | kinesin family member 15 |
| BF396319 | — | Transcribed locus |
| BF397627 | — | Transcribed locus |
| BF397719 | Plekhg2_predicted | pleckstrin homology domain containing, family G (with |
| | | RhoGef domain) member 2 (predicted) |
| BF398050 | Chd6_predicted | chromodomain helicase DNA binding protein 6 |
| | | (predicted) |
| BF398091 | — | CDNA clone IMAGE: 7374368 |
| BF398122 | — | Synaptogenesis-related mRNA sequence 6 |
| BF398614 | — | Transcribed locus |
| BF399329 | — | — |
| BF399587 | — | Transcribed locus |
| BF399855 | Serpinb10 | serine (or cysteine) peptidase inhibitor, clade B |
| | | (ovalbumin), member 10 |
| BF401586 | — | Transcribed locus |
| BF402012 | Zbtb20_predicted | zinc finger and BTB domain containing 20 (predicted) |
| BF404462 | — | Transcribed locus |
| BF405616 | — | Transcribed locus |
| BF406973 | RGD1311490_predicted | similar to DKFZP434P1750 protein (predicted) |
| BF408105 | — | — |
| BF409092 | — | Transcribed locus |
| BF410101 | — | Transcribed locus |
| BF410240 | — | — |
| BF410953 | — | — |
| BF411408 | — | — |
| BF414018 | Tmem97 | transmembrane protein 97 |
| BF414285 | — | Transcribed locus |
| BF415030 | Pigp_predicted | phosphatidylinositol glycan, class P (predicted) |
| BF416343 | LOC681423 /// | Nipped-B homolog (Drosophila) /// similar to delangin |
| | NIPBL | isoform A |
| BF416764 | LOC363326 | hypothetical LOC363326 |
| BF417048 | LOC499300 | similar to protein tyrosine phosphatase, receptor type, C |
| | | polypeptide-associated protein |
| BF417079 | — | Transcribed locus |
| BF417479 | Dhcr24 | 24-dehydrocholesterol reductase |
| BF417582 | Myo1g | myosin IG |
| BF417625 | — | Transcribed locus |
| BF418025 | Grap | GRB2-related adaptor protein |
| BF418487 | Med31_predicted | mediator of RNA polymerase II transcription, subunit 31 |
| | | homolog (yeast) (predicted) |
| BF418522 | — | Transcribed locus |
| BF418649 | RGD1305645_predicted | similar to RIKEN cDNA 1500015O10 (predicted) |
| BF419134 | Acbd3 | acyl-Coenzyme A binding domain containing 3 |
| BF420720 | RGD1563128_predicted | similar to ADP-ribosylation-like factor 12 protein |
| | | (predicted) |
| BF420722 | Nfix | nuclear factor I/X |
| BF420803 | — | Transcribed locus |
| BF521859 | Tnnt3 | troponin T3, skeletal, fast |
| BF522317 | — | Transcribed locus |
| BF523248 | Ankrd11_predicted | ankyrin repeat domain 11 (predicted) |
| BF524010 | — | — |
| BF542615 | Ptplad2_predicted | protein tyrosine phosphatase-like A domain containing 2 |
| | | (predicted) |
| BF545080 | Cox6b2 | cytochrome c oxidase subunit VIb testes-specific isoform |
| | | precursor |
| BF545930 | — | Transcribed locus |
| BF547251 | LOC685707 /// | neuron navigator 1 (predicted) /// similar to neuron |
| | Nav1_predicted | navigator 1 |
| BF548081 | — | — |
| BF553172 | — | Transcribed locus |
| BF554283 | Ogfrl1 | opioid growth factor receptor-like 1 |
| BF555968 | Nedd9 | neural precursor cell expressed, developmentally down- |
| | | regulated gene 9 |
| BF555972 | RGD1359529 | similar to chromosome 1 open reading frame 63 |
| BF556622 | — | — |
| BF558056 | — | — |
| BF558512 | — | Transcribed locus |
| BF558946 | LOC688717 | similar to CG33714-PB, isoform B |
| BF561454 | Fgl2 | fibrinogen-like 2 |
| BF562507 | Sorl1 | sortilin-related receptor, LDLR class A repeats-containing |
| BF565167 | Rdx | Radixin |
| BF565756 | — | Transcribed locus |
| BG371683 | — | Transcribed locus, moderately similar to NP_598678.1 |
| | | mondoA isoform 2 [Mus musculus] |
| BG371843 | Pkp1_predicted | plakophilin 1 (predicted) |
| BG372056 | — | Transcribed locus |
| BG372342 | RGD1311019_predicted | similar to hypothetical protein DKFZp434H2010 |
| | | (predicted) |
| BG372602 | RGD1306601_predicted | similar to cDNA sequence BC019755 (predicted) |
| BG372891 | — | Transcribed locus |
| BG372987 | — | — |
| BG373166 | Elf5_predicted | E74-like factor 5 (predicted) |
| BG373580 | Chchd1_predicted | coiled-coil-helix-coiled-coil-helix domain containing 1 |
| | | (predicted) |
| BG374493 | Cyp2j10_predicted | cytochrome P450, family 2, subfamily j, polypeptide 10 |
| | | (predicted) |
| BG375315 | Sdc1 | syndecan 1 |
| BG376030 | RGD1306107_predicted | similar to chromosome 1 open reading frame 2 (predicted) |
| BG376644 | Rsbn1l_predicted | round spermatid basic protein 1-like (predicted) |
| BG377116 | Atp6v0a4_predicted | ATPase, H+ transporting, lysosomal V0 subunit A isoform |
| | | 4 (predicted) |
| BG377979 | LOC690528 | similar to POU domain class 2, associating factor 1 (B- |
| | | cell-specific coactivator OBF-1) (OCT binding factor 1) |
| | | (BOB-1) (BOB1) (OCA-B) |
| BG378166 | Map4k2_predicted | mitogen activated protein kinase kinase kinase kinase 2 |
| | | (predicted) |
| BG378310 | Scel_predicted | sciellin (predicted) |
| BG378588 | Mybpc2_predicted | myosin binding protein C, fast-type (predicted) |
| BG378607 | LOC362350 /// Tcrb | T-cell receptor beta chain /// similar to T-cell receptor |
| | | beta-2 chain C region |
| BG378613 | Agpat3_predicted | 1-acylglycerol-3-phosphate O-acyltransferase 3 |
| | | (predicted) |
| BG378874 | Wbp5_predicted | WW domain binding protein 5 (predicted) |
| BG378912 | Wdr47 | WD repeat domain 47 |
| BG379338 | Rrm2 | ribonucleotide reductase M2 |
| BG380122 | Serpinb6b | serine (or cysteine) proteinase inhibitor, clade B, member |
| | | 6b |
| BG380816 | LOC291411 | similar to Potential phospholipid-transporting ATPase IIB |
| BG381555 | — | CDNA clone IMAGE: 7388962 |
| BG662519 | Ank | Progressive ankylosis homolog (mouse) |
| BG663208 | Jak1 | Janus kinase 1 |
| BG664221 | Ogn_predicted | osteoglycin (predicted) |
| BG665051 | — | — |
| BG665934 | Oact2 | O-acyltransferase (membrane bound) domain containing 2 |
| BG668816 | Etnk1_predicted | ethanolamine kinase 1 (predicted) |
| BG668988 | Elovl4_predicted | elongation of very long chain fatty acids (FEN1/Elo2, |
| | | SUR4/Elo3, yeast)-like 4 (predicted) |
| BG670310 | Tgfa | transforming growth factor alpha |
| BG670822 | — | Non-coding RNA expressed in the brain, repeat sequence, |
| | | clone 2 /// Non-coding RNA expressed in the brain, repeat |
| | | sequence, clone 7 /// Non-coding RNA expressed in the |
| | | brain, repeat sequence, clone 3 /// Non-coding RNA |
| | | expressed in the brain, repeat sequence, clone 12 |
| BG671521 | Hspca | heat shock protein 1, alpha |
| BG671686 | — | — |
| BG672572 | — | Transcribed locus |
| BG673248 | — | Transcribed locus |
| BI274308 | Ttc7b_predicted | tetratricopeptide repeat domain 7B (predicted) |
| BI274326 | Muc1 | mucin 1, transmembrane |
| BI274480 | Fnbp4 | formin binding protein 4 |
| BI274533 | RGD1309534 | similar to RIKEN cDNA 4931406C07 |
| BI275633 | Pygm | muscle glycogen phosphorylase |
| BI275677 | — | — |
| BI275765 | Uhrf2_predicted | ubiquitin-like, containing PHD and RING finger domains |
| | | 2 (predicted) |
| BI275827 | — | Transcribed locus |
| BI275873 | Centb1_predicted | centaurin, beta 1 (predicted) |
| BI276075 | — | Transcribed locus |
| BI276959 | LOC679341 /// | similar to Calsequestrin-1 precursor (Calsequestrin, |
| | LOC686019 | skeletal muscle isoform) |
| BI277513 | LOC360975 | similar to oxoglutarate dehydrogenase (lipoamide) |
| BI277545 | Myh1 /// Myh2 | myosin, heavy polypeptide 1, skeletal muscle, adult /// |
| | | myosin, heavy polypeptide 2, skeletal muscle, adult |
| BI278180 | — | Transcribed locus |
| BI278186 | Crct1_predicted | cysteine-rich C-terminal 1 (predicted) |
| BI278721 | Calm4_predicted | calmodulin 4 (predicted) |
| BI278952 | — | — |
| BI279486 | LOC494538 | ABP beta |
| BI279605 | Krt1-19 | keratin complex 1, acidic, gene 19 |
| BI279646 | Krt1-14 | keratin complex 1, acidic, gene 14 |
| BI279663 | Dsc2 | desmocollin 2 |
| BI279786 | Myo1d | myosin ID |
| BI280124 | RGD1565500_predicted | RGD1565500 (predicted) |
| BI282076 | Prdx4 | peroxiredoxin 4 |
| BI282114 | — | Endogenous retrovirus mRNA, partial sequence |
| BI282164 | Cyb561_predicted | cytochrome b-561 (predicted) |
| BI282567 | Klk8 | kallikrein 8 (neuropsin/ovasin) |
| BI282568 | Krtdap | keratinocyte differentiation associated protein |
| BI282576 | RGD1310935_predicted | similar to Dermal papilla derived protein 7 (predicted) |
| BI282584 | RGD1560328_predicted | similar to UPF0197 protein C11orf10 homolog (predicted) |
| BI282694 | RGD1565037_predicted | similar to selenoprotein SelM (predicted) |
| BI282819 | — | Transcribed locus |
| BI282860 | LOC685652 | similar to keratin 6L |
| BI282914 | Kctd14_predicted | potassium channel tetramerisation domain containing 14 |
| | | (predicted) |
| BI283346 | Klk10 | kallikrein 10 |
| BI283648 | — | Transcribed locus, moderately similar to XP_919392.1 |
| | | PREDICTED: hypothetical protein XP_914199 |
| | | [Mus musculus] |
| BI285064 | — | Transcribed locus |
| BI285065 | — | Transcribed locus |
| BI285092 | LOC682044 /// | hypothetical protein LOC682044 /// hypothetical protein |
| | LOC684972 | LOC684972 |
| BI285443 | RGD1560859_predicted | similar to 2300003P22Rik protein (predicted) |
| BI285676 | — | Transcribed locus |
| BI285700 | Hspcb | heat shock 90 kDa protein 1, beta |
| BI285951 | RGD1308734 | similar to RIKEN cDNA 1100001H23 |
| BI286012 | Krt1-18 | keratin complex 1, acidic, gene 18 |
| BI286166 | LOC298795 /// | similar to 14-3-3 protein sigma /// stratifin (predicted) |
| | Sfn_predicted | |
| BI286340 | RGD1305779_predicted | similar to NSE1 (predicted) |
| BI286387 | LOC499660 | similar to Cornifin A (Small proline-rich protein 1A) |
| | | (SPR1A) (SPRR1) |
| BI286389 | Ka17 | type I keratin KA17 |
| BI286396 | Perp_predicted | PERP, TP53 apoptosis effector (predicted) |
| BI286411 | — | Ultraviolet B radiation-activated UV103 mRNA, partial |
| | | sequence |
| BI286411 | LOC683295 | similar to keratin complex 2, basic, gene 6a |
| BI286421 | — | Transcribed locus, strongly similar to NP_001007709.1 |
| | | hypothetical protein LOC315126 [Rattus norvegicus] |
| BI288162 | — | Transcribed locus |
| BI288898 | LOC680692 /// | similar to Golgi phosphoprotein 2 (Golgi membrane |
| | LOC682869 | protein GP73) |
| BI289371 | Rhbdl6_predicted | rhomboid, veinlet-like 6 (Drosophila) (predicted) |
| BI289386 | Bcor_predicted | Bcl6 interacting corepressor (predicted) |
| BI289641 | RGD1566064_predicted | similar to KIAA1096 protein (predicted) |
| BI289662 | LOC690326 | hypothetical protein LOC690326 |
| BI289840 | — | Transcribed locus |
| BI290053 | — | Transcribed locus |
| BI290551 | Fnbp1 | Formin binding protein 1 |
| BI290737 | — | Transcribed locus |
| BI290909 | Scap1 | src family associated phosphoprotein 1 |
| BI291230 | — | — |
| BI291604 | LOC687609 | similar to ras homolog gene family, member f |
| BI291798 | — | Immunoglobulin delta heavy chain transmembrane form |
| | | (IgD) |
| BI291804 | — | Transcribed locus |
| BI292034 | — | — |
| BI292036 | LOC683839 /// | hypothetical protein LOC683839 /// hypothetical protein |
| | LOC688495 | LOC688495 |
| BI292056 | — | Transcribed locus |
| BI292090 | Cldn8 | claudin 8 |
| BI292231 | Lpo_predicted | lactoperoxidase (predicted) |
| BI292758 | — | Transcribed locus |
| BI293796 | Nckap1l_predicted | NCK associated protein 1 like (predicted) |
| BI294141 | Angpt4_predicted | angiopoietin 4 (predicted) |
| BI294158 | — | Transcribed locus |
| BI294178 | — | Transcribed locus |
| BI294722 | — | Transcribed locus, weakly similar to XP_487464.2 |
| | | PREDICTED: similar to zinc finger protein 85, related |
| | | sequence 1 [Mus musculus] |
| BI294788 | — | Transcribed locus |
| BI295614 | — | Transcribed locus, strongly similar to NP_001041317.1 |
| | | BCL2-associated transcription factor 1 [Rattus norvegicus] |
| BI295900 | Dlat | dihydrolipoamide S-acetyltransferase (E2 component of |
| | | pyruvate dehydrogenase complex) |
| BI296153 | Acaca | acetyl-coenzyme A carboxylase alpha |
| BI296641 | — | Transcribed locus |
| BI297744 | RGD1310507 | similar to RIKEN cDNA 1300017J02 |
| BI299761 | — | Transcribed locus |
| BI301117 | — | Transcribed locus |
| BI301495 | LOC688276 | similar to epidermodysplasia verruciformis 2 |
| BI302005 | Mlf1_predicted | myeloid leukemia factor 1 (predicted) |
| BI303106 | Traf3_predicted | Tnf receptor-associated factor 3 (predicted) |
| BI395810 | Npy1r | neuropeptide Y receptor Y1 |
| BM382988 | — | Transcribed locus |
| BM383065 | RGD1310587 | similar to hypothetical protein FLJ14146 |
| BM383423 | Ehf_predicted | ets homologous factor (predicted) |
| BM383428 | LOC367976 /// | minichromosome maintenance deficient 3 (S. cerevisiae) |
| | Mcm3_predicted | (predicted) /// similar to DNA replication licensing factor |
| | | MCM3 (DNA polymerase alpha holoenzyme-associated |
| | | protein P1) (P1-MCM3) |
| BM383464 | — | Transcribed locus |
| BM383995 | — | Transcribed locus |
| BM384008 | Arhgap5 | Rho GTPase activating protein 5 |
| BM385071 | RGD1563060_predicted | similar to AVLV472 (predicted) |
| BM385951 | — | Transcribed locus |
| BM386010 | RGD1359684 | similar to T-cell receptor alpha chain precursor V and C |
| | | regions (TRA29) |
| BM386036 | — | Transcribed locus, strongly similar to XP_345296.2 |
| | | PREDICTED: similar to AVIEF [Rattus norvegicus] |
| BM386334 | Ankrd23_predicted | ankyrin repeat domain 23 (predicted) |
| BM386413 | Trim2 | tripartite motif protein 2 |
| BM387106 | Amid_predicted | Apoptosis-inducing factor (AIF)-like mitochondrion- |
| | | associated inducer of death (predicted) |
| BM387112 | — | Transcribed locus |
| BM387125 | — | Transcribed locus |
| BM387197 | — | — |
| BM387754 | — | Transcribed locus |
| BM387797 | LOC360619 | similar to gasdermin 1 |
| BM387946 | Zap70 | zeta-chain (TCR) associated protein kinase 70 |
| BM388077 | RGD1562629_predicted | similar to neurobeachin (predicted) |
| BM388319 | Gimap7 | GTPase, IMAP family member 7 |
| BM388334 | Spink5_predicted | serine protease inhibitor, Kazal type 5 (predicted) |
| BM388738 | — | — |
| BM389005 | Gpr68_predicted | G protein-coupled receptor 68 (predicted) |
| BM389254 | Anxa8 | annexin A8 |
| BM389444 | — | Transcribed locus |
| BM389513 | LOC688090 /// | RT1 class II, locus Bb /// similar to RT1 class II |
| | RT1-Bb | histocompatibility antigen, B-1 beta chain precursor |
| | | (RT1.B-beta(1)) |
| BM389585 | Capn13 | calpain 13 |
| BM390176 | Spink5_predicted | serine protease inhibitor, Kazal type 5 (predicted) |
| BM390226 | — | Transcribed locus |
| BM390227 | Bcel11b_predicted | B-cell leukemia/lymphoma 11B (predicted) |
| BM390324 | Eppb9_predicted | endothelial precursor protein B9 (predicted) |
| BM390325 | RGD1562868_predicted | similar to squamous cell carcinoma antigen 2 (predicted) |
| BM390378 | RGD1562732_predicted | similar to Glutathione S-transferase, theta 3 (predicted) |
| BM390399 | Hmgcr | 3-hydroxy-3-methylglutaryl-Coenzyme A reductase |
| BM390440 | — | Transcribed locus |
| BM390872 | RGD1560293_predicted | similar to RIKEN cDNA 1200013B08 (predicted) |
| BM390965 | RGD1305592 | similar to RIKEN cDNA 2900092E17 |
| BM391096 | — | Transcribed locus |
| BM391169 | Myh4 | myosin, heavy polypeptide 4 |
| BM391471 | Atf5 | activating transcription factor 5 |
| BM391538 | Frzb | frizzled-related protein |
| BM392106 | Cald1 | caldesmon 1 |
| BM392321 | Hipk2_predicted | homeodomain interacting protein kinase 2 (predicted) |
| D13555 | Cd3z | CD3 antigen, zeta polypeptide |
| D50306 | Slc15a1 | solute carrier family 15 (oligopeptide transporter), member 1 |
| D88586 | Ear11 | eosinophil-associated, ribonuclease A family, member 11 |
| H31078 | — | — |
| J00710 | Csn1s1 | casein alpha s1 |
| J02612 | Ugt1a1 /// | UDP glycosyltransferase 1 family, polypeptide A1 /// |
| | Ugt1a10 /// | UDP glycosyltransferase 1 family, polypeptide A6 /// |
| | Ugt1a2 /// | UDP glycosyltransferase 1 family, polypeptide A7 /// |
| | Ugt1a3 /// | UDP glycosyltransferase 1 family, polypeptide A8 /// |
| | Ugt1a5 /// | UDP glycosyltransferase 1 family polypeptide A2 /// |
| | Ugt1a6 /// | UDP glycosyltransferase 1 family polypeptide A3 /// |
| | Ugt1a7 /// | UDP glycosyltransferase 1 family polypeptide A10 /// |
| | Ugt1a8 | UDP glycosyltransferase 1 family, polypeptide A5 |
| J02705 | Ocm | Oncomodulin |
| J03867 | Cyb5r3 | cytochrome b5 reductase 3 |
| L17138 | Hsd3b6 | hydroxy-delta-5-steroid dehydrogenase, 3 beta- and steroid |
| | | delta-isomerase 6 |
| L19933 | Ptprd | protein tyrosine phosphatase, receptor type, D |
| L22654 | IgG-2a | gamma-2a immunoglobulin heavy chain |
| M10072 | Ptprc | protein tyrosine phosphatase, receptor type, C |
| M13706 | Prkcb1 | protein kinase C, beta 1 |
| M17153 | Fcer1a | Fc receptor, IgE, high affinity I, alpha polypeptide |
| M24024 | RT1-Aw2 | RT1 class Ib, locus Aw2 |
| M30596 | Me1 | malic enzyme 1 |
| M61142 | Thop1 | thimet oligopeptidase 1 |
| NM_012488 | A2m | alpha-2-macroglobulin |
| NM_012502 | Ar | androgen receptor |
| NM_012524 | Cebpa | CCAAT/enhancer binding protein (C/EBP), alpha |
| NM_012532 | Cp | Ceruloplasmin |
| NM_012555 | Ets1 | v-ets erythroblastosis virus E26 oncogene homolog 1 |
| | | (avian) |
| NM_012559 | Fgg | fibrinogen, gamma polypeptide |
| NM_012594 | Lalba | lactalbumin, alpha |
| NM_012600 | Me1 | malic enzyme 1 |
| NM_012640 | Rbp2 | retinol binding protein 2, cellular |
| NM_012646 | RT1-N1 /// | RT1 class Ib gene, H2-TL-like, grc region (N1) /// |
| | RT1-N2 /// | RT1 class Ib gene, H2-TL-like, grc region (N3) /// |
| | RT1-N3 | RT1 class Ib gene, H2-TL-like, grc region(N2) |
| NM_012703 | Thrsp | thyroid hormone responsive protein |
| NM_012745 | Klrd1 | killer cell lectin-like receptor, subfamily D, member 1 |
| NM_012760 | Plagl1 | pleiomorphic adenoma gene-like 1 |
| NM_012777 | Apod | apolipoprotein D |
| NM_012794 | Glycam1 | glycosylation dependent cell adhesion molecule 1 |
| NM_012812 | Cox6a2 | cytochrome c oxidase, subunit VIa, polypeptide 2 |
| NM_012824 | Apoc1 | apolipoprotein C-I |
| NM_012888 | Tshr | thyroid stimulating hormone receptor |
| NM_012893 | Actg2 | actin, gamma 2 |
| NM_012938 | Ctse | cathepsin E |
| NM_012949 | Eno3 | enolase 3, beta |
| NM_013026 | Sdc1 | syndecan 1 |
| NM_013041 | Sycp3 | synaptonemal complex protein 3 |
| NM_013092 | Cma1 | chymase 1, mast cell |
| NM_013134 | Hmgcr | 3-hydroxy-3-methylglutaryl-Coenzyme A reductase |
| NM_013169 | Cd3d | CD3 antigen delta polypeptide |
| NM_013186 | Kcnb1 | potassium voltage gated channel, Shab-related subfamily, |
| | | member 1 |
| NM_013200 | Cpt1b | carnitine palmitoyltransferase 1b, muscle |
| NM_016986 | Acadm | acetyl-Coenzyme A dehydrogenase, medium chain |
| NM_016998 | Cpa1 | carboxypeptidase A1 |
| NM_017028 | Mx2 | myxovirus (influenza virus) resistance 2 |
| NM_017120 | Csn2 | casein beta |
| NM_017124 | Cd37 | CD37 antigen |
| NM_017156 | Cyp2b15 | cytochrome P450, family 2, subfamily b, polypeptide 15 |
| NM_017206 | Slc6a6 | solute carrier family 6 (neurotransmitter transporter, |
| | | taurine), member 6 |
| NM_017259 | Btg2 | B-cell translocation gene 2, anti-proliferative |
| NM_017328 | Pgam2 | phosphoglycerate mutase 2 |
| NM_019131 | Tpm1 | tropomyosin 1, alpha |
| NM_019171 | LOC685608 /// | salivary protein 1 /// hypothetical protein LOC685608 /// |
| | LOC688807 /// Spt1 | hypothetical protein LOC688807 |
| NM_019175 | Klk6 | kallikrein 6 |
| NM_019206 | Stk10 | serine/threonine kinase 10 |
| NM_019212 | Acta1 | actin, alpha 1, skeletal muscle |
| NM_019225 | Slc1a3 | solute carrier family 1 (glial high affinity glutamate |
| | | transporter), member 3 |
| NM_019238 | Fdft1 | farnesyl diphosphate farnesyl transferase 1 |
| NM_019240 | Gjb3 | gap junction membrane channel protein beta 3 |
| NM_019241 | Gjb5 | gap junction membrane channel protein beta 5 |
| NM_019282 | Grem1 | gremlin 1 homolog, cysteine knot superfamily |
| | | (Xenopus laevis) |
| NM_019291 | Ca2 | carbonic anhydrase 2 |
| NM_019295 | Cd5 | CD5 antigen |
| NM_019334 | Pitx2 | paired-like homeodomain transcription factor 2 |
| NM_019371 | Egln3 | EGL nine homolog 3 (C. elegans) |
| NM_020072 | Acpp | acid phosphatase, prostate |
| NM_021694 | Arhgef1 | Rho guanine nucleotide exchange factor (GEF) 1 |
| NM_021760 | Col5a3 | procollagen, type V, alpha 3 |
| NM_022193 | Acaca | acetyl-coenzyme A carboxylase alpha |
| NM_022268 | Pygl | liver glycogen phosphorylase |
| NM_022282 | Dlgh2 | discs, large homolog 2 (Drosophila) |
| NM_022389 | Dhcr7 | 7-dehydrocholesterol reductase |
| NM_022392 | Insig1 | insulin induced gene 1 |
| NM_022582 | Lgals7 | lectin, galactose binding, soluble 7 |
| NM_022603 | Fgfbp1 | fibroblast growth factor binding protein 1 |
| NM_022628 | Nphs1 | nephrosis 1 homolog, nephrin (human) |
| NM_022705 | Thedc1 | thioesterase domain containing 1 |
| NM_023021 | Kcnn4 | potassium intermediate/small conductance calcium- |
| | | activated channel, subfamily N, member 4 |
| NM_023092 | Myo1c | myosin IC |
| NM_023987 | Birc3 | baculoviral IAP repeat-containing 3 |
| NM_024147 | Evl | Ena-vasodilator stimulated phosphoprotein |
| NM_024364 | Hr | hairless homolog (mouse) |
| NM_024390 | Hpgd | hydroxyprostaglandin dehydrogenase 15 (NAD) |
| NM_024398 | Aco2 | aconitase 2, mitochondrial |
| NM_030852 | Mia1 | melanoma inhibitory activity 1 |
| NM_030853 | Lat | linker for activation of T cells |
| NM_030863 | Msn | Moesin |
| NM_031031 | Gatm | glycine amidinotransferase (L-arginine:glycine |
| | | amidinotransferase) |
| NM_031337 | St3gal5 | ST3 beta-galactoside alpha-2,3-sialyltransferase 5 |
| NM_031538 | Cd8a | CD8 antigen, alpha chain |
| NM_031562 | Csn10 | casein kappa |
| NM_031598 | Pla2g2a | phospholipase A2, group IIA (platelets, synovial fluid) |
| NM_053328 | Bhlhb2 | basic helix-loop-helix domain containing, class B2 |
| NM_053388 | Gjb6 | gap junction membrane channel protein beta 6 |
| NM_053587 | S100a9 | S100 calcium binding protein A9 (calgranulin B) |
| NM_053623 | Acsl4 | acyl-CoA synthetase long-chain family member 4 |
| NM_053633 | Egr2 | early growth response 2 |
| NM_053669 | Aps | adaptor protein with pleckstrin homology and src |
| | | homology 2 domains |
| NM_053688 | Pde6h | phosphodiesterase 6H, cGMP-specific, cone, gamma |
| NM_053751 | Wap | whey acidic protein |
| NM_053806 | Kcnk6 /// | potassium channel, subfamily K, member 6 /// similar to |
| | LOC501662 | potassium channel, subfamily K, member 6 |
| NM_053822 | S100a8 | S100 calcium binding protein A8 (calgranulin A) |
| NM_053887 | Map3k1 | mitogen activated protein kinase kinase kinase 1 |
| NM_053999 | Ppp2r2a | protein phosphatase 2 (formerly 2A), regulatory subunit B |
| | | (PR 52), alpha isoform |
| NM_058213 | Atp2a1 | ATPase, Ca++ transporting, cardiac muscle, fast twitch 1 |
| NM_080770 | Scgb2a1 /// | secretoglobin, family 2A, member 1 /// |
| | Scgb2a2 | secretoglobin, family 2A, member 2 |
| NM_080787 | Dgka | diacylglycerol kinase, alpha |
| NM_080906 | Ddit4 | DNA-damage-inducible transcript 4 |
| NM_130411 | Coro1a | coronin, actin binding protein 1A |
| NM_130429 | Lef1 | lymphoid enhancer binding factor 1 |
| NM_133424 | Actn3 | actinin alpha 3 |
| NM_133513 | Muc10 | mucin 10, submandibular gland salivary mucin |
| NM_133522 | Sstr3 | somatostatin receptor 3 |
| NM_133533 | Cd79b | CD79B antigen |
| NM_133561 | Brp44l | brain protein 44-like |
| NM_133566 | Cst6 | cystatin E/M |
| NM_133572 | Cdc25b | cell division cycle 25 homolog B (S. pombe) |
| NM_134334 | Ctsd | cathepsin D |
| NM_134382 | Elovl5 | ELOVL family member 5, elongation of long chain fatty |
| | | acids (yeast) |
| NM_134389 | Acsbg1 | acyl-CoA synthetase bubblegum family member 1 |
| U13253 | Fabp5 | fatty acid binding protein 5, epidermal |
| U23407 | Crabp2 | cellular retinoic acid binding protein 2 |
| U35025 | Acvr1c | activin A receptor, type IC |
| U54791 | Cxcr4 | chemokine (C-X-C motif) receptor 4 |
| U67914 | Cpa3 | carboxypeptidase A3 |
| X74293 | Itga7 | integrin alpha 7 |
| X74294 | Itga7 | integrin alpha 7 |

B. Liver

| | | |
|---|---|---|
| AA817761 | Adh1 | alcohol dehydrogenase 1 (class I) |
| AA818759 | RGD1560745_predicted | similar to OTTHUMP00000018508 (predicted) |
| AA859049 | Es2 | esterase 2 |
| AA859663 | LOC301113 | similar to solute carrier family 25 (mitochondrial carrier; |
| | | phosphate carrier), member 23 |
| AA900547 | Nfx1 | Nuclear transcription factor, X-box binding 1 |
| AA901337 | Spic_predicted | Spi-C transcription factor (Spi-1/PU.1 related) (predicted) |
| AA945955 | Ogn_predicted | osteoglycin (predicted) |
| AA955091 | Itga6 | integrin, alpha 6 |
| AA955771 | — | — |
| AA957990 | — | Transcribed locus |
| AA997683 | Aldh1b1 | aldehyde dehydrogenase 1 family, member B1 |
| AA997980 | — | Transcribed locus |
| AA998264 | LOC307495 | similar to biliverdin reductase B (flavin reductase |
| | | (NADPH)) |
| AF411318 | Mt1a | metallothionein 1a |
| AI045015 | RGD1560736_predicted | similar to solute carrier family 9 (sodium/hydrogen |
| | | exchanger), isoform 9 (predicted) |
| AI045533 | LOC679725 | Similar to MASK-4E-BP3 protein |
| AI060139 | Atxn2_predicted | Ataxin 2 (predicted) |
| AI060227 | Rnd3 | Rho family GTPase 3 |
| AI071547 | RGD1308329_predicted | Similar to KIAA0869 protein (predicted) |
| AI073272 | Tor1b | torsin family 1, member B |
| AI102035 | — | Transcribed locus |
| AI137045 | — | Transcribed locus |
| AI137640 | Cldn1 | claudin 1 |
| AI146237 | — | Transcribed locus |
| AI176583 | Mrvldc1 | MARVEL (membrane-associating) domain containing 1 |
| AI180253 | RGD1563825_predicted | similar to ENSANGP00000020885 (predicted) |
| AI228307 | Mlc1_predicted | megalencephalic leukoencephalopathy with subcortical |
| | | cysts 1 homolog (human) (predicted) |
| AI232328 | Hgd | homogentisate 1, 2-dioxygenase |
| AI232806 | — | Transcribed locus |
| AI406662 | Sec63_predicted | SEC63-like (S. cerevisiae) (predicted) |
| AI410749 | — | Transcribed locus |
| AI411693 | LOC299458 /// | similar to immunoglobulin heavy chain 6 (Igh-6) /// |
| | LOC366747 /// | similar to Ig H-chain V-region precursor /// |
| | LOC678701 /// | similar to Ig heavy chain V region MC101 precursor /// |
| | RGD1359202 | hypothetical protein LOC678701 |
| AI535089 | Zfp131 | zinc finger protein 131 |
| AI547628 | LOC367485 /// | similar to spermatogenesis associated glutamate (E)-rich |
| | LOC501235 /// | protein 4d /// similar to RIKEN cDNA 5031410I06 |
| | LOC501339 /// | |
| | LOC688687 /// | |
| | LOC689870 /// | |
| | LOC690439 /// | |
| | LOC690577 /// | |
| | LOC691712 | |
| AI555855 | — | Transcribed locus |
| AI556941 | Slc39a4_predicted | solute carrier family 39 (zinc transporter), member 4 |
| | | (predicted) |
| AW435376 | Lrp5_predicted | low density lipoprotein receptor-related protein 5 |
| | | (predicted) |
| AW522341 | — | Transcribed locus |
| AW525662 | Cdc27 | cell division cycle 27 homolog (S. cerevisiae) |
| AW530264 | — | Transcribed locus |
| AW533569 | Clca2_predicted | chloride channel, calcium activated, family member 2 |
| | | (predicted) |
| AW916957 | — | Transcribed locus, strongly similar to XP_579758.1 |
| | | PREDICTED: hypothetical protein XP_579758 |
| | | [Rattus norvegicus] |
| BE097702 | — | — |
| BE100625 | Emr1 | EGF-like module containing, mucin-like, hormone |
| | | receptor-like sequence 1 |
| BE108648 | — | Transcribed locus |
| BE114778 | Cgn_predicted | cingulin (predicted) |
| BE116152 | Elovl6 | ELOVL family member 6, elongation of long chain fatty |
| | | acids (yeast) |
| BE119164 | — | — |
| BE121383 | Ncam2 | neural cell adhesion molecule 2 |
| BE329244 | — | — |
| BE349751 | — | — |
| BF282112 | — | Transcribed locus |
| BF284288 | — | Transcribed locus |
| BF389489 | Tbc1d12_predicted | TBC1D12: TBC1 domain family, member 12 (predicted) |
| BF393945 | — | Transcribed locus |
| BF394166 | Gpm6a | glycoprotein m6a |
| BF396857 | Elovl6 | ELOVL family member 6, elongation of long chain fatty |
| | | acids (yeast) |
| BF397095 | LOC681234 | similar to debranching enzyme homolog 1 |
| BF401705 | — | Transcribed locus |
| BF403483 | — | Transcribed locus, moderately similar to NP_997413.2 |
| | | oxysterol binding protein-like 1 [Mus musculus] |
| BF404957 | — | Transcribed locus |
| BF414655 | Maoa | Monoamine oxidase A |
| BF418138 | — | Transcribed locus |
| BG377595 | LOC367746 | similar to Spindlin-like protein 2 (SPIN-2) |
| BG378056 | — | Transcribed locus |
| BG378637 | Hrasls3 | HRAS like suppressor 3 |
| BI273908 | — | Transcribed locus |
| BI275030 | RGD1308694_predicted | similar to hypothetical protein MGC47256 (predicted) |
| BI276183 | — | Transcribed locus |
| BI277600 | LOC362972 | similar to adiponutrin |
| BI279384 | — | Transcribed locus |
| BI282114 | — | Endogenous retrovirus mRNA, partial sequence |
| BI285941 | — | Transcribed locus |
| BI289506 | — | Transcribed locus |
| BI290154 | RGD1309578 | similar to Aa2-174 |
| BI290769 | RGD1309634_predicted | hypothetical LOC305452 (predicted) |
| BI290794 | — | Transcribed locus, strongly similar to XP_347073.1 |
| | | PREDICTED: hypothetical protein XP_347072 |
| | | [Rattus norvegicus] |
| BI293584 | Tmem55a | transmembrane protein 55A |
| BI297291 | — | Transcribed locus |
| BI301687 | — | Transcribed locus |
| BM382879 | Pura_predicted | Purine rich element binding protein A (predicted) |
| BM386775 | Aim1l_predicted | absent in melanoma 1-like (predicted) |
| L19180 | Ptprd | protein tyrosine phosphatase, receptor type, D |
| M10094 | RT1-Aw2 | RT1 class Ib, locus Aw2 |
| M24024 | RT1-Aw2 | RT1 class Ib, locus Aw2 |
| M30596 | Me1 | malic enzyme 1 |
| M63991 | Serpina7 | serine (or cysteine) peptidase inhibitor, clade A (alpha-1 |
| | | antipeptidase, antitrypsin), member 7 |
| NM_012600 | Me1 | malic enzyme 1 |
| NM_012646 | RT1-N1 /// | RT1 class Ib gene, H2-TL-like, grc region (N1) /// |
| | RT1-N2 /// | RT1 class Ib gene, H2-TL-like, grc region (N3) /// |
| | RT1-N3 | RT1 class Ib gene, H2-TL-like, grc region(N2) |
| NM_013068 | Fabp2 | fatty acid binding protein 2, intestinal |
| NM_017077 | Foxa3 | forkhead box A3 |
| NM_017149 | Meox2 | mesenchyme homeobox 2 |
| NM_019203 | Tsx | testis specific X-linked gene |
| NM_021653 | Dio1 | deiodinase, iodothyronine, type I |
| NM_023969 | Edg7 | putative G protein-coupled receptor snGPCR32 |
| NM_024147 | Evl | Ena-vasodilator stimulated phosphoprotein |
| NM_031561 | RGD1562323_predicted | similar to fatty acid translocase/CD36 (predicted) |
| NM_031641 | Sult4a1 | sulfotransferase family 4A, member 1 |
| NM_053639 | Ltc4s | leukotriene C4 synthase |
| NM_053962 | Sds | serine dehydratase |
| NM_053999 | Ppp2r2a | protein phosphatase 2 (formerly 2A), regulatory subunit B |
| | | (PR 52), alpha isoform |
| NM_133525 | RGD620382 | Nucleoside 2-deoxyribosyltransferase domain containing |
| | | protein RGD620382 |
| NM_133547 | Sult1c2 /// Sult1c2a | sulfotransferase family, cytosolic, 1C, member 2 /// |
| | | sulfotransferase family, cytosolic, 1C, member 2a |
| NM_134379 | UST4r | integral membrane transport protein UST4r |
| Z78279 | Col1a1 | procollagen, type 1, alpha 1 |

C. Quadricep

| | | |
|---|---|---|
| AA800285 | — | Transcribed locus |
| AA901290 | — | Transcribed locus |
| AA998276 | — | Transcribed locus |
| AF368860 | LOC680367 | similar to Urinary protein 3 precursor (RUP-3) |
| AI013568 | RGD1311123 | Similar to 1300013J15Rik protein |
| AI169092 | Thrsp | thyroid hormone responsive protein |
| AI170665 | Chac1_predicted | ChaC, cation transport regulator-like 1 (E. coli) (predicted) |
| AI179334 | Fasn | fatty acid synthase |
| AI231999 | LOC689256 | similar to Tumor protein D53 (mD53) (Tumor protein |
| | | D52-like 1) |
| AI237143 | Rexo4 | REX4, RNA exonuclease 4 homolog (S. cerevisiae) |
| AI237251 | — | — |
| AI502757 | Cst7_predicted | cystatin F (leukocystatin) (predicted) |
| AI555855 | — | Transcribed locus |
| AI579422 | Bex1 | brain expressed X-linked 1 |
| AW251280 | — | — |
| AW530219 | Ryr1 | ryanodine receptor 1, skeletal muscle |
| AW533490 | — | — |
| AW534519 | — | Transcribed locus |
| AW535897 | — | Transcribed locus |
| AW536022 | — | Transcribed locus |
| BE102861 | — | Transcribed locus |
| BE106279 | — | Transcribed locus |
| BE118382 | Nek9_predicted | NIMA (never in mitosis gene a)-related kinase 9 |
| | | (predicted) |
| BE118557 | — | — |
| BE118639 | Tpr | translocated promoter region |
| BF281984 | LOC360570 | similar to myosin XVIIIa |
| BF284535 | — | Transcribed locus |
| BF290088 | Nkx2-3_predicted | NK2 transcription factor related, locus 3 (Drosophila) |
| | | (predicted) |
| BF404113 | — | Transcribed locus |
| BF409456 | LOC688018 | similar to SH3-domain binding protein 3 |
| BF409997 | — | Transcribed locus |
| BF524010 | — | — |
| BF559640 | — | Transcribed locus |
| BF559746 | Dyrk2_predicted | dual-specificity tyrosine-(Y)-phosphorylation regulated |
| | | kinase 2 (predicted) |
| BF564825 | LOC500591 | similar to calmodulin-binding transcription activator 1 |
| BF565278 | Dnaja4 | DnaJ (Hsp40) homolog, subfamily A, member 4 |
| BF568007 | LOC501069 | similar to golgi autoantigen golgin subtype a4; tGolgin-1 |
| BI275261 | — | — |
| BI282114 | — | Endogenous retrovirus mRNA, partial sequence |
| H31802 | — | — |
| J00710 | Csn1s1 | casein alpha s1 |
| J02585 | Scd1 | stearoyl-Coenzyme A desaturase 1 |
| L81174 | Ankrd1 | ankyrin repeat domain 1 (cardiac muscle) |
| M24024 | RT1-Aw2 | RT1 class Ib, locus Aw2 |
| NM_012594 | Lalba | lactalbumin, alpha |
| NM_012703 | Thrsp | thyroid hormone responsive protein |
| NM_013220 | Ankrd1 | ankyrin repeat domain 1 (cardiac muscle) |
| NM_017332 | Fasn | fatty acid synthase |
| NM_053381 | Atp1b4 | ATPase, (Na+)/K+ transporting, beta 4 polypeptide |
| NM_134326 | Alb | Albumin |

D. Adipose + Liver*

| | | |
|---|---|---|
| AA955771 | — | — |
| AF411318 | Mt1a | metallothionein 1a |
| AI137640 | Cldn1 | claudin 1 |
| AI555855 | — | Transcribed locus |
| AW533569 | Clca2_predicted | chloride channel, calcium activated, family member 2 |
| | | (predicted) |
| BE116152 | Elovl6 | ELOVL family member 6, elongation of long chain fatty |
| | | acids (yeast) |
| BE329244 | — | — |
| BI282114 | — | Endogenous retrovirus mRNA, partial sequence |
| M24024 | RT1-Aw2 | RT1 class Ib, locus Aw2 |
| M30596 | Me1 | malic enzyme 1 |
| NM_012600 | Me1 | malic enzyme 1 |
| NM_012646 | RT1-N1 /// RT1-N2 /// | RT1 class Ib gene, H2-TL-like, grc region (N1) /// RT1 |
| | RT1-N3 | class Ib gene, H2-TL-like, grc region (N3) /// RT1 class Ib |
| | | gene, H2-TL-like, grc region(N2) |
| NM_024147 | Evl | Ena-vasodilator stimulated phosphoprotein |
| NM_053999 | Ppp2r2a | protein phosphatase 2 (formerly 2A), regulatory subunit B |
| | | (PR 52), alpha isoform |

E. Adipose + Quadricep*

| | | |
|---|---|---|
| AA901290 | — | Transcribed locus |
| AI555855 | — | Transcribed locus |
| AW251280 | — | — |
| BE102861 | — | Transcribed locus |
| BF524010 | — | — |
| BI282114 | — | Endogenous retrovirus mRNA, partial sequence |
| J00710 | Csn1s1 | casein alpha s1 |
| M24024 | RT1-Aw2 | RT1 class Ib, locus Aw2 |
| NM_012594 | Lalba | lactalbumin, alpha |
| NM_012703 | Thrsp | thyroid hormone responsive protein |

F. Liver + Quadricep*

| | | |
|---|---|---|
| AI555855 | — | Transcribed locus |
| BI282114 | — | Endogenous retrovirus mRNA, partial sequence |
| M24024 | RT1-Aw2 | RT1 class Ib, locus Aw2 |

G. Adipose + Liver + Quadricep*

| | | |
|---|---|---|
| AI555855 | — | Transcribed locus |
| M24024 | RT1-Aw2 | RT1 class Ib, locus Aw2 |
| BI282114 | — | Endogenous retrovirus mRNA, partial sequence |

*“Adipose + Liver”, for example refers to genes that are differentially expressed in both adipose and liver tissue in response to all three treatments as compared with the control. “Adipose + Quadricep, ” “Liver + Quadricep, ” and “Adipose + Liver + Quadricep” are defined analogously.

Biological pathway analysis was conducted using commercially available mapping programs and public databases. The major pathways represented by the differentially expressed genes in adipose, liver, and muscle are set forth below in Tables 7, 8 and 9. As stated above, these genes were found to be differentially expressed as a result any one of the three treatments.

TABLE 7
Biochemical Pathways Associated with Genes Differentially Expressed in Adipose
Tissue as a Result of CLA Supplementation, High Protein Diet and Increased Exercise

| | | |
|---|---|---|
| Public ID | Gene Symbol | Gene Title |

Cholesterol Biosynthetic Pathway

| | | |
|---|---|---|
| AW530769 | Fdft1 | farnesyl diphosphate farnesyl transferase 1 |
| BF417479 | Dhcr24 | 24-dehydrocholesterol reductase |
| BM390399 | Hmgcr | 3-hydroxy-3-methylglutaryl-Coenzyme A reductase |
| J03867 | Cyb5r3 | cytochrome b5 reductase 3 |
| NM_013134 | Hmgcr | 3-hydroxy-3-methylglutaryl-Coenzyme A reductase |
| NM_019238 | Fdft1 | farnesyl diphosphate farnesyl transferase 1 |
| NM_022389 | Dhcr7 | 7-dehydrocholesterol reductase |

Statin Pathway

| | | |
|---|---|---|
| AI502114 | Abca1 | ATP-binding cassette, sub-family A (ABC1), member 1 |
| AI548897 | Soat1 | Sterol O-acyltransferase 1 |
| BM390399 | Hmgcr | 3-hydroxy-3-methylglutaryl-Coenzyme A reductase |
| NM_012824 | Apoc1 | apolipoprotein C-I |
| NM_013134 | Hmgcr | 3-hydroxy-3-methylglutaryl-Coenzyme A reductase |

Adipogenesis

| | | |
|---|---|---|
| AI072892 | Frzb | frizzled-related protein |
| AI235414 | Rora_predicted | RAR-related orphan receptor alpha (predicted) |
| AI407163 | Prlr | Prolactin receptor |
| BE100543 | Ncor1 | nuclear receptor co-repressor 1 |
| BE106199 | Rora_predicted | RAR-related orphan receptor alpha (predicted) |
| BE118901 | Gata3 | GATA binding protein 3 |
| BM391538 | Frzb | frizzled-related protein |
| NM_012524 | Cebpa | CCAAT/enhancer binding protein (C/EBP), alpha |
| NM_053633 | Egr2 | early growth response 2 |

Apoptosis

| | | |
|---|---|---|
| NM_022582 | Lgals7 | lectin, galactose binding, soluble 7 |
| NM_080906 | Ddit4 | DNA-damage-inducible transcript 4 |
| NM_019371 | Egln3 | EGL nine homolog 3 (C. elegans) |
| NM_053388 | Gjb6 | gap junction membrane channel protein beta 6 |
| NM_053887 | Map3k1 | mitogen activated protein kinase kinase kinase 1 |
| NM_019282 | Grem1 | gremlin 1 homolog, cysteine knot superfamily (Xenopus |
| | | laevis) |
| AF002251 | Rassf5 | Ras association (RalGDS/AF-6) domain family 5 |
| U35025 | Acvr1c | activin A receptor, type IC |
| AI136555 | Cipar1 | castration induced prostatic apoptosis-related protein 1 |
| BF417479 | Dhcr24 | 24-dehydrocholesterol reductase |
| AI233243 | Rhot2 | ras homolog gene family, member T2 |
| AI169601 | Tnfrsf14 | tumor necrosis factor receptor superfamily, member 14 |
| | | (herpesvirus entry mediator) |
| AW433947 | Tnfrsf5 | tumor necrosis factor receptor superfamily, member 5 |
| BM383464 | RT1-Aw2 | RT1 class Ib, locus Aw2 |
| AI137137 | Lck | lymphocyte protein tyrosine kinase |
| AI044631 | Cd3g | CD3 antigen, gamma polypeptide |
| NM_013041 | Sycp3 | synaptonemal complex protein 3 |
| NM_012594 | Lalba | lactalbumin, alpha |
| BE112895 | Pea15 | phosphoprotein enriched in astrocytes 15 |
| AI599423 | Gadd45g | growth arrest and DNA-damage-inducible 45 gamma |

Cell Motility

| | | |
|---|---|---|
| AA875132 | Tpm1 | Tropomyosin 1, alpha |
| AI045155 | Ccr6 | chemokine (C-C motif) receptor 6 |
| AW920881 | Actr2 LOC301861 | ARP2 actin-related protein 2 homolog (yeast) similar to |
| | LOC685707 | ARP2 actin-related protein 2 homolog (yeast) (predicted) |
| BF547251 | Nav1_predicted | neuron navigator 1 (predicted) similar to neuron navigator 1 |
| BM392106 | Cald1 | caldesmon 1 |
| NM_019131 | Tpm1 | tropomyosin 1, alpha |
| NM_024147 | Evl | Ena-vasodilator stimulated phosphoprotein |
| NM_030863 | Msn | moesin |
| NM_053887 | Map3k1 | mitogen activated protein kinase kinase kinase 1 |
| NM_130411 | Coro1a | coronin, actin binding protein 1A |

Mitochondrial Fatty Acid Betaoxidation

| | | |
|---|---|---|
| NM_016986 | Acadm | acetyl-Coenzyme A dehydrogenase, medium chain |
| NM_013200 | Cpt1b | carnitine palmitoyltransferase 1b, muscle |
| NM_053623 | Acsl4 | acyl-CoA synthetase long-chain family member 4 |

Fatty Acid Biosynthesis

| | | |
|---|---|---|
| BI296153 | Acaca | acetyl-coenzyme A carboxylase alpha |
| NM_022193 | Acaca | acetyl-coenzyme A carboxylase alpha |
| NM_022705 | Thedc1 | thioesterase domain containing 1 |

Fatty Acid Metabolism

| | | |
|---|---|---|
| AA848820 | Hpgd | hydroxyprostaglandin dehydrogenase 15 (NAD) |
| BE116152 | Elovl6 | ELOVL family member 6, elongation of long chain fatty |
| | | acids (yeast) |
| NM_013200 | Cpt1b | carnitine palmitoyltransferase 1b, muscle |
| NM_016986 | Acadm | acetyl-Coenzyme A dehydrogenase, medium chain |
| NM_024390 | Hpgd | hydroxyprostaglandin dehydrogenase 15 (NAD) |
| NM_053623 | Acsl4 | acyl-CoA synthetase long-chain family member 4 |
| NM_134382 | Elovl5 | ELOVL family member 5, elongation of long chain fatty |
| | | acids (yeast) |

Glycolysis

| | | |
|---|---|---|
| NM_017328 | Pgam2 | phosphoglycerate mutase 2 |
| AI411413 | LOC685778 Pdha1 | pyruvate dehydrogenase E1 alpha 1 pyruvate |
| | | dehydrogenase E1 alpha 1 pseudogene |
| NM_012949 | Eno3 | enolase 3, beta |
| BI295900 | Dlat | dihydrolipoamide S-acetyltransferase (E2 component of |
| | | pyruvate dehydrogenase complex) |

Regulation of Cell Proliferation

| | | |
|---|---|---|
| AA859235 | Cdca7 | cell division cycle associated 7 |
| AA875609 | Smarca2 | SWI/SNF related, matrix associated, actin dependent |
| | | regulator of chromatin, subfamily a, member 2 |
| AW251860 | Tcf7_predicted | transcription factor 7, T-cell specific (predicted) |
| BE097933 | Mitf | microphthalmia-associated transcription factor |
| BE117736 | Tgfb2 | Transforming growth factor, beta 2 |
| BF288177 | Csnk2a1 | casein kinase II, alpha 1 polypeptide |
| NM_019334 | Pitx2 | paired-like homeodomain transcription factor 2 |
| NM_019371 | Egln3 | EGL nine homolog 3 (C. elegans) |
| NM_022389 | Dhcr7 | 7-dehydrocholesterol reductase |

Inflammation, Immunity and Stress Response*

| | | |
|---|---|---|
| AA817742 | Ms4a1_predicted | membrane-spanning 4-domains, subfamily A, member 1 |
| | | (predicted) |
| AA900477 | Vav2_predicted | Vav2 oncogene (predicted) |
| AA925583 | Blnk | B-cell linker |
| AA996885 | Ccl19_predicted | chemokine (C-C motif) ligand 19 (predicted) |
| AA998516 | Ccna2 | cyclin A2 |
| AF002251 | Rassf5 | Ras association (RalGDS/AF-6) domain family 5 |
| AF255614 | Magi3 | membrane associated guanylate kinase, WW and PDZ |
| | | domain containing 3 |
| AI009823 | Sectm1b | secreted and transmembrane 1B |
| AI009944 | Txk | TXK tyrosine kinase |
| AI010476 | Rac2 | RAS-related C3 botulinum substrate 2 |
| AI012081 | Rhoh | ras homolog gene family, member H |
| AI012250 | RT1-Aw2 | RT1 class Ib, locus Aw2 |
| AI044622 | MGC125004 | similar to T cell receptor interacting molecule |
| AI044631 | Cd3g | CD3 antigen, gamma polypeptide |
| AI137137 | Lck | lymphocyte protein tyrosine kinase |
| AI137640 | Cldn1 | claudin 1 |
| AI169601 | Tnfrsf14 | tumor necrosis factor receptor superfamily, member 14 |
| | | (herpesvirus entry mediator) |
| AI171093 | PRKCQ | protein kinase C, theta |
| AI176755 | Ptpn22_predicted | protein tyrosine phosphatase, non-receptor type 22 |
| | | (lymphoid) (predicted) |
| AI177373 | Cr2_predicted | complement receptor 2 (predicted) |
| AI178808 | Il2rg | interleukin 2 receptor, gamma (severe combined |
| | | immunodeficiency) |
| AI407163 | Prlr | Prolactin receptor |
| AI549199 | Ptger4 | Prostaglandin E receptor 4 (subtype EP4) |
| AI556056 | Cr2_predicted | complement receptor 2 (predicted) |
| AI599423 | Gadd45g | growth arrest and DNA-damage-inducible 45 gamma |
| AI639117 | Cfb | complement factor B |
| AW251860 | Tcf7_predicted | transcription factor 7, T-cell specific (predicted) |
| AW433947 | Tnfrsf5 | tumor necrosis factor receptor superfamily, member 5 |
| AW526982 | Tlr2 | toll-like receptor 2 |
| AW532114 | Rasgrp2_predicted | RAS guanyl releasing protein 2 (calcium and DAG- |
| | | regulated) (predicted) |
| AW532179 | LOC500449 | similar to SHP2-interacting transmembrane adaptor |
| | | protein |
| AW915948 | Cd3e_predicted | CD3 antigen, epsilon polypeptide (predicted) |
| AW920881 | Actr2 LOC301861 | ARP2 actin-related protein 2 homolog (yeast) similar to |
| | | ARP2 actin-related protein 2 homolog (yeast) (predicted) |
| BE096652 | Ly6g6c | lymphocyte antigen 6 complex, locus G6C |
| BE109926 | Pik3cd_predicted | phosphatidylinositol 3-kinase catalytic delta polypeptide |
| | | (predicted) |
| BE115061 | Lef1 | Lymphoid enhancer binding factor 1 |
| BE117044 | Cd8a | CD8 antigen, alpha chain |
| BE328951 | Cldn4 | claudin 4 |
| BF288109 | RT1-Aw2 | RT1 class Ib, locus Aw2 |
| BF288130 | Ptprc | protein tyrosine phosphatase, receptor type, C |
| BF288177 | Csnk2a1 | casein kinase II, alpha 1 polypeptide |
| BF290113 | RGD1311584 | similar to stem cell adaptor protein STAP-1 |
| BF386742 | Catna1 | Catenin (cadherin-associated protein), alpha 1 |
| BF401586 | Prkcb1 | Protein kinase C, beta 1 |
| BF418025 | Grap | GRB2-related adaptor protein |
| BF555968 | Nedd9 | neural precursor cell expressed, developmentally down- |
| | | regulated gene 9 |
| BG378166 | Map4k2_predicted | mitogen activated protein kinase kinase kinase kinase 2 |
| | | (predicted) |
| BG663208 | Jak1 | Janus kinase 1 |
| BI274326 | Muc1 | mucin 1, transmembrane |
| BI285700 | Hspcb | heat shock 90 kDa protein 1, beta |
| BI290909 | Scap1 | src family associated phosphoprotein 1 |
| BI292090 | Cldn8 | claudin 8 |
| BM383464 | RT1-Aw2 | RT1 class Ib, locus Aw2 |
| BM387946 | Zap70 | zeta-chain (TCR) associated protein kinase 70 |
| BM389005 | Gpr68_predicted | G protein-coupled receptor 68 (predicted) |
| BM389513 | LOC688090 RT1-Bb | RT1 class II, locus Bb similar to RT1 class II |
| | | histocompatibility antigen, B-1 beta chain precursor |
| | | (RT1.B-beta(1)) |
| D13555 | Cd3z | CD3 antigen, zeta polypeptide |
| J02612 | Ugt1a1 Ugt1a10 Ugt1a2 | UDP glycosyltransferase 1 family, polypeptide A1 UDP |
| | Ugt1a3 Ugt1a5 Ugt1a6 | glycosyltransferase 1 family, polypeptide A6 UDP |
| | Ugt1a7 Ugt1a8 | glycosyltransferase 1 family, polypeptide A7 UDP |
| | | glycosyltransferase 1 family, polypeptide A8 UDP |
| | | glycosyltransferase 1 family polypeptide A2 UDP glycosy |
| M10072 | Ptprc | protein tyrosine phosphatase, receptor type, C |
| M13706 | Prkcb1 | protein kinase C, beta 1 |
| M17153 | Fcer1a | Fc receptor, IgE, high affinity I, alpha polypeptide |
| M24024 | RT1-Aw2 | RT1 class Ib, locus Aw2 |
| NM_012488 | A2m | alpha-2-macroglobulin |
| NM_012555 | Ets1 | v-ets erythroblastosis virus E26 oncogene homolog 1 |
| | | (avian) |
| NM_012559 | Fgg | fibrinogen, gamma polypeptide |
| NM_012938 | Ctse | cathepsin E |
| NM_013169 | Cd3d | CD3 antigen delta polypeptide |
| NM_017028 | Mx2 | myxovirus (influenza virus) resistance 2 |
| NM_019175 | Klk6 | kallikrein 6 |
| NM_019295 | Cd5 | CD5 antigen |
| NM_024147 | Evl | Ena-vasodilator stimulated phosphoprotein |
| NM_030853 | Lat | linker for activation of T cells |
| NM_030863 | Msn | moesin |
| NM_031031 | Gatm | glycine amidinotransferase (L-arginine: glycine |
| | | amidinotransferase) |
| NM_031538 | Cd8a | CD8 antigen, alpha chain |
| NM_053587 | S100a9 | S100 calcium binding protein A9 (calgranulin B) |
| NM_053669 | Aps | adaptor protein with pleckstrin homology and src |
| | | homology 2 domains |
| NM_053822 | S100a8 | S100 calcium binding protein A8 (calgranulin A) |
| NM_053887 | Map3k1 | mitogen activated protein kinase kinase kinase 1 |
| NM_053999 | Ppp2r2a | protein phosphatase 2 (formerly 2A), regulatory subunit B |
| | | (PR 52), alpha isoform |
| NM_130429 | Lef1 | lymphoid enhancer binding factor 1 |
| NM_133424 | Actn3 | actinin alpha 3 |
| NM_133533 | Cd79b | CD79B antigen |
| U13253 | Fabp5 | fatty acid binding protein 5, epidermal |
| U35025 | Acvr1c | activin A receptor, type IC |
| U54791 | Cxcr4 | chemokine (C-X-C motif) receptor 4 |

Multicellular Organismal Development

| | | |
|---|---|---|
| AI176755 | Ptpn22_predicted | protein tyrosine phosphatase, non-receptor type 22 |
| | | (lymphoid) (predicted) |
| AI179988 | Enc1 | ectodermal-neural cortex 1 |
| AI502837 | — | Transcribed locus |
| AI599423 | Gadd45g | growth arrest and DNA-damage-inducible 45 gamma |
| AW532165 | Sema3d | sema domain, immunoglobulin domain (Ig), short basic |
| | | domain, secreted, (semaphorin) 3D |
| BE097933 | Mitf | microphthalmia-associated transcription factor |
| BE126475 | Prrx1 | paired related homeobox 1 |
| BF283556 | Plxnc1_predicted | plexin C1 (predicted) |
| BF417479 | Dhcr24 | 24-dehydrocholesterol reductase |
| BF547251 | LOC685707 /// | neuron navigator 1 (predicted) /// similar to neuron |
| | Nav1_predicted | navigator 1 |
| BF553172 | — | Transcribed locus |
| BG662519 | Ank | Progressive ankylosis homolog (mouse) |
| BI285064 | — | Transcribed locus |
| BI290551 | Fnbp1 | Formin binding protein 1 |
| NM_019282 | Grem1 | gremlin 1 homolog, cysteine knot superfamily (Xenopus |
| | | laevis) |
| NM_019334 | Pitx2 | paired-like homeodomain transcription factor 2 |
| NM_031031 | Gatm | glycine amidinotransferase (L-arginine: glycine |
| | | amidinotransferase) |
| U23407 | Crabp2 | cellular retinoic acid binding protein 2 |

Regulation of Apoptosis

| | | |
|---|---|---|
| AA945727 | Card11_predicted | caspase recruitment domain family, member 11 |
| | | (predicted) |
| AI044631 | Cd3g | CD3 antigen, gamma polypeptide |
| AW251860 | Tcf7_predicted | transcription factor 7, T-cell specific (predicted) |
| AW915948 | Cd3e_predicted | CD3 antigen, epsilon polypeptide (predicted) |
| BE097933 | Mitf | microphthalmia-associated transcription factor |
| BE112895 | Pea15 | phosphoprotein enriched in astrocytes 15 |
| BE117736 | Tgfb2 | Transforming growth factor, beta 2 |
| BF417479 | Dhcr24 | 24-dehydrocholesterol reductase |
| NM_012594 | Lalba | lactalbumin, alpha |
| NM_013041 | Sycp3 | synaptonemal complex protein 3 |
| NM_023987 | Birc3 | baculoviral IAP repeat-containing 3 |
| NM_133424 | Actn3 | actinin alpha 3 |
| U35025 | Acvr1c | activin A receptor, type IC |

*Category includes the subcategories of Rn T-cell receptor, Rn B-cell receptor, leukocyte transendothelial migration, tight junction, adherens junction, antigen processing, response to unfolded proteins, response to wounding, response to external stimulus, inflammatory response, immune response, T-cell activation and Rn IL-4.

TABLE 8
Biochemical Pathways Associated with Genes Differentially
Expressed in Liver Tissue as a Result of CLA Supplementation,
High Protein Diet and Increased Exercise

| | | |
|---|---|---|
| Public ID | Gene Symbol | Gene Title |

PPAR Signaling Pathway

| | | |
|---|---|---|
| NM_013068 | Fabp2 | fatty acid binding protein 2, |
| | | intestinal |
| NM_012600 | Me1 | malic enzyme 1 |
| M30596 | Me1 | malic enzyme 1 |

Fatty Acid Metabolism

| | | |
|---|---|---|
| BE116152 | Elovl6 | ELOVL family member 6, |
| | | elongation of long chain fatty |
| | | acids (yeast) |
| BF396857 | Elovl6 | ELOVL family member 6, |
| | | elongation of long chain fatty |
| | | acids (yeast) |
| NM_013068 | Fabp2 | fatty acid binding protein 2, |
| | | intestinal |
| NM_031561 | RGD1562323_predicted | similar to fatty |
| | | acid translocase/CD36 |
| | | (predicted) |

TABLE 9
Biochemical Pathways Associated with Genes Differentially Expressed In Muscle Tissue
as a Result of CLA Supplementation, High Protein Diet and Increased Exercise

| | | |
|---|---|---|
| Public ID | Gene Symbol | Gene Title |

Lipid Metabolism

| | | |
|---|---|---|
| AI169092 | Thrsp | thyroid hormone responsive protein |
| J02585 | Scd1 | stearoyl-Coenzyme A desaturase 1 |
| NM_012703 | Thrsp | thyroid hormone responsive protein |

### Example 4

This example sets forth the further analysis of gene expression profiles in liver, muscle and/or adipose of the control and test animals comprising the high protein treatment described in Example 1 at day 60 and initially analyzed in Example 2.

Table 10 sets forth database identifiers and the names of the genes found to be differentially expressed in the high protein treatment.

TABLE 10
Genes Differentially Expressed in Three Tissues Indidually, or in Two or
More Tissues, or in all Three Tissues as a Result of a High Protein Diet

| | | |
|---|---|---|
| Public ID | Gene Symbol | Gene Title |

A. Adipose

| | | |
|---|---|---|
| AA799328 | RGD1560913_predicted | similar to expressed sequence AW413625 (predicted) |
| AA799400 | B3galt3 | UDP-Gal: betaGlcNAc beta 1,3-galactosyltransferase, |
| | | polypeptide 3 |
| AA799471 | LOC688173 | similar to Telethonin (Titin cap protein) |
| AA799557 | — | Transcribed locus, strongly similar to XP_424899.1 |
| | | PREDICTED: similar to Creatine kinase, sarcomeric |
| | | mitochondrial precursor (S-MtCK) (Mib-CK) (Basic-type |
| | | mitochondrial creatine kinase) [Gallus gallus] |
| AA799981 | Prkch | protein kinase C, eta |
| AA799996 | Gng10 | guanine nucleotide binding protein (G protein), gamma 10 |
| AA800004 | Sept4 | septin 4 |
| AA800031 | Fhl2 | four and a half LIM domains 2 |
| AA800240 | Hadha | hydroxyacyl-Coenzyme A dehydrogenase/3-ketoacyl- |
| | | Coenzyme A thiolase/enoyl-Coenzyme A hydratase |
| | | (trifunctional protein), alpha subunit |
| AA800245 | Trim54 | tripartite motif-containing 54 |
| AA800249 | Mypn_predicted | myopalladin (predicted) |
| AA800587 | Gpx2 | glutathione peroxidase 2 |
| AA800701 | LOC683626 | similar to limb-bud and heart |
| AA800711 | — | — |
| AA800750 | — | — |
| AA800892 | RGD1563599_predicted | similar to putative SH3BGR protein (predicted) |
| AA801220 | — | Transcribed locus |
| AA817708 | LOC310190 | similar to hypothetical protein FLJ11127 |
| AA817742 | Ms4a1_predicted | membrane-spanning 4-domains, subfamily A, member 1 |
| | | (predicted) |
| AA817907 | RGD1566355_predicted | similar to cell division cycle 2-like 1 (predicted) |
| AA817956 | Grem2_predicted | gremlin 2 homolog, cysteine knot superfamily (Xenopus |
| | | laevis) (predicted) |
| AA818020 | RGD1309660_predicted | similar to hypothetical protein MGC33214 (predicted) |
| AA818055 | — | Transcribed locus |
| AA818098 | — | Transcribed locus |
| AA818143 | — | Transcribed locus |
| AA818262 | Angptl4 | angiopoietin-like 4 |
| AA818334 | Srpx2_predicted | sushi-repeat-containing protein, X-linked 2 (predicted) |
| AA818521 | Thbd | Thrombomodulin |
| AA818664 | — | Transcribed locus |
| AA818807 | Emp2 | epithelial membrane protein 2 |
| AA818849 | RGD1310093_predicted | similar to hypothetical protein FLJ11354 (predicted) |
| AA818954 | — | Transcribed locus |
| AA819034 | isg12(b) | putative ISG12(b) protein |
| AA819134 | Dapp1_predicted | dual adaptor for phosphotyrosine and 3-phosphoinositides 1 |
| | | (predicted) |
| AA819268 | — | Transcribed locus |
| AA819776 | Hspca /// LOC498264 /// | heat shock protein 1, alpha /// similar to heat shock protein 1, |
| | LOC500299 /// | alpha |
| | LOC691091 | |
| AA819788 | Rtp4_predicted | receptor transporter protein 4 (predicted) |
| AA819804 | Zfp91 | zinc finger protein 91 |
| AA848738 | LOC362792 | similar to phospholipase D family, member 4 |
| AA848820 | Hpgd | hydroxyprostaglandin dehydrogenase 15 (NAD) |
| AA848821 | LOC684681 | similar to Histone H1.2 (H1 VAR.1) (H1c) |
| AA848944 | — | Transcribed locus |
| AA848980 | LOC498205 | similar to DnaJ (Hsp40) homolog, subfamily C, member 14 |
| AA849552 | LOC683381 | similar to Paired amphipathic helix protein Sin3b |
| | | (Transcriptional corepressor Sin3b) (Histone deacetylase |
| | | complex subunit Sin3b) |
| AA849706 | — | Transcribed locus |
| AA849719 | — | Transcribed locus |
| AA850195 | Pank1_predicted | pantothenate kinase 1 (predicted) |
| AA850618 | Sorl1 | sortilin-related receptor, LDLR class A repeats-containing |
| AA850773 | — | — |
| AA850780 | RGD1564560_predicted | similar to RCK (predicted) |
| AA851046 | — | — |
| AA851282 | Ss18 | synovial sarcoma translocation, Chromosome 18 |
| AA851313 | — | — |
| AA851345 | — | Transcribed locus |
| AA858521 | — | Transcribed locus |
| AA858591 | — | Transcribed locus, strongly similar to XP_996369.1 |
| | | PREDICTED: similar to 40S ribosomal protein S27-like |
| | | protein [Mus musculus] |
| AA858815 | — | Transcribed locus |
| AA859029 | — | Transcribed locus |
| AA859235 | Cdca7 | cell division cycle associated 7 |
| AA859277 | — | Transcribed locus |
| AA859508 | RGD1564315_predicted | similar to RIKEN cDNA 9330161F08 (predicted) |
| AA859545 | RGD1305455 | similar to hypothetical protein FLJ10925 |
| AA859652 | — | Transcribed locus |
| AA859902 | — | Transcribed locus |
| AA859919 | Churc1_predicted | churchill domain containing 1 (predicted) |
| AA859982 | Dhx36_predicted | DEAH (Asp-Glu-Ala-His) box polypeptide 36 (predicted) |
| AA866419 | — | Transcribed locus |
| AA874782 | RGD1566234_predicted | similar to Grb10 protein (predicted) |
| AA875002 | Lrrc8 | leucine-rich repeat-containing 8 |
| AA875123 | — | Transcribed locus |
| AA875132 | — | Transcribed locus |
| AA875143 | LOC368062 | similar to Ankyrin repeat and IBR domain-containing |
| | | protein 1 |
| AA875261 | Fblim1 | filamin binding LIM protein 1 |
| AA875438 | RGD1309414_predicted | similar to KIAA0913 protein (predicted) |
| AA875609 | Smarca2 | SWI/SNF related, matrix associated, actin dependent |
| | | regulator of chromatin, subfamily a, member 2 |
| AA875647 | — | Transcribed locus |
| AA891634 | — | Transcribed locus, weakly similar to XP_343521.3 |
| | | PREDICTED: similar to Discs large homolog 5 (Placenta |
| | | and prostate DLG) (Discs large protein P-dlg) [Rattus |
| | | norvegicus] |
| AA891664 | Mfng | manic fringe homolog (Drosophila) |
| AA891826 | Tacstd2 | tumor-associated calcium signal transducer 2 |
| AA892234 | Mgst3_predicted | microsomal glutathione S-transferase 3 (predicted) |
| AA892765 | — | — |
| AA892778 | — | Transcribed locus |
| AA893004 | — | Transcribed locus |
| AA893192 | — | Transcribed locus |
| AA893195 | — | Transcribed locus |
| AA893384 | Irf3 | interferon regulatory factor 3 |
| AA893670 | — | Transcribed locus |
| AA893871 | — | Transcribed locus |
| AA894233 | Amid_predicted | apoptosis-inducing factor (AIF)-like mitochondrion- |
| | | associated inducer of death (predicted) |
| AA894262 | Sept1 | septin 1 |
| AA894336 | Ly96 | lymphocyte antigen 96 |
| AA899923 | Eml4_predicted | echinoderm microtubule associated protein like 4 (predicted) |
| AA900322 | — | Transcribed locus |
| AA900477 | Vav2_predicted | Vav2 oncogene (predicted) |
| AA901088 | RGD1561302_predicted | similar to Hypothetical protein A030013D21 (predicted) |
| AA901290 | — | Transcribed locus |
| AA901341 | Slc2a3 | Solute carrier family 2 (facilitated glucose transporter), |
| | | member 3 |
| AA923988 | — | Transcribed locus |
| AA924312 | — | Transcribed locus |
| AA924350 | LOC686634 /// | hypothetical protein LOC686634 /// hypothetical protein |
| | LOC690768 | LOC690768 |
| AA924459 | RGD1560293_predicted | similar to RIKEN cDNA 1200013B08 (predicted) |
| AA925303 | — | — |
| AA925583 | Blnk | B-cell linker |
| AA925804 | Pmpcb | peptidase (mitochondrial processing) beta |
| AA925924 | Crlf1_predicted | cytokine receptor-like factor 1 (predicted) |
| AA926072 | — | Transcribed locus |
| AA926082 | LOC500046 | similar to hypothetical protein FLJ21986 |
| AA926180 | LOC684489 | similar to angiopoietin-like 1 |
| AA926313 | — | Transcribed locus |
| AA943056 | RGD1562563_predicted | similar to RIKEN cDNA G430041M01 (predicted) |
| AA943147 | Herc6 | potential ubiquitin ligase |
| AA943808 | — | Transcribed locus |
| AA944073 | Rpl41 | ribosomal protein L41 |
| AA944162 | Olfml2a_predicted | olfactomedin-like 2A (predicted) |
| AA944304 | Tpd52_predicted | tumor protein D52 (predicted) |
| AA944325 | Itgb5 | Integrin, beta 5 |
| AA944380 | Nudt7_predicted | nudix (nucleoside diphosphate linked moiety X)-type motif 7 |
| | | (predicted) |
| AA944574 | — | Transcribed locus |
| AA944588 | — | Transcribed locus |
| AA944812 | Hsf2 | Heat shock factor 2 |
| AA945568 | RGD1565360_predicted | similar to homolog of Human holocarboxylase synthetase |
| | | gene HLCS (predicted) |
| AA945727 | Card11_predicted | caspase recruitment domain family, member 11 (predicted) |
| AA945737 | Cxcr4 | chemokine (C-X-C motif) receptor 4 |
| AA945909 | Cd3e_predicted | CD3 antigen, epsilon polypeptide (predicted) |
| AA945955 | Ogn_predicted | osteoglycin (predicted) |
| AA946353 | Satb1 | Special AT-rich sequence binding protein 1 |
| AA946430 | Syvn1 | synovial apoptosis inhibitor 1, synoviolin |
| AA955213 | RGD1309362 | similar to interferon-inducible GTPase |
| AA955354 | Stat4 | signal transducer and activator of transcription 4 |
| AA955494 | — | Transcribed locus |
| AA955691 | — | Transcribed locus |
| AA955771 | — | — |
| AA956317 | — | Transcribed locus, strongly similar to XP_982636.1 |
| | | PREDICTED: similar to stromal membrane-associated |
| | | protein 1 isoform 5 [Mus musculus] |
| AA956714 | Arid1a_predicted | AT rich interactive domain 1A (Swi1 like) (predicted) |
| AA957260 | Rrm2 | ribonucleotide reductase M2 |
| AA957585 | Ehd3 | EH-domain containing 3 |
| AA957811 | — | Transcribed locus |
| AA958001 | Cthrc1 | collagen triple helix repeat containing 1 |
| AA963069 | — | Transcribed locus |
| AA963276 | Etv1_predicted | ets variant gene 1 (predicted) |
| AA963364 | LOC680419 | Similar to Ras suppressor protein 1 |
| AA963477 | — | Transcribed locus |
| AA963975 | — | Transcribed locus |
| AA964114 | Klc1 | kinesin light chain 1 |
| AA964146 | LOC498662 | similar to RIKEN cDNA 2610019F03 |
| AA964219 | Lipg | lipase, endothelial |
| AA964419 | Capzb | Capping protein (actin filament) muscle Z-line, beta |
| AA964492 | — | Transcribed locus |
| AA965084 | Tnn_predicted | tenascin N (predicted) |
| AA965147 | — | Transcribed locus |
| AA965250 | LOC314964 | similar to PHD finger protein 20-like 1 isoform 1 |
| AA996417 | — | — |
| AA996557 | LOC678701 /// | similar to immunoglobulin heavy chain 6 (Igh-6) /// |
| | RGD1359202 | hypothetical protein LOC678701 |
| AA996717 | RGD1307583_predicted | LOC361111 (predicted) |
| AA996804 | — | Transcribed locus |
| AA996869 | — | Transcribed locus |
| AA996885 | Ccl19_predicted | chemokine (C-C motif) ligand 19 (predicted) |
| AA996921 | — | Transcribed locus |
| AA996927 | LOC690795 | similar to H2A histone family, member J |
| AA996943 | Phactr1 | phosphatase and actin regulator 1 |
| AA997187 | — | Transcribed locus |
| AA997710 | Pcdh19_predicted | protocadherin 19 (predicted) |
| AA997873 | — | Transcribed locus |
| AA997880 | — | Transcribed locus |
| AA998001 | — | Transcribed locus |
| AA998043 | LOC314964 | similar to PHD finger protein 20-like 1 isoform 1 |
| AA998516 | Ccna2 | cyclin A2 |
| AA998540 | LOC313934 | similar to Intersectin-2 (SH3 domain-containing protein 1B) |
| | | (SH3P18) (SH3P18-like WASP-associated protein) |
| AA998903 | — | CDNA clone IMAGE: 7374368 |
| AA998979 | LOC684857 /// | similar to Autophagy-specific gene 2 CG1241-PA |
| | LOC689688 | |
| AA998997 | — | Transcribed locus |
| AB012232 | Nfib | nuclear factor I/B |
| AB013454 | Slc34a1 | solute carrier family 34 (sodium phosphate), member 1 |
| AB020615 | Prkci | protein kinase C, iota |
| AB025017 | Zfp36 | zinc finger protein 36 |
| AB035507 | Mcam | melanoma cell adhesion molecule |
| AB039823 | LOC259244 /// | alpha-2u globulin PGCL3 /// alpha-2u globulin PGCL5 /// |
| | LOC259245 /// | alpha-2u globulin PGCL1 /// alpha-2u globulin PGCL4 /// |
| | LOC259246 /// | major urinary protein 5 /// alpha-2u globulin PGCL2 /// |
| | LOC298109 /// | alpha2u globulin /// alpha-2u-globulin (L type) /// major |
| | LOC298111 /// | urinary protein 4 /// alpha 2U globulin /// similar to alpha-2u |
| | LOC298116 /// | globulin PGCL2 /// similar to alpha-2u-globulin (L type) /// |
| | LOC366380 /// | similar to alpha-2u globulin PGCL4 isoform 1 /// similar to |
| | LOC500473 /// | major urinary protein 5 /// similar to alpha2u globulin /// |
| | LOC502954 /// | similar to Major urinary protein precursor (MUP) (Alpha-2u- |
| | LOC681426 /// | globulin) (Alpha(2)-euglobulin) (Allergen Rat n 1) (Rat n I) |
| | LOC682731 /// | /// similar to major urinary protein 4 |
| | LOC685482 /// | |
| | LOC685577 /// | |
| | LOC688457 /// | |
| | LOC688547 /// Mup4 /// | |
| | Mup5 /// Obp3 | |
| AB039826 | LOC259245 /// Mup5 | alpha-2u globulin PGCL5 /// major urinary protein 5 |
| AB039828 | LOC259244 /// | alpha-2u globulin PGCL3 /// alpha-2u globulin PGCL5 /// |
| | LOC259245 /// | alpha-2u globulin PGCL1 /// alpha-2u globulin PGCL4 /// |
| | LOC259246 /// | major urinary protein 5 /// alpha-2u globulin PGCL2 /// |
| | LOC298109 /// | alpha2u globulin /// alpha-2u-globulin (L type) /// major |
| | LOC298111 /// | urinary protein 4 /// alpha 2U globulin /// similar to alpha-2u |
| | LOC298116 /// | globulin PGCL2 /// similar to alpha-2u-globulin (L type) /// |
| | LOC366380 /// | similar to alpha-2u globulin PGCL4 isoform 1 /// similar to |
| | LOC500473 /// | major urinary protein 5 /// similar to alpha2u globulin /// |
| | LOC502954 /// | similar to Major urinary protein precursor (MUP) (Alpha-2u- |
| | LOC681426 /// | globulin) (Alpha(2)-euglobulin) (Allergen Rat n 1) (Rat n I) |
| | LOC682731 /// | /// similar to major urinary protein 4 |
| | LOC685482 /// | |
| | LOC685577 /// | |
| | LOC688457 /// | |
| | LOC688547 /// Mup4 /// | |
| | Mup5 /// Obp3 | |
| AB048730 | Ptges | prostaglandin E synthase |
| AB052846 | Sc5d | sterol-C5-desaturase (fungal ERG3, delta-5-desaturase) |
| | | homolog (S. cerevisae) |
| AB060652 | Nfia | nuclear factor I/A |
| AB070350 | Chp /// | calcium binding protein p22 /// similar to calcium binding |
| | RGD1564956_predicted | protein P22 (predicted) |
| | /// | |
| | RGD1565588_predicted | |
| AF002251 | Rassf5 | Ras association (RalGDS/AF-6) domain family 5 |
| AF002281 | Pdlim3 | PDZ and LIM domain 3 |
| AF022247 | Cubn | cubilin (intrinsic factor-cobalamin receptor) |
| AF035963 | Havcr1 | kidney injury molecule 1 |
| AF040750 | Gprk6 | G protein-coupled receptor kinase 6 |
| AF057025 | Tlr4 | toll-like receptor 4 |
| AF062594 | Nap1l1 | nucleosome assembly protein 1-like 1 |
| AF065147 | Cd44 | CD44 antigen |
| AF065438 | Lgals3bp | lectin, galactoside-binding, soluble, 3 binding protein |
| AF068860 | Defb1 | defensin beta 1 |
| AF077354 | Hspa4 | heat shock protein 4 |
| AF081196 | Rasgrp1 | RAS guanyl releasing protein 1 |
| AF093139 | Nxf1 | nuclear RNA export factor 1 |
| AF095585 | Pdlim7 | PDZ and LIM domain 7 |
| AF104399 | Cited1 | Cbp/p300-interacting transactivator with Glu/Asp-rich |
| | | carboxy-terminal domain 1 |
| AF129400 | Fxyd2 | FXYD domain-containing ion transport regulator 2 |
| AF159103 | Tnfaip6 | tumor necrosis factor alpha induced protein 6 |
| AF169637 | Lilrb3 /// LOC683446 /// | leukocyte immunoglobulin-like receptor, subfamily B (with |
| | LOC683463 /// | TM and ITIM domains), member 3 /// similar to killer |
| | LOC690948 /// | activatory receptor-like protein p91D (predicted) /// similar |
| | LOC690955 /// | to paired-Ig-like receptor A11 /// similar to paired-Ig-like |
| | RGD1562625_predicted | receptor B |
| AF201331 | Ace | angiotensin I converting enzyme (peptidyl-dipeptidase A) 1 |
| AF220558 | Trdn | Triadin |
| AF228917 | Zdhhc2 | zinc finger, DHHC domain containing 2 |
| AF245172 | Gda | guanine deaminase |
| AF253064 | Cklf | chemokine-like factor |
| AF254801 | Ngef | neuronal guanine nucleotide exchange factor |
| AF255614 | Magi3 | membrane associated guanylate kinase, WW and PDZ |
| | | domain containing 3 |
| AF260582 | LOC257650 | Hippyragranin |
| AF283276 | Gabbr1 | gamma-aminobutyric acid (GABA) B receptor 1 |
| AF321837 | Rgs2 | regulator of G-protein signaling 2 |
| AF335571 | Eml2 | echinoderm microtubule associated protein like 2 |
| AF370889 | Tpm1 | Tropomyosin 1, alpha |
| AF372216 | Tpm1 | tropomyosin 1, alpha |
| AF399874 | Tnnt1 | troponin T1, skeletal, slow |
| AF400662 | Cacna2d1 | calcium channel, voltage-dependent, alpha2/delta subunit 1 |
| AF411318 | Mt1a | metallothionein 1a |
| AI007639 | — | Transcribed locus |
| AI008151 | Dcp1a_predicted | DCP1 decapping enzyme homolog A (S. cerevisiae) |
| | | (predicted) |
| AI008369 | Coro7 | coronin 7 |
| AI008409 | — | CDNA clone IMAGE: 7321089 |
| AI008646 | — | — |
| AI008792 | — | Transcribed locus |
| AI009074 | Ogt | O-linked N-acetylglucosamine (GlcNAc) transferase (UDP- |
| | | N-acetylglucosamine:polypeptide-N-acetylglucosaminyl |
| | | transferase) |
| AI009657 | Cs | Citrate synthase |
| AI009791 | — | Transcribed locus |
| AI009823 | Sectm1b | secreted and transmembrane 1B |
| AI009944 | Txk | TXK tyrosine kinase |
| AI010107 | — | Transcribed locus |
| AI010237 | — | Transcribed locus |
| AI010427 | Cdkn1a | cyclin-dependent kinase inhibitor 1A |
| AI010476 | Rac2 | RAS-related C3 botulinum substrate 2 |
| AI01101 | RGD1560320_predicted | similar to brain specific protein 4 (predicted) |
| AI011713 | — | CDNA clone IMAGE: 7320582 |
| AI011757 | Fcgr3a | Fc fragment of IgG, low affinity IIIa, receptor |
| AI011920 | Rcsd1_predicted | RCSD domain containing 1 (predicted) |
| AI012081 | Rhoh | ras homolog gene family, member H |
| AI012247 | — | Transcribed locus, strongly similar to XP_892770.1 |
| | | PREDICTED: similar to Tyrosine-protein kinase FLK [Mus |
| | | musculus] |
| AI012250 | — | Transcribed locus |
| AI012518 | — | Transcribed locus |
| AI012520 | — | — |
| AI012590 | — | Transcribed locus |
| AI012603 | — | Transcribed locus |
| AI012884 | — | Transcribed locus |
| AI013044 | — | Transcribed locus |
| AI029113 | Apeg3 | antisense paternally expressed gene 3 |
| AI029445 | — | — |
| AI029460 | — | Transcribed locus |
| AI029631 | Igl@ | Immunoglobulin light chain, lambda gene cluster |
| AI029798 | — | Transcribed locus |
| AI029975 | — | Transcribed locus, strongly similar to NP_038482.3 ATP- |
| | | binding cassette 1, sub-family A, member 1 [Mus musculus] |
| AI030021 | — | Transcribed locus |
| AI030120 | LOC499094 | similar to zinc finger protein 61 |
| AI030203 | Irx3_predicted | Iroquois related homeobox 3 (Drosophila) (predicted) |
| AI030349 | — | CDNA clone IMAGE: 7379585 |
| AI030465 | — | — |
| AI030701 | Sox4_predicted | SRY-box containing gene 4 (predicted) |
| AI030853 | Calml3 | calmodulin-like 3 |
| AI031033 | RGD1311447_predicted | LOC363276 (predicted) |
| AI043579 | — | Transcribed locus |
| AI043752 | — | Transcribed locus |
| AI043753 | — | Transcribed locus |
| AI043800 | LOC311078 | Similar to T-Brain-1 |
| AI043817 | RGD1565884_predicted | Similar to Pellino protein homolog 2 (Pellino 2) (predicted) |
| AI044125 | Dpep2 | dipeptidase 2 |
| AI044222 | Cxcl9 | chemokine (C-X-C motif) ligand 9 |
| AI044316 | — | Transcribed locus |
| AI044494 | — | Transcribed locus |
| AI044500 | Ctdspl_predicted | CTD (carboxy-terminal domain, RNA polymerase II, |
| | | polypeptide A) small phosphatase-like (predicted) |
| AI044622 | MGC125004 | similar to T cell receptor interacting molecule |
| AI044631 | Cd3g | CD3 antigen, gamma polypeptide |
| AI044632 | — | — |
| AI044651 | Loxl2_predicted | lysyl oxidase-like 2 (predicted) |
| AI044661 | Pi16_predicted | protease inhibitor 16 (predicted) |
| AI044784 | — | Transcribed locus |
| AI044859 | RGD1311732 | similar to hypothetical protein MGC15407 |
| AI044869 | Pla2g2d | phospholipase A2, group IID |
| AI045021 | — | Transcribed locus, strongly similar to XP_001061227.1 |
| | | PREDICTED: similar to interleukin 17D precursor [Rattus |
| | | norvegicus] |
| AI045116 | — | Transcribed locus |
| AI045155 | Ccr6 | chemokine (C-C motif) receptor 6 |
| AI045201 | Tnfrsf21_predicted | tumor necrosis factor receptor superfamily, member 21 |
| | | (predicted) |
| AI045228 | Ap1s2_predicted | adaptor-related protein complex 1, sigma 2 subunit |
| | | (predicted) |
| AI045311 | Bin2a | beta-galactosidase-like protein |
| AI045321 | — | Transcribed locus |
| AI045459 | Ccnt2_predicted | Cyclin T2 (predicted) |
| AI045904 | — | — |
| AI045965 | RGD1311456_predicted | similar to RIKEN cDNA B230380D07 (predicted) |
| AI058292 | Sox7_predicted | SRY-box containing gene 7 (predicted) |
| AI058307 | Phemx | pan hematopoietic expression |
| AI059078 | RGD1309969 | similar to RIKEN cDNA 2600010E01 |
| AI059204 | Cpne8_predicted | copine VIII (predicted) |
| AI059295 | RGD1561653_predicted | similar to HECT domain containing 1 (predicted) |
| AI059468 | LOC308990 | hypothetical protein LOC308990 |
| AI059486 | Dennd2d_predicted | DENN/MADD domain containing 2D (predicted) |
| AI059850 | LOC681012 | similar to Estlp-like protein B |
| AI059914 | Etv1_predicted | ets variant gene 1 (predicted) |
| AI060043 | — | Transcribed locus |
| AI060117 | — | Transcribed locus |
| AI060225 | RGD1306534_predicted | similar to P-Rex1 (predicted) |
| AI060231 | Fkbp7_predicted | FK506 binding protein 7 (predicted) |
| AI070061 | RGD1304626_predicted | similar to KIAA1128 protein (predicted) |
| AI070558 | — | — |
| AI070944 | Foxq1 | Forkhead box Q1 |
| AI070976 | Lrp4 | low density lipoprotein receptor-related protein 4 |
| AI070991 | — | Transcribed locus |
| AI071148 | Tns | Tensin |
| AI071227 | RGD1563603_predicted | similar to DNA repair protein RAD51 homolog 1 (predicted) |
| AI071253 | — | Transcribed locus |
| AI071395 | — | Transcribed locus |
| AI071569 | Il7r_predicted | interleukin 7 receptor (predicted) |
| AI071649 | — | Transcribed locus |
| AI071674 | — | Transcribed locus |
| AI071686 | — | Transcribed locus |
| AI071722 | Mrps18c_predicted | mitochondrial ribosomal protein S18C (predicted) |
| AI072042 | — | — |
| AI072252 | RGD1565169_predicted | similar to apolipoprotein B48 receptor (predicted) |
| AI072663 | — | Transcribed locus |
| AI072892 | Frzb | frizzled-related protein |
| AI073001 | — | Transcribed locus |
| AI073064 | — | Transcribed locus |
| AI073219 | — | Transcribed locus |
| AI073272 | Tor1b | torsin family 1, member B |
| AI100827 | — | Transcribed locus |
| AI101388 | Ga1NAc4S6ST | N-acetylgalactosamine 4-sulfate 6-O-sulfotransferase |
| AI101416 | Cpm_predicted | carboxypeptidase M (predicted) |
| AI101440 | Usp38_predicted | ubiquitin specific protease 38 (predicted) |
| AI101583 | — | Transcribed locus, strongly similar to NP_071858.2 transient |
| | | receptor potential cation channel, subfamily V, member 6 |
| | | [Mus musculus] |
| AI101659 | — | — |
| AI101886 | Hnrph3_predicted | heterogeneous nuclear ribonucleoprotein H3 (2H9) |
| | | (predicted) |
| AI101952 | MGC125215 | multiple coiled-coil GABABR1-binding protein |
| AI102061 | Gria3 | glutamate receptor, ionotropic, AMPA3 (alpha 3) |
| AI102073 | Mal2 | mal, T-cell differentiation protein 2 |
| AI102190 | — | Transcribed locus |
| AI102248 | Plxnd1_predicted | plexin D1 (predicted) |
| AI102482 | RGD1561521_predicted | similar to 1110014F24Rik protein (predicted) |
| AI102517 | — | Transcribed locus |
| AI102519 | Tyrobp | Tyro protein tyrosine kinase binding protein |
| AI102530 | Nab2 | Ngfi-A binding protein 2 |
| AI102627 | LOC681849 | similar to Protein C6orf142 homolog |
| AI102732 | MGC109340 | similar to Microsomal signal peptidase 23 kDa subunit |
| | | (SPase 22 kDa subunit) (SPC22/23) |
| AI102760 | — | — |
| AI102790 | Bcat1 | branched chain aminotransferase 1, cytosolic |
| AI102833 | — | Transcribed locus |
| AI102838 | Ivd | isovaleryl coenzyme A dehydrogenase |
| AI102906 | — | Transcribed locus |
| AI103040 | — | Transcribed locus |
| AI103218 | LOC688228 | similar to Myosin light polypeptide 4 (Myosin light chain 1, |
| | | atrial isoform) |
| AI103408 | — | Transcribed locus |
| AI103918 | Hdac7a | histone deacetylase 7A |
| AI103939 | LOC363091 | similar to hypothetical protein FLJ30973 |
| AI104238 | Gzma | granzyme A |
| AI104326 | — | — |
| AI104533 | Ttn | titin |
| AI104913 | Tmod1 | tropomodulin 1 |
| AI104922 | — | Transcribed locus |
| AI105042 | Cabc1 | chaperone, ABC1 activity of bc1 complex like (S. pombe) |
| AI105117 | Ubtf | upstream binding transcription factor, RNA polymerase I |
| AI105369 | LOC691455 | similar to calmodulin-like 4 |
| AI111965 | — | Transcribed locus |
| AI112158 | B3gnt1_predicted | UDP-GlcNAc: betaGal beta-1,3-N- |
| | | acetylglucosaminyltransferase 1 (predicted) |
| AI112986 | Josd3 | Josephin domain containing 3 |
| AI136136 | LOC689364 | Similar to chromatin modifying protein 1B |
| AI136314 | RGD1311086 | similar to RIKEN cDNA 2610029K21 |
| AI136525 | Degs2 | degenerative spermatocyte homolog 2 (Drosophila), lipid |
| | | desaturase |
| AI136555 | Cipar1 | castration induced prostatic apoptosis-related protein 1 |
| AI137045 | — | Transcribed locus |
| AI137113 | Tmed5 | transmembrane emp24 protein transport domain containing 5 |
| AI137137 | Lck | lymphocyte protein tyrosine kinase |
| AI137157 | LOC500592 | similar to tumor necrosis factor receptor superfamily, |
| | | member 25 |
| AI137163 | Il16 | interleukin 16 |
| AI137506 | LOC362774 | similar to serine proteinase inhibitor A11 gene |
| | | (SERPINA11) |
| AI137602 | — | CDNA clone IMAGE: 7383152 |
| AI137640 | Cldn1 | claudin 1 |
| AI137672 | Cd69 | CD69 antigen |
| AI137791 | — | Transcribed locus |
| AI137898 | LOC685742 | Hypothetical protein LOC685742 |
| AI137931 | Itga6 | integrin, alpha 6 |
| AI144822 | Rcor1_predicted | REST corepressor 1 (predicted) |
| AI144946 | Dynlrb2_predicted | dynein light chain roadblock-type 2 (predicted) |
| AI144948 | — | Transcribed locus |
| AI145081 | Mcm4 | minichromosome maintenance deficient 4 homolog (S. cerevisiae) |
| AI145398 | — | Transcribed locus |
| AI145497 | — | Transcribed locus |
| AI145562 | RGD1310384_predicted | hypothetical LOC289530 (predicted) |
| AI145951 | — | Transcribed locus |
| AI146037 | Map3k7_predicted | Mitogen activated protein kinase kinase kinase 7 (predicted) |
| AI146108 | — | Transcribed locus |
| AI146147 | RGD1311723_predicted | similar to KIAA1731 protein (predicted) |
| AI169104 | Cxcl4 | chemokine (C-X-C motif) ligand 4 |
| AI169367 | RGD1309676 | similar to RIKEN cDNA 5730469M10 |
| AI169601 | Tnfrsf14 | tumor necrosis factor receptor superfamily, member 14 |
| | | (herpesvirus entry mediator) |
| AI170002 | LOC689134 | similar to Protein transport protein SEC61 gamma subunit |
| AI170193 | Dscr1 | Down syndrome critical region homolog 1 (human) |
| AI170356 | — | — |
| AI170362 | Nfkb2 | nuclear factor of kappa light polypeptide gene enhancer in |
| | | B-cells 2, p49/p100 |
| AI170387 | Cxc19 | chemokine (C-X-C motif) ligand 9 |
| AI170535 | Tp53i11_predicted | tumor protein p53 inducible protein 11 (predicted) |
| AI170541 | — | Transcribed locus |
| AI170755 | LOC682245 | similar to Acylphosphatase, muscle type isozyme |
| | | (Acylphosphate phosphohydrolase) |
| AI170987 | — | Transcribed locus |
| AI171093 | Prkcq | protein kinase C, theta |
| AI171288 | RGD1306739_predicted | similar to RIKEN cDNA 1700040L02 (predicted) |
| AI171327 | RGD1563438_predicted | similar to novel protein of unknown function (DUF423) |
| | | family member (predicted) |
| AI171553 | RGD1307396_predicted | similar to RIKEN cDNA 6330406115 (predicted) |
| AI171653 | — | Transcribed locus, moderately similar to XP_342453.2 |
| | | PREDICTED: titin [Rattus norvegicus] |
| AI171654 | Trp53inp2 | tumor protein p53 inducible nuclear protein 2 |
| AI171856 | — | Transcribed locus |
| AI171994 | RGD1306911_predicted | similar to CG10671-like (predicted) |
| AI172033 | Rere | arginine-glutamic acid dipeptide (RE) repeats |
| AI172110 | — | — |
| AI172141 | Ank1 | ankyrin 1, erythroid |
| AI172263 | Rnpc1_predicted | RNA-binding region (RNP1, RRM) containing 1 (predicted) |
| AI175346 | — | Transcribed locus |
| AI175539 | Pvalb | Parvalbumin |
| AI175668 | — | — |
| AI175728 | — | — |
| AI175732 | Vegfa | Vascular endothelial growth factor A |
| AI175779 | — | Transcribed locus, weakly similar to XP_984565.1 |
| | | PREDICTED: hypothetical protein LOC66961 [Mus |
| | | musculus] |
| AI175908 | — | Transcribed locus |
| AI176129 | LOC686611 | similar to Protein FAM60A (Tera protein) |
| AI176172 | — | Transcribed locus |
| AI176327 | RGD1562408_predicted | similar to SH2 domain protein 1A (Signaling lymphocyte |
| | | activation molecule-associated protein) (predicted) |
| AI176393 | Col4A1 | procollagen, type IV, alpha 1 |
| AI176540 | RGD1307844_predicted | Similar to 106 kDa O-GlcNAc transferase-interacting protein |
| | | (predicted) |
| AI176565 | Acss1_predicted | acyl-CoA synthetase short-chain family member 1 |
| | | (predicted) |
| AI176646 | — | Transcribed locus |
| AI176677 | — | Transcribed locus |
| AI176732 | Trem2_predicted | triggering receptor expressed on myeloid cells 2 (predicted) |
| AI176755 | Ptpn22_predicted | protein tyrosine phosphatase, non-receptor type 22 |
| | | (lymphoid) (predicted) |
| AI176913 | Pgm2 | phosphoglucomutase 2 |
| AI177083 | Pcqap_predicted | positive cofactor 2, multiprotein complex, glutamine/Q-rich- |
| | | associated protein (predicted) |
| AI177099 | Prss23 | protease, serine, 23 |
| AI177373 | Cr2_predicted | Complement receptor 2 (predicted) |
| AI177403 | LOC497796 /// | Ly49 inhibitory receptor 9 /// immunoreceptor Ly49si1 /// |
| | LOC502907 /// | immunoreceptor Ly49si2 /// immunoreceptor Ly49si3 /// |
| | LOC690045 /// | Ly49 inhibitory receptor 5 /// hypothetical protein |
| | LOC690097 /// Ly49i5 | LOC497796 /// similar to immunoreceptor Ly49si3 |
| | /// Ly49i9 /// Ly49si1 /// | (predicted) /// similar to immunoreceptor Ly49si1 /// similar |
| | Ly49si2 /// Ly49si3 /// | to immunoreceptor Ly49si3 |
| | RGD1561306_predicted | |
| | /// | |
| | RGD1563110_predicted | |
| AI177484 | Rps24 | ribosomal protein S24 |
| AI177589 | Sorl1 | sortilin-related receptor, LDLR class A repeats-containing |
| AI177645 | RGD1310168_predicted | similar to cDNA sequence BC032204 (predicted) |
| AI177743 | — | Transcribed locus, strongly similar to XP_573486.1 |
| | | PREDICTED: hypothetical protein XP_573486 [Rattus |
| | | norvegicus] |
| AI177746 | — | Transcribed locus |
| AI177934 | LOC474169 | pre-eosinophil-associated ribonuclease-2 |
| AI178160 | Myo9b | myosin IXb |
| AI178214 | Rassf3_predicted | Ras association (RalGDS/AF-6) domain family 3 (predicted) |
| AI178222 | — | Transcribed locus |
| AI178243 | Donson | downstream neighbor of SON |
| AI178339 | Cytor4 | similar to cytokine receptor related protein 4 |
| AI178384 | Klhl6_predicted | kelch-like 6 (Drosophila) (predicted) |
| AI178618 | — | — |
| AI178689 | LOC691898 | hypothetical protein LOC691898 |
| AI178693 | RGD1305754_predicted | similar to 3110080A02Rik protein (predicted) |
| AI178784 | RGD1310174_predicted | hypothetical LOC298504 (predicted) |
| AI178808 | Il2rg | interleukin 2 receptor, gamma (severe combined |
| | | immunodeficiency) |
| AI178914 | AA926063 | AA926063gene |
| AI179227 | Cybasc3 | cytochrome b, ascorbate dependent 3 |
| AI179334 | Fasn | fatty acid synthase |
| AI179609 | RGD1561062_predicted | similar to RIKEN cDNA 5730593F17 (predicted) |
| AI179665 | RGD1305755 | Similar to RIKEN cDNA 5033406L14 |
| AI179886 | RGD1310352 | similar to HTGN29 protein; keratinocytes associated |
| | | transmembrane protein 2 |
| AI179988 | Enc1 | ectodermal-neural cortex 1 |
| AI180300 | LOC499331 | similar to hypothetical protein D030056L22 |
| AI180352 | Herc1_predicted | hect (homologous to the E6-AP (UBE3A) carboxyl |
| | | terminus) domain and RCC1 (CHC1)-like domain (RLD) 1 |
| | | (predicted) |
| AI180370 | RGD1304592_predicted | similar to KIAA0528 protein (predicted) |
| AI227638 | Satb1 | special AT-rich sequence binding protein 1 |
| AI227800 | Kifap3_predicted | kinesin-associated protein 3 (predicted) |
| AI227930 | Wdr48_predicted | WD repeat domain 48 (predicted) |
| AI228039 | — | — |
| AI228076 | — | Transcribed locus |
| AI228417 | Sema4a | sema domain, immunoglobulin domain (Ig), transmembrane |
| | | domain (TM) and short cytoplasmic domain, (semaphorin) |
| | | 4A |
| AI228630 | Wdr23 | WD repeat domain 23 |
| AI229167 | Hip2_predicted | huntingtin interacting protein 2 (predicted) |
| AI229409 | — | Transcribed locus |
| AI229612 | LOC686980 | similar to arsenate resistance protein 2 |
| AI229699 | RGD1561665_predicted | similar to spatial-delta (predicted) |
| AI230334 | Tceal8 | transcription elongation factor A (SII)-like 8 |
| AI230466 | — | — |
| AI230554 | — | Transcribed locus |
| AI230591 | RGD1565540_predicted | similar to ctla-2-beta protein (141 AA) (predicted) |
| AI230669 | — | Transcribed locus |
| AI230766 | RGD1310423 | similar to hypothetical protein FLJ31737 |
| AI231223 | Cldnd1 | Claudin domain containing 1 |
| AI231438 | Cndp1 | carnosine dipeptidase 1 (metallopeptidase M20 family) |
| AI231787 | — | Transcribed locus |
| AI231826 | — | Transcribed locus |
| AI232289 | RGD1307812 | similar to transcription factor (p38 interacting protein) |
| AI232407 | — | — |
| AI232414 | Pxmp4 | peroxisomal membrane protein 4 |
| AI232716 | LOC368066 | similar to indolethylamine N-methyltransferase |
| AI233143 | Rab2l | RAB2, member RAS oncogene family-like |
| AI233243 | Rhot2 | ras homolog gene family, member T2 |
| AI233361 | — | — |
| AI233902 | — | Transcribed locus |
| AI234022 | Gtf3c1 | general transcription factor III C 1 |
| AI234095 | Smap1l | stromal membrane-associated protein 1-like |
| AI234943 | Sf3b1 | Splicing factor 3b, subunit 1 |
| AI235220 | LOC498178 /// | similar to SET domain-containing protein |
| | LOC687538 /// | |
| | LOC689820 | |
| AI235414 | — | — |
| AI235431 | Tnpo2_predicted | transportin 2 (importin 3, karyopherin beta 2b) (predicted) |
| AI235468 | Dst_predicted | dystonin (predicted) |
| AI235474 | Prkwnk1 | Protein kinase, lysine deficient 1 |
| AI235528 | — | Transcribed locus |
| AI235774 | Mcm3ap_predicted | minichromosome maintenance deficient 3 (S. cerevisiae) |
| | | associated protein (predicted) |
| AI236141 | LOC686590 | similar to IQ motif and Sec7 domain 1 |
| AI236229 | LOC681858 /// | similar to RNA binding motif protein 24 |
| | LOC690139 | |
| AI236455 | Anxa1 | annexin A1 |
| AI236521 | LOC691849 | hypothetical protein LOC691849 |
| AI237082 | Hivep1 | human immunodeficiency virus type I enhancer binding |
| | | protein 1 |
| AI237098 | Uxt | ubiquitously expressed transcript |
| AI237143 | Rexo4 | REX4, RNA exonuclease 4 homolog (S. cerevisiae) |
| AI237192 | Fut4 | fucosyltransferase 4 |
| AI237227 | — | Transcribed locus |
| AI237640 | LOC683446 /// | similar to killer activatory receptor-like protein p91D |
| | RGD1562625_predicted | (predicted) /// similar to paired-Ig-like receptor A11 |
| AI237698 | — | Transcribed locus |
| AI385291 | Blk | B lymphoid kinase |
| AI406275 | Cbx7 | chromobox homolog 7 |
| AI406386 | Lmo4 | LIM domain only 4 |
| AI406565 | Smpx | small muscle protein, X-linked |
| AI406660 | — | — |
| AI406775 | — | Transcribed locus |
| AI406854 | — | Transcribed locus |
| AI406967 | — | Transcribed locus |
| AI407107 | Rnf44 | Ring finger protein 44 |
| AI407163 | — | Transcribed locus |
| AI407239 | — | Transcribed locus |
| AI407261 | Ubp1_predicted | upstream binding protein 1 (predicted) |
| AI407418 | RGD1562596_predicted | similar to mKIAA0159 protein (predicted) |
| AI407458 | Aldh6a1 | aldehyde dehydrogenase family 6, subfamily A1 |
| AI407487 | — | Transcribed locus |
| AI407688 | Ubtf | upstream binding transcription factor, RNA polymerase I |
| AI407827 | Pik3cd_predicted | phosphatidylinositol 3-kinase catalytic delta polypeptide |
| | | (predicted) |
| AI407985 | RGD1561797_predicted | RGD1561797 (predicted) |
| AI407992 | — | Transcribed locus |
| AI408081 | RGD1307597_predicted | similar to mKIAA0317 protein (predicted) |
| AI408164 | RGD1306176 | similar to FCRL |
| AI408286 | Ms4a7_predicted | membrane-spanning 4-domains, subfamily A, member 7 |
| | | (predicted) |
| AI408289 | Cd22_predicted | CD22 antigen (predicted) |
| AI408306 | Slc44a2_predicted | Solute carrier family 44, member 2 (predicted) |
| AI408343 | RGD1562552_predicted | similar to hypothetical protein LOC340061 (predicted) |
| AI408425 | Tlr7_predicted | toll-like receptor 7 (predicted) |
| AI408440 | MGC108823 /// | similar to interferon-inducible GTPase /// similar to |
| | RGD1559715_predicted | MGC108823 protein (predicted) |
| AI408443 | Psrc2 | proline/serine-rich coiled-coil 2 |
| AI408597 | — | Transcribed locus |
| AI408598 | Git2 | G protein-coupled receptor kinase-interactor 2 |
| AI408613 | — | — |
| AI408674 | LOC688261 | similar to 5(3)-deoxyribonucleotidase, cytosolic type |
| | | (Cytosolic 5,3-pyrimidine nucleotidase) (Deoxy-5- |
| | | nucleotidase 1) (dNT-1) |
| AI408827 | LOC300191 | similar to RIKEN cDNA 4930570C03 |
| AI408833 | Hnrpa1 | heterogeneous nuclear ribonucleoprotein A1 |
| AI408839 | Rwdd2_predicted | RWD domain containing 2 (predicted) |
| AI408948 | Ca2 | carbonic anhydrase 2 |
| AI409142 | LOC679811 | similar to RIKEN cDNA D930015E06 |
| AI409173 | — | Transcribed locus |
| AI409180 | Mrpl52_predicted | mitochondrial ribosomal protein L52 (predicted) |
| AI409262 | Rhebl1 | Ras homolog enriched in brain like 1 |
| AI409460 | RGD1561781_predicted | similar to RIKEN cDNA 2600017H02 (predicted) |
| AI409545 | LOC689938 | similar to NADH dehydrogenase (ubiquinone) 1, |
| | | subcomplex unknown, 1 |
| AI409634 | Best5 | Best5 protein |
| AI409635 | — | Transcribed locus |
| AI409904 | — | Transcribed locus |
| AI410068 | Fcer2a | Fc receptor, IgE, low affinity II, alpha polypeptide |
| AI410861 | — | Transcribed locus |
| AI411149 | — | Transcribed locus |
| AI411185 | — | Transcribed locus |
| AI411227 | RGD1563485_predicted | similar to mKIAA0534 protein (predicted) |
| AI411326 | LOC303057 /// Slu7 | similar to step II splicing factor SLU7; DNA segment, Chr |
| | | 11, ERATO Doi 730, expressed; DNA segment, Chr 3, |
| | | Brigham & Womens Genetics 0878 expressed /// step II |
| | | splicing factor SLU7 (S. cerevisiae) |
| AI411413 | LOC685778 /// Pdha1 | pyruvate dehydrogenase E1 alpha 1 /// pyruvate |
| | | dehydrogenase E1 alpha 1 pseudogene |
| AI411425 | Esd | esterase D/formylglutathione hydrolase |
| AI411436 | Gadd45gip1 | growth arrest and DNA-damage-inducible, gamma |
| | | interacting protein 1 |
| AI411527 | Wfdc2 | WAP four-disulfide core domain 2 |
| AI411563 | Fgd2_predicted | FYVE, RhoGEF and PH domain containing 2 (predicted) |
| AI411693 | LOC299458 /// | similar to immunoglobulin heavy chain 6 (Igh-6) /// similar |
| | LOC366747 /// | to Ig H-chain V-region precursor /// similar to Ig heavy chain |
| | LOC678701 /// | V region MC101 precursor /// hypothetical protein |
| | RGD1359202 | LOC678701 |
| AI411747 | Art2b | ADP-ribosyltransferase 2b |
| AI411753 | — | Transcribed locus |
| AI411774 | LOC680611 /// | similar to B-cell leukemia/lymphoma 3 |
| | LOC686871 | |
| AI411809 | — | Transcribed locus |
| AI412136 | Tmprss2 | transmembrane protease, serine 2 |
| AI412155 | Cry1 | cryptochrome 1 (photolyase-like) |
| AI412189 | Igha_mapped /// | immunoglobulin heavy chain (alpha polypeptide) (mapped) |
| | LO366772 | /// similar to immunoglobulin heavy chain |
| AI412244 | Tbc1d14 | TBC1 domain family, member 14 |
| AI412322 | Acat2 | acetyl-Coenzyme A acetyltransferase 2 |
| AI412781 | LOC680281 /// | similar to RIKEN cDNA 4930555G01 (predicted) /// similar |
| | LOC685769 /// | to Discs large homolog 5 (Placenta and prostate DLG) |
| | LOC690576 /// | (Discs large protein P-dlg) /// hypothetical protein |
| | LOC691414 /// | LOC691414 |
| | RGD1563862_predicted | |
| AI412813 | — | Transcribed locus |
| AI412866 | RGD1306100_predicted | similar to RRP22 (predicted) |
| AI412969 | RGD1307215 | similar to protein phosphatase 1, regulatory (inhibitory) |
| | | subunit 1C; thymocyte ARPP; DNA segment, Chr 9, |
| | | Brigham & Womens Genetics 1012 expressed |
| AI453967 | RGD1306176 | similar to FCRL |
| AI454224 | — | Transcribed locus |
| AI454790 | — | Transcribed locus |
| AI454905 | Tusc3 | tumor suppressor candidate 3 |
| AI454911 | Scye1 | small inducible cytokine subfamily E, member 1 |
| AI500952 | Sla | Src-like adaptor |
| AI501127 | — | — |
| AI501187 | Dsg1c_predicted /// | desmoglein 1 gamma (predicted) /// similar to Desmoglein-1 |
| | LOC679010 | alpha precursor (Dsg1-alpha) (Desmoglein-1) (Desmosomal |
| | | glycoprotein I) (DG1) (DGI) |
| AI501709 | C1qtnf7_predicted | C1q and tumor necrosis factor related protein 7 (predicted) |
| AI501853 | Ptplad2_predicted | protein tyrosine phosphatase-like A domain containing 2 |
| | | (predicted) |
| AI502114 | Abca1 | ATP-binding cassette, sub-family A (ABC1), member 1 |
| AI502349 | — | Transcribed locus |
| AI502443 | LOC680726 | Similar to RNA binding motif, single stranded interacting |
| | | protein 3 isoform 1 |
| AI502459 | — | — |
| AI502837 | — | Transcribed locus |
| AI502930 | Eif3s8 | eukaryotic translation initiation factor 3, subunit 8, 110 kDa |
| AI511408 | — | — |
| AI535104 | Plxnc1_predicted | plexin C1 (predicted) |
| AI535113 | — | Transcribed locus |
| AI547876 | — | Transcribed locus |
| AI547937 | — | Transcribed locus |
| AI548117 | — | — |
| AI548667 | — | Transcribed locus |
| AI548779 | RGD1562251_predicted | similar to DNA segment, Chr 11, ERATO Doi 461, |
| | | expressed (predicted) |
| AI548897 | Soat1 | Sterol O-acyltransferase 1 |
| AI548940 | RGD1561963_predicted | similar to Dedicator of cytokinesis protein 10 (Protein |
| | | zizimin 3) (predicted) |
| AI549009 | Prkag3_predicted | protein kinase, AMP-activated, gamma 3 non-catatlytic |
| | | subunit (predicted) |
| AI549199 | Ptger4 | Prostaglandin E receptor 4 (subtype EP4) |
| AI549209 | — | Transcribed locus |
| AI549335 | Dock11 | dedicator of cytokinesis 11 |
| AI549393 | — | Transcribed locus |
| AI549450 | Cd209b | CD209b antigen |
| AI549469 | Ptk7_predicted | PTK7 protein tyrosine kinase 7 (predicted) |
| AI555257 | RGD1562305_predicted | similar to suprabasal-specific protein suprabasin (predicted) |
| AI555336 | Mllt6_predicted | myeloid/lymphoid or mixed lineage-leukemia translocation |
| | | to 6 homolog (Drosophila) (predicted) |
| AI555447 | RGD1565975_predicted | RGD1565975 (predicted) |
| AI555526 | — | Transcribed locus |
| AI555855 | — | Transcribed locus |
| AI556056 | Cr2_predicted | complement receptor 2 (predicted) |
| AI556235 | RGD1310061 | similar to hypothetical protein FLJ10154 |
| AI556333 | RGD1564257_predicted | similar to hypothetical protein FLJ32825 (predicted) |
| AI556638 | — | — |
| AI556940 | Tmc4 | transmembrane channel-like gene family 4 |
| AI556957 | Aff4_predicted | AF4/FMR2 family, member 4 (predicted) |
| AI575072 | Strn4_predicted | striatin, calmodulin binding protein 4 (predicted) |
| AI575255 | — | Transcribed locus |
| AI575264 | Ddx58_predicted | DEAD (Asp-Glu-Ala-Asp) box polypeptide 58 (predicted) |
| AI575442 | Ryr1 | ryanodine receptor 1, skeletal muscle |
| AI575608 | LOC297481 | similar to eukaryotic translation initiation factor 4E member 3 |
| AI576002 | — | Transcribed locus |
| AI576009 | Higd1b_predicted | HIG1 domain family, member 1B (predicted) |
| AI576184 | — | Transcribed locus |
| AI576518 | — | Transcribed locus |
| AI577508 | LOC684425 | similar to Adenylosuccinate synthetase isozyme 1 |
| | | (Adenylosuccinate synthetase, muscle isozyme) (IMP-- |
| | | aspartate ligase 1) (AdSS 1) (AMPSase 1) |
| AI577567 | Rasal2_predicted | RAS protein activator like 2 (predicted) |
| AI577848 | Trio | Triple functional domain (PTPRF interacting) |
| AI578087 | Tm4sf1_predicted | transmembrane 4 superfamily member 1 (predicted) |
| AI578098 | LOC683220 /// | similar to CD209 antigen |
| | LOC688750 | |
| AI578268 | Ssh3 | slingshot homolog 3 (Drosophila) |
| AI578566 | Ubn1_predicted | ubinuclein 1 (predicted) |
| AI579376 | RGD1309220 | similar to RIKEN cDNA 4632411B12 |
| AI598307 | Slc45a3_predicted | solute carrier family 45, member 3 (predicted) |
| AI598971 | Perp_predicted | PERP, TP53 apoptosis effector (predicted) |
| AI599048 | — | Transcribed locus |
| AI599133 | LOC363060 | similar to RIKEN cDNA 1600029D21 |
| AI599232 | — | Transcribed locus |
| AI599423 | Gadd45g | growth arrest and DNA-damage-inducible 45 gamma |
| AI599463 | Dguok_predicted | deoxyguanosine kinase (predicted) |
| AI599794 | — | Transcribed locus, moderately similar to XP_580071.1 |
| | | PREDICTED: hypothetical protein XP_580071 [Rattus |
| | | norvegicus] |
| AI600030 | RGD1306494_predicted | similar to Expressed sequence AW146242 (predicted) |
| AI600061 | Add1 | adducin 1 (alpha) |
| AI638971 | Ocln | Occluding |
| AI638986 | Neb_predicted | nebulin (predicted) |
| AI639108 | — | — |
| AI639117 | Cfb | complement factor B |
| AI639241 | LOC684318 /// | ubiquitin specific protease 12 (predicted) /// similar to |
| | RGD1561481_predicted | ubiquitin specific protease 12 (predicted) /// similar to |
| | /// Usp12_predicted | ubiquitin specific protease 12 |
| AI639268 | Dhrs7c_predicted | dehydrogenase/reductase (SDR family) member 7C |
| | | (predicted) |
| AI639331 | — | — |
| AI639401 | Thh_predicted | Trichohyalin (predicted) |
| AI639412 | LOC306805 | similar to asporin precursor |
| AI639521 | — | Transcribed locus |
| AI706508 | Pprc1_predicted | peroxisome proliferative activated receptor, gamma, |
| | | coactivator-related 1 (predicted) |
| AI706673 | Wasl | Wiskott-Aldrich syndrome-like (human) |
| AI709455 | — | Transcribed locus |
| AI709793 | — | Transcribed locus |
| AI710051 | Sc5d | sterol-C5-desaturase (fungal ERG3, delta-5-desaturase) |
| | | homolog (S. cerevisae) |
| AI710604 | — | Transcribed locus |
| AI710682 | Tnnc1 | troponin C type 1 (slow) |
| AI712791 | Slc6a17 | solute carrier family 6 (neurotransmitter transporter), |
| | | member 17 |
| AI713204 | Mgll | Monoglyceride lipase |
| AI713965 | Irx3_predicted | Iroquois related homeobox 3 (Drosophila) (predicted) |
| AI713966 | Igfbp3 | insulin-like growth factor binding protein 3 |
| AI714002 | Mki67_predicted | antigen identified by monoclonal antibody Ki-67 (predicted) |
| AI715202 | RT1-Bb | RT1 class II, locus Bb |
| AI715411 | MGC108778 | Similar to RIKEN cDNA 1810057C19 |
| AI715999 | LOC688864 | similar to transmembrane protein 61 |
| AI716125 | C2 | complement component 2 |
| AI716456 | Tiam1 | T-cell lymphoma invasion and metastasis 1 |
| AI716887 | RGD1561064_predicted | similar to myozenin 1 (predicted) |
| AI716969 | RGD1305774_predicted | hypothetical LOC287353 (predicted) |
| AI716980 | Capn9 | calpain 9 (nCL-4) |
| AI716987 | Dsg1c_predicted /// | desmoglein 1 gamma (predicted) /// similar to Desmoglein-1 |
| | LOC679010 | alpha precursor (Dsg1-alpha) (Desmoglein-1) (Desmosomal |
| | | glycoprotein I) (DG1) (DGI) |
| AI717047 | Cml5 | camello-like 5 |
| AI717081 | — | Transcribed locus |
| AI717163 | — | Transcribed locus |
| AI717186 | RGD1306300_predicted | similar to CG9643-PA (predicted) |
| AI717476 | Pygm | muscle glycogen phosphorylase |
| AI717644 | RGD1563764_predicted | similar to PHD finger protein 14 isoform 1 (predicted) |
| AI717736 | LOC503164 | hypothetical protein LOC503164 |
| AI764088 | Myo1f_predicted | myosin IF (predicted) |
| AI948410 | — | — |
| AJ011116 | Nos3 | nitric oxide synthase 3, endothelial cell |
| AJ011811 | Cldn7 | claudin 7 |
| AJ243304 | Trdn | triadin |
| AJ243338 | RT1-CE5 | RT1 class I, CE5 |
| AJ243973 | RT1-S3 | RT1 class Ib, locus S3 |
| AJ243974 | RT1-S3 | RT1 class Ib, locus S3 |
| AJ245707 | Phyh2 | phytanoyl-CoA 2-hydroxylase 2 |
| AW141642 | RGD1306067 | similar to chromosome 20 open reading frame 6 |
| AW142500 | Sept6_predicted | septin 6 (predicted) |
| AW142773 | — | Transcribed locus |
| AW142796 | RGD1566282_predicted | similar to RIKEN cDNA D330045A20 (predicted) |
| AW142978 | Traf3ip3 | TRAF3 interacting protein 3 |
| AW143154 | Mkl1_predicted | megakaryoblastic leukemia (translocation) 1 (predicted) |
| AW144132 | — | Transcribed locus |
| AW144193 | — | Transcribed locus |
| AW144216 | Enpep | glutamyl aminopeptidase |
| AW144509 | Dsp | desmoplakin |
| AW144823 | Sorl1 | sortilin-related receptor, LDLR class A repeats-containing |
| AW251280 | — | — |
| AW251324 | LOC680308 | similar to Bifunctional methylenetetrahydrofolate |
| | | dehydrogenase/cyclohydrolase, mitochondrial precursor |
| AW251647 | — | Transcribed locus |
| AW251860 | Tcf7_predicted | transcription factor 7, T-cell specific (predicted) |
| AW252115 | RGD1562105_predicted | similar to Rho-GTPase-activating protein 25 (predicted) |
| AW252232 | — | Transcribed locus |
| AW252401 | LOC362564 | hypothetical LOC362564 |
| AW252428 | — | Transcribed locus |
| AW252511 | Ddx24 | DEAD (Asp-Glu-Ala-Asp) box polypeptide 24 |
| AW254450 | RGD1310433_predicted | similar to mKIAAI757 protein (predicted) |
| AW433901 | Celsr1 | cadherin EGF LAG seven-pass G-type receptor 1 |
| AW433947 | Tnfrsf5 | tumor necrosis factor receptor superfamily, member 5 |
| AW434166 | Nicn1 | nicolin 1 |
| AW434961 | Hfe2 | hemochromatosis type 2 (juvenile) homolog (human) |
| AW434972 | Rbm25_predicted /// | RNA binding motif protein 25 (predicted) /// similar to RNA |
| | RGD1565486_predicted | binding motif protein 25 (predicted) |
| AW435211 | — | Transcribed locus |
| AW435343 | Tm4sf1_predicted | transmembrane 4 superfamily member 1 (predicted) |
| AW435407 | — | Transcribed locus |
| AW435415 | — | Transcribed locus |
| AW435479 | RGD1561211_predicted | similar to Amyloid beta (A4) precursor-like protein 1 |
| | | (predicted) |
| AW522166 | — | Transcribed locus |
| AW522341 | — | Transcribed locus |
| AW522357 | — | Transcribed locus |
| AW522661 | Lnx1_predicted | ligand of numb-protein X 1 (predicted) |
| AW522944 | Ndph_predicted | Norrie disease homolog (human) (predicted) |
| AW523423 | — | — |
| AW523620 | — | Transcribed locus, moderately similar to XP_418639.1 |
| | | PREDICTED: similar to metalloprotease-disintegrin [Gallus |
| | | gallus] |
| AW523955 | — | Transcribed locus |
| AW523958 | — | Transcribed locus |
| AW524037 | — | Transcribed locus |
| AW524146 | Pcdh20_predicted | protocadherin 20 (predicted) |
| AW524173 | — | Transcribed locus |
| AW524239 | — | Transcribed locus |
| AW524359 | — | Transcribed locus |
| AW524694 | LOC500591 | similar to calmodulin-binding transcription activator 1 |
| AW525235 | Suv420h2_predicted | suppressor of variegation 4-20 homolog 2 (Drosophila) |
| | | (predicted) |
| AW525288 | — | Transcribed locus |
| AW525315 | — | — |
| AW525662 | Cdc27 | cell division cycle 27 homolog (S. cerevisiae) |
| AW525862 | — | — |
| AW526014 | — | Transcribed locus |
| AW526015 | RGD1564241_predicted | similar to zinc finger protein 426 (predicted) |
| AW526072 | — | Transcribed locus |
| AW526087 | LOC680047 /// | similar to protocadherin beta 9 |
| | LOC683183 | |
| AW526268 | RGD1561817_predicted | Similar to Traf2 and NCK interacting kinase, splice variant 4 |
| | | (predicted) |
| AW526551 | RGD1307724_predicted | similar to NCAG1 (predicted) |
| AW526631 | Sorl1 | sortilin-related receptor, LDLR class A repeats-containing |
| AW526714 | — | Transcribed locus |
| AW526982 | Tlr2 | toll-like receptor 2 |
| AW527159 | — | Transcribed locus |
| AW527270 | RGD1309360 | hypothetical LOC294715 |
| AW527313 | Rdx | radixin |
| AW527403 | Sh3yl1_predicted | Sh3 domain YSC-like 1 (predicted) |
| AW528106 | — | Transcribed locus |
| AW528353 | — | Transcribed locus, strongly similar to XP_579759.1 |
| | | PREDICTED: hypothetical protein XP_579759 [Rattus |
| | | norvegicus] |
| AW528448 | — | Transcribed locus |
| AW528482 | — | Transcribed locus |
| AW528765 | — | Transcribed locus, strongly similar to XP_341205.2 |
| | | PREDICTED: similar to LNXp80 [Rattus norvegicus] |
| AW528853 | RGD1309403_predicted | similar to hypothetical protein FLJ12661 (predicted) |
| AW529415 | — | Transcribed locus |
| AW529736 | Wfdc5_predicted | WAP four-disulfide core domain 5 (predicted) |
| AW530415 | — | Transcribed locus |
| AW530423 | — | Transcribed locus |
| AW530507 | — | Transcribed locus |
| AW530769 | Fdft1 | farnesyl diphosphate farnesyl transferase 1 |
| AW530812 | RGD1311224_predicted | similar to fatty acid desaturase 2; linoleoyl-CoA desaturase |
| | | (delta-6-desaturase)-like 2; delta-6 fatty acid desaturase |
| | | (predicted) |
| AW531135 | — | Transcribed locus |
| AW531805 | Ifit3 | interferon-induced protein with tetratricopeptide repeats 3 |
| AW531880 | — | Transcribed locus |
| AW531902 | Zfhx4_predicted | zinc finger homeodomain 4 (predicted) |
| AW532114 | Rasgrp2_predicted | RAS guanyl releasing protein 2 (calcium and DAG- |
| | | regulated) (predicted) |
| AW532165 | Sema3d | sema domain, immunoglobulin domain (Ig), short basic |
| | | domain, secreted, (semaphorin) 3D |
| AW532179 | LOC500449 | similar to SHP2-interacting transmembrane adaptor protein |
| AW532214 | — | Transcribed locus |
| AW532414 | RGD1565549_predicted | similar to polybromo-1 (predicted) |
| AW532525 | Srrm2_predicted | serine/arginine repetitive matrix 2 (predicted) |
| AW532618 | — | Transcribed locus |
| AW532634 | — | Transcribed locus |
| AW533203 | Agpat3_predicted | 1-acylglycerol-3-phosphate O-acyltransferase 3 (predicted) |
| AW533234 | LOC688915 | similar to cardiomyopathy associated 5 |
| AW533569 | Clca2_predicted | chloride channel, calcium activated, family member 2 |
| | | (predicted) |
| AW533848 | Mybpc2_predicted | myosin binding protein C, fast-type (predicted) |
| AW533923 | LOC500015 | similar to mKIAA2005 protein |
| AW533998 | — | Transcribed locus |
| AW534046 | — | — |
| AW534142 | — | Transcribed locus, strongly similar to NP_075544.1 leucine |
| | | zipper- and sterile alpha motif-containing kinase isoform 1 |
| | | [Mus musculus] |
| AW534148 | — | — |
| AW534218 | RGD1310722_predicted | similar to RIKEN cDNA D130059P03 gene (predicted) |
| AW534277 | Atp2b1 | ATPase, Ca++ transporting, plasma membrane 1 |
| AW534554 | — | — |
| AW534561 | — | Transcribed locus |
| AW534728 | Ewsr1 | Ewing sarcoma breakpoint region 1 |
| AW534807 | Elk4_predicted | ELK4, member of ETS oncogene family (predicted) |
| AW534965 | RGD1560358_predicted | similar to cell division cycle and apoptosis regulator 1 |
| | | (predicted) |
| AW535052 | Smc2l1_predicted | SMC2 structural maintenance of chromosomes 2-like 1 |
| | | (yeast) (predicted) |
| AW535067 | Ephb1 | Eph receptor B1 |
| AW535122 | LOC679020 /// | similar to nucleoredoxin |
| | LOC689232 | |
| AW535232 | RGD1561145_predicted | similar to novel protein (predicted) |
| AW535305 | RGD1305145 | similar to CG6796-PA |
| AW915049 | — | Transcribed locus |
| AW915083 | — | Transcribed locus, strongly similar to XP_996556.1 |
| | | PREDICTED: similar to Dual specificity protein |
| | | phosphatase CDC14A (CDC14 cell division cycle 14 |
| | | homolog A) isoform 1 [Mus musculus] |
| AW915152 | Echdc1 | enoyl Coenzyme A hydratase domain containing 1 |
| AW915157 | LOC685462 | similar to EMI domain containing 1 |
| AW915435 | — | — |
| AW915948 | Cd3e_predicted | CD3 antigen, epsilon polypeptide (predicted) |
| AW916093 | — | Transcribed locus |
| AW916957 | — | Transcribed locus, strongly similar to XP_579758.1 |
| | | PREDICTED: hypothetical protein XP_579758 [Rattus |
| | | norvegicus] |
| AW917731 | Tbc1d1_predicted | TBC1 domain family, member 1 (predicted) |
| AW917933 | — | Transcribed locus |
| AW918029 | Gimap1 /// Gimap5 | GTPase, IMAP family member 5 /// GTPase, IMAP family |
| | | member 1 |
| AW918387 | Abca1 | ATP-binding cassette, sub-family A (ABC1), member 1 |
| AW918480 | — | Transcribed locus |
| AW918614 | Spnb2 | spectrin beta 2 |
| AW918981 | — | Transcribed locus |
| AW919180 | Pygm | muscle glycogen phosphorylase |
| AW919569 | MGC156825 | similar to MIR-interacting saposin-like protein precursor |
| | | (Transmembrane protein 4) (Putative secreted protein |
| | | ZSIG9) |
| AW919577 | Tcrb | T-cell receptor beta chain |
| AW920000 | LOC362587 | similar to microfilament and actin filament cross-linker |
| | | protein isoform b |
| AW920026 | LOC364393 /// | PHD finger protein 11 /// similar to RIKEN cDNA |
| | LOC684778 /// Phf11 | 4933417L10 /// hypothetical protein LOC684778 |
| AW920039 | RGD1311584 | similar to stem cell adaptor protein STAP-1 |
| AW920881 | Actr2 /// LOC301861 | ARP2 actin-related protein 2 homolog (yeast) /// similar to |
| | | ARP2 actin-related protein 2 homolog (yeast) (predicted) |
| AW920944 | — | Transcribed locus |
| AW921158 | — | — |
| AW921279 | LOC683839 /// | hypothetical protein LOC683839 /// hypothetical protein |
| | LOC688495 | LOC688495 |
| AW921478 | Ms4a11_predicted | membrane-spanning 4-domains, subfamily A, member 11 |
| | | (predicted) |
| AY082657 | Kcnip1 | Kv channel-interacting protein 1 |
| BE095824 | Ccl6 | chemokine (C-C motif) ligand 6 |
| BE096367 | — | Transcribed locus |
| BE096453 | Ube1l_predicted | ubiquitin-activating enzyme E1-like (predicted) |
| BE096523 | G1p2_predicted | interferon, alpha-inducible protein (clone IFI-15K) |
| | | (predicted) |
| BE096652 | Ly6g6c | lymphocyte antigen 6 complex, locus G6C |
| BE096750 | RGD1305928_predicted | hypothetical LOC300207 (predicted) |
| BE096812 | — | Transcribed locus |
| BE097574 | Kcnab1 | potassium voltage-gated channel, shaker-related subfamily, |
| | | beta member 1 |
| BE097933 | Mitf | microphthalmia-associated transcription factor |
| BE097945 | — | Transcribed locus |
| BE098153 | Slc24a3 | solute carrier family 24 (sodium/potassium/calcium |
| | | exchanger), member 3 |
| BE098186 | Anks1_predicted | ankyrin repeat and SAM domain containing 1 (predicted) |
| BE098532 | LOC303057 | similar to step II splicing factor SLU7; DNA segment, Chr |
| | | 11, ERATO Doi 730, expressed; DNA segment, Chr 3, |
| | | Brigham & Womens Genetics 0878 expressed |
| BE098713 | Igsf3_predicted | immunoglobulin superfamily, member 3 (predicted) |
| BE098726 | — | Transcribed locus |
| BE098732 | Uhrf1 | ubiquitin-like, containing PHD and RING finger domains, 1 |
| BE099038 | — | — |
| BE099050 | Nfib | nuclear factor I/B |
| BE099838 | — | Non-coding RNA expressed in the brain, repeat sequence, |
| | | clone 12 |
| BE100015 | Arhgap27 | Rho GTPase activating protein 27 |
| BE100543 | Ncor1 | nuclear receptor co-repressor 1 |
| BE100811 | Epb4.1l4a_predicted | erythrocyte protein band 4.1-like 4a (predicted) |
| BE101133 | — | — |
| BE102265 | — | — |
| BE102340 | — | — |
| BE102358 | — | Transcribed locus, moderately similar to XP_237998.3 |
| | | PREDICTED: similar to mKIAA0183 protein [Rattus |
| | | norvegicus] |
| BE102861 | — | Transcribed locus |
| BE103057 | — | Transcribed locus |
| BE103561 | Ccnj_predicted | cyclin J (predicted) |
| BE103689 | Zfhx4_predicted | zinc finger homeodomain 4 (predicted) |
| BE103745 | Sort1 | Sortilin 1 |
| BE103975 | — | Transcribed locus, strongly similar to NP_001035488.1 SET |
| | | domain containing 1B [Mus musculus] |
| BE104164 | — | Transcribed locus |
| BE104167 | — | Transcribed locus |
| BE104421 | — | Transcribed locus |
| BE104506 | RGD1307284_predicted | similar to protein kinase, lysine deficient 1; kinase deficient |
| | | protein (predicted) |
| BE104517 | — | Transcribed locus |
| BE104676 | — | Transcribed locus, strongly similar to NP_001029332.1 |
| | | hypothetical protein LOC619476 [Rattus norvegicus] |
| BE104961 | Cenpj_predicted | centromere protein J (predicted) |
| BE105050 | — | Transcribed locus |
| BE105268 | Pcnxl3 | pecanex-like 3 (Drosophila) |
| BE105494 | LOC294762 | similar to poly (ADP-ribose) polymerase family, member 8 |
| BE105498 | — | Transcribed locus |
| BE105565 | Rbaf600 | ZUBR1 |
| BE105705 | LOC688144 /// | similar to ankyrin repeat domain 40 |
| | LOC690586 | |
| BE105855 | Cyb5r2 | cytochrome b5 reductase 2 |
| BE106199 | Rora_predicted | RAR-related orphan receptor alpha (predicted) |
| BE106353 | Tsga2 | testis specific gene A2 |
| BE106791 | — | Transcribed locus |
| BE106891 | — | Transcribed locus |
| BE107024 | — | Transcribed locus |
| BE107173 | — | — |
| BE107282 | Vof16 | ischemia related factor vof-16 |
| BE107346 | Eif5 | eukaryotic translation initiation factor 5 |
| BE107351 | — | Transcribed locus |
| BE107400 | LOC690606 | similar to MANSC domain containing 1 |
| BE107859 | RGD1311595 | similar to KIAA2026 protein |
| BE108006 | Aebp2_predicted | AE binding protein 2 (predicted) |
| BE108131 | Wdfy1 | WD repeat and FYVE domain containing 1 |
| BE108135 | — | Transcribed locus |
| BE108165 | Cep1_predicted | centrosomal protein 1 (predicted) |
| BE108221 | Coro1c_predicted | coronin, actin binding protein 1C (predicted) |
| BE108294 | — | Transcribed locus |
| BE108367 | RGD1560834_predicted | similar to FRBZ1 protein (FRBZ1) (predicted) |
| BE108409 | — | Transcribed locus |
| BE108587 | Nisch | nischarin |
| BE108716 | — | Transcribed locus |
| BE108758 | — | Transcribed locus |
| BE108849 | — | Transcribed locus |
| BE108893 | — | Transcribed locus |
| BE108905 | Nap1l1 | Nucleosome assembly protein 1-like 1 |
| BE109260 | — | Transcribed locus |
| BE109322 | Plk4_predicted | polo-like kinase 4 (Drosophila) (predicted) |
| BE109605 | Sf1 | splicing factor 1 |
| BE109730 | — | Transcribed locus |
| BE109926 | Pik3cd_predicted | phosphatidylinositol 3-kinase catalytic delta polypeptide |
| | | (predicted) |
| BE110067 | — | Transcribed locus |
| BE110332 | RGD1307829_predicted | similar to chromosome 14 open reading frame 6 (predicted) |
| | /// Rnase11 | /// ribonuclease 11 |
| BE110369 | Arhgef18_predicted | rho/rac guanine nucleotide exchange factor (GEF) 18 |
| | | (predicted) |
| BE110674 | LOC686259 /// | hypothetical protein LOC686259 /// hypothetical protein |
| | LOC690243 | LOC690243 |
| BE110723 | Mphosph1_predicted | M-phase phosphoprotein 1 (predicted) |
| BE111310 | Ky_predicted | kyphoscoliosis (predicted) |
| BE111378 | Rpl3l_predicted | ribosomal protein L3-like (predicted) |
| BE111631 | Zfp91 | zinc finger protein 91 |
| BE111669 | Gcn1l1_predicted /// | GCN1 general control of amino-acid synthesis 1-like 1 |
| | LOC690632 | (yeast) (predicted) /// similar to GCN1 general control of |
| | | amino-acid synthesis 1-like 1 |
| BE111692 | — | Transcribed locus |
| BE111706 | Marcks | myristoylated alanine rich protein kinase C substrate |
| BE111722 | LOC498279 | similar to NADH dehydrogenase (ubiquinone) Fe—S protein 2 |
| BE112093 | Zfp91 | zinc finger protein 91 |
| BE112202 | Arrdc1 | arrestin domain containing 1 |
| BE112261 | Ctdspl_predicted | CTD (carboxy-terminal domain, RNA polymerase II, |
| | | polypeptide A) small phosphatase-like (predicted) |
| BE112453 | — | — |
| BE112895 | Pea15 | phosphoprotein enriched in astrocytes 15 |
| BE112927 | Cyfip2_predicted | cytoplasmic FMR1 interacting protein 2 (predicted) |
| BE112948 | RGD1566282_predicted | similar to RIKEN cDNA D330045A20 (predicted) |
| BE113005 | — | Transcribed locus, moderately similar to NP_001028560.1 |
| | | hypothetical protein LOC320003 [Mus musculus] |
| BE113144 | — | Transcribed locus, strongly similar to XP_579782.1 |
| | | PREDICTED: hypothetical protein XP_579782 [Rattus |
| | | norvegicus] |
| BE113247 | — | — |
| BE113263 | Mppe1_predicted | metallophosphoesterase 1 (predicted) |
| BE113270 | Igfbp5 | insulin-like growth factor binding protein 5 |
| BE113419 | Srpk2_predicted | serine/arginine-rich protein specific kinase 2 (predicted) |
| BE113571 | — | Transcribed locus |
| BE113753 | RGD1562949_predicted | similar to mKIAA0259 protein (predicted) |
| BE113958 | Nr2f2 | Nuclear receptor subfamily 2, group F, member 2 |
| BE113961 | — | Transcribed locus |
| BE114751 | — | — |
| BE115061 | Lef1 | Lymphoid enhancer binding factor 1 |
| BE115155 | Rhpn2_predicted | rhophilin, Rho GTPase binding protein 2 (predicted) |
| BE115262 | — | Transcribed locus |
| BE115432 | Utrn | Utrophin |
| BE115465 | Sf3b1 | splicing factor 3b, subunit 1 |
| BE115521 | RGD1310433_predicted | similar to mKIAA1757 protein (predicted) |
| BE115594 | Rfx5_predicted | regulatory factor X, 5 (influences HLA class II expression) |
| | | (predicted) |
| BE115612 | LOC679629 /// | similar to smoothelin, like |
| | LOC687788 | |
| BE115641 | — | — |
| BE116021 | Slc5a3 | solute carrier family 5 (inositol transporters), member 3 |
| BE116084 | Xlkd1_predicted | extracellular link domain-containing 1 (predicted) |
| BE116111 | — | Transcribed locus |
| BE116127 | — | — |
| BE116140 | — | Transcribed locus |
| BE116152 | Elovl6 | ELOVL family member 6, elongation of long chain fatty |
| | | acids (yeast) |
| BE116383 | — | Transcribed locus |
| BE116698 | RGD1564964_predicted | similar to WD repeat domain 11 protein (predicted) |
| BE116750 | Arhgap9 | Rho GTPase activating protein 9 |
| BE116774 | — | — |
| BE116855 | — | CDNA clone IMAGE: 7384525 |
| BE117044 | Cd8a | CD8 antigen, alpha chain |
| BE117215 | Nek7_predicted | NIMA (never in mitosis gene a)-related expressed kinase 7 |
| | | (predicted) |
| BE117217 | — | Transcribed locus |
| BE117361 | LOC681423 /// NIPBL | Nipped-B homolog (Drosophila) /// similar to delangin |
| | | isoform A |
| BE117736 | Tgfb2 | Transforming growth factor, beta 2 |
| BE117773 | Eif5b /// LOC689581 | eukaryotic translation initiation factor 5B /// similar to |
| | | Eukaryotic translation initiation factor 5B (eIF-5B) |
| | | (Translation initiation factor IF-2) |
| BE117804 | Cbfa2t1_predicted | CBFA2T1 identified gene homolog (human) (predicted) |
| BE117891 | Mll5 | myeloid/lymphoid or mixed-lineage leukemia 5 (trithorax |
| | | homolog, Drosophila) |
| BE118096 | — | Transcribed locus |
| BE118102 | RGD1307055_predicted | similar to hypothetical protein FLJ22490 (predicted) |
| BE118107 | — | — |
| BE118116 | — | Transcribed locus |
| BE118197 | — | Transcribed locus |
| BE118548 | Gtpbp3 | GTP binding protein 3 |
| BE118580 | — | Transcribed locus |
| BE118651 | Bmpr2 | bone morphogenic protein receptor, type II (serine/threonine |
| | | kinase) |
| BE118704 | — | Transcribed locus |
| BEI 18866 | LOC363849 | similar to histone cell cycle regulation defective homolog A |
| | | isoform 1 |
| BE118901 | Gata3 | GATA binding protein 3 |
| BE118929 | — | Transcribed locus |
| BE119167 | — | Transcribed locus |
| BE119699 | — | Transcribed locus |
| BE120370 | — | Transcribed locus |
| BE120415 | — | Transcribed locus |
| BE120660 | — | Transcribed locus |
| BE120816 | — | Transcribed locus |
| BE120823 | Smc4l1 | SMC4 structural maintenance of chromosomes 4-like 1 |
| | | (yeast) |
| BE120852 | — | Transcribed locus |
| BE120904 | — | Transcribed locus |
| BE126475 | Prrx1 | paired related homeobox 1 |
| BE328934 | RGD1310958_predicted | similar to RIKEN cDNA C130090K23 (predicted) |
| BE328951 | Cldn4 | claudin 4 |
| BE329013 | Smarca4 | SWI/SNF related, matrix associated, actin dependent |
| | | regulator of chromatin, subfamily a, member 4 |
| BE329196 | — | Transcribed locus |
| BE329244 | — | — |
| BE349699 | Tspan1 | tetraspanin 1 |
| BF281337 | Krt2-8 | keratin complex 2, basic, gene 8 |
| BF281697 | LOC681190 | similar to potassium voltage-gated channel, Isk-related |
| | | family, member 1-like |
| BF281701 | — | Transcribed locus |
| BF281987 | Cxcl11 | chemokine (C-X-C motif) ligand 11 |
| BF282187 | — | Transcribed locus |
| BF282190 | Il22ra2 | interleukin 22 receptor, alpha 2 |
| BF282228 | Ltb | lymphotoxin B |
| BF282471 | Lcp2 | lymphocyte cytosolic protein 2 |
| BF282632 | Tspan4 | tetraspanin 4 |
| BF282978 | RGD1307222_predicted | similar to mKIAA0664 protein (predicted) |
| BF283053 | Atp6v1c2 | ATPase, H+ transporting, V1 subunit C, isoform 2 |
| BF283070 | Cyp7b1 | cytochrome P450, family 7, subfamily b, polypeptide 1 |
| BF283079 | Sf1 | splicing factor 1 |
| BF283238 | RGD1307506_predicted | similar to RIKEN cDNA 2310016C16 (predicted) |
| BF283341 | — | Transcribed locus |
| BF283556 | Plxnc1_predicted | plexin C1 (predicted) |
| BF283610 | — | Transcribed locus |
| BF283642 | LOC363060 | similar to RIKEN cDNA 1600029D21 |
| BF283675 | — | Transcribed locus |
| BF283692 | Krt2-5 | keratin complex 2, basic, gene 5 |
| BF283779 | RGD1565496_predicted | similar to Butyrate-induced transcript 1 (predicted) |
| BF283797 | MGC116327 | similar to MK-5 type 2 |
| BF283924 | Lamp3 | lysosomal-associated membrane protein 3 |
| BF284168 | — | CDNA clone IMAGE: 7460165 |
| BF284224 | LOC365842 | similar to CDC-like kinase 2 |
| BF284262 | Irf8 | Interferon regulatory factor 8 |
| BF284295 | Tor3a | torsin family 3, member A |
| BF284523 | Abca1 | ATP-binding cassette, sub-family A (ABC1), member 1 |
| BF284716 | Ankrd15 | ankyrin repeat domain 15 |
| BF284889 | Actn2_predicted | actinin alpha 2 (predicted) |
| BF285345 | Arrb2 | arrestin, beta 2 |
| BF285350 | Apobec2_predicted | apolipoprotein B editing complex 2 (predicted) |
| BF285466 | LOC680259 | Hypothetical protein LOC680259 |
| BF285731 | — | Transcribed locus |
| BF285996 | LOC301334 | similar to Rho guanine nucleotide exchange factor 4 isoform a |
| BF287135 | Brd4 | bromodomain containing 4 |
| BF287629 | Il27ra_predicted | interleukin 27 receptor, alpha (predicted) |
| BF288000 | — | — |
| BF288101 | Snn | stannin |
| BF288109 | — | Transcribed locus |
| BF288130 | Ptprc | protein tyrosine phosphatase, receptor type, C |
| BF288177 | Csnk2a1 | casein kinase II, alpha 1 polypeptide |
| BF288243 | Klf2_predicted | Kruppel-like factor 2 (lung) (predicted) |
| BF288361 | — | — |
| BF288533 | — | Transcribed locus |
| BF289020 | Rab24 | RAB24, member RAS oncogene family |
| BF289150 | — | Transcribed locus |
| BF289368 | Lbp | lipopolysaccharide binding protein |
| BF290109 | — | — |
| BF290113 | RGD1311584 | similar to stem cell adaptor protein STAP-1 |
| BF290181 | — | — |
| BF290301 | LOC498662 | similar to RIKEN cDNA 2610019F03 |
| BF290410 | — | Transcribed locus |
| BF290784 | — | — |
| BF386238 | — | Transcribed locus |
| BF386742 | — | Transcribed locus |
| BF387238 | RGD1311117_predicted | similar to KIAA1409 protein (predicted) |
| BF387865 | — | Transcribed locus |
| BF388220 | RGD1308251_predicted | similar to RIKEN cDNA 2810405K02 (predicted) |
| BF388585 | Znf292 | zinc finger protein 292 |
| BF389056 | — | Transcribed locus |
| BF389793 | Crabp1 | cellular retinoic acid binding protein 1 |
| BF389889 | — | Transcribed locus |
| BF390077 | — | Transcribed locus |
| BF390257 | RGD1309863 | similar to hypothetical protein DKFZp434K1421 |
| BF390510 | RGD1311155 | similar to RIKEN cDNA 9230117N10 |
| BF390952 | — | Transcribed locus |
| BF391127 | Arid4a_predicted | AT rich interactive domain 4A (Rbp1 like) (predicted) |
| BF392234 | Bbx_predicted | bobby sox homolog (Drosophila) (predicted) |
| BF392577 | Akap8l | A kinase (PRKA) anchor protein 8-like |
| BF393607 | LOC689147 | hypothetical protein LOC689147 |
| BF393825 | Dcir3 | dendritic cell inhibitory receptor 3 |
| BF393994 | — | Transcribed locus |
| BF394055 | Depdc2_predicted | DEP domain containing 2 (predicted) |
| BF394095 | — | Transcribed locus |
| BF395095 | RGD1564875_predicted | similar to mKIAA0613 protein (predicted) |
| BF395171 | Ehd4 | EH-domain containing 4 |
| BF395317 | Ms4a11_predicted | membrane-spanning 4-domains, subfamily A, member 11 |
| | | (predicted) |
| BF395615 | Atg16l1_predicted | ATG16 autophagy related 16-like 1 (S. cerevisiae) |
| | | (predicted) |
| BF396210 | Trim59_predicted | tripartite motif-containing 59 (predicted) |
| BF396282 | Kif15 | kinesin family member 15 |
| BF396303 | — | Transcribed locus |
| BF396316 | — | Transcribed locus |
| BF396319 | — | Transcribed locus |
| BF396350 | Irak1_predicted | interleukin-1 receptor-associated kinase 1 (predicted) |
| BF396495 | — | Transcribed locus |
| BF396501 | — | Transcribed locus |
| BF396623 | — | Transcribed locus, strongly similar to XP_346904.2 |
| | | PREDICTED: hypothetical protein XP_346903 [Rattus |
| | | norvegicus] |
| BF396633 | — | Transcribed locus |
| BF396857 | Elovl6 | ELOVL family member 6, elongation of long chain fatty |
| | | acids (yeast) |
| BF397627 | — | Transcribed locus |
| BF397719 | Plekhg2_predicted | pleckstrin homology domain containing, family G (with |
| | | RhoGef domain) member 2 (predicted) |
| BF397766 | — | — |
| BF397936 | RGD1307526 | similar to modulator of estrogen induced transcription |
| BF398050 | Chd6_predicted | chromodomain helicase DNA binding protein 6 (predicted) |
| BF398091 | — | CDNA clone IMAGE: 7374368 |
| BF398122 | — | Synaptogenesis-related mRNA sequence 6 |
| BF398168 | — | Transcribed locus |
| BF398245 | Trps1_predicted | trichorhinophalangeal syndrome I (predicted) |
| BF398408 | — | Transcribed locus |
| BF398435 | — | Transcribed locus |
| BF398454 | — | — |
| BF398513 | RGD1563803_predicted | Similar to Lmnb2 protein (predicted) |
| BF398614 | — | Transcribed locus |
| BF399329 | — | — |
| BF399517 | — | Transcribed locus |
| BF399587 | — | Transcribed locus |
| BF399855 | Serpinb10 | serine (or cysteine) peptidase inhibitor, clade B (ovalbumin), |
| | | member 10 |
| BF400995 | RGD1308019_predicted | similar to hypothetical protein FLJ20245 (predicted) |
| BF401109 | — | Transcribed locus |
| BF401586 | — | Transcribed locus |
| BF402012 | Zbtb20_predicted | zinc finger and BTB domain containing 20 (predicted) |
| BF402387 | — | Transcribed locus |
| BF403434 | — | Transcribed locus |
| BF404261 | RGD1561602_predicted | similar to nemo like kinase (predicted) |
| BF404462 | — | Transcribed locus |
| BF404935 | — | Transcribed locus |
| BF405339 | — | Transcribed locus |
| BF405616 | — | Transcribed locus |
| BF405906 | — | — |
| BF406562 | Tmem1_predicted | transmembrane protein 1 (predicted) |
| BF406973 | RGD1311490_predicted | similar to DKFZP434P1750 protein (predicted) |
| BF407551 | — | — |
| BF407782 | — | Transcribed locus |
| BF408105 | — | — |
| BF408445 | Gpx7_predicted | glutathione peroxidase 7 (predicted) |
| BF408465 | Myct1_predicted | myc target 1 (predicted) |
| BF408757 | — | Transcribed locus |
| BF408881 | RGD1307981_predicted | similar to cisplatin resistance-associated overexpressed |
| | | protein (predicted) |
| BF408990 | Srrm2_predicted | serine/arginine repetitive matrix 2 (predicted) |
| BF409007 | — | Transcribed locus |
| BF409092 | — | Transcribed locus |
| BF409213 | — | Transcribed locus |
| BF409820 | — | Transcribed locus |
| BF410101 | — | Transcribed locus |
| BF410240 | — | — |
| BF410366 | — | Transcribed locus |
| BF410504 | GPR34_predicted | G-protein-coupled receptor GPR34 (predicted) |
| BF410953 | — | — |
| BF411017 | LOC304000 | cell adhesion molecule JCAM |
| BF411036 | Irf7 | interferon regulatory factor 7 |
| BF411402 | RGD1311662_predicted | similar to open reading frame 9 |
| BF411408 | — | — |
| BF411859 | — | Transcribed locus |
| BF412954 | — | Transcribed locus |
| BF413246 | — | Transcribed locus, weakly similar to XP_990808.1 |
| | | PREDICTED: hypothetical protein [Mus musculus] |
| BF413374 | — | Transcribed locus |
| BF414000 | — | Transcribed locus |
| BF414018 | Tmem97 | transmembrane protein 97 |
| BF414285 | — | Transcribed locus |
| BF414649 | Mrpl52_predicted | mitochondrial ribosomal protein L52 (predicted) |
| BF415030 | Pigp_predicted | phosphatidylinositol glycan, class P (predicted) |
| BF415080 | RGD1305202_predicted | similar to Protein C20orf160 (predicted) |
| BF415114 | Zbtb20_predicted | zinc finger and BTB domain containing 20 (predicted) |
| BF415195 | Gcn5l2_predicted | GCN5 general control of amino acid synthesis-like 2 (yeast) |
| | | (predicted) |
| BF415512 | — | Transcribed locus |
| BF415825 | — | Transcribed locus |
| BF415852 | LOC299907 | similar to Ext1 |
| BF416214 | — | Transcribed locus |
| BF416343 | LOC681423 /// NIPBL | Nipped-B homolog (Drosophila) /// similar to delangin |
| | | isoform A |
| BF416764 | LOC363326 | hypothetical LOC363326 |
| BF416786 | Fmnl1_predicted | formin-like 1 (predicted) |
| BF416979 | Spsb1_predicted | sp1A/ryanodine receptor domain and SOCS box containing 1 |
| | | (predicted) |
| BF417048 | LOC499300 | similar to protein tyrosine phosphatase, receptor type, C |
| | | polypeptide-associated protein |
| BF417079 | — | Transcribed locus |
| BF417216 | LOC686132 | similar to hemicentin 1 |
| BF417252 | RGD1305350_predicted | similar to RIKEN cDNA 2510039O18 (predicted) |
| BF417363 | Hmha1_predicted | histocompatibility (minor) HA-1 (predicted) |
| BF417385 | MGC109519 | similar to tropomyosin 1, embryonic fibroblast - rat |
| BF417479 | Dhcr24 | 24-dehydrocholesterol reductase |
| BF417582 | Myo1g | myosin IG |
| BF417625 | — | Transcribed locus |
| BF417784 | — | Transcribed locus |
| BF418025 | Grap | GRB2-related adaptor protein |
| BF418487 | Med31_predicted | mediator of RNA polymerase II transcription, subunit 31 |
| | | homolog (yeast) (predicted) |
| BF418522 | — | Transcribed locus |
| BF418596 | Ss18 | synovial sarcoma translocation, Chromosome 18 |
| BF418649 | RGD1305645_predicted | similar to RIKEN cDNA 1500015O10 (predicted) |
| BF418812 | Tceal1 | transcription elongation factor A (SII)-like 1 |
| BF418957 | C1qa | complement component 1, q subcomponent, alpha |
| | | polypeptide |
| BF419134 | Acbd3 | acyl-Coenzyme A binding domain containing 3 |
| BF419319 | Oasl1 | 2′-5′ oligoadenylate synthetase-like 1 |
| BF419899 | Ccl7 | chemokine (C-C motif) ligand 7 |
| BF419995 | Myl2 | myosin, light polypeptide 2, regulatory, cardiac, slow |
| BF420720 | RGD1563128_predicted | similar to ADP-ribosylation-like factor 12 protein (predicted) |
| BF420722 | Nfix | nuclear factor I/X |
| BF420803 | — | Transcribed locus |
| BF420807 | — | — |
| BF420810 | Hrc | histidine rich calcium binding protein |
| BF521859 | Tnnt3 | troponin T3, skeletal, fast |
| BF522317 | — | Transcribed locus |
| BF522436 | RGD1306067 | similar to chromosome 20 open reading frame 6 |
| BF522954 | Il18r1_predicted | interleukin 18 receptor 1 (predicted) |
| BF523248 | Ankrd11_predicted | ankyrin repeat domain 11 (predicted) |
| BF524010 | — | — |
| BF525282 | Hsph1 | heat shock 105 kDa/110 kDa protein 1 |
| BF542615 | Ptplad2_predicted | protein tyrosine phosphatase-like A domain containing 2 |
| | | (predicted) |
| BF543289 | RGD1304592_predicted | similar to KIAA0528 protein (predicted) |
| BF544982 | Gmip_predicted | Gem-interacting protein (predicted) |
| BF545080 | Cox6b2 | cytochrome c oxidase subunit VIb testes-specific isoform |
| | | precursor |
| BF545268 | Cr2_predicted | complement receptor 2 (predicted) |
| BF545930 | — | Transcribed locus |
| BF545988 | Tmc4 | transmembrane channel-like gene family 4 |
| BF546899 | RGD1307396_predicted | similar to RIKEN cDNA 6330406I15 (predicted) |
| BF547251 | LOC685707 /// | neuron navigator 1 (predicted) /// similar to neuron navigator 1 |
| | Nav1_predicted | |
| BF548081 | — | — |
| BF549535 | — | Transcribed locus, weakly similar to XP_999107.1 |
| | | PREDICTED: similar to zinc finger protein 700 [Mus |
| | | musculus] |
| BF550315 | Trps1_predicted | trichorhinophalangeal syndrome I (predicted) |
| BF550555 | Lig4_predicted | Ligase IV, DNA, ATP-dependent (predicted) |
| BF551187 | — | Transcribed locus |
| BF551457 | LOC501619 | similar to 40S ribosomal protein S29 |
| BF551686 | Ap1s2_predicted | adaptor-related protein complex 1, sigma 2 subunit |
| | | (predicted) |
| BF552772 | LOC499094 | similar to zinc finger protein 61 |
| BF552973 | Myot_predicted | myotilin (predicted) |
| BF553172 | — | Transcribed locus |
| BF553613 | Txndc4 | thioredoxin domain containing 4 (endoplasmic reticulum) |
| BF554283 | Ogfrl1 | opioid growth factor receptor-like 1 |
| BF554746 | Rtn4rl1 | reticulon 4 receptor-like 1 |
| BF555727 | Efna3 | ephrin A3 |
| BF555968 | Nedd9 | neural precursor cell expressed, developmentally down- |
| | | regulated gene 9 |
| BF555972 | RGD1359529 | similar to chromosome 1 open reading frame 63 |
| BF555973 | — | Transcribed locus |
| BF556622 | — | — |
| BF556812 | — | Transcribed locus |
| BF557813 | — | Transcribed locus |
| BF558056 | — | — |
| BF558075 | RGD1305464 | similar to DKFZP434H132 protein |
| BF558512 | — | Transcribed locus |
| BF558732 | Baz1b | bromodomain adjacent to zinc finger domain protein 1B |
| BF558946 | LOC688717 | similar to CG33714-PB, isoform B |
| BF560693 | — | — |
| BF560932 | Sox4_predicted | SRY-box containing gene 4 (predicted) |
| BF560938 | Zfp503_predicted | zinc finger protein 503 (predicted) |
| BF561077 | — | Transcribed locus |
| BF561454 | Fgl2 | fibrinogen-like 2 |
| BF561717 | Pdha1 | pyruvate dehydrogenase E1 alpha 1 |
| BF562507 | Sorl1 | sortilin-related receptor, LDLR class A repeats-containing |
| BF564217 | Ang1 | angiogenin, ribonuclease A family, member 1 |
| BF565167 | Rdx | radixin |
| BF565675 | Arsa | arylsulfatase A |
| BF565718 | — | Transcribed locus |
| BF565756 | — | Transcribed locus |
| BF565850 | — | Transcribed locus |
| BF566051 | — | Transcribed locus |
| BF567833 | Tmod2 | tropomodulin 2 |
| BG057565 | Bat1a | HLA-B-associated transcript 1A |
| BG371544 | — | Transcribed locus |
| BG371683 | — | Transcribed locus, moderately similar to NP_598678.1 |
| | | mondoA isoform 2 [Mus musculus] |
| BG371843 | Pkp1_predicted | plakophilin 1 (predicted) |
| BG372056 | — | Transcribed locus |
| BG372184 | — | Transcribed locus |
| BG372243 | — | Transcribed locus |
| BG372342 | RGD1311019_predicted | similar to hypothetical protein DKFZp434H2010 (predicted) |
| BG372602 | RGD1306601_predicted | similar to cDNA sequence BC019755 (predicted) |
| BG372703 | Fnbp4 | formin binding protein 4 |
| BG372891 | — | Transcribed locus |
| BG372903 | Rnpc2 | RNA-binding region (RNP1, RRM) containing 2 |
| BG372987 | — | — |
| BG373000 | — | Transcribed locus |
| BG373166 | Elf5_predicted | E74-like factor 5 (predicted) |
| BG373352 | B3gnt1_predicted | UDP-GlcNAc: betaGal beta-1,3-N- |
| | | acetylglucosaminyltransferase 1 (predicted) |
| BG373576 | Prmt6_predicted | protein arginine N-methyltransferase 6 (predicted) |
| BG373580 | Chchd1_predicted | coiled-coil-helix-coiled-coil-helix domain containing 1 |
| | | (predicted) |
| BG373799 | — | Transcribed locus |
| BG374483 | Per3 | period homolog 3 (Drosophila) |
| BG374493 | Cyp2j10_predicted | cytochrome P450, family 2, subfamily j, polypeptide 10 |
| | | (predicted) |
| BG374639 | — | Transcribed locus |
| BG375315 | Sdc1 | syndecan 1 |
| BG375352 | Ca5b | carbonic anhydrase VB, mitochondrial |
| BG375362 | Ltbp4 | latent transforming growth factor beta binding protein 4 |
| BG375798 | — | Transcribed locus |
| BG376030 | RGD1306107_predicted | similar to chromosome 1 open reading frame 2 (predicted) |
| BG376644 | Rsbn1l_predicted | round spermatid basic protein 1-like (predicted) |
| BG377116 | Atp6v0a4_predicted | ATPase, H+ transporting, lysosomal V0 subunit A isoform 4 |
| | | (predicted) |
| BG377332 | — | — |
| BG377504 | Foxi1_predicted | forkhead box I1 (predicted) |
| BG377979 | LOC690528 | similar to POU domain class 2, associating factor 1 (B-cell- |
| | | specific coactivator OBF-1) (OCT binding factor 1) (BOB-1) |
| | | (BOB1) (OCA-B) |
| BG378166 | Map4k2_predicted | mitogen activated protein kinase kinase kinase kinase 2 |
| | | (predicted) |
| BG378238 | — | Transcribed locus |
| BG378310 | Scel_predicted | sciellin (predicted) |
| BG378463 | RGD1563547_predicted | RGD1563547 (predicted) |
| BG378588 | Mybpc2_predicted | myosin binding protein C, fast-type (predicted) |
| BG378607 | LOC362350 /// Tcrb | T-cell receptor beta chain /// similar to T-cell receptor beta-2 |
| | | chain C region |
| BG378613 | Agpat3_predicted | 1-acylglycerol-3-phosphate O-acyltransferase 3 (predicted) |
| BG378630 | Plac8_predicted | placenta-specific 8 (predicted) |
| BG378709 | — | — |
| BG378709 | — | — |
| BG378760 | Srpk1 | Serine/arginine-rich protein specific kinase 1 |
| BG378874 | Wbp5_predicted | WW domain binding protein 5 (predicted) |
| BG378885 | Hmga1 | high mobility group AT-hook 1 |
| BG378912 | Wdr47 | WD repeat domain 47 |
| BG379188 | Siglec5_predicted | sialic acid binding Ig-like lectin 5 (predicted) |
| BG379338 | Rrm2 | ribonucleotide reductase M2 |
| BG379358 | Xpc_predicted | xeroderma pigmentosum, complementation group C |
| | | (predicted) |
| BG379394 | — | Transcribed locus |
| BG380122 | Serpinb6b | serine (or cysteine) proteinase inhibitor, clade B, member 6b |
| BG380534 | Tle1_predicted | transducin-like enhancer of split 1, homolog of Drosophila |
| | | E(spl) (predicted) |
| BG380672 | LOC682058 | similar to nucleolar protein with MIF4G domain 1 |
| BG380684 | Rtn2 | reticulon 2 (Z-band associated protein) |
| BG380735 | — | Transcribed locus |
| BG380816 | LOC291411 | similar to Potential phospholipid-transporting ATPase IIB |
| BG381555 | — | CDNA clone IMAGE: 7388962 |
| BG381583 | RGD1565118_predicted | similar to mKIAA0843 protein (predicted) |
| BG662519 | Ank | Progressive ankylosis homolog (mouse) |
| BG662875 | Pea15 | phosphoprotein enriched in astrocytes 15 |
| BG663128 | Tnnc2 | troponin C type 2 (fast) |
| BG663208 | Jak1 | Janus kinase 1 |
| BG663284 | Fgl2 | fibrinogen-like 2 |
| BG663444 | Wbp4 | WW domain binding protein 4 |
| BG663460 | Pafah1b1 | platelet-activating factor acetylhydrolase, isoform Ib, alpha |
| | | subunit 45 kDa |
| BG663483 | Pcdha1 /// Pcdha10 /// | protocadherin alpha 4 /// protocadherin alpha 13 /// |
| | Pcdha11 /// Pcdha12 /// | protocadherin alpha 10 /// protocadherin alpha 12 /// |
| | Pcdha13 /// Pcdha2 /// | protocadherin alpha 3 /// protocadherin alpha 8 /// |
| | Pcdha3 /// Pcdha4 /// | protocadherin alpha 1 /// protocadherin alpha 2 /// |
| | Pcdha5 /// Pcdha6 /// | protocadherin alpha 5 /// protocadherin alpha 6 /// |
| | Pcdha7 /// Pcdha8 /// | protocadherin alpha 7 /// protocadherin alpha 9 /// |
| | Pcdha9 /// Pcdhac1 /// | protocadherin alpha subfamily C, 1 /// protocadherin alpha |
| | Pcdhac2 | subfamily C, 2 /// protocadherin alpha 11 |
| BG663870 | RGD1310061 | similar to hypothetical protein FLJ10154 |
| BG664011 | Pxmp4 | peroxisomal membrane protein 4 |
| BG664123 | Cyp51 | cytochrome P450, subfamily 51 |
| BG664221 | Ogn_predicted | osteoglycin (predicted) |
| BG664660 | Tnfaip6 | tumor necrosis factor alpha induced protein 6 |
| BG665051 | — | — |
| BG665568 | — | Transcribed locus |
| BG665934 | Oact2 | O-acyltransferase (membrane bound) domain containing 2 |
| BG666095 | RGD1306274 | similar to hypothetical protein BC002942 |
| BG666306 | Thbd | thrombomodulin |
| BG666882 | — | Transcribed locus |
| BG666933 | Cst3 | cystatin C |
| BG668062 | RGD1566320_predicted | RGD1566320 (predicted) |
| BG668493 | Stmn2 | stathmin-like 2 |
| BG668724 | Ncoa5_predicted | nuclear receptor coactivator 5 (predicted) |
| BG668816 | Etnk1_predicted | ethanolamine kinase 1 (predicted) |
| BG668988 | Elovl4_predicted | elongation of very long chain fatty acids (FEN1/Elo2, |
| | | SUR4/Elo3, yeast)-like 4 (predicted) |
| BG669096 | Mpz | myelin protein zero |
| BG669749 | — | Transcribed locus |
| BG670310 | Tgfa | transforming growth factor alpha |
| BG670822 | — | Non-coding RNA expressed in the brain, repeat sequence, |
| | | clone 2 /// Non-coding RNA expressed in the brain, repeat |
| | | sequence, clone 7 /// Non-coding RNA expressed in the |
| | | brain, repeat sequence, clone 3 /// Non-coding RNA |
| | | expressed in the brain, repeat sequence, clone 12 |
| BG671050 | — | — |
| BG671521 | Hspca | heat shock protein 1, alpha |
| BG671686 | — | — |
| BG671734 | — | Transcribed locus, strongly similar to XP_927381.1 |
| | | PREDICTED: hypothetical protein LOC69784 [Mus |
| | | musculus] |
| BG672259 | LOC691397 /// | similar to PI-3-kinase-related kinase SMG-1 isoform 2 |
| | RGD1563508_predicted | (predicted) /// similar to PI-3-kinase-related kinase SMG-1 |
| BG672517 | RGD1308373 | similar to DKFZP566K1924 protein |
| BG672572 | — | Transcribed locus |
| BG673248 | — | Transcribed locus |
| BG673380 | Ppp2r5a_predicted | protein phosphatase 2, regulatory subunit B (B56), alpha |
| | | isoform (predicted) |
| BI273721 | — | Transcribed locus |
| BI273815 | RGD1309747_predicted | similar to KIAA0947 protein (predicted) |
| BI273908 | — | Transcribed locus |
| BI273954 | — | Transcribed locus |
| BI274054 | Folr2_predicted | folate receptor 2 (fetal) (predicted) |
| BI274094 | LOC313641 | perlecan |
| BI274189 | Rxrb | retinoid X receptor beta |
| BI274216 | — | Transcribed locus |
| BI274308 | Ttc7b_predicted | tetratricopeptide repeat domain 7B (predicted) |
| BI274326 | Muc1 | mucin 1, transmembrane |
| BI274438 | Fam38a_predicted | family with sequence similarity 38, member A (predicted) |
| BI274471 | — | Transcribed locus |
| BI274480 | Fnbp4 | formin binding protein 4 |
| BI274481 | Fnbp1 | Formin binding protein 1 |
| BI274516 | LOC304396 | similar to hypothetical protein DKFZp434K1815 |
| BI274533 | RGD1309534 | similar to RIKEN cDNA 4931406C07 |
| BI274548 | Rtel1 | regulator of telomere elongation helicase 1 |
| BI274615 | — | — |
| BI274778 | — | Transcribed locus |
| BI275119 | L3mbtl3_predicted | l(3)mbt-like 3 (Drosophila) (predicted) |
| BI275261 | — | — |
| BI275485 | Sema3b_predicted | sema domain, immunoglobulin domain (Ig), short basic |
| | | domain, secreted, (semaphorin) 3B (predicted) |
| BI275633 | Pygm | muscle glycogen phosphorylase |
| BI275677 | — | — |
| BI275765 | Uhrf2_predicted | ubiquitin-like, containing PHD and RING finger domains 2 |
| | | (predicted) |
| BI275827 | — | Transcribed locus |
| BI275873 | Centb1_predicted | centaurin, beta 1 (predicted) |
| BI275966 | Sipa1 | signal-induced proliferation-associated gene 1 |
| BI276075 | — | Transcribed locus |
| BI276216 | Tyki_predicted | thymidylate kinase family LPS-inducible member |
| | | (predicted) |
| BI276252 | Mical1_predicted | microtubule associated monoxygenase, calponin and LIM |
| | | domain containing 1 (predicted) |
| BI276959 | LOC679341 /// | similar to Calsequestrin-1 precursor (Calsequestrin, skeletal |
| | LOC686019 | muscle isoform) |
| BI276972 | Heatr1_predicted | HEAT repeat containing 1 (predicted) |
| BI276974 | Vars2l | valyl-tRNA synthetase 2-like |
| BI277513 | LOC360975 | similar to oxoglutarate dehydrogenase (lipoamide) |
| BI277545 | Myh1 /// Myh2 | myosin, heavy polypeptide 1, skeletal muscle, adult /// |
| | | myosin, heavy polypeptide 2, skeletal muscle, adult |
| BI277586 | Myh2 | myosin, heavy polypeptide 2, skeletal muscle, adult |
| BI277805 | RGD1562335_predicted | similar to mKIAA1931 protein (predicted) |
| BI278180 | — | Transcribed locus |
| BI278186 | Crct1_predicted | cysteine-rich C-terminal 1 (predicted) |
| BI278333 | — | Transcribed locus, weakly similar to NP_742040.1 mannan- |
| | | binding lectin serine protease 2 [Rattus norvegicus] |
| BI278404 | RGD1562885_predicted | similar to RIKEN cDNA 2300002M23 (predicted) |
| BI278423 | Nbeal2_predicted | neurobeachin-like 2 (predicted) |
| BI278559 | Clec4a1 | C-type lectin domain family 4, member a1 |
| BI278590 | — | Transcribed locus |
| BI278614 | Bmp1 | bone morphogenetic protein 1 |
| BI278687 | Pltp_predicted | phospholipid transfer protein (predicted) |
| BI278721 | Calm4_predicted | calmodulin 4 (predicted) |
| BI278952 | — | — |
| BI279191 | Leng8 | leukocyte receptor cluster (LRC) member 8 |
| BI279216 | Ube2l6 | ubiquitin-conjugating enzyme E2L 6 |
| BI279384 | — | Transcribed locus |
| BI279486 | LOC494538 | ABP beta |
| BI279526 | RT1-Db1 | RT1 class II, locus Db1 |
| BI279562 | Baiap2 | brain-specific angiogenesis inhibitor 1-associated protein 2 |
| BI279605 | Krt1-19 | keratin complex 1, acidic, gene 19 |
| BI279646 | Krt1-14 | keratin complex 1, acidic, gene 14 |
| BI279663 | Dsc2 | desmocollin 2 |
| BI279680 | LOC684050 | similar to procollagen C-endopeptidase enhancer 2 |
| BI279694 | Trim29_predicted | tripartite motif protein 29 (predicted) |
| BI279760 | Pgk1 | phosphoglycerate kinase 1 |
| BI279786 | Myo1d | myosin ID |
| BI280124 | RGD1565500_predicted | RGD1565500 (predicted) |
| BI281680 | Cldn5 | claudin 5 |
| BI282076 | Prdx4 | peroxiredoxin 4 |
| BI282114 | — | Endogenous retrovirus mRNA, partial sequence |
| BI282130 | LOC687682 /// | similar to Disco-interacting protein 2 homolog |
| | LOC690211 | |
| BI282164 | Cyb561_predicted | cytochrome b-561 (predicted) |
| BI282272 | RGD1304587 | similar to RIKEN cDNA 2310033P09 |
| BI282281 | LOC500372 | similar to Stress-70 protein, mitochondrial precursor (75 kDa |
| | | glucose regulated protein) (GRP 75) (Peptide-binding |
| | | protein 74) (PBP74) (MTHSP70) (Mortalin) |
| BI282513 | Stfa3_predicted | stefin A3 (predicted) |
| BI282567 | Klk8 | kallikrein 8 (neuropsin/ovasin) |
| BI282568 | Krtdap | keratinocyte differentiation associated protein |
| BI282576 | RGD1310935_predicted | similar to Dermal papilla derived protein 7 (predicted) |
| BI282584 | RGD1560328_predicted | similar to UPF0197 protein C11orf10 homolog (predicted) |
| BI282694 | RGD1565037_predicted | similar to selenoprotein SelM (predicted) |
| BI282719 | RGD1565055_predicted | similar to PDZ-domain protein scribble (predicted) |
| BI282755 | — | Transcribed locus |
| BI282819 | — | Transcribed locus |
| BI282860 | LOC685652 | similar to keratin 6L |
| BI282883 | LOC312678 | similar to Retinoblastoma-binding protein 2 (RBBP-2) |
| BI282914 | Kctd14_predicted | potassium channel tetramerisation domain containing 14 |
| | | (predicted) |
| BI282920 | Ccl21b | chemokine (C-C motif) ligand 21b (serine) |
| BI282944 | LOC301126 | similar to scaffold attachment factor B2 |
| BI282960 | Gpsn2 | glycoprotein, synaptic 2 |
| BI283060 | Flna_predicted | filamin, alpha (predicted) |
| BI283128 | LOC682360 | hypothetical protein LOC682360 |
| BI283139 | LOC679693 | similar to Mediator of RNA polymerase II transcription |
| | | subunit 12 (Thyroid hormone receptor-associated protein |
| | | complex 230 kDa component) (Trap230) (Activator- |
| | | recruited cofactor 240 kDa component) (ARC240) (CAG |
| | | repeat protein 45) (OPA-containing protein) |
| BI283346 | Klk10 | kallikrein 10 |
| BI283648 | — | Transcribed locus, moderately similar to XP_919392.1 |
| | | PREDICTED: hypothetical protein XP_914299 [Mus |
| | | musculus] |
| BI283695 | — | Transcribed locus |
| BI283728 | — | Transcribed locus |
| BI283851 | Ppil1 | peptidylprolyl isomerase (cyclophilin)-like 1 |
| BI284430 | Tmed3 | transmembrane emp24 domain containing 3 |
| BI285064 | — | Transcribed locus |
| BI285065 | — | Transcribed locus |
| BI285092 | LOC682044 /// | hypothetical protein LOC682044 /// hypothetical protein |
| | LOC684972 | LOC684972 |
| BI285329 | LOC498178 /// | similar to SET domain-containing protein |
| | LOC687538 /// | |
| | LOC689820 | |
| BI285346 | Ppp4r1 | protein phosphatase 4, regulatory subunit 1 |
| BI285402 | Ddx17 | DEAD (Asp-Glu-Ala-Asp) box polypeptide 17 |
| BI285443 | RGD1560859_predicted | similar to 2300003P22Rik protein (predicted) |
| BI285494 | Ifitm3 | interferon induced transmembrane protein 3 |
| BI285676 | — | Transcribed locus |
| BI285700 | Hspcb | heat shock 90 kDa protein 1, beta |
| BI285902 | RGD1308696 | similar to hypothetical protein D11Ertd497e |
| BI285941 | — | Transcribed locus |
| BI285951 | RGD1308734 | similar to RIKEN cDNA 1100001H23 |
| BI285965 | Usp48 | ubiquitin specific protease 48 |
| BI286012 | Krt1-18 | keratin complex 1, acidic, gene 18 |
| BI286166 | LOC298795 /// | similar to 14-3-3 protein sigma /// stratifin (predicted) |
| | Sfn_predicted | |
| BI286340 | RGD1305779_predicted | similar to NSE1 (predicted) |
| BI286364 | — | Transcribed locus |
| BI286387 | LOC499660 | similar to Cornifin A (Small proline-rich protein 1A) |
| | | (SPR1A) (SPRR1) |
| BI286389 | Ka17 | type I keratin KA17 |
| BI286396 | Perp_predicted | PERP, TP53 apoptosis effector (predicted) |
| BI286411 | — | Ultraviolet B radiation-activated UV103 mRNA, partial |
| | | sequence |
| BI286411 | LOC683295 | similar to keratin complex 2, basic, gene 6a |
| BI286421 | — | Transcribed locus, strongly similar to NP_001007709.1 |
| | | hypothetical protein LOC315126 [Rattus norvegicus] |
| BI287007 | RGD1560606_predicted | similar to down-regulated in metastasis (predicted) |
| BI287330 | RGD1561312_predicted | similar to type 1 protein phosphatase targeting subunit |
| | | RGL/GM (predicted) |
| BI287702 | Usp28_predicted | ubiquitin specific protease 28 (predicted) |
| BI288055 | — | Transcribed locus, moderately similar to NP_036691.2 |
| | | fibrinogen, gamma polypeptide [Rattus norvegicus] |
| BI288162 | — | Transcribed locus |
| BI288619 | Jun | Jun oncogene |
| BI288833 | Dact2_predicted | dapper homolog 2, antagonist of beta-catenin (xenopus) |
| | | (predicted) |
| BI288898 | LOC680692 /// | similar to Golgi phosphoprotein 2 (Golgi membrane protein |
| | LOC682869 | GP73) |
| BI289103 | Slc44a4 | solute carrier family 44, member 4 |
| BI289110 | — | Transcribed locus |
| BI289371 | Rhbdl6_predicted | rhomboid, veinlet-like 6 (Drosophila) (predicted) |
| BI289386 | Bcor_predicted | Bcl6 interacting corepressor (predicted) |
| BI289517 | RGD1562778_predicted | similar to tripartite motif protein 50 (predicted) |
| BI289527 | Tmod4_predicted | tropomodulin 4 (predicted) |
| BI289641 | RGD1566064_predicted | similar to KIAA1096 protein (predicted) |
| BI289662 | LOC690326 | hypothetical protein LOC690326 |
| BI289840 | — | Transcribed locus |
| BI289867 | Slc16a6 | solute carrier family 16 (monocarboxylic acid transporters), |
| | | member 6 |
| BI290053 | — | Transcribed locus |
| BI290161 | Agpat3_predicted | 1-acylglycerol-3-phosphate O-acyltransferase 3 (predicted) |
| BI290551 | Fnbp1 | Formin binding protein 1 |
| BI290633 | LOC306805 | Similar to asporin precursor |
| BI290737 | — | Transcribed locus |
| BI290787 | — | — |
| BI290909 | Scap1 | src family associated phosphoprotein 1 |
| BI291212 | — | Transcribed locus |
| BI291230 | — | — |
| BI291434 | Pfkm | phosphofructokinase, muscle |
| BI291600 | RGD1309450_predicted | similar to KIAA2010 protein (predicted) |
| BI291604 | LOC687609 | similar to ras homolog gene family, member f |
| BI291798 | — | Immunoglobulin delta heavy chain transmembrane form |
| | | (IgD) |
| BI291804 | — | Transcribed locus |
| BI291875 | Clca3_predicted | chloride channel calcium activated 3 (predicted) |
| BI291927 | RT1-A2 /// RT1-A3 | RT1 class Ia, locus A2 /// RT1 class I, A3 |
| BI292034 | — | — |
| BI292036 | LOC683839 /// | hypothetical protein LOC683839 /// hypothetical protein |
| | LOC688495 | LOC688495 |
| BI292056 | — | Transcribed locus |
| BI292067 | — | Transcribed locus |
| BI292090 | Cldn8 | claudin 8 |
| BI292231 | Lpo_predicted | lactoperoxidase (predicted) |
| BI292687 | — | Transcribed locus |
| BI292758 | — | Transcribed locus |
| BI293047 | Bmper_predicted | BMP-binding endothelial regulator (predicted) |
| BI293248 | — | Transcribed locus |
| BI293796 | Nckap1l_predicted | NCK associated protein 1 like (predicted) |
| BI293987 | Rbm5 | RNA binding motif protein 5 |
| BI294084 | Ccl24 | chemokine (C-C motif) ligand 24 |
| BI294122 | Gpr23_predicted | G protein-coupled receptor 23 (predicted) |
| BI294141 | Angpt4_predicted | angiopoietin 4 (predicted) |
| BI294158 | — | Transcribed locus |
| BI294178 | — | Transcribed locus |
| BI294556 | Stk17b | serine/threonine kinase 17b (apoptosis-inducing) |
| BI294721 | RGD1562617_predicted | similar to RAB7-like protein (predicted) |
| BI294722 | — | Transcribed locus, weakly similar to XP_487464.2 |
| | | PREDICTED: similar to zinc finger protein 85, related |
| | | sequence 1 [Mus musculus] |
| BI294737 | — | — |
| BI294787 | Maml1_predicted | mastermind like 1 (Drosophila) (predicted) |
| BI294788 | — | Transcribed locus |
| BI294789 | RGD1308331_predicted | similar to Trithorax homolog 2 (Mixed lineage leukemia |
| | | gene homolog 2 protein) (predicted) |
| BI294811 | RGD1311381_predicted | similar to hypothetical protein FLJ20037 (predicted) |
| BI294855 | Limd2 | LIM domain containing 2 |
| BI294907 | Ncoa3 | nuclear receptor coactivator 3 |
| BI294910 | Car8 | carbonic anhydrase 8 |
| BI294976 | — | Transcribed locus |
| BI295047 | RGD1309550 | similar to hypothetical protein D12Ertd771e |
| BI295614 | — | Transcribed locus, strongly similar to NP_001041317.1 |
| | | BCL2-associated transcription factor 1 [Rattus norvegicus] |
| BI295700 | Mll | myeloid/lymphoid or mixed-lineage leukemia |
| BI295832 | Crebl1 | cAMP responsive element binding protein-like 1 |
| BI295864 | Tmeff1 | transmembrane protein with EGF-like and two follistatin- |
| | | like domains 1 |
| BI295900 | Dlat | dihydrolipoamide S-acetyltransferase (E2 component of |
| | | pyruvate dehydrogenase complex) |
| BI295930 | Clec14a | C-type lectin domain family 14, member a |
| BI295947 | RGD1564924_predicted | similar to leiomodin 3 (fetal) (predicted) |
| BI295964 | Slamf9_predicted | SLAM family member 9 (predicted) |
| BI296029 | Camta2_predicted | calmodulin binding transcription activator 2 (predicted) |
| BI296153 | Acaca | acetyl-coenzyme A carboxylase alpha |
| BI296353 | LOC501039 | similar to solute carrier family 25, member 36 |
| BI296499 | — | — |
| BI296577 | Pex5_predicted | peroxisome biogenesis factor 5 (predicted) |
| BI296579 | — | Transcribed locus |
| BI296586 | LOC499014 | similar to zinc finger, DHHC domain containing 14 |
| BI296626 | LOC679725 /// | similar to MASK-4E-BP3 protein |
| | LOC682955 | |
| BI296641 | — | Transcribed locus |
| BI296696 | Cables1_predicted | Cdk5 and Abl enzyme substrate 1 (predicted) |
| BI296717 | — | Transcribed locus |
| BI296754 | Ddx17 | DEAD (Asp-Glu-Ala-Asp) box polypeptide 17 |
| BI296868 | RGD1566401_predicted | similar to GTL2, imprinted maternally expressed |
| | | untranslated (predicted) |
| BI296989 | Cdk10 | cyclin-dependent kinase (CDC2-like) 10 |
| BI296990 | — | Transcribed locus |
| BI297744 | RGD1310507 | similar to RIKEN cDNA 1300017J02 |
| BI298306 | — | Transcribed locus |
| BI299761 | — | Transcribed locus |
| BI300393 | — | Transcribed locus, strongly similar to NP_848878.1 |
| | | hypothetical protein LOC319530 [Mus musculus] |
| BI300470 | — | Transcribed locus |
| BI300502 | — | Transcribed locus |
| BI300794 | — | Transcribed locus |
| BI301117 | — | Transcribed locus |
| BI301125 | LOC682926 /// | similar to chromatin modifying protein 1B |
| | LOC689364 | |
| BI301280 | RGD1561004_predicted | similar to AMME syndrome candidate gene 1 protein |
| | | homolog (predicted) |
| BI301453 | Ckmt1 | creatine kinase, mitochondrial 1, ubiquitous |
| BI301465 | — | Transcribed locus |
| BI301467 | LOC363009 | similar to CG31653-PA |
| BI301495 | LOC688276 | similar to epidermodysplasia verruciformis 2 |
| BI302005 | Mlf1_predicted | myeloid leukemia factor 1 (predicted) |
| BI302669 | Stno_predicted | strawberry notch homolog (Drosophila) (predicted) |
| BI302694 | RGD1561555_predicted | similar to CDNA sequence BC022692 (predicted) |
| BI303078 | — | Transcribed locus |
| BI303106 | Traf3_predicted | Tnf receptor-associated factor 3 (predicted) |
| BI303187 | Sc65 | Synaptonemal complex protein SC65 |
| BI303187 | Sc65 | synaptonemal complex protein SC65 |
| BI303253 | — | CDNA clone IMAGE: 7365313 |
| BI303636 | — | Transcribed locus |
| BI304009 | Lox | lysyl oxidase |
| BI304019 | — | Transcribed locus |
| BI389176 | Lnx2_predicted | ligand of numb-protein X 2 (predicted) |
| BI395810 | Npy1r | neuropeptide Y receptor Y1 |
| BI395849 | — | — |
| BM382988 | — | Transcribed locus |
| BM383065 | RGD1310587 | similar to hypothetical protein FLJ14146 |
| BM383395 | Mknk2 | MAP kinase-interacting serine/threonine kinase 2 |
| BM383423 | Ehf_predicted | ets homologous factor (predicted) |
| BM383428 | LOC367976 /// | minichromosome maintenance deficient 3 (S. cerevisiae) |
| | Mcm3_predicted | (predicted) /// similar to DNA replication licensing factor |
| | | MCM3 (DNA polymerase alpha holoenzyme-associated |
| | | protein P1) (P1-MCM3) |
| BM383442 | LOC363009 | similar to CG31653-PA |
| BM383464 | — | Transcribed locus |
| BM383531 | LOC682651 /// | similar to Metallothionein-2 (MT-2) (Metallothionein-II) |
| | LOC689415 | (MT-II) |
| BM383809 | Mlstd2 | male sterility domain containing 2 |
| BM383953 | LOC367171 | microtubule-associated protein 4 |
| BM383972 | — | Transcribed locus |
| BM383995 | — | Transcribed locus |
| BM384008 | Arhgap5 | Rho GTPase activating protein 5 |
| BM384116 | LOC691397 /// | similar to PI-3-kinase-related kinase SMG-1 isoform 2 |
| | RGD1563508_predicted | (predicted) /// similar to PI-3-kinase-related kinase SMG-1 |
| BM384446 | Aof1_predicted | amine oxidase, flavin containing 1 (predicted) |
| BM384731 | RGD1309944_predicted | similar to RIKEN cDNA 0610009K11 (predicted) |
| BM384872 | Usp48 | ubiquitin specific protease 48 |
| BM384937 | MGC108776 | Snf7 homologue associated with Alix 3 |
| BM385061 | — | Transcribed locus |
| BM385071 | RGD1563060_predicted | similar to AVLV472 (predicted) |
| BM385117 | — | Transcribed locus |
| BM385221 | Ddx17 | DEAD (Asp-Glu-Ala-Asp) box polypeptide 17 |
| BM385244 | — | Transcribed locus |
| BM385286 | Lpin1 | lipin 1 |
| BM385476 | Bst2 | bone marrow stromal cell antigen 2 |
| BM385502 | Ka15 | type I keratin KA15 |
| BM385735 | Stac3_predicted | SH3 and cysteine rich domain 3 (predicted) |
| BM385779 | RGD1311307 | similar to 1300014I06Rik protein |
| BM385790 | Klf2_predicted | Kruppel-like factor 2 (lung) (predicted) |
| BM385804 | LOC360910 /// | similar to ATP-binding cassette transporter ABCG3 /// |
| | LOC360997 | similar to ATP-binding cassette, sub-family G (WHITE), |
| | | member 3 |
| BM385870 | — | Transcribed locus |
| BM385950 | Sbf1_predicted | SET binding factor 1 (predicted) |
| BM385951 | — | Transcribed locus |
| BM386010 | RGD1359684 | similar to T-cell receptor alpha chain precursor V and C |
| | | regions (TRA29) |
| BM386036 | — | Transcribed locus, strongly similar to XP_345296.2 |
| | | PREDICTED: similar to AVIEF [Rattus norvegicus] |
| BM386119 | Galnt3 | UDP-N-acetyl-alpha-D-galactosamine: polypeptide N- |
| | | acetylgalactosaminyltransferase 3 |
| BM386334 | Ankrd23_predicted | ankyrin repeat domain 23 (predicted) |
| BM386413 | Trim2 | tripartite motif protein 2 |
| BM386789 | Rgs1 | regulator of G-protein signaling 1 |
| BM387006 | Scamp2 | secretory carrier membrane protein 2 |
| BM387106 | Amid_predicted | Apoptosis-inducing factor (AIF)-like mitochondrion- |
| | | associated inducer of death (predicted) |
| BM387112 | — | Transcribed locus |
| BM387125 | — | Transcribed locus |
| BM387133 | — | — |
| BM387197 | — | — |
| BM387384 | Baz1a_predicted | bromodomain adjacent to zinc finger domain, 1A (predicted) |
| BM387419 | — | — |
| BM387754 | — | Transcribed locus |
| BM387797 | LOC360619 | similar to gasdermin 1 |
| BM387813 | — | Transcribed locus |
| BM387863 | Itgb3bp | integrin beta 3 binding protein (beta3-endonexin) |
| BM387914 | Krt25A | keratin 25A |
| BM387946 | Zap70 | zeta-chain (TCR) associated protein kinase 70 |
| BM388077 | RGD1562629_predicted | similar to neurobeachin (predicted) |
| BM388319 | Gimap7 | GTPase, IMAP family member 7 |
| BM388328 | RGD1311126_predicted | similar to RIKEN cDNA 4922503N01 (predicted) |
| BM388334 | Spink5_predicted | serine protease inhibitor, Kazal type 5 (predicted) |
| BM388416 | RGD1304621_predicted | similar to Hypothetical protein KIAA0539 (predicted) |
| BM388525 | F13a1 | coagulation factor XIII, A1 subunit |
| BM388719 | Rpl7 | Ribosomal protein L7 |
| BM388725 | — | Transcribed locus |
| BM388738 | — | — |
| BM388814 | LOC313707 | similar to exosome component 10 |
| BM388975 | Nfrkb_predicted | nuclear factor related to kappa B binding protein (predicted) |
| BM389001 | Col9a3_predicted | procollagen, type IX, alpha 3 (predicted) |
| BM389005 | Gpr68_predicted | G protein-coupled receptor 68 (predicted) |
| BM389207 | Brd4 | bromodomain containing 4 |
| BM389208 | Gimap4 | GTPase, IMAP family member 4 |
| BM389214 | RGD1559565_predicted | similar to EF hand domain containing 1 (predicted) |
| BM389223 | Pdhb | pyruvate dehydrogenase (lipoamide) beta |
| BM389240 | RGD1564142_predicted | similar to chromosome 10 open reading frame 64 (predicted) |
| BM389254 | Anxa8 | annexin A8 |
| BM389426 | Pdgfrb | platelet derived growth factor receptor, beta polypeptide |
| BM389444 | — | Transcribed locus |
| BM389513 | LOC688090 /// RT1-Bb | RT1 class II, locus Bb /// similar to RT1 class II |
| | | histocompatibility antigen, B-1 beta chain precursor (RT1.B- |
| | | beta(1)) |
| BM389585 | Capn13 | calpain 13 |
| BM389644 | Rhoj | ras homolog gene family, member J |
| BM389813 | — | — |
| BM389831 | Serpinb11_predicted | serine (or cysteine) peptidase inhibitor, clade B (ovalbumin), |
| | | member 11 (predicted) |
| BM389967 | RGD1308398 | similar to hypothetical protein MGC37548 |
| BM390000 | Sema3f_predicted | sema domain, immunoglobulin domain (Ig), short basic |
| | | domain, secreted, (semaphorin) 3 F (predicted) |
| BM390176 | Spink5_predicted | serine protease inhibitor, Kazal type 5 (predicted) |
| BM390226 | — | Transcribed locus |
| BM390227 | Bcl11b_predicted | B-cell leukemia/lymphoma 11B (predicted) |
| BM390320 | — | Transcribed locus, strongly similar to XP_580137.1 |
| | | PREDICTED: hypothetical protein XP_580137 [Rattus |
| | | norvegicus] |
| BM390324 | Eppb9_predicted | endothelial precursor protein B9 (predicted) |
| BM390325 | RGD1562868_predicted | similar to squamous cell carcinoma antigen 2 (predicted) |
| BM390378 | RGD1562732_predicted | similar to Glutathione S-transferase, theta 3 (predicted) |
| BM390399 | Hmgcr | 3-hydroxy-3-methylglutaryl-Coenzyme A reductase |
| BM390440 | — | Transcribed locus |
| BM390571 | Sult2b1_predicted | sulfotransferase family, cytosolic, 2B, member 1 (predicted) |
| BM390574 | Lss | Lanosterol synthase |
| BM390774 | Peci /// RGD1310224 | peroxisomal delta3, delta2-enoyl-Coenzyme A isomerase /// |
| | | similar to RIKEN cDNA 1810022C23 |
| BM390827 | Ppp1r3c | protein phosphatase 1, regulatory (inhibitor) subunit 3C |
| BM390872 | RGD1560293_predicted | similar to RIKEN cDNA 1200013B08 (predicted) |
| BM390965 | RGD1305592 | similar to RIKEN cDNA 2900092E17 |
| BM391096 | — | Transcribed locus |
| BM391100 | Muc4 | mucin 4 |
| BM391119 | Chd3 | chromodomain helicase DNA binding protein 3 |
| BM391169 | Myh4 | myosin, heavy polypeptide 4 |
| BM391278 | — | CDNA clone IMAGE: 7930328 |
| BM391283 | Etnk1_predicted | Ethanolamine kinase 1 (predicted) |
| BM391302 | Kdelr3_predicted | KDEL (Lys-Asp-Glu-Leu) endoplasmic reticulum protein |
| | | retention receptor 3 (predicted) |
| BM391303 | — | — |
| BM391354 | LOC287274 | sedlin-like |
| BM391471 | Atf5 | activating transcription factor 5 |
| BM341538 | Frzb | frizzled-related protein |
| BM391631 | Fcgr1 | Fc receptor, IgG, high affinity I |
| BM391750 | Usp40 | ubiquitin specific protease 40 |
| BM391823 | RGD1563309_predicted | similar to diacylglycerol kinase, delta 130 kDa isoform 1 |
| | | (predicted) |
| BM391896 | — | Transcribed locus |
| BM392106 | Cald1 | caldesmon 1 |
| BM392203 | Dapk2 | death-associated kinase 2 |
| BM392321 | Hipk2_predicted | homeodomain interacting protein kinase 2 (predicted) |
| D10831 | Sell | selectin, lymphocyte |
| D13555 | Cd3z | CD3 antigen, zeta polypeptide |
| D29960 | Hspb6 | heat shock protein, alpha-crystallin-related, B6 |
| D50306 | Slc15a1 | solute carrier family 15 (oligopeptide transporter), member 1 |
| D83948 | Rbm10 | RNA binding motif protein 10 |
| D86817 | Vamp4_predicted | vesicle-associated membrane protein 4 (predicted) |
| D87927 | Gm1960 | gene model 1960, (NCBI) |
| D88586 | Ear11 | eosinophil-associated, ribonuclease A family, member 11 |
| D88672 | Pld2 | phospholipase D2 |
| H31078 | — | — |
| H33235 | Cs | citrate synthase |
| H34497 | LOC684513 /// | similar to SWI/SNF-related matrix-associated actin- |
| | LOC685179 | dependent regulator of chromatin c2 isoform b /// similar to |
| | | SWI/SNF-related matrix-associated actin-dependent |
| | | regulator of chromatin c2 |
| H34775 | Riok3_predicted | RIO kinase 3 (yeast) (predicted) |
| J00710 | Csn1s1 | casein alpha s1 |
| J00741 | LOC641523 | immunoglobulin delta heavy chain constant region |
| J02585 | Scd1 | stearoyl-Coenzyme A desaturase 1 |
| J02612 | Ugt1a1 /// Ugt1a10 /// | UDP glycosyltransferase 1 family, polypeptide A1 /// UDP |
| | Ugt1a2 /// Ugt1a3 /// | glycosyltransferase 1 family, polypeptide A6 /// UDP |
| | Ugt1a5 /// Ugt1a6 /// | glycosyltransferase 1 family, polypeptide A7 /// UDP |
| | Ugt1a7 /// Ugt1a8 | glycosyltransferase 1 family, polypeptide A8 /// UDP |
| | | glycosyltransferase 1 family polypeptide A2 /// UDP |
| | | glycosyltransferase 1 family polypeptide A3 /// UDP |
| | | glycosyltransferase 1 family polypeptide A10 /// UDP |
| | | glycosyltransferase 1 family, polypeptide A5 |
| J02705 | Ocm | oncomodulin |
| J02811 | Ampd1 | adenosine monophosphate deaminase 1 (isoform M) |
| J03867 | Cyb5r3 | cytochrome b5 reductase 3 |
| J04035 | Eln | elastin |
| L07316 | Dpep1 | dipeptidase 1 (renal) |
| L08447 | Cd3z | CD3 antigen, zeta polypeptide |
| L14782 | Lyn | Yamaguchi sarcoma viral (v-yes-1) oncogene homolog |
| L17138 | Hsd3b6 | hydroxy-delta-5-steroid dehydrogenase, 3 beta- and steroid |
| | | delta-isomerase 6 |
| L19933 | Ptprd | protein tyrosine phosphatase, receptor type, D |
| L22654 | IgG-2a | gamma-2a immunoglobulin heavy chain |
| L25527 | Sele | selectin, endothelial cell |
| L81169 | Oxtr | oxytocin receptor |
| M10072 | Ptprc | protein tyrosine phosphatase, receptor type, C |
| M13706 | Prkcb1 | protein kinase C, beta 1 |
| M15481 | Igf1 | insulin-like growth factor 1 |
| M17153 | Fcer1a | Fc receptor, IgE, high affinity I, alpha polypeptide |
| M24024 | RT1-Aw2 | RT1 class Ib, locus Aw2 |
| M25804 | Nr1d1 | nuclear receptor subfamily 1, group D, member 1 |
| M30596 | Me1 | malic enzyme 1 |
| M37568 | Hoxc6 /// Hoxc8 | homeo box C6 /// homeo box C8 |
| M57668 | Prlr | prolactin receptor |
| M61142 | Thop1 | thimet oligopeptidase 1 |
| M83681 | Rab3d | RAB3D, member RAS oncogene family |
| M85035 | Gria2 | glutamate receptor, ionotropic, AMPA2 |
| M88469 | Spon1 | spondin 1 |
| M94288 | Nolc1 | nucleolar and coiled-body phosphoprotein 1 |
| NM_012488 | A2m | alpha-2-macroglobulin |
| NM_012493 | Afp | alpha-fetoprotein |
| NM_012497 | Aldoc | aldolase C |
| NM_012502 | Ar | androgen receptor |
| NM_012516 | C4bpa | complement component 4 binding protein, alpha |
| NM_012524 | Cebpa | CCAAT/enhancer binding protein (C/EBP), alpha |
| NM_012530 | Ckm | creatine kinase, muscle |
| NM_012531 | Comt | catechol-O-methyltransferase |
| NM_012532 | Cp | ceruloplasmin |
| NM_012554 | Eno1 | enolase 1, alpha |
| NM_012555 | Ets1 | v-ets erythroblastosis virus E26 oncogene homolog 1 (avian) |
| NM_012559 | Fgg | fibrinogen, gamma polypeptide |
| NM_012561 | Fst | follistatin |
| NM_012594 | Lalba | lactalbumin, alpha |
| NM_012600 | Me1 | malic enzyme 1 |
| NM_012605 | Mylpf | myosin light chain, phosphorylatable, fast skeletal muscle |
| NM_012606 | Myl3 | myosin, light polypeptide 3 |
| NM_012640 | Rbp2 | retinol binding protein 2, cellular |
| NM_012646 | RT1-N1 /// RT1-N2 /// | RT1 class Ib gene, H2-TL-like, grc region (N1) /// RT1 class |
| | RT1-N3 | Ib gene, H2-TL-like, grc region (N3) /// RT1 class Ib gene, |
| | | H2-TL-like, grc region(N2) |
| NM_012652 | Slc9a1 | solute carrier family 9, member 1 |
| NM_012660 | Eef1a2 | eukaryotic translation elongation factor 1 alpha 2 |
| NM_012663 | Vamp2 | vesicle-associated membrane protein 2 |
| NM_012684 | Vcsa1 /// Vcsa2 | variable coding sequence A1 /// variable coding sequence A2 |
| NM_012703 | Thrsp | thyroid hormone responsive protein |
| NM_012733 | Rbp1 | retinol binding protein 1, cellular |
| NM_012744 | Pc | Pyruvate carboxylase |
| NM_012745 | Klrd1 | killer cell lectin-like receptor, subfamily D, member 1 |
| NM_012755 | Fyn | fyn proto-oncogene |
| NM_012760 | Plagl1 | pleiomorphic adenoma gene-like 1 |
| NM_012777 | Apod | apolipoprotein D |
| NM_012779 | Aqp5 | aquaporin 5 |
| NM_012786 | Cox8h | Cytochrom c oxidase subunit VIII-H (heart/muscle) |
| NM_012794 | Glycam1 | glycosylation dependent cell adhesion molecule 1 |
| NM_012812 | Cox6a2 | cytochrome c oxidase, subunit VIa, polypeptide 2 |
| NM_012824 | Apoc1 | apolipoprotein C-I |
| NM_012836 | Cpd | carboxypeptidase D |
| NM_012851 | Hsd17b1 | hydroxysteroid (17-beta) dehydrogenase 1 |
| NM_012855 | Jak3 | Janus kinase 3 |
| NM_012888 | Tshr | thyroid stimulating hormone receptor |
| NM_012893 | Actg2 | actin, gamma 2 |
| NM_012914 | Atp2a3 | ATPase, Ca++ transporting, ubiquitous |
| NM_012915 | Atpif1 | ATPase inhibitory factor 1 |
| NM_012925 | Cd59 | CD59 antigen |
| NM_012938 | Ctse | cathepsin E |
| NM_012941 | Cyp51 | cytochrome P450, subfamily 51 |
| NM_012949 | Eno3 | enolase 3, beta |
| NM_012993 | Nrd1 | nardilysin, N-arginine dibasic convertase 1 |
| NM_013002 | Pcp4 | Purkinje cell protein 4 |
| NM_013020 | Rabggta | Rab geranylgeranyl transferase, a subunit |
| NM_013026 | Sdc1 | syndecan 1 |
| NM_013028 | Shox2 | short stature homeobox 2 |
| NM_013041 | Sycp3 | synaptonemal complex protein 3 |
| NM_013060 | Id2 | inhibitor of DNA binding 2 |
| NM_013085 | Plau | plasminogen activator, urokinase |
| NM_013092 | Cma1 | chymase 1, mast cell |
| NM_013127 | Cd38 | CD38 antigen |
| NM_013134 | Hmgcr | 3-hydroxy-3-methylglutaryl-Coenzyme A reductase |
| NM_013169 | Cd3d | CD3 antigen delta polypeptide |
| NM_013172 | Myf6 | myogenic factor 6 |
| NM_013186 | Kcnb1 | potassium voltage gated channel, Shab-related subfamily, |
| | | member 1 |
| NM_013187 | Plcg1 | phospholipase C, gamma 1 |
| NM_013194 | Myh9 | myosin, heavy polypeptide 9, non-muscle |
| NM_013200 | Cpt1b | carnitine palmitoyltransferase 1b, muscle |
| NM_016986 | Acadm | acetyl-Coenzyme A dehydrogenase, medium chain |
| NM_016987 | Acly | ATP citrate lyase |
| NM_016998 | Cpa1 | carboxypeptidase A1 |
| NM_017006 | G6pdx | glucose-6-phosphate dehydrogenase X-linked |
| NM_017015 | Gusb | glucuronidase, beta |
| NM_017028 | Mx2 | myxovirus (influenza virus) resistance 2 |
| NM_017048 | Slc4a2 | solute carrier family 4, member 2 |
| NM_017066 | Ptn | pleiotrophin |
| NM_017120 | Csn2 | casein beta |
| NM_017124 | Cd37 | CD37 antigen |
| NM_017130 | Neu2 | neuraminidase 2 |
| NM_017156 | Cyp2b15 | cytochrome P450, family 2, subfamily b, polypeptide 15 |
| NM_017174 | Pla2g5 | phospholipase A2, group V |
| NM_017185 | Tnni2 | troponin I type 2 (skeletal, fast) |
| NM_017200 | Tfpi | tissue factor pathway inhibitor |
| NM_017206 | Slc6a6 | solute carrier family 6 (neurotransmitter transporter, taurine), |
| | | member 6 |
| NM_017225 | Pctp | phosphatidylcholine transfer protein |
| NM_017240 | Myh6 /// Myh7 | myosin, heavy polypeptide 6, cardiac muscle, alpha /// |
| | | myosin, heavy polypeptide 7, cardiac muscle, beta |
| NM_017240 | Myh7 | myosin, heavy polypeptide 7, cardiac muscle, beta |
| NM_017249 | Mbc2 | membrane bound C2 domain containing protein |
| NM_017259 | Btg2 | B-cell translocation gene 2, anti-proliferative |
| NM_017260 | Alox5ap | arachidonate 5-lipoxygenase activating protein |
| NM_017261 | Gria2 | glutamate receptor, ionotropic, AMPA2 |
| NM_017268 | Hmgcs1 | 3-hydroxy-3-methylglutaryl-Coenzyme A synthase 1 |
| NM_017307 | Slc25a1 | solute carrier family 25, member 1 |
| NM_017311 | Atp5g1 | ATP synthase, H+ transporting, mitochondrial F0 complex, |
| | | subunit c (subunit 9), isoform 1 |
| NM_017313 | Rab3ip | RAB3A interacting protein |
| NM_017320 | Ctss | cathepsin S |
| NM_017328 | Pgam2 | phosphoglycerate mutase 2 |
| NM_017332 | Fasn | fatty acid synthase |
| NM_019131 | Tpm1 | tropomyosin 1, alpha |
| NM_019161 | RGD1566401_predicted | similar to GTL2, imprinted maternally expressed |
| | | untranslated (predicted) |
| NM_019171 | LOC685608 /// | salivary protein 1 /// hypothetical protein LOC685608 /// |
| | LOC688807 /// Spt1 | hypothetical protein LOC688807 |
| NM_019175 | Klk6 | kallikrein 6 |
| NM_019193 | Sox10 | SRY-box containing gene 10 |
| NM_019205 | Ccl11 | chemokine (C-C motif) ligand 11 |
| NM_019206 | Stk10 | serine/threonine kinase 10 |
| NM_019212 | Acta1 | actin, alpha 1, skeletal muscle |
| NM_019221 | Trp63 | transformation related protein 63 |
| NM_019225 | Slc1a3 | solute carrier family 1 (glial high affinity glutamate |
| | | transporter), member 3 |
| NM_019238 | Fdft1 | farnesyl diphosphate farnesyl transferase 1 |
| NM_019240 | Gjb3 | gap junction membrane channel protein beta 3 |
| NM_019241 | Gjb5 | gap junction membrane channel protein beta 5 |
| NM_019282 | Grem1 | gremlin 1 homolog, cysteine knot superfamily (Xenopus |
| | | laevis) |
| NM_019291 | Ca2 | carbonic anhydrase 2 |
| NM_019295 | Cd5 | CD5 antigen |
| NM_019311 | Inpp5d | inositol polyphosphate-5-phosphatase D |
| NM_019334 | Pitx2 | paired-like homeodomain transcription factor 2 |
| NM_019370 | Enpp3 | ectonucleotide pyrophosphatase/phosphodiesterase 3 |
| NM_019371 | Egln3 | EGL nine homolog 3 (C. elegans) |
| NM_019384 | Scaf1 | SR-related CTD-associated factor 1 |
| NM_020072 | Acpp | acid phosphatase, prostate |
| NM_020099 | Leprot | leptin receptor overlapping transcript |
| NM_020104 | Myl1 | myosin, light polypeptide 1 |
| NM_020542 | Ccr1 | chemokine (C-C motif) receptor 1 |
| NM_021581 | Sc65 | synaptonemal complex protein SC65 |
| NM_021588 | Mb | myoglobin |
| NM_021694 | Arhgef1 | Rho guanine nucleotide exchange factor (GEF) 1 |
| NM_021698 | F13a1 | coagulation factor XIII, A1 subunit |
| NM_021744 | Cd14 | CD14 antigen |
| NM_021759 | Lypd3 | Ly6/Plaur domain containing 3 |
| NM_021760 | Col5a3 | procollagen, type V, alpha 3 |
| NM_021769 | Sult1d1 | sulfotransferase family 1D, member 1 |
| NM_021866 | Ccr2 | chemokine (C-C motif) receptor 2 |
| NM_022193 | Acaca | acetyl-coenzyme A carboxylase alpha |
| NM_022229 | Hspd1 | heat shock protein 1 (chaperonin) |
| NM_022268 | Pygl | liver glycogen phosphorylase |
| NM_022282 | Dlgh2 | discs, large homolog 2 (Drosophila) |
| NM_022389 | Dhcr7 | 7-dehydrocholesterol reductase |
| NM_022392 | Insig1 | insulin induced gene 1 |
| NM_022396 | Gng11 | guanine nucleotide binding protein (G protein), gamma 11 |
| NM_022582 | Lgals7 | lectin, galactose binding, soluble 7 |
| NM_022592 | Tkt | transketolase |
| NM_022596 | Golga2 | cis-Golgi matrix protein GM130 |
| NM_022603 | Fgfbp1 | fibroblast growth factor binding protein 1 |
| NM_022610 | Icos | inducible T-cell co-stimulator |
| NM_022628 | Nphs1 | nephrosis 1 homolog, nephrin (human) |
| NM_022676 | Ppp1r1a | protein phosphatase 1, regulatory (inhibitor) subunit 1A |
| NM_022681 | Adnp | activity-dependent neuroprotective protein |
| NM_022686 | LOC680097 /// | similar to germinal histone H4 gene |
| | LOC684887 | |
| NM_022705 | Thedc1 | thioesterase domain containing 1 |
| NM_022799 | Nucks | nuclear ubiquitous casein kinase and cyclin-dependent |
| | | kinase substrate |
| NM_022860 | B4galnt1 | beta-1,4-N-acetyl-galactosaminyl transferase 1 |
| NM_022935 | Abp1 | amiloride binding protein 1 (amine oxidase, copper- |
| | | containing) |
| NM_022952 | Ap2s1 | adaptor-related protein complex 2, sigma 1 subunit |
| NM_023021 | Kcnn4 | potassium intermediate/small conductance calcium-activated |
| | | channel, subfamily N, member 4 |
| NM_023025 | Cyp2j3 /// Cyp2j4 | cytochrome P450, family 2, subfamily J, polypeptide 4 /// |
| | | cytochrome P450, family 2, subfamily j, polypeptide 3 |
| NM_023092 | Myo1c | myosin IC |
| NM_023987 | Birc3 | baculoviral IAP repeat-containing 3 |
| NM_024002 | Secisbp2 | SECIS binding protein 2 |
| NM_024131 | Ddt | D-dopachrome tautomerase |
| NM_024147 | Evl | Ena-vasodilator stimulated phosphoprotein |
| NM_024162 | Fabp3 | fatty acid binding protein 3 |
| NM_024364 | Hr | hairless homolog (mouse) |
| NM_024390 | Hpgd | hydroxyprostaglandin dehydrogenase 15 (NAD) |
| NM_024398 | Aco2 | aconitase 2, mitochondrial |
| NM_030585 | Rabep2 | rabaptin, RAB GTPase binding effector protein 2 |
| NM_030836 | Arts1 | type 1 tumor necrosis factor receptor shedding |
| | | aminopeptidase regulator |
| NM_030852 | Mia1 | melanoma inhibitory activity 1 |
| NM_030853 | Lat | linker for activation of T cells |
| NM_030863 | Msn | moesin |
| NM_030865 | Myoc | myocilin |
| NM_030989 | Tp53 | tumor protein p53 |
| NM_030994 | Itga1 | integrin alpha 1 |
| NM_031020 | Mapk14 | mitogen activated protein kinase 14 |
| NM_031031 | Gatm | glycine amidinotransferase (L-arginine: glycine |
| | | amidinotransferase) |
| NM_031051 | Mif | macrophage migration inhibitory factor |
| NM_031085 | Prkch | protein kinase C, eta |
| NM_031116 | Ccl5 | chemokine (C-C motif) ligand 5 |
| NM_031143 | Dgkz | diacylglycerol kinase zeta |
| NM_031144 | Actb | actin, beta |
| NM_031327 | Cyr61 | cysteine rich protein 61 |
| NM_031337 | St3gal5 | ST3 beta-galactoside alpha-2,3-sialyltransferase 5 |
| NM_031360 | Smpd2 | sphingomyelin phosphodiesterase 2, neutral |
| NM_031530 | Ccl2 | chemokine (C-C motif) ligand 2 |
| NM_031538 | Cd8a | CD8 antigen, alpha chain |
| NM_031544 | Ampd3 | adenosine monophosphate deaminase 3 |
| NM_031559 | Cpt1a | carnitine palmitoyltransferase 1a, liver |
| NM_031562 | Csn10 | casein kappa |
| NM_031598 | Pla2g2a | phospholipase A2, group IIA (platelets, synovial fluid) |
| NM_031646 | Ramp2 | receptor (calcitonin) activity modifying protein 2 |
| NM_031699 | Cldn1 | claudin 1 |
| NM_031700 | Cldn3 | claudin 3 |
| NM_031715 | Pfkm | phosphofructokinase, muscle |
| NM_031766 | Cpz | carboxypeptidase Z |
| NM_031771 | Thbd | thrombomodulin |
| NM_031778 | Kcns3 | potassium voltage-gated channel, delayed-rectifier, |
| | | subfamily S, member 3 |
| NM_031781 | Apba3 | amyloid beta (A4) precursor protein-binding, family A, |
| | | member 3 |
| NM_031786 | Trim3 | tripartite motif protein 3 |
| NM_031801 | Deaf1 | deformed epidermal autoregulatory factor 1 (Drosophila) |
| NM_031817 | Omd | osteomodulin |
| NM_031827 | Vamp8 | vesicle-associated membrane protein 8 |
| NM_031837 | Sept9 | septin 9 |
| NM_031840 | Fdps | farnesyl diphosphate synthase |
| NM_031841 | Scd2 | stearoyl-Coenzyme A desaturase 2 |
| NM_031971 | Hspa1a /// Hspa1b | heat shock 70 kD protein 1A /// heat shock 70 kD protein 1B |
| | | (mapped) |
| NM_033230 | Akt1 | thymoma viral proto-oncogene 1 |
| NM_053322 | Pom210 | nuclear pore membrane glycoprotein 210 |
| NM_053328 | Bhlhb2 | basic helix-loop-helix domain containing, class B2 |
| NM_053339 | Acox3 | acyl-Coenzyme A oxidase 3, pristanoyl |
| NM_053355 | Ikbkb | inhibitor of kappaB kinase beta |
| NM_053372 | RGD1563818_predicted | secretory leukocyte peptidase inhibitor /// similar to |
| | /// Slpi | secretory leukocyte protease inhibitor (predicted) |
| NM_053388 | Gjb6 | gap junction membrane channel protein beta 6 |
| NM_053433 | Fmo3 | flavin containing monooxygenase 3 |
| NM_053535 | Enpp1 | ectonucleotide pyrophosphatase/phosphodiesterase 1 |
| NM_053539 | Idi1 | isopentenyl-diphosphate delta isomerase |
| NM_053540 | Cox17 | cytochrome c oxidase, subunit XVII assembly protein |
| | | homolog (yeast) |
| NM_053584 | — | — |
| NM_053587 | S100a9 | S100 calcium binding protein A9 (calgranulin B) |
| NM_053591 | Dpep1 | dipeptidase 1 (renal) |
| NM_053618 | Bbs2 | Bardet-Biedl syndrome 2 homolog (human) |
| NM_053623 | Acsl4 | acyl-CoA synthetase long-chain family member 4 |
| NM_053633 | Egr2 | early growth response 2 |
| NM_053639 | Ltc4s | leukotriene C4 synthase |
| NM_053669 | Aps | adaptor protein with pleckstrin homology and src homology |
| | | 2 domains |
| NM_053674 | Phyh | phytanoyl-CoA hydroxylase |
| NM_053688 | Pde6h | phosphodiesterase 6H, cGMP-specific, cone, gamma |
| NM_053714 | Ank | progressive ankylosis homolog (mouse) |
| NM_053747 | Ubqln1 | ubiquilin 1 |
| NM_053751 | Wap | whey acidic protein |
| NM_053806 | Kcnk6 /// LOC501662 | potassium channel, subfamily K, member 6 /// similar to |
| | | potassium channel, subfamily K, member 6 |
| NM_053819 | Timp1 | tissue inhibitor of metallopeptidase 1 |
| NM_053822 | S100a8 | S100 calcium binding protein A8 (calgranulin A) |
| NM_053826 | Pdk1 | pyruvate dehydrogenase kinase, isoenzyme 1 |
| NM_053827 | Plod1 | procollagen-lysine, 2-oxoglutarate 5-dioxygenase 1 |
| NM_053843 | Fcgr3 | Fc receptor, IgG, low affinity III |
| NM_053847 | Map3k8 | mitogen-activated protein kinase kinase kinase 8 |
| NM_053864 | Vcp | valosin-containing protein |
| NM_053883 | Dusp6 | dual specificity phosphatase 6 |
| NM_053885 | Rere | arginine-glutamic acid dipeptide (RE) repeats |
| NM_053887 | Map3k1 | mitogen activated protein kinase kinase kinase 1 |
| NM_053904 | Oplah | 5-oxoprolinase (ATP-hydrolysing) |
| NM_053953 | Il1r2 | interleukin 1 receptor, type II |
| NM_053960 | Ccr5 | chemokine (C-C motif) receptor 5 |
| NM_053999 | Ppp2r2a | protein phosphatase 2 (formerly 2A), regulatory subunit B |
| | | (PR 52), alpha isoform |
| NM_057144 | Csrp3 | cysteine and glycine-rich protein 3 |
| NM_057151 | Ccl17 | chemokine (C-C motif) ligand 17 |
| NM_057187 | Grifin | galectin-related inter-fiber protein |
| NM_057191 | Kbtbd10 | kelch repeat and BTB (POZ) domain containing 10 |
| NM_057208 | Tpm3 | tropomyosin 3, gamma |
| NM_058213 | Atp2a1 | ATPase, Ca++ transporting, cardiac muscle, fast twitch 1 |
| NM_080399 | Ddit41 | DNA-damage-inducible transcript 4-like |
| NM_080770 | Scgb2a1 /// Scgb2a2 | secretoglobin, family 2A, member 1 /// secretoglobin, family |
| | | 2A, member 2 |
| NM_080787 | Dgka | diacylglycerol kinase, alpha |
| NM_080906 | Ddit4 | DNA-damage-inducible transcript 4 |
| NM_130399 | Ada | adenosine deaminase |
| NM_130409 | Cfh | complement component factor H |
| NM_130411 | Coro1a | coronin, actin binding protein 1A |
| NM_130421 | Lcp2 | lymphocyte cytosolic protein 2 |
| NM_130429 | Lef1 | lymphoid enhancer binding factor 1 |
| NM_130433 | Acaa2 | acetyl-Coenzyme A acyltransferase 2 (mitochondrial 3- |
| | | oxoacyl-Coenzyme A thiolase) |
| NM_130741 | Lcn2 | lipocalin 2 |
| NM_130744 | Cygb | cytoglobin |
| NM_130755 | Cs | citrate synthase |
| NM_133303 | Bhlhb3 | basic helix-loop-helix domain containing, class B3 |
| NM_133320 | Ndel1 | nudE nuclear distribution gene E homolog like 1 (A. nidulans) |
| NM_133392 | Stk17b | serine/threonine kinase 17b (apoptosis-inducing) |
| NM_133416 | Bcl2a1 | B-cell leukemia/lymphoma 2 related protein A1 |
| NM_133418 | Slc25a10 | solute carrier family 25 (mitochondrial carrier; dicarboxylate |
| | | transporter), member 10 |
| NM_133421 | Lkap | limkain b1 |
| NM_133424 | Actn3 | actinin alpha 3 |
| NM_133513 | Muc10 | mucin 10, submandibular gland salivary mucin |
| NM_133522 | Sstr3 | somatostatin receptor 3 |
| NM_133524 | Tcfe2a | transcription factor E2a |
| NM_133533 | Cd79b | CD79B antigen |
| NM_133542 | Igsf6 | immunoglobulin superfamily, member 6 |
| NM_133551 | Pla2g4a | phospholipase A2, group IVA (cytosolic, calcium- |
| | | dependent) |
| NM_133561 | Brp44l | brain protein 44-like |
| NM_133566 | Cst6 | cystatin E/M |
| NM_133572 | Cdc25b | cell division cycle 25 homolog B (S. pombe) |
| NM_133600 | Slc31a1 | solute carrier family 31 (copper transporters), member 1 |
| NM_134326 | Alb | albumin |
| NM_134334 | Ctsd | cathepsin D |
| NM_134351 | Mat2a | methionine adenosyltransferase II, alpha |
| NM_134361 | Xcl1 | chemokine (C motif) ligand 1 |
| NM_134382 | Elovl5 | ELOVL family member 5, elongation of long chain fatty |
| | | acids (yeast) |
| NM_134389 | Acsbg1 | acyl-CoA synthetase bubblegum family member 1 |
| NM_138850 | Fap | fibroblast activation protein |
| S75280 | Hspa9a_predicted | heat shock 70 kDa protein 9A (predicted) |
| U13253 | Fabp5 | fatty acid binding protein 5, epidermal |
| U17565 | Mcm6 | minichromosome maintenance deficient 6 (MIS5 homolog, |
| | | S. pombe) (S. cerevisiae) |
| U22520 | Cxcl10 | chemokine (C-X-C motif) ligand 10 |
| U23056 | Ceacam1 /// Ceacam10 | CEA-related cell adhesion molecule 1 /// CEA-related cell |
| | | adhesion molecule 10 |
| U23407 | Crabp2 | cellular retinoic acid binding protein 2 |
| U24150 | Tsc2 | tuberous sclerosis 2 |
| U25684 | Tmsbl1 | thymosin beta-like protein 1 |
| U25746 | Ddx46 | DEAD (Asp-Glu-Ala-Asp) box polypeptide 46 |
| U28830 | Ralbp1 | ralA binding protein 1 |
| U35025 | Acvr1c | activin A receptor, type IC |
| U44948 | Csrp2 | cysteine and glycine-rich protein 2 |
| U50449 | RT1-CE16 | RT1 class I, CE16 |
| U53475 | Rab8b | RAB8B, member RAS oncogene family |
| U54791 | Cxcr4 | chemokine (C-X-C motif) receptor 4 |
| U56824 | Klra5 /// Ly49s7 | killer cell lectin-like receptor, subfamily A, member 5 /// |
| | | Ly49 stimulatory receptor 7 |
| U67914 | Cpa3 | carboxypeptidase A3 |
| U67915 | Mcpt1 | mast cell protease 1 |
| U78857 | Dclk1 | doublecortin-like kinase 1 |
| U86635 | Gstm5 | glutathione S-transferase, mu 5 |
| X04440 | Prkcb1 | protein kinase C, beta 1 |
| X16262 | RGD1564935_predicted | similar to Myh11 protein (predicted) |
| X57764 | Ednrb | endothelin receptor type B |
| X63744 | Slc1a3 | solute carrier family 1 (glial high affinity glutamate |
| | | transporter), member 3 |
| X74293 | Itga7 | integrin alpha 7 |
| X74294 | Itga7 | integrin alpha 7 |
| X77815 | Vcsa2 | variable coding sequence A2 |
| X77818 | — | — |
| X83537 | Mmp14 | matrix metallopeptidase 14 (membrane-inserted) |
| X97445 | Slc16a7 | solute carrier family 16 (monocarboxylic acid transporters), |
| | | member 7 |
| Z18877 | Oas1 | 2′,5′-oligoadenylate synthetase 1, 40/46 kDa |

B. Liver

| | | |
|---|---|---|
| AA799328 | RGD1560913_predicted | similar to expressed sequence AW413625 (predicted) |
| AA799700 | Sephs2 | selenophosphate synthetase 2 |
| AA800212 | Atp2a2 | ATPase, Ca++ transporting, cardiac muscle, slow twitch 2 |
| AA817761 | Adh1 | alcohol dehydrogenase 1 (class I) |
| AA818262 | Angptl4 | angiopoietin-like 4 |
| AA818759 | RGD1560745_predicted | similar to OTTHUMP00000018508 (predicted) |
| AA818819 | — | Transcribed locus |
| AA818900 | LOC304138 | similar to cysteine and tyrosine-rich protein 1 |
| AA819250 | RGD1560915_predicted | similar to Hypothetical protein MGC30714 (predicted) |
| AA849966 | — | — |
| AA850751 | — | — |
| AA858645 | Chd1l_predicted | chromodomain helicase DNA binding protein 1-like |
| | | (predicted) |
| AA858930 | Pde4b | Phosphodiesterase 4B, cAMP specific |
| AA858993 | Ugt2b | UDP glycosyltransferase 2 family, polypeptide B |
| AA859029 | — | Transcribed locus |
| AA859049 | Es2 | esterase 2 |
| AA859496 | Gch | GTP cyclohydrolase 1 |
| AA859663 | LOC301113 | similar to solute carrier family 25 (mitochondrial carrier; |
| | | phosphate carrier), member 23 |
| AA874941 | Adfp | Adipose differentiation related protein |
| AA891362 | Hadhsc | L-3-hydroxyacyl-Coenzyme A dehydrogenase, short chain |
| AA891834 | Col4a5_predicted | collagen, type IV, alpha 5 (predicted) |
| AA892813 | LOC680445 | Similar to muscleblind-like 2 isoform 1 |
| AA892845 | Hexb | hexosaminidase B |
| AA893169 | Timp3 | tissue inhibitor of metalloproteinase 3 (Sorsby fundus |
| | | dystrophy, pseudoinflammatory) |
| AA893192 | — | Transcribed locus |
| AA893529 | — | — |
| AA899326 | — | Transcribed locus |
| AA899371 | Pwp1_predicted | PWP1 homolog (S. cerevisiae) (predicted) |
| AA899481 | — | Transcribed locus |
| AA899721 | Mte1 | mitochondrial acyl-CoA thioesterase 1 |
| AA900547 | Nfx1 | Nuclear transcription factor, X-box binding 1 |
| AA900645 | — | Transcribed locus |
| AA901337 | Spic_predicted | Spi-C transcription factor (Spi-1/PU.1 related) (predicted) |
| AA924350 | LOC686634 /// | hypothetical protein LOC686634 /// hypothetical protein |
| | LOC690768 | LOC690768 |
| AA924380 | Zfp131 | zinc finger protein 131 |
| AA924588 | RGD1304748 | similar to cDNA sequence BC006662 |
| AA924620 | Rab40b_predicted | Rab40b, member RAS oncogene family (predicted) |
| AA926109 | — | Transcribed locus |
| AA926305 | — | Transcribed locus |
| AA945268 | — | Transcribed locus |
| AA945604 | — | Transcribed locus |
| AA945955 | Ogn_predicted | osteoglycin (predicted) |
| AA946294 | — | — |
| AA955091 | Itga6 | integrin, alpha 6 |
| AA955771 | — | — |
| AA955879 | — | Transcribed locus |
| AA957585 | Ehd3 | EH-domain containing 3 |
| AA957990 | — | Transcribed locus |
| AA963524 | Slc35c2 | solute carrier family 35, member C2 |
| AA963797 | Sulf2 | sulfatase 2 |
| AA997115 | RGD1311859_predicted | similar to hypothetical protein DKFZp434H0115 (predicted) |
| AA997683 | Aldh1b1 | aldehyde dehydrogenase 1 family, member B1 |
| AA997980 | — | Transcribed locus |
| AA998264 | LOC307495 | similar to biliverdin reductase B (flavin reductase (NADPH)) |
| AA998903 | — | CDNA clone IMAGE: 7374368 |
| AB013454 | Slc34a1 | solute carrier family 34 (sodium phosphate), member 1 |
| AB019693 | Keg1 | kidney expressed gene 1 |
| AB020967 | Trib3 | tribbles homolog 3 (Drosophila) |
| AB030829 | Ca3 | carbonic anhydrase 3 |
| AB047324 | Slc16a10 | solute carrier family 16 (monocarboxylic acid transporters), |
| | | member 10 |
| AB052294 | Abcc8 | ATP-binding cassette, sub-family C (CFTR/MRP), member 8 |
| AF065161 | Cish | cytokine inducible SH2-containing protein |
| AF072411 | Cd36 /// LOC685953 /// | cd36 antigen /// similar to fatty acid translocase/CD36 |
| | RGD1562323_predicted | (predicted) /// similar to CD36 antigen |
| AF079864 | Olr59 | olfactory receptor 59 |
| AF090134 | Lin7a | lin-7 homolog a (C. elegans) |
| AF111099 | Lphn1 | latrophilin 1 |
| AF202733 | Pde4b | phosphodiesterase 4B, cAMP specific |
| AF311886 | LOC266761 | cytochrome P450-like protein |
| AF335281 | Steap3 | STEAP family member 3 |
| AF411318 | Mt1a | metallothionein 1a |
| AI008441 | Pgd | phosphogluconate dehydrogenase (mapped) |
| AI010322 | Mbnl3_predicted | muscleblind-like 3 (Drosophila) (predicted) |
| AI010839 | Kcnma1 | potassium large conductance calcium-activated channel, |
| | | subfamily M, alpha member 1 |
| AI013299 | RGD1565184_predicted | ventricular zone expressed PH domain homolog 1 |
| | /// Veph1 | (zebrafish) /// similar to VEPH isoform A (predicted) |
| AI013567 | Glt25d1_predicted | glycosyltransferase 25 domain containing 1 (predicted) |
| AI044253 | — | Transcribed locus |
| AI044343 | RGD1560170_predicted | similar to cofactor required for Sp1 transcriptional |
| | | activation, subunit 2, 150 kDa (predicted) |
| AI045015 | RGD1560736_predicted | similar to solute carrier family 9 (sodium/hydrogen |
| | | exchanger), isoform 9 (predicted) |
| AI045116 | — | Transcribed locus |
| AI045533 | LOC679725 | Similar to MASK-4E-BP3 protein |
| AI045767 | Ank | progressive ankylosis homolog (mouse) |
| AI058895 | — | Transcribed locus |
| AI059204 | Cpne8_predicted | copine VIII (predicted) |
| AI060043 | — | Transcribed locus |
| AI060139 | Atxn2_predicted | Ataxin 2 (predicted) |
| AI060227 | Rnd3 | Rho family GTPase 3 |
| AI071547 | RGD1308329_predicted | Similar to KIAA0869 protein (predicted) |
| AI072107 | Akr1c6 | aldo-keto reductase family 1, member C6 |
| AI072892 | Frzb | frizzled-related protein |
| AI073272 | Tor1b | torsin family 1, member B |
| AI101330 | Ncald | neurocalcin delta |
| AI101727 | — | Transcribed locus |
| AI102035 | — | Transcribed locus |
| AI102061 | Gria3 | glutamate receptor, ionotropic, AMPA3 (alpha 3) |
| AI102258 | — | Transcribed locus |
| AI103389 | — | — |
| AI104238 | Gzma | granzyme A |
| AI110520 | — | Transcribed locus |
| AI111413 | Spsb4_predicted | splA/ryanodine receptor domain and SOCS box containing 4 |
| | | (predicted) |
| AI113090 | — | Transcribed locus |
| AI113261 | LOC687696 | similar to AMSH-family protein |
| AI136684 | siat7D | alpha-2,6-sialyltransferase ST6GalNAc IV |
| AI137045 | — | Transcribed locus |
| AI137310 | — | Transcribed locus |
| AI137640 | Cldn1 | claudin 1 |
| AI138048 | Dscr1l1 | Down syndrome critical region gene 1-like 1 |
| AI146237 | — | Transcribed locus |
| AI168953 | Sult1c2a | sulfotransferase family, cytosolic, 1C, member 2a |
| AI169331 | Gstm2 | glutathione S-transferase, mu 2 |
| AI170076 | RGD1562829_predicted | similar to RAS-like, estrogen-regulated, growth-inhibitor |
| | | (predicted) |
| AI171727 | Nnmt_predicted | nicotinamide N-methyltransferase (predicted) |
| AI176041 | Pir | Pirin |
| AI176172 | — | Transcribed locus |
| AI176481 | LOC684841 | similar to CG31613-PA |
| AI176583 | Mrvldc1 | MARVEL (membrane-associating) domain containing 1 |
| AI177143 | — | Transcribed locus |
| AI177358 | Slc25a25 | solute carrier family 25 (mitochondrial carrier, phosphate |
| | | carrier), member 25 |
| AI178784 | RGD1310174_predicted | hypothetical LOC298504 (predicted) |
| AI179334 | Fasn | fatty acid synthase |
| AI179886 | RGD1310352 | similar to HTGN29 protein; keratinocytes associated |
| | | transmembrane protein 2 |
| AI180253 | RGD1563825_predicted | similar to ENSANGP00000020885 (predicted) |
| AI180398 | — | Transcribed locus |
| AI228307 | Mlc1_predicted | megalencephalic leukoencephalopathy with subcortical cysts |
| | | 1 homolog (human) (predicted) |
| AI229240 | — | Transcribed locus |
| AI230709 | Igf2bp3 | insulin-like growth factor 2, binding protein 3 |
| AI231218 | — | Transcribed locus |
| AI231999 | LOC689256 | similar to Tumor protein D53 (mD53) (Tumor protein D52- |
| | | like 1) |
| AI232036 | Atp1b1 | ATPase, Na+/K+ transporting, beta 1 polypeptide |
| AI232328 | Hgd | homogentisate 1,2-dioxygenase |
| AI232414 | Pxmp4 | peroxisomal membrane protein 4 |
| AI232716 | LOC368066 | similar to indolethylamine N-methyltransferase |
| AI232723 | — | — |
| AI232806 | — | Transcribed locus |
| AI233769 | — | Transcribed locus |
| AI234919 | Eprs | glutamyl-prolyl-tRNA synthetase |
| AI235527 | — | Transcribed locus |
| AI236188 | — | Transcribed locus |
| AI237079 | — | Transcribed locus |
| AI237143 | Rexo4 | REX4, RNA exonuclease 4 homolog (S. cerevisiae) |
| AI385229 | — | — |
| AI406386 | Lmo4 | LIM domain only 4 |
| AI406660 | — | — |
| AI406662 | Sec63_predicted | SEC63-like (S. cerevisiae) (predicted) |
| AI407074 | LOC690634 | similar to F-box only protein 3 isoform 1 |
| AI407487 | — | Transcribed locus |
| AI408556 | Fdx1 | Ferredoxin 1 |
| AI408557 | — | Transcribed locus |
| AI408948 | Ca2 | carbonic anhydrase 2 |
| AI409048 | — | Transcribed locus |
| AI410221 | Sat2_predicted | spermidine/spermine N1-acetyl transferase 2 (predicted) |
| AI410749 | — | Transcribed locus |
| AI411141 | — | — |
| AI411227 | RGD1563485_predicted | similar to mKIAA0534 protein (predicted) |
| AI411294 | — | Transcribed locus |
| AI411345 | LOC680409 /// | similar to Proline oxidase, mitochondrial precursor (Proline |
| | LOC682565 | dehydrogenase) |
| AI411693 | LOC299458 /// | similar to immunoglobulin heavy chain 6 (Igh-6) /// similar |
| | LOC366747 /// | to Ig H-chain V-region precursor /// similar to Ig heavy chain |
| | LOC678701 /// | V region MC101 precursor /// hypothetical protein |
| | RGD1359202 | LOC678701 |
| AI411947 | Igh-1a | immunoglobulin heavy chain 1a (serum IgG2a) |
| AI412161 | — | Transcribed locus |
| AI412189 | Igha_mapped /// | immunoglobulin heavy chain (alpha polypeptide) (mapped) |
| | LOC366772 | /// similar to immunoglobulin heavy chain |
| AI454611 | Fmo5 | flavin containing monooxygenase 5 |
| AI500781 | — | Transcribed locus |
| AI535089 | Zfp131 | zinc finger protein 131 |
| AI547468 | — | Transcribed locus |
| AI547628 | LOC367485 /// | similar to spermatogenesis associated glutamate (E)-rich |
| | LOC501235 /// | protein 4d /// similar to RIKEN cDNA 5031410I06 |
| | LOC501339 /// | |
| | LOC688687 /// | |
| | LOC689870 /// | |
| | LOC690439 /// | |
| | LOC690577 /// | |
| | LOC691712 | |
| AI548117 | — | — |
| AI548708 | — | Transcribed locus, strongly similar to XP_696468.1 |
| | | PREDICTED: similar to related RAS viral (r-ras) oncogene |
| | | homolog 2 (predicted) [Danio rerio] |
| AI555855 | — | Transcribed locus |
| AI556333 | RGD1564257_predicted | similar to hypothetical protein FLJ32825 (predicted) |
| AI556941 | Slc39a4_predicted | solute carrier family 39 (zinc transporter), member 4 |
| | | (predicted) |
| AI575616 | — | Transcribed locus |
| AI577508 | LOC684425 | similar to Adenylosuccinate synthetase isozyme 1 |
| | | (Adenylosuccinate synthetase, muscle isozyme) (IMP-- |
| | | aspartate ligase 1) (AdSS 1) (AMPSase 1) |
| AI598315 | Slc39a10_predicted | solute carrier family 39 (zinc transporter), member 10 |
| | | (predicted) |
| AI599423 | Gadd45g | growth arrest and DNA-damage-inducible 45 gamma |
| AI599463 | Dguok_predicted | deoxyguanosine kinase (predicted) |
| AI600057 | Crim1_predicted | cysteine-rich motor neuron 1 (predicted) |
| AI603217 | Gnb5 | guanine nucleotide binding protein, beta 5 |
| AI639108 | — | — |
| AI639241 | LOC684318 /// | ubiquitin specific protease 12 (predicted) /// similar to |
| | RGD1561481_predicted | ubiquitin specific protease 12 (predicted) /// similar to |
| | /// Usp12_predicted | ubiquitin specific protease 12 |
| AI639457 | — | Transcribed locus |
| AI703807 | Crim1_predicted | cysteine-rich motor neuron 1 (predicted) |
| AI715156 | — | Transcribed locus |
| AI715202 | RT1-Bb | RT1 class II, locus Bb |
| AI715484 | — | Transcribed locus |
| AI716248 | RGD1311155 | similar to RIKEN cDNA 9230117N10 |
| AI716500 | — | Transcribed locus |
| AJ243338 | RT1-CE5 | RT1 class I, CE5 |
| AJ249701 | RT1-A2 /// RT1-A3 /// | RT1 class Ib, locus Aw2 /// RT1 class Ia, locus A2 /// RT1 |
| | RT1-Aw2 | class I, A3 |
| AW143798 | Ccnd1 | Cyclin D1 |
| AW144193 | — | Transcribed locus |
| AW251313 | RGD1559797_predicted | similar to CDNA sequence BC014699 (predicted) |
| AW251647 | — | Transcribed locus |
| AW252232 | — | Transcribed locus |
| AW434782 | RGD1305020_predicted | similar to Hepatocellular carcinoma-associated antigen 58 |
| | | homolog (predicted) |
| AW435376 | Lrp5_predicted | low density lipoprotein receptor-related protein 5 (predicted) |
| AW521621 | — | Transcribed locus |
| AW522341 | — | Transcribed locus |
| AW524532 | — | Transcribed locus |
| AW525342 | Usp1 | ubiquitin specific peptdiase 1 |
| AW525662 | Cdc27 | cell division cycle 27 homolog (S. cerevisiae) |
| AW526014 | — | Transcribed locus |
| AW526910 | — | Transcribed locus |
| AW527403 | Sh3yl1_predicted | Sh3 domain YSC-like 1 (predicted) |
| AW528213 | Gdpd1_predicted | glycerophosphodiester phosphodiesterase domain containing |
| | | 1 (predicted) |
| AW530264 | — | Transcribed locus |
| AW530361 | Ppp1r3c | protein phosphatase 1, regulatory (inhibitor) subunit 3C |
| AW530647 | Slc25a30 | solute carrier family 25, member 30 |
| AW533234 | LOC688915 | similar to cardiomyopathy associated 5 |
| AW533569 | Clca2_predicted | chloride channel, calcium activated, family member 2 |
| | | (predicted) |
| AW533965 | Nars | Asparaginyl-tRNA synthetase |
| AW916358 | Sptlc1_predicted | serine palmitoyltransferase, long chain base subunit 1 |
| | | (predicted) |
| AW916957 | — | Transcribed locus, strongly similar to XP_579758.1 |
| | | PREDICTED: hypothetical protein XP_5779758 [Rattus |
| | | norvegicus] |
| AW920026 | LOC364393 /// | PHD finger protein 11 /// similar to RIKEN cDNA |
| | LOC684778 /// Phfl1 | 4933417L10 /// hypothetical protein LOC684778 |
| AW920487 | Casrl1 /// LOC365807 | calcium-sensing receptor like 1 /// similar to putative |
| | | pheromone receptor V2R2 |
| AY044251 | Il13ra1 | interleukin 13 receptor, alpha 1 |
| AY082609 | Abcb1a /// Abcb1b | ATP-binding cassette, sub-family B (MDR/TAP), member |
| | | 1B /// ATP-binding cassette, sub-family B (MDR/TAP), |
| | | member 1A |
| BE096457 | — | Transcribed locus |
| BE097095 | — | Transcribed locus |
| BE097185 | — | Transcribed locus |
| BE097702 | — | — |
| BE097947 | — | Transcribed locus |
| BE098143 | — | Transcribed locus |
| BE098160 | — | Transcribed locus, weakly similar to XP_001158686.1 |
| | | PREDICTED: SA hypertension-associated homolog isoform |
| | | 3 [Pan troglodytes] |
| BE098827 | — | Transcribed locus |
| BE100625 | Emr1 | EGF-like module containing, mucin-like, hormone receptor- |
| | | like sequence 1 |
| BE101298 | — | Transcribed locus |
| BE102861 | — | Transcribed locus |
| BE104060 | Igfbp5 | insulin-like growth factor binding protein 5 |
| BE104552 | Elavl1_predicted | ELAV (embryonic lethal, abnormal vision, Drosophila)-like |
| | | 1 (Hu antigen R) (predicted) |
| BE104961 | Cenpj_predicted | centromere protein J (predicted) |
| BE105916 | — | — |
| BE106199 | Rora_predicted | RAR-related orphan receptor alpha (predicted) |
| BE106376 | LOC687219 /// | similar to zinc finger protein 84 (HPF2) |
| | LOC691170 | |
| BE106921 | — | Transcribed locus |
| BE107075 | — | Transcribed locus, strongly similar to NP_056335.1 |
| | | membrane associated guanylate kinase, WW and PDZ |
| | | domain containing 1 isoform a [Homo sapiens] |
| BE107313 | RGD1310271_predicted | similar to hypothetical protein MGC45873 (predicted) |
| BE107450 | Nrep | Neuronal regeneration related protein |
| BE108158 | Unc5d_predicted | unc-5 homolog D (C. elegans) (predicted) |
| BE108176 | — | — |
| BE108354 | — | Transcribed locus |
| BE108376 | — | Transcribed locus, strongly similar to NP_795961.2 |
| | | hypothetical protein LOC319719 [Mus musculus] |
| BE108648 | — | Transcribed locus |
| BE108745 | LOC680182 /// | nucleosome binding protein 1 (predicted) /// similar to |
| | LOC681284 /// | Nucleosome binding protein 1 (Nucleosome binding protein |
| | Nsbp1_predicted | 45) (NBP-45) (GARP45 protein) |
| BE109520 | LOC619558 | hypothetical protein LOC619558 |
| BE109875 | — | Transcribed locus |
| BE110171 | LOC368158 /// | similar to Nuclear receptor ROR-gamma (Nuclear receptor |
| | RGD1562025_predicted | RZR-gamma) (Thymus orphan receptor) (TOR) /// similar to |
| | | leucine rich repeat neuronal 6D (predicted) |
| BE110921 | — | Transcribed locus |
| BE111310 | Ky_predicted | kyphoscoliosis (predicted) |
| BE113268 | Pkp2 | plakophilin 2 |
| BE113419 | Srpk2_predicted | serine/arginine-rich protein specific kinase 2 (predicted) |
| BE114005 | RGD1564024_predicted | similar to KIAA1913 (predicted) |
| BE114778 | Cgn_predicted | cingulin (predicted) |
| BE115309 | Gldc_predicted | glycine decarboxylase (predicted) |
| BE115481 | Cbfa2t3_predicted | core-binding factor, runt domain, alpha subunit 2; |
| | | translocated to, 3 (predicted) |
| BE115496 | — | Transcribed locus |
| BE115823 | — | — |
| BE116152 | Elovl6 | ELOVL family member 6, elongation of long chain fatty |
| | | acids (yeast) |
| BE116364 | Bmp5_predicted | bone morphogenetic protein 5 (predicted) |
| BE116408 | — | Transcribed locus |
| BE118653 | — | Transcribed locus |
| BE119164 | — | — |
| BE120352 | — | Transcribed locus |
| BE120522 | — | Transcribed locus |
| BE120930 | — | Transcribed locus |
| BE121026 | — | Transcribed locus |
| BE121383 | Ncam2 | neural cell adhesion molecule 2 |
| BE121400 | LOC681284 | similar to Nucleosome binding protein 1 (Nucleosome |
| | | binding protein 45) (NBP-45) (GARP45 protein) |
| BE329244 | — | — |
| BE349658 | — | — |
| BE349669 | Cav2 | caveolin 2 |
| BE349670 | — | Transcribed locus |
| BE349751 | — | — |
| BF282112 | — | Transcribed locus |
| BF282228 | Ltb | lymphotoxin B |
| BF282495 | Enpp6_predicted | Ectonucleotide pyrophosphatase/phosphodiesterase 6 |
| | | (predicted) |
| BF282636 | RGD1305457 | similar to RIKEN cDNA 1700023M03 |
| BF282722 | — | Transcribed locus |
| BF283070 | Cyp7b1 | cytochrome P450, family 7, subfamily b, polypeptide 1 |
| BF283779 | RGD1565496_predicted | similar to Butyrate-induced transcript 1 (predicted) |
| BF284175 | Pla2g12a_predicted | phospholipase A2, group XIIA (predicted) |
| BF284288 | — | Transcribed locus |
| BF284700 | — | Transcribed locus |
| BF287122 | — | Transcribed locus |
| BF287593 | Coch_predicted | coagulation factor C homolog (Limulus polyphemus) |
| | | (predicted) |
| BF288218 | RGD1309007_predicted | similar to RIKEN cDNA 2610034E18 gene (predicted) |
| BF288295 | — | Transcribed locus |
| BF288361 | — | — |
| BF289154 | — | Transcribed locus |
| BF290890 | RGD1310769_predicted | similar to HSPC288 (predicted) |
| BF291026 | — | Transcribed locus, strongly similar to XP_577836.1 |
| | | PREDICTED: similar to RIKEN cDNA 4933424N09 |
| | | [Rattus norvegicus] |
| BF386242 | Paqr9_predicted | progestin and adipoQ receptor family member IX (predicted) |
| BF386770 | — | Transcribed locus |
| BF386852 | Abca8b_predicted | ATP-binding cassette, sub-family A (ABC1), member 8b |
| | | (predicted) |
| BF389489 | Tbc1d12_predicted | TBC1D12: TBC1 domain family, member 12 (predicted) |
| BF390510 | RGD1311155 | similar to RIKEN cDNA 9230117N10 |
| BF391129 | — | Transcribed locus |
| BF391308 | — | Transcribed locus |
| BF391556 | RGD1310710_predicted | similar to RIKEN cDNA 2700091N06 (predicted) |
| BF391820 | — | Transcribed locus |
| BF392285 | — | Transcribed locus |
| BF392577 | Akap8l | A kinase (PRKA) anchor protein 8-like |
| BF393056 | — | Transcribed locus |
| BF393945 | — | Transcribed locus |
| BF394158 | — | Transcribed locus |
| BF394166 | Gpm6a | glycoprotein m6a |
| BF394600 | — | Transcribed locus |
| BF395791 | — | Transcribed locus |
| BF396135 | Stard4_predicted | StAR-related lipid transfer (START) domain containing 4 |
| | | (predicted) |
| BF396622 | — | Transcribed locus, strongly similar to XP_982134.1 |
| | | PREDICTED: similar to SET binding factor 2 [Mus |
| | | musculus] |
| BF396857 | Elovl6 | ELOVL family member 6, elongation of long chain fatty |
| | | acids (yeast) |
| BF397095 | LOC681234 | similar to debranching enzyme homolog 1 |
| BF397926 | Gmps | guanine monphosphate synthetase |
| BF398091 | — | CDNA clone IMAGE: 7374368 |
| BF398168 | — | Transcribed locus |
| BF398558 | — | Transcribed locus |
| BF400594 | Odz2 | Odd Oz/ten-m homolog 2 (Drosophila) |
| BF400669 | — | Transcribed locus |
| BF401106 | — | Transcribed locus |
| BF401529 | — | Transcribed locus |
| BF401705 | — | Transcribed locus |
| BF403483 | — | Transcribed locus, moderately similar to NP_997413.2 |
| | | oxysterol binding protein-like 1 [Mus musculus] |
| BF403998 | Cib2 | calcium and integrin binding family member 2 |
| BF404957 | — | Transcribed locus |
| BF406487 | LOC684448 /// | similar to transmembrane 6 superfamily member 2 |
| | LOC689029 | |
| BF408099 | Pcdh18_predicted | protocadherin 18 (predicted) |
| BF408445 | Gpx7_predicted | glutathione peroxidase 7 (predicted) |
| BF411794 | — | Transcribed locus |
| BF412036 | — | Transcribed locus |
| BF413105 | — | Transcribed locus |
| BF413433 | Mll | myeloid/lymphoid or mixed-lineage leukemia |
| BF414160 | Igf2bp3 | Insulin-like growth factor 2, binding protein 3 |
| BF414655 | Maoa | Monoamine oxidase A |
| BF414677 | Znf213_predicted | zinc finger protein 213 (predicted) |
| BF414998 | RGD1306105_predicted | similar to RIKEN cDNA 2610318G18 (predicted) |
| BF416266 | RGD1309948 | similar to CG11737-PA |
| BF416465 | — | Transcribed locus |
| BF417289 | — | Transcribed locus |
| BF417649 | — | — |
| BF418138 | — | Transcribed locus |
| BF419663 | — | Transcribed locus |
| BF420260 | — | — |
| BF420465 | Kif16b_predicted | kinesin family member 16B (predicted) |
| BF543574 | Irx1_predicted /// | Iroquois related homeobox 1 (Drosophila) (predicted) /// |
| | LOC501463 | similar to Iroquois-class homeodomain protein IRX-1 |
| | | (Iroquois homeobox protein 1) (Homeodomain protein |
| | | IRXA1) |
| BF545930 | — | Transcribed locus |
| BF549748 | LOC498796 | hypothetical protein LOC498796 |
| BF553076 | — | Transcribed locus |
| BF554138 | RGD1563179_predicted | similar to BAZF (predicted) |
| BF555448 | Mte1 | Mitochondrial acyl-CoA thioesterase 1 |
| BF555947 | — | Transcribed locus |
| BF557891 | Irf6_predicted | interferon regulatory factor 6 (predicted) |
| BF563411 | — | Transcribed locus |
| BF565675 | Arsa | arylsulfatase A |
| BG371721 | — | Transcribed locus |
| BG372602 | RGD1306601_predicted | similar to cDNA sequence BC019755 (predicted) |
| BG372643 | RGD1308967_predicted | similar to hypothetical protein MGC16491 (predicted) |
| BG374683 | — | Ig active lambda2-like chain mRNA, 3′ end |
| BG375362 | Ltbp4 | latent transforming growth factor beta binding protein 4 |
| BG375383 | RGD1308116_predicted | similar to hypothetical protein MGC42105 (predicted) |
| BG376410 | Tacstd1 | tumor-associated calcium signal transducer 1 |
| BG376813 | — | — |
| BG377595 | LOC367746 | similar to Spindlin-like protein 2 (SPIN-2) |
| BG377996 | LOC680248 /// | similar to Diphosphoinositol polyphosphate |
| | LOC683238 | phosphohydrolase 3 alpha (DIPP-3 alpha) (DIPP3 alpha) |
| | | (Diadenosine 5,5-P1,P6-hexaphosphate hydrolase 3 alpha) |
| | | (Nucleoside diphosphate-linked moiety X motif 10) (Nudix |
| | | motif 10) |
| BG378056 | — | Transcribed locus |
| BG378637 | Hrasls3 | HRAS like suppressor 3 |
| BG379394 | — | Transcribed locus |
| BG380581 | — | Transcribed locus |
| BG380860 | — | Transcribed locus |
| BG381311 | Slc13a5 | solute carrier family 13 (sodium-dependent citrate |
| | | transporter), member 5 |
| BG662490 | Abhd3_predicted | abhydrolase domain containing 3 (predicted) |
| BG662519 | Ank | Progressive ankylosis homolog (mouse) |
| BG664011 | Pxmp4 | peroxisomal membrane protein 4 |
| BG664221 | Ogn_predicted | osteoglycin (predicted) |
| BG666635 | — | Transcribed locus |
| BG666735 | Elovl2_predicted /// | elongation of very long chain fatty acids (FEN1/Elo2, |
| | LOC682492 | SUR4/Elo3, yeast)-like 2 (predicted) /// similar to Elongation |
| | | of very long chain fatty acids protein 2 |
| BG669396 | LOC680201 /// | similar to zinc finger protein 322a |
| | LOC684943 | |
| BG670960 | MGC95152 | similar to B230212L03Rik protein |
| BG673602 | — | — |
| BI273908 | — | Transcribed locus |
| BI274408 | Dscr1l1 | Down syndrome critical region gene 1-like 1 |
| BI275030 | RGD1308694_predicted | similar to hypothetical protein MGC47256 (predicted) |
| BI275467 | — | — |
| BI275560 | — | Transcribed locus |
| BI275815 | Tmem106a | transmembrane protein 106A |
| BI275894 | — | Transcribed locus |
| BI276183 | — | Transcribed locus |
| BI277482 | MGC112775 | similar to map kinase interacting kinase |
| BI277527 | Bckdk | branched chain ketoacid dehydrogenase kinase |
| BI277600 | LOC362972 | similar to adiponutrin |
| BI278180 | — | Transcribed locus |
| BI279030 | — | Transcribed locus |
| BI279202 | RGD1565709_predicted | similar to ovostatin-2 (predicted) |
| BI279384 | — | Transcribed locus |
| BI281668 | — | Transcribed locus |
| BI281738 | Tspyl4 | TSPY-like 4 |
| BI282024 | LOC690845 | similar to 40S ribosomal protein S4, X isoform |
| BI282114 | — | Endogenous retrovirus mRNA, partial sequence |
| BI282211 | Acsm3 | acyl-CoA synthetase medium-chain family member 3 |
| BI282488 | Acaa2 | Acetyl-Coenzyme A acyltransferase 2 (mitochondrial 3- |
| | | oxoacyl-Coenzyme A thiolase) |
| BI283159 | Scpep1 | serine carboxypeptidase 1 |
| BI283223 | RGD1562168_predicted | similar to retinoid binding protein 7 (predicted) |
| BI283882 | Gpi | glucose phosphate isomerase |
| BI284363 | Vcpip1 | valosin containing protein (p97)/p47 complex interacting |
| | | protein 1 |
| BI285494 | Ifitm3 | interferon induced transmembrane protein 3 |
| BI285576 | Gsn | Gelsolin |
| BI285616 | Adfp | adipose differentiation related protein |
| BI285941 | — | Transcribed locus |
| BI288517 | Hnf4a | hepatocyte nuclear factor 4, alpha |
| BI288540 | Plxnb1_predicted | plexin B1 (predicted) |
| BI288580 | rCG_34031 | similar to kynurenine formamidase |
| BI288945 | RGD1305713 | similar to RIKEN cDNA 3110040N11 |
| BI289032 | — | — |
| BI289085 | Vnn1 | vanin 1 |
| BI289506 | — | Transcribed locus |
| BI289620 | Crim1_predicted | cysteine-rich motor neuron 1 (predicted) |
| BI289723 | LOC310721 | similar to 4930431B09Rik protein |
| BI290154 | RGD1309578 | similar to Aa2-174 |
| BI290769 | RGD1309634_predicted | hypothetical LOC305452 (predicted) |
| BI290794 | — | Transcribed locus, strongly similar to XP_347073.1 |
| | | PREDICTED: hypothetical protein XP_347072 [Rattus |
| | | norvegicus] |
| BI291842 | — | Transcribed locus |
| BI292055 | — | Transcribed locus |
| BI292056 | — | Transcribed locus |
| BI293584 | Tmem55a | transmembrane protein 55A |
| BI295569 | RGD1564089_predicted | similar to peroxisomal long chain acyl-CoA thioesterase Ia |
| | | (predicted) |
| BI295739 | — | Transcribed locus, moderately similar to NP_005132.2 |
| | | fibrinogen, beta chain preproprotein [Homo sapiens] |
| BI295961 | Tjp3_predicted | tight junction protein 3 (predicted) |
| BI296153 | Acaca | acetyl-coenzyme A carboxylase alpha |
| BI296275 | Mmd2_predicted | monocyte to macrophage differentiation-associated 2 |
| | | (predicted) |
| BI296384 | — | Transcribed locus |
| BI296388 | — | Transcribed locus, strongly similar to XP_574380.1 |
| | | PREDICTED: poliovirus receptor-related 2 (herpesvirus |
| | | entry mediator B) (predicted) [Rattus norvegicus] |
| BI296463 | Tpcn2_predicted | two pore segment channel 2 (predicted) |
| BI296494 | — | Transcribed locus |
| BI296524 | — | Transcribed locus |
| BI297291 | — | Transcribed locus |
| BI300997 | Sult1c2 /// Sult1c2a | sulfotransferase family, cytosolic, 1C, member 2 /// |
| | | sulfotransferase family, cytosolic, 1C, member 2a |
| BI301687 | — | Transcribed locus |
| BI395849 | — | — |
| BM382879 | Pura_predicted | Purine rich element binding protein A (predicted) |
| BM383209 | RGD1561153_predicted | similar to myosin-VIIb (predicted) |
| BM383212 | — | Transcribed locus |
| BM383531 | LOC682651 /// | similar to Metallothionein-2 (MT-2) (Metallothionein-II) |
| | LOC689415 | (MT-II) |
| BM383683 | RGD1310710_predicted | similar to RIKEN cDNA 2700091N06 (predicted) |
| BM384088 | — | Transcribed locus |
| BM384752 | Kdelc2 | KDEL (Lys-Asp-Glu-Leu) containing 2 |
| BM384823 | — | Transcribed locus |
| BM385772 | — | Transcribed locus |
| BM386280 | — | — |
| BM386775 | Aim1l_predicted | absent in melanoma 1-like (predicted) |
| BM387172 | — | Transcribed locus, strongly similar to XP_233470.2 |
| | | PREDICTED: similar to 6330530A05Rik protein [Rattus |
| | | norvegicus] |
| BM387283 | Trim14_predicted | tripartite motif protein 14 (predicted) |
| BM387449 | — | Transcribed locus |
| BM387813 | — | Transcribed locus |
| BM388141 | — | — |
| BM388719 | Rpl7 | Ribosomal protein L7 |
| BM389786 | — | Transcribed locus |
| BM390238 | — | Transcribed locus |
| BM390378 | RGD1562732_predicted | similar to Glutathione S-transferase, theta 3 (predicted) |
| BM390571 | Sult2b1_predicted | sulfotransferase family, cytosolic, 2B, member 1 (predicted) |
| BM391206 | Hist1h2bh | histone cluster 1, H2bh |
| BM391471 | Atf5 | activating transcription factor 5 |
| BM391823 | RGD1563309_predicted | similar to diacylglycerol kinase, delta 130 kDa isoform 1 |
| | | (predicted) |
| BM986536 | Hist1h4b | histone cluster 1, H4b |
| D00252 | Got1 | glutamate oxaloacetate transaminase 1 |
| H33577 | — | Transcribed locus |
| H33845 | — | Transcribed locus |
| H35736 | Slc25a30 | Solute carrier family 25, member 30 |
| J02585 | Scd1 | stearoyl-Coenzyme A desaturase 1 |
| J05499 | Gls2 | glutaminase 2 (liver, mitochondrial) |
| K02422 | Cyp1a2 | cytochrome P450, family 1, subfamily a, polypeptide 2 |
| L06804 | Lhx2_predicted | LIM homeobox protein 2 (predicted) |
| L12458 | Lyz | Lysozyme |
| L19180 | Ptprd | protein tyrosine phosphatase, receptor type, D |
| L19181 | Ptprd | protein tyrosine phosphatase, receptor type, D |
| L19933 | Ptprd | protein tyrosine phosphatase, receptor type, D |
| L22655 | LOC500180 | similar to IG KAPPA CHAIN V-V REGION K2 |
| | | PRECURSOR |
| L26525 | — | — |
| L46791 | Ces3 /// LOC291863 | carboxylesterase 3 /// carboxylesterase-like |
| L48060 | Prlr | prolactin receptor |
| M10094 | RT1-Aw2 | RT1 class Ib, locus Aw2 |
| M13506 | Udpgtr2 | liver UDP-glucuronosyltransferase, phenobarbital-inducible |
| | | form |
| M14137 | Atp1b1 | ATPase, Na+/K+ transporting, beta 1 polypeptide |
| M17685 | Pklr | pyruvate kinase, liver and red blood cell |
| M18340 | Tat | tyrosine aminotransferase |
| M24024 | RT1-Aw2 | RT1 class Ib, locus Aw2 |
| M24327 | Mt1a | metallothionein 1a |
| M25804 | Nr1d1 | nuclear receptor subfamily 1, group D, member 1 |
| M27882 | Spink1 | serine protease inhibitor, Kazal type 1 |
| M27883 | Spink3 | serine protease inhibitor, Kazal type 3 |
| M30596 | Me1 | malic enzyme 1 |
| M57668 | Prlr | prolactin receptor |
| M63991 | Serpina7 | serine (or cysteine) peptidase inhibitor, clade A (alpha-1 |
| | | antipeptidase, antitrypsin), member 7 |
| M64755 | Csad | cysteine sulfinic acid decarboxylase |
| NM_012522 | Cbs | cystathionine beta synthase |
| NM_012524 | Cebpa | CCAAT/enhancer binding protein (C/EBP), alpha |
| NM_012531 | Comt | catechol-O-methyltransferase |
| NM_012552 | Ela1 | elastase 1, pancreatic |
| NM_012600 | Me1 | malic enzyme 1 |
| NM_012603 | Myc | Myelocytomatosis viral oncogene homolog (avian) |
| NM_012624 | Pklr | pyruvate kinase, liver and red blood cell |
| NM_012645 | RT1-Aw2 | RT1 class Ib, locus Aw2 |
| NM_012646 | RT1-N1 /// RT1-N2 /// | RT1 class Ib gene, H2-TL-like, grc region (N1) /// RT1 class |
| | RT1-N3 | Ib gene, H2-TL-like, grc region (N3) /// RT1 class Ib gene, |
| | | H2-TL-like, grc region(N2) |
| NM_012674 | Spink1 | serine protease inhibitor, Kazal type 1 |
| NM_012695 | Smp2a | rat senescence marker protein 2A gene, exons 1 and 2 |
| NM_012737 | Apoa4 | apolipoprotein A-IV |
| NM_012742 | Foxa1 | forkhead box A1 |
| NM_012744 | Pc | Pyruvate carboxylase |
| NM_012753 | Cyp17a1 | cytochrome P450, family 17, subfamily a, polypeptide 1 |
| NM_012798 | Mal | myelin and lymphocyte protein, T-cell differentiation protein |
| NM_012816 | Amacr | alpha-methylacyl-CoA racemase |
| NM_012907 | Apobec1 | apolipoprotein B editing complex 1 |
| NM_013027 | Sepw1 | selenoprotein W, muscle 1 |
| NM_013068 | Fabp2 | fatty acid binding protein 2, intestinal |
| NM_013084 | Acadsb | acyl-Coenzyme A dehydrogenase, short/branched chain |
| NM_013095 | Smad3 | MAD homolog 3 (Drosophila) |
| NM_013144 | Igfbp1 | insulin-like growth factor binding protein 1 |
| NM_016987 | Acly | ATP citrate lyase |
| NM_017019 | Il1a | interleukin 1 alpha |
| NM_017025 | Ldha | lactate dehydrogenase A |
| NM_017077 | Foxa3 | forkhead box A3 |
| NM_017106 | Clcn5 | chloride channel 5 |
| NM_017111 | Slco1a1 | solute carrier organic anion transporter family, member 1a1 |
| NM_017125 | Cd63 | CD63 antigen |
| NM_017128 | Inhba | inhibin beta-A |
| NM_017149 | Meox2 | mesenchyme homeobox 2 |
| NM_017159 | Hal | histidine ammonia lyase |
| NM_017161 | Adora2b | adenosine A2B receptor |
| NM_017206 | Slc6a6 | solute carrier family 6 (neurotransmitter transporter, taurine), |
| | | member 6 |
| NM_017229 | Pde3b | phosphodiesterase 3B |
| NM_017235 | Hsd17b7 | hydroxysteroid (17-beta) dehydrogenase 7 |
| NM_017245 | Eef2 | eukaryotic translation elongation factor 2 |
| NM_017332 | Fasn | fatty acid synthase |
| NM_017357 | Hpcal4 | hippocalcin-like 4 |
| NM_019203 | Tsx | testis specific X-linked gene |
| NM_019216 | Gdf15 | growth differentiation factor 15 |
| NM_019291 | Ca2 | carbonic anhydrase 2 |
| NM_019292 | Ca3 | carbonic anhydrase 3 |
| NM_019314 | Kcnn2 | potassium intermediate/small conductance calcium-activated |
| | | channel, subfamily N, member 2 |
| NM_019323 | Mcpt10 /// Mcpt8 /// | mast cell protease 8 /// mast cell protease 10 /// mast cell |
| | Mcpt8l2 /// Mcpt9 | protease 9 /// mast cell protease 8-like 2 |
| NM_020072 | Acpp | acid phosphatase, prostate |
| NM_020540 | Gstm4 | glutathione S-transferase M4 |
| NM_021577 | Asl | Argininosuccinate lyase |
| NM_021653 | Dio1 | deiodinase, iodothyronine, type I |
| NM_021664 | Dnase2b | deoxyribonuclease II beta |
| NM_021744 | Cd14 | CD14 antigen |
| NM_022224 | Pter | phosphotriesterase related |
| NM_022280 | Lrat | lecithin-retinol acyltransferase (phosphatidylcholine-retinol- |
| | | O-acyltransferase) |
| NM_022407 | Aldh1a1 | aldehyde dehydrogenase family 1, member A1 |
| NM_022521 | Oat | ornithine aminotransferase |
| NM_022542 | Rhob | ras homolog gene family, member B |
| NM_022617 | Mpeg1 | macrophage expressed gene 1 |
| NM_022686 | LOC680097 /// | similar to germinal histone H4 gene |
| | LOC684887 | |
| NM_023025 | Cyp2j3 /// Cyp2j4 | cytochrome P450, family 2, subfamily J, polypeptide 4 /// |
| | | cytochrome P450, family 2, subfamily j, polypeptide 3 |
| NM_023951 | Og9x | OG9 homeobox gene |
| NM_023969 | Edg7 | putative G protein-coupled receptor snGPCR32 |
| NM_024127 | Gadd45a | growth arrest and DNA-damage-inducible 45 alpha |
| NM_024147 | Evl | Ena-vasodilator stimulated phosphoprotein |
| NM_024381 | Gyk | glycerol kinase |
| NM_030994 | Itga1 | integrin alpha 1 |
| NM_031039 | Gpt1 | glutamic pyruvic transaminase 1, soluble |
| NM_031073 | Ntf3 | neurotrophin 3 |
| NM_031074 | Nup98 | nucleoporin 98 |
| NM_031154 | Gstm3 | glutathione S-transferase, mu type 3 |
| NM_031330 | Asl | Argininosuccinate lyase |
| NM_031533 | Ugt2b | UDP glycosyltransferase 2 family, polypeptide B |
| NM_031543 | Cyp2e1 | cytochrome P450, family 2, subfamily e, polypeptide 1 |
| NM_031548 | Scnn1a | sodium channel, nonvoltage-gated 1 alpha |
| NM_031559 | Cpt1a | carnitine palmitoyltransferase 1a, liver |
| NM_031561 | RGD1562323_predicted | similar to fatty acid translocase/CD36 (predicted) |
| NM_031605 | Cyp4a8 | cytochrome P450, family 4, subfamily a, polypeptide 8 |
| NM_031641 | Sult4a1 | sulfotransferase family 4A, member 1 |
| NM_031741 | Slc2a5 | solute carrier family 2, member 5 |
| NM_031841 | Scd2 | stearoyl-Coenzyme A desaturase 2 |
| NM_031970 | Hspb1 | heat shock 27 kDa protein 1 |
| NM_031971 | Hspa1a /// Hspa1b | heat shock 70 kD protein 1A /// heat shock 70 kD protein 1B |
| | | (mapped) |
| NM_053299 | Ubd | ubiquitin D |
| NM_053328 | Bhlhb2 | basic helix-loop-helix domain containing, class B2 |
| NM_053380 | Slc34a2 | solute carrier family 34 (sodium phosphate), member 2 |
| NM_053584 | — | — |
| NM_053587 | S100a9 | S100 calcium binding protein A9 (calgranulin B) |
| NM_053639 | Ltc4s | leukotriene C4 synthase |
| NM_053714 | Ank | progressive ankylosis homolog (mouse) |
| NM_053754 | Abcg5 | ATP-binding cassette, sub-family G (WHITE), member 5 |
| NM_053962 | Sds | serine dehydratase |
| NM_053977 | Cdh17 | cadherin 17 |
| NM_053999 | Ppp2r2a | protein phosphatase 2 (formerly 2A), regulatory subunit B |
| | | (PR 52), alpha isoform |
| NM_057133 | Nr0b2 | nuclear receptor subfamily 0, group B, member 2 |
| NM_057208 | Tpm3 | tropomyosin 3, gamma |
| NM_080773 | Chrm1 | cholinergic receptor, muscarinic 1 |
| NM_130422 | Casp12 | caspase 12 |
| NM_130430 | Psmd9 | proteasome (prosome, macropain) 26S subunit, non-ATPase, 9 |
| NM_130433 | Acaa2 | acetyl-Coenzyme A acyltransferase 2 (mitochondrial 3- |
| | | oxoacyl-Coenzyme A thiolase) |
| NM_130752 | Fgf21 | fibroblast growth factor 21 |
| NM_133413 | Cysltr2 | cysteinyl leukotriene receptor 2 |
| NM_133525 | RGD620382 | Nucleoside 2-deoxyribosyltransferase domain containing |
| | | protein RGD620382 |
| NM_133526 | Tspan8 | tetraspanin 8 |
| NM_133538 | Il13ra2 | interleukin 13 receptor, alpha 2 |
| NM_133547 | Sult1c2 /// Sult1c2a | sulfotransferase family, cytosolic, 1C, member 2 /// |
| | | sulfotransferase family, cytosolic, 1C, member 2a |
| NM_133586 | Ces2 | carboxylesterase 2 (intestine, liver) |
| NM_133614 | Slc25a21 | solute carrier family 25 (mitochondrial oxodicarboxylate |
| | | carrier), member 21 |
| NM_134372 | Acmsd | 2-amino-3-carboxymuconate-6-semialdehyde decarboxylase |
| NM_134379 | UST4r | integral membrane transport protein UST4r |
| NM_139255 | LOC246120 | RDCR-0918-3 protein |
| NM_139258 | Bmf | Bcl2 modifying factor |
| U36476 | Mmp9 | matrix metallopeptidase 9 |
| U46118 | Cyp3a9 | cytochrome P450, family 3, subfamily a, polypeptide 9 |
| U88294 | Cpt1a | carnitine palmitoyltransferase 1a, liver |
| Y09333 | Mte1 | mitochondrial acyl-CoA thioesterase 1 |
| Y09945 | Slc22a9 | solute carrier family 22 (organic anion/cation transporter), |
| | | member 9 |
| Z78279 | Col1a1 | procollagen, type 1, alpha 1 |

C. Quadricep

| | | |
|---|---|---|
| AA799328 | RGD1560913_predicted | similar to expressed sequence AW413625 (predicted) |
| AA800285 | — | Transcribed locus |
| AA800750 | — | — |
| AA818732 | — | Transcribed locus |
| AA819034 | isg12(b) | putative ISG12(b) protein |
| AA849966 | — | — |
| AA850780 | RGD1564560_predicted | similar to RCK (predicted) |
| AA866223 | — | — |
| AA875438 | RGD1309414_predicted | similar to KIAA0913 protein (predicted) |
| AA892854 | LOC498335 | similar to Small inducible cytokine B13 precursor |
| | | (CXCL13) (B lymphocyte chemoattractant) (CXC |
| | | chemokine BLC) |
| AA900536 | — | Transcribed locus |
| AA901178 | RGD1304595_predicted | similar to RIKEN cDNA 6330416G13 gene (predicted) |
| AA901290 | — | Transcribed locus |
| AA946467 | — | Transcribed locus |
| AA965076 | RGD1305179_predicted | similar to Nedd4 binding protein 1 (predicted) |
| AA998276 | — | Transcribed locus |
| AB001382 | Spp1 | secreted phosphoprotein 1 |
| AB035650 | Usf2 | upstream transcription factor 2 |
| AF065161 | Cish | cytokine inducible SH2-containing protein |
| AF198441 | Rup2 | urinary protein 2 |
| AF220558 | Trdn | triadin |
| AF368860 | LOC680367 | similar to Urinary protein 3 precursor (RUP-3) |
| AI008409 | — | CDNA clone IMAGE: 7321089 |
| AI009530 | MGC72614 | hypothetical LOC310540 |
| AI010831 | RGD1561425_predicted | similar to RIKEN cDNA 4832428D23 gene (predicted) |
| AI012630 | Smarcad1_predicted | SWI/SNF-related, matrix-associated actin-dependent |
| | | regulator of chromatin, subfamily a, containing DEAD/H |
| | | box 1′ (predicted) |
| AI013568 | RGD1311123 | Similar to 1300013J15Rik protein |
| A1029745 | Chodl_predicted | chondrolectin (predicted) |
| AI030168 | LOC367902 | similar to ALEX3 protein |
| AI030203 | Irx3_predicted | Iroquois related homeobox 3 (Drosophila) (predicted) |
| AI044273 | — | Transcribed locus |
| AI044549 | — | Transcribed locus |
| AI044784 | — | Transcribed locus |
| AI045857 | RGD1565099_predicted | similar to BTEB3 protein (predicted) |
| AI045904 | — | — |
| AI059437 | — | — |
| AI070365 | — | Transcribed locus |
| AI071674 | — | Transcribed locus |
| AI102732 | MGC109340 | similar to Microsomal signal peptidase 23 kDa subunit |
| | | (SPase 22 kDa subunit) (SPC22/23) |
| AI104783 | — | Transcribed locus |
| AI105018 | Cmya1_predicted | cardiomyopathy associated 1 (predicted) |
| AI137988 | — | Transcribed locus |
| AI145077 | — | Transcribed locus |
| AI145240 | Akap9 | A kinase (PRKA) anchor protein (yotiao) 9 |
| AI169080 | LOC690040 /// | protein tyrosine phosphatase-like (proline instead of catalytic |
| | Ptplb_predicted | arginine), member b (predicted) /// similar to protein tyrosine |
| | | phosphatase-like (proline instead of catalytic arginine), |
| | | member b |
| AI169092 | Thrsp | thyroid hormone responsive protein |
| AI169331 | Gstm2 | glutathione S-transferase, mu 2 |
| AI170377 | — | Transcribed locus |
| AI170665 | Chac1_predicted | ChaC, cation transport regulator-like 1 (E. coli) (predicted) |
| AI170690 | LOC501126 | similar to hypothetical protein MGC26733 |
| AI172110 | — | — |
| AI172311 | RGD1310022 | similar to RIKEN cDNA 2610204K14 |
| AI172334 | — | — |
| AI175045 | — | Transcribed locus |
| AI177358 | Slc25a25 | solute carrier family 25 (mitochondrial carrier, phosphate |
| | | carrier), member 25 |
| AI177934 | LOC474169 | pre-eosinophil-associated ribonuclease-2 |
| AI179334 | Fasn | fatty acid synthase |
| AI179886 | RGD1310352 | similar to HTGN29 protein; keratinocytes associated |
| | | transmembrane protein 2 |
| AI179988 | Enc1 | ectodermal-neural cortex 1 |
| AI180253 | RGD1563825_predicted | similar to ENSANGP00000020885 (predicted) |
| AI230048 | Dbp | D site albumin promoter binding protein |
| AI231999 | LOC689256 | similar to Tumor protein D53 (mD53) (Tumor protein D52- |
| | | like 1) |
| AI232414 | Pxmp4 | peroxisomal membrane protein 4 |
| AI233530 | — | — |
| AI235468 | Dst_predicted | dystonin (predicted) |
| AI236142 | Hdac10 | histone deacetylase 10 |
| AI237143 | Rexo4 | REX4, RNA exonuclease 4 homolog (S. cerevisiae) |
| AI237251 | — | — |
| AI406386 | Lmo4 | LIM domain only 4 |
| AI406684 | RGD1562672_predicted | similar to Arx homeoprotein (predicted) |
| AI406939 | G0s2 | G0/G1 switch gene 2 |
| AI407339 | Irgm | immunity-related GTPase family, M |
| AI408099 | Akr1b10 | aldo-keto reductase family 1, member B10 (aldose |
| | | reductase) |
| AI408498 | Col7a1_predicted | procollagen, type VII, alpha 1 (predicted) |
| AI412065 | Grhpr_predicted | glyoxylate reductase/hydroxypyruvate reductase (predicted) |
| AI412218 | Slc6a8 | solute carrier family 6 (neurotransmitter transporter, |
| | | creatine), member 8 |
| AI501537 | — | Transcribed locus |
| AI501693 | — | Transcribed locus |
| AI502757 | Cst7_predicted | cystatin F (leukocystatin) (predicted) |
| AI535506 | Smtnl1_predicted | smoothelin-like 1 (predicted) |
| AI548667 | — | Transcribed locus |
| AI555855 | — | Transcribed locus |
| AI574994 | — | Transcribed locus |
| AI578611 | — | Transcribed locus |
| AI579422 | Bex1 | brain expressed X-linked 1 |
| AI599463 | Dguok_predicted | deoxyguanosine kinase (predicted) |
| AI638990 | — | Transcribed locus |
| AI639318 | Ret | ret proto-oncogene |
| AI710284 | — | — |
| AI713274 | Syt7 | synaptotagmin VII |
| AI715202 | RT1-Bb | RT1 class II, locus Bb |
| AJ249701 | RT1-A2 /// RT1-A3 /// | RT1 class Ib, locus Aw2 /// RT1 class Ia, locus A2 /// RT1 |
| | RT1-Aw2 | class I, A3 |
| AW251280 | — | — |
| AW433899 | Rbm17 | RNA binding motif protein 17 |
| AW520914 | Casq2 | calsequestrin 2 |
| AW521704 | RGD1308371_predicted | hypothetical LOC293114 (predicted) |
| AW521752 | — | Transcribed locus |
| AW522341 | — | Transcribed locus |
| AW525342 | Usp1 | ubiquitin specific peptdiase 1 |
| AW527159 | — | Transcribed locus |
| AW530219 | Ryr1 | ryanodine receptor 1, skeletal muscle |
| AW530361 | Ppp1r3c | protein phosphatase 1, regulatory (inhibitor) subunit 3C |
| AW533490 | — | — |
| AW534218 | RGD1310722_predicted | similar to RIKEN cDNA D130059P03 gene (predicted) |
| AW534519 | — | Transcribed locus |
| AW535196 | RGD1310383_predicted | similar to T-cell activation protein phosphatase 2C |
| | | (predicted) |
| AW535541 | Svil_predicted | Supervillin (predicted) |
| AW535897 | — | Transcribed locus |
| AW536022 | — | Transcribed locus |
| AW918670 | Flnc_predicted | filamin C, gamma (actin binding protein 280) (predicted) |
| AW920063 | Kcne2 | Potassium voltage-gated channel, Isk-related family, |
| | | member 2 |
| BE096522 | — | — |
| BE097574 | Kcnab1 | potassium voltage-gated channel, shaker-related subfamily, |
| | | beta member 1 |
| BE097672 | LOC679974 /// | similar to transcription elongation factor A (SII)-like 3 |
| | RGD1562504_predicted | (predicted) /// similar to transcription elongation factor A |
| | | (SII)-like 3 |
| BE098532 | LOC303057 | similar to step II splicing factor SLU7; DNA segment, Chr |
| | | 11, ERATO Doi 730, expressed; DNA segment, Chr 3, |
| | | Brigham & Womens Genetics 0878 expressed |
| BE100840 | Gng11 | guanine nucleotide binding protein (G protein), gamma 11 |
| BE100915 | Tmem42_predicted | Transmembrane protein 42 (predicted) |
| BE100932 | — | Transcribed locus |
| BE102861 | — | Transcribed locus |
| BE104375 | LOC499745 | Similar to Notch-regulated ankyrin repeat protein |
| BE104457 | — | Transcribed locus |
| BE106279 | — | Transcribed locus |
| BE107587 | RGD1562949_predicted | similar to mKIAA0259 protein (predicted) |
| BE108208 | — | Transcribed locus |
| BE108716 | — | Transcribed locus |
| BE109369 | Plekhc1 | Pleckstrin homology domain containing, family C (with |
| | | FERM domain) member 1 |
| BE109675 | — | Transcribed locus |
| BE111349 | — | — |
| BE112451 | RGD1561027_predicted | similar to PACRG (predicted) |
| BE113460 | — | Transcribed locus |
| BE114634 | Mocs3_predicted | Molybdenum cofactor synthesis 3 (predicted) |
| BE115520 | — | — |
| BE116867 | — | Transcribed locus |
| BE117891 | Mll5 | myeloid/lymphoid or mixed-lineage leukemia 5 (trithorax |
| | | homolog, Drosophila) |
| BE118330 | RGD1560397_predicted | similar to RNA-binding protein Musashi2-S (predicted) |
| BE118382 | Nek9_predicted | NIMA (never in mitosis gene a)-related kinase 9 (predicted) |
| BE118557 | — | — |
| BE118639 | Tpr | translocated promoter region |
| BE126475 | Prrx1 | paired related homeobox 1 |
| BE329244 | — | — |
| BE349751 | — | — |
| BE349808 | Zdhhc9 | zinc finger, DHHC domain containing 9 |
| BF281984 | LOC360570 | similar to myosin XVIIIa |
| BF282876 | RGD1308952 | similar to mKIAA0665 protein |
| BF283384 | — | Transcribed locus |
| BF283420 | — | Transcribed locus, moderately similar to XP_001000045.1 |
| | | PREDICTED: similar to WD repeat domain 66 [Mus |
| | | musculus] |
| BF284035 | — | — |
| BF284535 | — | Transcribed locus |
| BF284876 | — | Transcribed locus |
| BF288243 | Klf2_predicted | Kruppel-like factor 2 (lung) (predicted) |
| BF288683 | Adhfe1 | alcohol dehydrogenase, iron containing, 1 |
| BF289725 | Lrpprc | leucine-rich PPR-motif containing |
| BF290088 | Nkx2-3_predicted | NK2 transcription factor related, locus 3 (Drosophila) |
| | | (predicted) |
| BF386502 | — | Transcribed locus |
| BF386877 | — | Transcribed locus |
| BF387694 | — | Transcribed locus |
| BF387767 | — | Transcribed locus |
| BF388757 | RGD1309104_predicted | similar to RIKEN cDNA 1700025G04 gene (predicted) |
| BF393261 | LOC246295 | glycine-, glutamate-, thienylcyclohexylpiperidine-binding |
| | | protein |
| BF397936 | RGD1307526 | similar to modulator of estrogen induced transcription |
| BF398031 | — | Transcribed locus |
| BF398435 | — | Transcribed locus |
| BF401902 | Rsn | restin (Reed-Steinberg cell-expressed intermediate filament- |
| | | associated protein) |
| BF404113 | — | Transcribed locus |
| BF405468 | LOC311548 | similar to RIKEN cDNA 4930509O20 |
| BF408444 | RGD1564043_predicted | Similar to RIKEN cDNA E430021N18 (predicted) |
| BF408611 | Sln | sarcolipin |
| BF409078 | — | Transcribed locus |
| BF409456 | LOC688018 | similar to SH3-domain binding protein 3 |
| BF409997 | — | Transcribed locus |
| BF410146 | Hspa2 | heat shock 70 kDa protein 2 |
| BF410555 | — | — |
| BF412297 | LOC686226 | similar to TSC22 domain family protein 4 (TSC22-related- |
| | | inducible leucine zipper protein 2) |
| BF415479 | RGD1306148_predicted | similar to KIAA0368 (predicted) |
| BF416871 | Abcb10 | ATP-binding cassette, sub-family B (MDR/TAP), member |
| | | 10 |
| BF417216 | LOC686132 | similar to hemicentin 1 |
| BF419602 | RGD1307729_predicted | similar to KIAA0853 protein (predicted) |
| BF522436 | RGD1306067 | similar to chromosome 20 open reading frame 6 |
| BF524010 | — | — |
| BF542281 | LOC681012 | similar to Est1p-like protein B |
| BF546659 | Syncrip | Synaptotagmin binding, cytoplasmic RNA interacting |
| | | protein |
| BF553211 | Eprs | glutamyl-prolyl-tRNA synthetase |
| BF559640 | — | Transcribed locus |
| BF559746 | Dyrk2_predicted | dual-specificity tyrosine-(Y)-phosphorylation regulated |
| | | kinase 2 (predicted) |
| BF561222 | — | Transcribed locus |
| BF564825 | LOC500591 | similar to calmodulin-binding transcription activator 1 |
| BF565278 | Dnaja4 | DnaJ (Hsp40) homolog, subfamily A, member 4 |
| BF566724 | RGD1305235 | similar to RIKEN cDNA 1700052N19 |
| BF568007 | LOC501069 | similar to golgi autoantigen golgin subtype a4; tGolgin-1 |
| BG371544 | — | Transcribed locus |
| BG373455 | — | Transcribed locus |
| BG375165 | — | — |
| BG375279 | RGD1565787_predicted | similar to RIKEN cDNA 1810065E05 (predicted) |
| BG375352 | Ca5b | carbonic anhydrase VB, mitochondrial |
| BG375480 | Iqub | IQ motif and ubiquitin domain containing |
| BG375603 | Asph_predicted | aspartate-beta-hydroxylase (predicted) |
| BG377396 | Slc22a12 | solute carrier family 22 (organic anion/cation transporter), |
| | | member 12 |
| BG378837 | — | Transcribed locus |
| BG380430 | RGD1564105_predicted | similar to RIKEN cDNA B130052G07 (predicted) |
| BI273836 | — | Transcribed locus |
| BI274605 | Ucp3 | uncoupling protein 3 (mitochondrial, proton carrier) |
| BI275261 | — | — |
| BI275292 | Angpt2 | angiopoietin 2 |
| BI275763 | Phyhd1 | phytanoyl-CoA dioxygenase domain containing 1 |
| BI277460 | Pck1 | phosphoenolpyruvate carboxykinase 1 |
| BI278180 | — | Transcribed locus |
| BI278550 | Npas2_predicted | neuronal PAS domain protein 2 (predicted) |
| BI278813 | Ckap4_predicted | cytoskeleton-associated protein 4 (predicted) |
| BI279347 | — | Transcribed locus |
| BI282114 | — | Endogenous retrovirus mRNA, partial sequence |
| BI282920 | Ccl21b | chemokine (C-C motif) ligand 21b (serine) |
| BI288579 | — | — |
| BI288589 | RGD1561597_predicted | similar to mKIAA0518 protein (predicted) |
| BI288619 | Jun | Jun oncogene |
| BI289559 | Sox6 | SRY-box containing gene 6 |
| BI291842 | — | Transcribed locus |
| BI294465 | — | Transcribed locus |
| BI295878 | Kat3 | kynurenine aminotransferase III |
| BI297744 | RGD1310507 | similar to RIKEN cDNA 1300017J02 |
| BI300470 | — | Transcribed locus |
| BI300794 | — | Transcribed locus |
| BI302283 | LOC690745 | MOCO sulphurase C-terminal domain containing-like |
| BI303199 | — | Transcribed locus |
| BM382843 | RGD1559885_predicted | similar to hypothetical gene supported by BC063892 |
| | | (predicted) |
| BM383683 | RGD1310710_predicted | similar to RIKEN cDNA 2700091N06 (predicted) |
| BM383757 | MGC105830 /// | similar to Ras-related protein Rab-1B /// RAB1B, member |
| | rCG_48149 | RAS oncogene family |
| BM385476 | Bst2 | bone marrow stromal cell antigen 2 |
| BM388738 | — | — |
| BM389513 | LOC688090 /// RT1-Bb | RT1 class II, locus Bb /// similar to RT1 class II |
| | | histocompatibility antigen, B-1 beta chain precursor (RT1.B- |
| | | beta(1)) |
| BM391248 | MGC108778 | similar to RIKEN cDNA 1810057C19 |
| BM391779 | — | Transcribed locus |
| H31112 | Sucla2_predicted | Succinate-Coenzyme A ligase, ADP-forming, beta subunit |
| | | (predicted) |
| H31802 | — | — |
| J00710 | Csn1s1 | casein alpha s1 |
| J02585 | Scd1 | stearoyl-Coenzyme A desaturase 1 |
| L35572 | Lhx5 | LIM homeobox protein 5 |
| L81174 | Ankrd1 | ankyrin repeat domain 1 (cardiac muscle) |
| M24024 | RT1-Aw2 | RT1 class Ib, locus Aw2 |
| M91450 | Cdx1 | caudal type homeo box 1 |
| NM_012531 | Comt | catechol-O-methyltransferase |
| NM_012594 | Lalba | lactalbumin, alpha |
| NM_012646 | RT1-N1 /// RT1-N2 /// | RT1 class Ib gene, H2-TL-like, grc region (N1) /// RT1 class |
| | RT1-N3 | Ib gene, H2-TL-like, grc region (N3) /// RT1 class Ib gene, |
| | | H2-TL-like, grc region(N2) |
| NM_012703 | Thrsp | thyroid hormone responsive protein |
| NM_012870 | Tnfrsf11b | tumor necrosis factor receptor superfamily, member 11b |
| | | (osteoprotegerin) |
| NM_013220 | Ankrd1 | ankyrin repeat domain 1 (cardiac muscle) |
| NM_017332 | Fasn | fatty acid synthase |
| NM_019161 | RGD1566401_predicted | similar to GTL2, imprinted maternally expressed |
| | | untranslated (predicted) |
| NM_019205 | Ccl11 | chemokine (C-C motif) ligand 11 |
| NM_022392 | Insig1 | insulin induced gene 1 |
| NM_022400 | Bcat2 | branched chain aminotransferase 2, mitochondrial |
| NM_022643 | Hist1h2ba | histone cluster 1, H2ba |
| NM_023025 | Cyp2j3 /// Cyp2j4 | cytochrome P450, family 2, subfamily J, polypeptide 4 /// |
| | | cytochrome P450, family 2, subfamily j, polypeptide 3 |
| NM_024131 | Ddt | D-dopachrome tautomerase |
| NM_024486 | Acvr1 | activin A receptor, type 1 |
| NM_030994 | Itga1 | integrin alpha 1 |
| NM_031327 | Cyr61 | cysteine rich protein 61 |
| NM_031598 | Pla2g2a | phospholipase A2, group IIA (platelets, synovial fluid) |
| NM_031971 | Hspa1a /// Hspa1b | heat shock 70 kD protein 1A /// heat shock 70 kD protein 1B |
| | | (mapped) |
| NM_053299 | Ubd | ubiquitiri D |
| NM_053355 | Ikbkb | inhibitor of kappaB kinase beta |
| NM_053381 | Atp1b4 | ATPase, (Na+)/K+ transporting, beta 4 polypeptide |
| NM_053551 | Pdk4 | pyruvate dehydrogenase kinase, isoenzyme 4 |
| NM_053665 | Akap1 | A kinase (PRKA) anchor protein 1 |
| NM_053750 | Nppc | natriuretic peptide precursor type C |
| NM_053885 | Rere | arginine-glutamic acid dipeptide (RE) repeats |
| NM_057187 | Grifin | galectin-related inter-fiber protein |
| NM_057208 | Tpm3 | tropomyosin 3, gamma |
| NM_133298 | Gpnmb | glycoprotein (transmembrane) nmb |
| NM_134326 | Alb | albumin |
| NM_134387 | Dcxr | dicarbonyl L-xylulose reductase |
| U56241 | Mafb | v-maf musculoaponeurotic fibrosarcoma oncogene family, |
| | | protein B (avian) |
| U86635 | Gstm5 | glutathione S-transferase, mu 5 |
| U92069 | Ucp3 | uncoupling protein 3 (mitochondrial, proton carrier) |
| X67108 | Bdnf | brain derived neurotrophic factor |

D. Adipose + Liver*

| | | |
|---|---|---|
| AA799328 | RGD1560913_predicted | similar to expressed sequence AW413625 (predicted) |
| AA818262 | Angptl4 | angiopoietin-like 4 |
| AA859029 | — | Transcribed locus |
| AA893192 | — | Transcribed locus |
| AA924350 | LOC686634 /// | hypothetical protein LOC686634 /// hypothetical protein |
| | LOC690768 | LOC690768 |
| AA945955 | Ogn_predicted | osteoglycin (predicted) |
| AA955771 | — | — |
| AA957585 | Ehd3 | EH-domain containing 3 |
| AA998903 | — | CDNA clone IMAGE: 7374368 |
| AB013454 | Slc34a1 | solute carrier family 34 (sodium phosphate), member 1 |
| AF411318 | Mt1a | metallothionein 1a |
| AI045116 | — | Transcribed locus |
| AI059204 | Cpne8_predicted | copine VIII (predicted) |
| AI060043 | — | Transcribed locus |
| AI072892 | Frzb | frizzled-related protein |
| AI073272 | Tor1b | torsin family 1, member B |
| AI102061 | Gria3 | glutamate receptor, ionotropic, AMPA3 (alpha 3) |
| AI104238 | Gzma | granzyme A |
| AI137045 | — | Transcribed locus |
| AI137640 | Cldn1 | claudin 1 |
| AI176172 | — | Transcribed locus |
| AI178784 | RGD1310174_predicted | hypothetical LOC298504 (predicted) |
| AI179334 | Fasn | fatty acid synthase |
| AI179886 | RGD1310352 | similar to HTGN29 protein; keratinocytes associated |
| | | transmembrane protein 2 |
| AI232414 | Pxmp4 | peroxisomal membrane protein 4 |
| AI232716 | LOC368066 | similar to indolethylamine N-methyltransferase |
| AI237143 | Rexo4 | REX4, RNA exonuclease 4 homolog (S. cerevisiae) |
| AI406386 | Lmo4 | LIM domain only 4 |
| AI406660 | — | — |
| AI407487 | — | Transcribed locus |
| AI408948 | Ca2 | carbonic anhydrase 2 |
| AI411227 | RGD1563485_predicted | similar to mKIAA0534 protein (predicted) |
| AI411693 | LOC299458 /// | similar to immunoglobulin heavy chain 6 (Igh-6) /// similar |
| | LOC366747 /// | to Ig H-chain V-region precursor /// similar to Ig heavy chain |
| | LOC678701 /// | V region MC101 precursor /// hypothetical protein |
| | RGD1359202 | LOC678701 |
| AI412189 | Igha_mapped /// | immunoglobulin heavy chain (alpha polypeptide) (mapped) |
| | LOC366772 | /// similar to immunoglobulin heavy chain |
| AI548117 | — | — |
| AI555855 | — | Transcribed locus |
| AI556333 | RGD1564257_predicted | similar to hypothetical protein FLJ32825 (predicted) |
| AI577508 | LOC684425 | similar to Adenylosuccinate synthetase isozyme 1 |
| | | (Adenylosuccinate synthetase, muscle isozyme) (IMP-- |
| | | aspartate ligase 1) (AdSS 1) (AMPSase 1) |
| AI599423 | Gadd45g | growth arrest and DNA-damage-inducible 45 gamma |
| AI599463 | Dguok_predicted | deoxyguanosine kinase (predicted) |
| AI639108 | — | — |
| AI639241 | LOC684318 /// | ubiquitin specific protease 12 (predicted) /// similar to |
| | RGD1561481_predicted | ubiquitin specific protease 12 (predicted) /// similar to |
| | /// Usp12_predicted | ubiquitin specific protease 12 |
| AI715202 | RT1-Bb | RT1 class II, locus Bb |
| AJ243338 | RT1-CE5 | RT1 class I, CE5 |
| AW144193 | — | Transcribed locus |
| AW251647 | — | Transcribed locus |
| AW252232 | — | Transcribed locus |
| AW522341 | — | Transcribed locus |
| AW525662 | Cdc27 | cell division cycle 27 homolog (S. cerevisiae) |
| AW526014 | — | Transcribed locus |
| AW527403 | Sh3yl1_predicted | Sh3 domain YSC-like 1 (predicted) |
| AW533234 | LOC688915 | similar to cardiomyopathy associated 5 |
| AW533569 | Clca2_predicted | chloride channel, calcium activated, family member 2 |
| | | (predicted) |
| AW916957 | — | Transcribed locus, strongly similar to XP_579758.1 |
| | | PREDICTED: hypothetical protein XP_579758 [Rattus |
| | | norvegicus] |
| AW920026 | LOC364393 /// | PHD finger protein 11 /// similar to RIKEN cDNA |
| | LOC684778 /// Phfl1 | 4933417L10 /// hypothetical protein LOC684778 |
| BE102861 | — | Transcribed locus |
| BE104961 | Cenpj_predicted | centromere protein J (predicted) |
| BE106199 | Rora_predicted | RAR-related orphan receptor alpha (predicted) |
| BE111310 | Ky_predicted | kyphoscoliosis (predicted) |
| BE113419 | Srpk2_predicted | serine/arginine-rich protein specific kinase 2 (predicted) |
| BE116152 | Elovl6 | ELOVL family member 6, elongation of long chain fatty |
| | | acids (yeast) |
| BE329244 | — | — |
| BF282228 | Ltb | lymphotoxin B |
| BF283070 | Cyp7b1 | cytochrome P450, family 7, subfamily b, polypeptide 1 |
| BF283779 | RGD1565496_predicted | similar to Butyrate-induced transcript 1 (predicted) |
| BF288361 | — | — |
| BF390510 | RGD1311155 | similar to RIKEN cDNA 9230117N10 |
| BF392577 | Akap8l | A kinase (PRKA) anchor protein 8-like |
| BF396857 | Elovl6 | ELOVL family member 6, elongation of long chain fatty |
| | | acids (yeast) |
| BF398091 | — | CDNA clone IMAGE: 7374368 |
| BF398168 | — | Transcribed locus |
| BF408445 | Gpx7_predicted | glutathione peroxidase 7 (predicted) |
| BF545930 | — | Transcribed locus |
| BF565675 | Arsa | arylsulfatase A |
| BG372602 | RGD1306601_predicted | similar to cDNA sequence BC019755 (predicted) |
| BG375362 | Ltbp4 | latent transforming growth factor beta binding protein 4 |
| BG379394 | — | Transcribed locus |
| BG662519 | Ank | Progressive ankylosis homolog (mouse) |
| BG664011 | Pxmp4 | peroxisomal membrane protein 4 |
| BG664221 | Ogn_predicted | osteoglycin (predicted) |
| BI273908 | — | Transcribed locus |
| BI278180 | — | Transcribed locus |
| BI279384 | — | Transcribed locus |
| BI282114 | — | Endogenous retrovirus mRNA, partial sequence |
| BI285494 | Ifitm3 | interferon induced transmembrane protein 3 |
| BI285941 | — | Transcribed locus |
| BI292056 | — | Transcribed locus |
| BI296153 | Acaca | acetyl-coenzyme A carboxylase alpha |
| BI395849 | — | — |
| BM383531 | LOC682651 /// | similar to Metallothionein-2 (MT-2) (Metallothionein-II) |
| | LOC689415 | (MT-II) |
| BM387813 | — | Transcribed locus |
| BM388719 | Rpl7 | Ribosomal protein L7 |
| BM390378 | RGD1562732_predicted | similar to Glutathione S-transferase, theta 3 (predicted) |
| BM390571 | Sult2b1_predicted | sulfotransferase family, cytosolic, 2B, member 1 (predicted) |
| BM391471 | Atf5 | activating transcription factor 5 |
| BM391823 | RGD1563309_predicted | similar to diacylglycerol kinase, delta 130 kDa isoform 1 |
| | | (predicted) |
| J02585 | Scd1 | stearoyl-Coenzyme A desaturase 1 |
| L19933 | Ptprd | protein tyrosine phosphatase, receptor type, D |
| M24024 | RT1-Aw2 | RT1 class Ib, locus Aw2 |
| M25804 | Nr1d1 | nuclear receptor subfamily 1, group D, member 1 |
| M30596 | Me1 | malic enzyme 1 |
| M57668 | Prlr | prolactin receptor |
| NM_012524 | Cebpa | CCAAT/enhancer binding protein (C/EBP), alpha |
| NM_012531 | Comt | catechol-O-methyltransferase |
| NM_012600 | Me1 | malic enzyme 1 |
| NM_012646 | RT1-N1 /// RT1-N2 /// | RT1 class Ib gene, H2-TL-like, grc region (N1) /// RT1 class |
| | RT1-N3 | Ib gene, H2-TL-like, grc region (N3) /// RT1 class Ib gene, |
| | | H2-TL-like, grc region(N2) |
| NM_012744 | Pc | Pyruvate carboxylase |
| NM_016987 | Acly | ATP citrate lyase |
| NM_017206 | Slc6a6 | solute carrier family 6 (neurotransmitter transporter, taurine), |
| | | member 6 |
| NM_017332 | Fasn | fatty acid synthase |
| NM_019291 | Ca2 | carbonic anhydrase 2 |
| NM_020072 | Acpp | acid phosphatase, prostate |
| NM_021744 | Cd14 | CD14 antigen |
| NM_022686 | LOC680097 /// | similar to germinal histone H4 gene |
| | LOC684887 | |
| NM_023025 | Cyp2j3 /// Cyp2j4 | cytochrome P450, family 2, subfamily J, polypeptide 4 /// |
| | | cytochrome P450, family 2, subfamily j, polypeptide 3 |
| NM_024147 | Evl | Ena-vasodilator stimulated phosphoprotein |
| NM_030994 | Itga1 | integrin alpha 1 |
| NM_031559 | Cpt1a | carnitine palmitoyltransferase 1a, liver |
| NM_031841 | Scd2 | stearoyl-Coenzyme A desaturase 2 |
| NM_031971 | Hspa1a /// Hspa1b | heat shock 70 kD protein 1A /// heat shock 70 kD protein 1B |
| | | (mapped) |
| NM_053328 | Bhlhb2 | basic helix-loop-helix domain containing, class B2 |
| NM_053584 | — | — |
| NM_053587 | S100a9 | S100 calcium binding protein A9 (calgranulin B) |
| NM_053639 | Ltc4s | leukotriene C4 synthase |
| NM_053714 | Ank | progressive ankylosis homolog (mouse) |
| NM_053999 | Ppp2r2a | protein phosphatase 2 (formerly 2A), regulatory subunit B |
| | | (PR 52), alpha isoform |
| NM_057208 | Tpm3 | tropomyosin 3, gamma |
| NM_130433 | Acaa2 | acetyl-Coenzyme A acyltransferase 2 (mitochondrial 3- |
| | | oxoacyl-Coenzyme A thiolase) |

E. Adipose + Quadricep*

| | | |
|---|---|---|
| AA799328 | RGD1560913_predicted | similar to expressed sequence AW413625 (predicted) |
| AA800750 | — | — |
| AA819034 | isg12(b) | putative ISG12(b) protein |
| AA850780 | RGD1564560_predicted | similar to RCK (predicted) |
| AA875438 | RGD1309414_predicted | similar to KIAA0913 protein (predicted) |
| AA901290 | — | Transcribed locus |
| AI008409 | — | CDNA clone IMAGE: 7321089 |
| AI030203 | Irx3_predicted | Iroquois related homeobox 3 (Drosophila) (predicted) |
| AI044784 | — | Transcribed locus |
| AI045904 | — | — |
| AI071674 | — | Transcribed locus |
| AI102732 | MGC109340 | similar to Microsomal signal peptidase 23 kDa subunit |
| | | (SPase 22 kDa subunit) (SPC22/23) |
| AI172110 | — | — |
| AI177934 | LOC474169 | pre-eosinophil-associated ribonuclease-2 |
| AI179334 | Fasn | fatty acid synthase |
| AI179886 | RGD1310352 | similar to HTGN29 protein; keratinocytes associated |
| | | transmembrane protein 2 |
| AI179988 | Enc1 | ectodermal-neural cortex 1 |
| AI232414 | Pxmp4 | peroxisomal membrane protein 4 |
| AI235468 | Dst_predicted | dystonin (predicted) |
| AI237143 | Rexo4 | REX4, RNA exonuclease 4 homolog (S. cerevisiae) |
| AI406386 | Lmo4 | LIM domain only 4 |
| AI548667 | — | Transcribed locus |
| AI555855 | — | Transcribed locus |
| AI599463 | Dguok_predicted | deoxyguanosine kinase (predicted) |
| AI715202 | RT1-Bb | RT1 class II, locus Bb |
| AW251280 | — | — |
| AW522341 | — | Transcribed locus |
| AW527159 | — | Transcribed locus |
| AW534218 | RGD1310722_predicted | similar to RIKEN cDNA D130059P03 gene (predicted) |
| BE097574 | Kcnab1 | potassium voltage-gated channel, shaker-related subfamily, |
| | | beta member 1 |
| BE098532 | LOC303057 | similar to step II splicing factor SLU7; DNA segment, Chr |
| | | 11, ERATO Doi 730, expressed; DNA segment, Chr 3, |
| | | Brigham & Womens Genetics 0878 expressed |
| BE102861 | — | Transcribed locus |
| BE108716 | — | Transcribed locus |
| BE117891 | Mll5 | myeloid/lymphoid or mixed-lineage leukemia 5 (trithorax |
| | | homolog, Drosophila) |
| BE126475 | Prrx1 | paired related homeobox 1 |
| BE329244 | — | — |
| BF288243 | Klf2_predicted | Kruppel-like factor 2 (lung) (predicted) |
| BF397936 | RGD1307526 | similar to modulator of estrogen induced transcription |
| BF398435 | — | Transcribed locus |
| BF417216 | LOC686132 | similar to hemicentin 1 |
| BF522436 | RGD1306067 | similar to chromosome 20 open reading frame 6 |
| BF524010 | — | — |
| BG371544 | — | Transcribed locus |
| BG375352 | Ca5b | carbonic anhydrase VB, mitochondrial |
| BI275261 | — | — |
| BI278180 | — | Transcribed locus |
| BI282114 | — | Endogenous retrovirus mRNA, partial sequence |
| BI282920 | Ccl21b | chemokine (C-C motif) ligand 21b (serine) |
| BI288619 | Jun | Jun oncogene |
| BI297744 | RGD1310507 | similar to RIKEN cDNA 1300017J02 |
| BI300470 | — | Transcribed locus |
| BI300794 | — | Transcribed locus |
| BM385476 | Bst2 | bone marrow stromal cell antigen 2 |
| BM388738 | — | — |
| BM389513 | LOC688090 /// RT1-Bb | RT1 class II, locus Bb /// similar to RT1 class II |
| | | histocompatibility antigen, B-1 beta chain precursor (RT1.B- |
| | | beta(1)) |
| J00710 | Csn1s1 | casein alpha s1 |
| J02585 | Scd1 | stearoyl-Coenzyme A desaturase 1 |
| M24024 | RT1-Aw2 | RT1 class Ib, locus Aw2 |
| NM_012531 | Comt | catechol-O-methyltransferase |
| NM_012594 | Lalba | lactalbumin, alpha |
| NM_012646 | RT1-N1 /// RT1-N2 /// | RT1 class Ib gene, H2-TL-like, grc region (N1) /// RT1 class |
| | RT1-N3 | Ib gene, H2-TL-like, grc region (N3) /// RT1 class Ib gene, |
| | | H2-TL-like, grc region(N2) |
| NM_012703 | Thrsp | thyroid hormone responsive protein |
| NM_017332 | Fasn | fatty acid synthase |
| NM_019161 | RGD1566401_predicted | similar to GTL2, imprinted maternally expressed |
| | | untranslated (predicted) |
| NM_019205 | Ccl11 | chemokine (C-C motif) ligand 11 |
| NM_022392 | Insig1 | insulin induced gene 1 |
| NM_023025 | Cyp2j3 /// Cyp2j4 | cytochrome P450, family 2, subfamily J, polypeptide 4 /// |
| | | cytochrome P450, family 2, subfamily j, polypeptide 3 |
| NM_024131 | Ddt | D-dopachrome tautomerase |
| NM_030994 | Itga1 | integrin alpha 1 |
| NM_031327 | Cyr61 | cysteine rich protein 61 |
| NM_031598 | Pla2g2a | phospholipase A2, group IIA (platelets, synovial fluid) |
| NM_031971 | Hspa1a /// Hspa1b | heat shock 70 kD protein 1A /// heat shock 70 kD protein 1B |
| | | (mapped) |
| NM_053355 | Ikbkb | inhibitor of kappaB kinase beta |
| NM_053885 | Rere | arginine-glutamic acid dipeptide (RE) repeats |
| NM_057187 | Grifin | galectin-related inter-fiber protein |
| NM_057208 | Tpm3 | tropomyosin 3, gamma |
| NM_134326 | Alb | albumin |
| U86635 | Gstm5 | glutathione S-transferase, mu 5 |

F. Liver + Quadricep*

| | | |
|---|---|---|
| AA799328 | RGD1560913_predicted | similar to expressed sequence AW413625 (predicted) |
| AA849966 | — | — |
| AF065161 | Cish | cytokine inducible SH2-containing protein |
| AI169331 | Gstm2 | glutathione S-transferase, mu 2 |
| AI177358 | Slc25a25 | solute carrier family 25 (mitochondrial carrier, phosphate |
| | | carrier), member 25 |
| AI179334 | Fasn | fatty acid synthase |
| AI179886 | RGD1310352 | similar to HTGN29 protein; keratinocytes associated |
| | | transmembrane protein 2 |
| AI180253 | RGD1563825_predicted | similar to ENSANGP00000020885 (predicted) |
| AI231999 | LOC689256 | similar to Tumor protein D53 (mD53) (Tumor protein D52- |
| | | like 1) |
| AI232414 | Pxmp4 | peroxisomal membrane protein 4 |
| AI237143 | Rexo4 | REX4, RNA exonuclease 4 homolog (S. cerevisiae) |
| AI406386 | Lmo4 | LIM domain only 4 |
| AI555855 | — | Transcribed locus |
| AI599463 | Dguok_predicted | deoxyguanosine kinase (predicted) |
| AI715202 | RT1-Bb | RT1 class II, locus Bb |
| AJ249701 | RT1-A2 /// RT1-A3 /// | RT1 class Ib, locus Aw2 /// RT1 class Ia, locus A2 /// RT1 |
| | RT1-Aw2 | class I, A3 |
| AW522341 | — | Transcribed locus |
| AW525342 | Usp1 | ubiquitin specific peptdiase 1 |
| AW530361 | Ppp1r3c | protein phosphatase 1, regulatory (inhibitor) subunit 3C |
| BE102861 | — | Transcribed locus |
| BE329244 | — | — |
| BE349751 | — | — |
| BI278180 | — | Transcribed locus |
| BI282114 | — | Endogenous retrovirus mRNA, partial sequence |
| BI291842 | — | Transcribed locus |
| BM383683 | RGD1310710_predicted | similar to RIKEN cDNA 2700091N06 (predicted) |
| J02585 | Scd1 | stearoyl-Coenzyme A desaturase 1 |
| M24024 | RT1-Aw2 | RT1 class Ib, locus Aw2 |
| NM_012531 | Comt | catechol-O-methyltransferase |
| NM_012646 | RT1-N1 /// RT1-N2 /// | RT1 class Ib gene, H2-TL-like, grc region (N1) /// RT1 class |
| | RT1-N3 | Ib gene, H2-TL-like, grc region (N3) /// RT1 class Ib gene, |
| | | H2-TL-like, grc region(N2) |
| NM_017332 | Fasn | fatty acid synthase |
| NM_023025 | Cyp2j3 /// Cyp2j4 | cytochrome P450, family 2, subfamily J, polypeptide 4 /// |
| | | cytochrome P450, family 2, subfamily j, polypeptide 3 |
| NM_030994 | Itga1 | integrin alpha 1 |
| NM_031971 | Hspa1a /// Hspa1b | heat shock 70 kD protein 1A /// heat shock 70 kD protein 1B |
| | | (mapped) |
| NM_053299 | Ubd | ubiquitin D |
| NM_057208 | Tpm3 | tropomyosin 3, gamma |

G. Adipose + Liver + Quadricep*

| | | |
|---|---|---|
| AA799328 | RGD1560913_predicted | similar to expressed sequence AW413625 (predicted) |
| AI179334 | Fasn | fatty acid synthase |
| AI179886 | RGD1310352 | similar to HTGN29 protein; keratinocytes associated |
| | | transmembrane protein 2 |
| AI232414 | Pxmp4 | peroxisomal membrane protein 4 |
| AI237143 | Rexo4 | REX4, RNA exonuclease 4 homolog (S. cerevisiae) |
| AI406386 | Lmo4 | LIM domain only 4 |
| AI555855 | — | Transcribed locus |
| AI599463 | Dguok_predicted | deoxyguanosine kinase (predicted) |
| AI715202 | RT1-Bb | RT1 class II, locus Bb |
| AW522341 | — | Transcribed locus |
| BE102861 | — | Transcribed locus |
| BE329244 | — | — |
| BI278180 | — | Transcribed locus |
| BI282114 | — | Endogenous retrovirus mRNA, partial sequence |
| J02585 | Scd1 | stearoyl-Coenzyme A desaturase 1 |
| M24024 | RT1-Aw2 | RT1 class Ib, locus Aw2 |
| NM_012531 | Comt | catechol-O-methyltransferase |
| NM_012646 | RT1-N1 /// RT1-N2 /// | RT1 class Ib gene, H2-TL-like, grc region (N1) /// RT1 class Ib |
| | RT1-N3 | gene, H2-TL-like, grc region (N3) /// RT1 class Ib gene, H2- |
| | | TL-like, grc region(N2) |
| NM_017332 | Fasn | fatty acid synthase |
| NM_023025 | Cyp2j3 /// Cyp2j4 | cytochrome P450, family 2, subfamily J, polypeptide 4 /// |
| | | cytochrome P450, family 2, subfamily j, polypeptide 3 |
| NM_030994 | Itga1 | integrin alpha 1 |
| NM_031971 | Hspa1a /// Hspa1b | heat shock 70 kD protein 1A /// heat shock 70 kD protein 1B |
| | | (mapped) |
| NM_057208 | Tpm3 | tropomyosin 3, gamma |

*“Adipose + Liver”, for example refers to genes that are differentially expressed in both adipose and liver tissue in response to high protein treatments as compared with the control. “Adipose + Quadricep,” “Liver + Quadricep,” and “Adipose + Liver + Quadricep” are defined analogously.

Biological pathway analysis was conducted using commercially available mapping programs and public databases. The major pathways represented by the differentially expressed genes in adipose, liver, and muscle are set forth below in Tables 11, 12 and 13. As stated above, these genes were found to be differentially expressed as a result of the high protein diet treatment.

TABLE 11
Biochemical Pathways Associated with Genes Differentially Expressed
in Adipose Tissue as a Result of High Protein Diet

| | | |
|---|---|---|
| Public ID | Gene Symbol | Gene Title |

Immune Response

| | | |
|---|---|---|
| AA817742 | Ms4a1_predicted | membrane-spanning 4-domains, subfamily A, member 1 |
| | | (predicted) |
| AA858815 | — | Transcribed locus |
| AA996885 | Ccl19_predicted | chemokine (C-C motif) ligand 19 (predicted) |
| AF057025 | Tlr4 | toll-like receptor 4 |
| AI009823 | Sectm1b | secreted and transmembrane 1B |
| AI011757 | Fcgr3a | Fc fragment of IgG, low affinity IIIa, receptor |
| AI012250 | — | Transcribed locus |
| AI029460 | — | Transcribed locus |
| AI044222 | Cxcl9 | chemokine (C-X-C motif) ligand 9 |
| AI104238 | Gzma | granzyme A |
| AI169104 | Cxcl4 | chemokine (C-X-C motif) ligand 4 |
| AI169601 | Tnfrsf14 | tumor necrosis factor receptor superfamily, member 14 |
| | | (herpesvirus entry mediator) |
| AI170387 | Cxcl9 | chemokine (C-X-C motif) ligand 9 |
| AI177403 | LOC497796 /// | Ly49 inhibitory receptor 9 /// |
| | LOC502907 /// | immunoreceptor Ly49si1 /// |
| | LOC690045 /// | immunoreceptor Ly49si2 /// |
| | LOC690097 /// | immunoreceptor Ly49si3 /// |
| | Ly49i5 /// | Ly49 inhibitory receptor 5 /// |
| | Ly49i9 /// | hypothetical protein LOC497796 /// |
| | Ly49si1 /// | similar to immunoreceptor Ly49si3 (predicted) /// |
| | Ly49si2 /// | similar to immunoreceptor Ly49si1 /// |
| | Ly49si3 /// | similar to immunoreceptor Ly49si3 |
| | RGD1561306_predicted /// | |
| | RGD1563110_predicted | |
| AI178808 | Il2rg | interleukin 2 receptor, gamma (severe combined |
| | | immunodeficiency) |
| AI236229 | LOC681858 /// | similar to RNA binding motif protein 24 |
| | LOC690139 | |
| AI549199 | Ptger4 | Prostaglandin E receptor 4 (subtype EP4) |
| AI599423 | Gadd45g | growth arrest and DNA-damage-inducible 45 gamma |
| AI715202 | RT1-Bb | RT1 class II, locus Bb |
| AJ243338 | RT1-CE5 | RT1 class I, CE5 |
| AJ243973 | RT1-S3 | RT1 class Ib, locus S3 |
| AJ243974 | RT1-S3 | RT1 class Ib, locus S3 |
| AW433947 | Tnfrsf5 | tumor necrosis factor receptor superfamily, member 5 |
| AW526982 | Tlr2 | toll-like receptor 2 |
| AW532179 | LOC500449 | similar to SHP2-interacting transmembrane adaptor protein |
| BE095824 | Ccl6 | chemokine (C-C motif) ligand 6 |
| BE096652 | Ly6g6c | lymphocyte antigen 6 complex, locus G6C |
| BE109730 | — | Transcribed locus |
| BE117044 | Cd8a | CD8 antigen, alpha chain |
| BF281987 | Cxcl11 | chemokine (C-X-C motif) ligand 11 |
| BF282228 | Ltb | lymphotoxin B |
| BF282471 | Lcp2 | lymphocyte cytosolic protein 2 |
| BF288109 | — | Transcribed locus |
| BF418957 | C1qa | complement component 1, q subcomponent, alpha polypeptide |
| BF419319 | Oasl1 | 2′-5′ oligoadenylate synthetase-like 1 |
| BF419899 | Ccl7 | chemokine (C-C motif) ligand 7 |
| BG057565 | Bat1a | HLA-B-associated transcript 1A |
| BG378166 | Map4k2_predicted | mitogen activated protein kinase kinase kinase kinase 2 |
| | | (predicted) |
| BI279526 | RT1-Db1 | RT1 class II, locus Db1 |
| BI282920 | Ccl21b | chemokine (C-C motif) ligand 21b (serine) |
| BI285494 | Ifitm3 | interferon induced transmembrane protein 3 |
| BI290909 | Scap1 | src family associated phosphoprotein 1 |
| BI291927 | RT1-A2 /// RT1-A3 | RT1 class Ia, locus A2 /// RT1 class I, A3 |
| BI294084 | Ccl24 | chemokine (C-C motif) ligand 24 |
| BM383464 | — | Transcribed locus |
| BM386789 | Rgs1 | regulator of G-protein signaling 1 |
| BM387813 | — | Transcribed locus |
| BM389513 | LOC688090 /// RT1-Bb | RT1 class II, locus Bb /// similar to RT1 class II |
| | | histocompatibility antigen, B-1 beta chain precursor |
| | | (RT1.B-beta(1)) |
| BM391631 | Fcgr1 | Fc receptor, IgG, high affinity I |
| D87927 | Gm1960 | gene model 1960, (NCBI) |
| M17153 | Fcer1a | Fc receptor, IgE, high affinity I, alpha polypeptide |
| M24024 | RT1-Aw2 | RT1 class Ib, locus Aw2 |
| NM_012493 | Afp | alpha-fetoprotein |
| NM_012516 | C4bpa | complement component 4 binding protein, alpha |
| NM_012555 | Ets1 | v-ets erythroblastosis virus E26 oncogene homolog 1 (avian) |
| NM_012755 | Fyn | fyn proto-oncogene |
| NM_012925 | Cd59 | CD59 antigen |
| NM_017028 | Mx2 | myxovirus (influenza virus) resistance 2 |
| NM_017320 | Ctss | cathepsin S |
| NM_019205 | Ccl11 | chemokine (C-C motif) ligand 11 |
| NM_021744 | Cd14 | CD14 antigen |
| NM_021866 | Ccr2 | chemokine (C-C motif) receptor 2 |
| NM_030853 | Lat | linker for activation of T cells |
| NM_031116 | Ccl5 | chemokine (C-C motif) ligand 5 |
| NM_031530 | Ccl2 | chemokine (C-C motif) ligand 2 |
| NM_031538 | Cd8a | CD8 antigen, alpha chain |
| NM_053843 | Fcgr3 | Fc receptor, IgG, low affinity III |
| NM_057151 | Ccl17 | chemokine (C-C motif) ligand 17 |
| NM_130399 | Ada | adenosine deaminase |
| NM_130421 | Lcp2 | lymphocyte cytosolic protein 2 |
| NM_133533 | Cd79b | CD79B antigen |
| NM_133542 | Igsf6 | immunoglobulin superfamily, member 6 |
| NM_134361 | Xcl1 | chemokine (C motif) ligand 1 |
| U22520 | Cxcl10 | chemokine (C-X-C motif) ligand 10 |
| U50449 | RT1-CE16 | RT1 class I, CE16 |
| Z18877 | Oas1 | 2′,5′-oligoadenylate synthetase 1, 40/46 kDa |

Inflammatory response

| | | |
|---|---|---|
| AF057025 | Tlr4 | toll-like receptor 4 |
| AI044869 | Pla2g2d | phospholipase A2, group IID |
| AI103918 | Hdac7a | histone deacetylase 7A |
| AI236229 | LOC681858 /// | similar to RNA binding motif protein 24 |
| | LOC690139 | |
| AI236455 | Anxa1 | annexin A1 |
| AI454911 | Scye1 | small inducible cytokine subfamily E, member 1 |
| AW433947 | Tnfrsf5 | Tumor necrosis factor receptor superfamily, member 5 |
| AW526982 | Tlr2 | toll-like receptor 2 |
| BE117044 | Cd8a | CD8 antigen, alpha chain |
| BF282228 | Ltb | lymphotoxin B |
| BF419899 | Ccl7 | chemokine (C-C motif) ligand 7 |
| BI282920 | Ccl21b | chemokine (C-C motif) ligand 21b (serine) |
| BI294084 | Ccl24 | chemokine (C-C motif) ligand 24 |
| BM383464 | — | Transcribed locus |
| BM389005 | Gpr68_predicted | G protein-coupled receptor 68 (predicted) |
| BM391303 | — | — |
| D87927 | Gm1960 | gene model 1960, (NCBI) |
| L25527 | Sele | selectin, endothelial cell |
| NM_012488 | A2m | Alpha-2-macroglobulin |
| NM_012559 | Fgg | fibrinogen, gamma polypeptide |
| NM_017260 | Alox5ap | arachidonate 5-lipoxygenase activating protein |
| NM_019205 | Ccl11 | chemokine (C-C motif) ligand 11 |
| NM_020542 | Ccr1 | chemokine (C-C motif) receptor 1 |
| NM_021744 | Cd14 | CD14 antigen |
| NM_021866 | Ccr2 | chemokine (C-C motif) receptor 2 |
| NM_024131 | Ddt | D-dopachrome tautomerase |
| NM_031051 | Mif | macrophage migration inhibitory factor |
| NM_031116 | Ccl5 | chemokine (C-C motif) ligand 5 |
| NM_031530 | Ccl2 | chemokine (C-C motif) ligand 2 |
| NM_031538 | Cd8a | CD8 antigen, alpha chain |
| NM_033230 | Akt1 | thymoma viral proto-oncogene 1 |
| NM_053587 | S100a9 | S100 calcium binding protein A9 (calgranulin B) |
| NM_053822 | S100a8 | S100 calcium binding protein A8 (calgranulin A) |
| NM_053843 | Fcgr3 | Fc receptor, IgG, low affinity III |
| U22520 | Cxcl10 | chemokine (C-X-C motif) ligand 10 |

Response to Stress

| | | |
|---|---|---|
| AA819776 | Hspca /// | heat shock protein 1, alpha /// similar to heat shock protein 1, alpha |
| | LOC498264 /// | |
| | LOC500299 /// | |
| | LOC691091 | |
| AI170535 | Tp53i11_predicted | Tumor protein p53 inducible protein 11 (predicted) |
| AI236229 | LOC681858 /// | similar to RNA binding motif protein 24 |
| | LOC690139 | |
| AI408948 | Ca2 | carbonic anhydrase 2 |
| AI599423 | Gadd45g | growth arrest and DNA-damage-inducible 45 gamma |
| BF288101 | Snn | stannin |
| BF417479 | Dhcr24 | 24-dehydrocholesterol reductase |
| BG378166 | Map4k2_predicted | mitogen activated protein kinase kinase kinase kinase 2 (predicted) |
| J02612 | Ugt1a1 /// | UDP glycosyltransferase 1 family, polypeptide A1 /// |
| | Ugt1a10 /// | UDP glycosyltransferase 1 family, polypeptide A6 /// |
| | Ugt1a2 /// | UDP glycosyltransferase 1 family, polypeptide A7 /// |
| | Ugt1a3 /// | UDP glycosyltransferase 1 family, polypeptide A8 /// |
| | Ugt1a5 /// | UDP glycosyltransferase 1 family polypeptide A2 /// |
| | Ugt1a6 /// | UDP glycosyltransferase 1 family polypeptide A3 /// |
| | Ugt1a7 /// | UDP glycosyltransferase 1 family polypeptide A10 /// |
| | Ugt1a8 | UDP glycosyltransferase 1 family, polypeptide A5 |
| NM_019291 | Ca2 | carbonic anhydrase 2 |
| NM_031020 | Mapk14 | mitogen activated protein kinase 14 |
| NM_031031 | Gatm | glycine amidinotransferase (L-arginine:glycine amidinotransferase) |
| NM_031971 | Hspa1a /// | heat shock 70 kD protein 1A /// heat shock 70 kD protein 1B (mapped) |
| | Hspa1b | |
| NM_053843 | Fcgr3 | Fc receptor, IgG, low affinity III |
| NM_053887 | Map3k1 | mitogen activated protein kinase kinase kinase 1 |
| NM_134326 | Alb | albumin |

Chemotaxis

| | | |
|---|---|---|
| AA996885 | Ccl19_predicted | chemokine (C-C motif) ligand 19 (predicted) |
| AF253064 | Cklf | chemokine-like factor |
| AI010476 | Rac2 | RAS-related C3 botulinum substrate 2 |
| AI045155 | Ccr6 | chemokine (C-C motif) receptor 6 |
| AI169104 | Cxcl4 | chemokine (C-X-C motif) ligand 4 |
| BE095824 | Ccl6 | chemokine (C-C motif) ligand 6 |
| BF414285 | — | Transcribed locus |
| BF419899 | Ccl7 | chemokine (C-C motif) ligand 7 |
| BI282920 | Ccl21b | chemokine (C-C motif) ligand 21b (serine) |
| BI294084 | Ccl24 | chemokine (C-C motif) ligand 24 |
| D87927 | Gm1960 | gene model 1960, (NCBI) |
| NM_013085 | Plau | plasminogen activator, urokinase |
| NM_019205 | Ccl11 | chemokine (C-C motif) ligand 11 |
| NM_031020 | Mapk14 | Mitogen activated protein kinase 14 |
| NM_031116 | Ccl5 | chemokine (C-C motif) ligand 5 |
| NM_031327 | Cyr61 | Cysteine rich protein 61 |
| NM_031530 | Ccl2 | chemokine (C-C motif) ligand 2 |
| NM_053822 | S100a8 | S100 calcium binding protein A8 (calgranulin A) |
| NM_053843 | Fcgr3 | Fc receptor, IgG, low affinity III |
| NM_057151 | Ccl17 | chemokine (C-C motif) ligand 17 |
| NM_134361 | Xcl1 | chemokine (C motif) ligand 1 |
| U22520 | Cxcl10 | chemokine (C-X-C motif) ligand 10 |
| U28830 | Ralbp1 | ralA binding protein 1 |

Response to Unfolded Protein

| | | |
|---|---|---|
| AA819776 | Hspca /// | heat shock protein 1, alpha /// similar to heat shock protein 1, alpha |
| | LOC498264 /// | |
| | LOC500299 /// | |
| | LOC691091 | |
| AF077354 | Hspa4 | heat shock protein 4 |
| AI073272 | Tor1b | torsin family 1, member B |
| AI639117 | Cfb | complement factor B |
| AI716125 | C2 | complement component 2 |
| BF525282 | Hsph1 | heat shock 105 kDa/110 kDa protein 1 |
| BF553613 | Txndc4 | thioredoxin domain containing 4 (endoplasmic reticulum) |
| BI285700 | Hspcb | heat shock 90 kDa protein 1, beta |
| BI289103 | Slc44a4 | solute carrier family 44, member 4 |
| BM383464 | — | Transcribed locus |
| NM_022229 | Hspd1 | heat shock protein 1 (chaperonin) |
| NM_031971 | Hspa1a /// | heat shock 70 kD protein 1A /// heat shock 70 kD protein 1B (mapped) |
| | Hspa1b | |

Defense Response

| | | |
|---|---|---|
| AF068860 | Defb1 | defensin beta 1 |
| AI236229 | LOC681858 /// | similar to RNA binding motif protein 24 |
| | LOC690139 | |
| AI639117 | Cfb | complement factor B |
| AI716125 | C2 | complement component 2 |
| BE118901 | Gata3 | GATA binding protein 3 |
| BI289103 | Slc44a4 | solute carrier family 44, member 4 |
| BM383464 | — | Transcribed locus |
| NM_012594 | Lalba | lactalbumin, alpha |
| NM_017028 | Mx2 | myxovirus (influenza virus) resistance 2 |
| NM_023021 | Kcnn4 | potassium intermediate/small conductance calcium-activated channel, |
| | | subfamily N, member 4 |
| NM_031971 | Hspa1a /// | heat shock 70 kD protein 1A /// heat shock 70 kD protein 1B (mapped) |
| | Hspa1b | |
| NM_053843 | Fcgr3 | Fc receptor, IgG, low affinity III |
| NM_053960 | Ccr5 | chemokine (C-C motif) receptor 5 |

Cell activation

| | | |
|---|---|---|
| AI044631 | Cd3g | CD3 antigen, gamma polypeptide |
| BF282228 | Ltb | lymphotoxin B |
| BM383464 | — | Transcribed locus |
| NM_053819 | Timp1 | tissue inhibitor of metallopeptidase 1 |
| NM_053843 | Fcgr3 | Fc receptor, IgG, low affinity III |
| U56824 | Klra5 /// | killer cell lectin-like receptor, subfamily A, member 5 /// |
| | Ly49s7 | Ly49 stimulatory receptor 7 |

Lymphocyte Activation

| | | |
|---|---|---|
| AA945909 | Cd3e_predicted | CD3 antigen, epsilon polypeptide (predicted) |
| AI044631 | Cd3g | CD3 antigen, gamma polypeptide |
| AW915948 | Cd3e_predicted | CD3 antigen, epsilon polypeptide (predicted) |
| U56824 | Klra5 /// | killer cell lectin-like receptor, subfamily A, member 5 /// |
| | Ly49s7 | Ly49 stimulatory receptor 7 |

Locomotory Behavior

| | | |
|---|---|---|
| AA926313 | — | Transcribed locus |
| BF389056 | — | Transcribed locus |
| BG662519 | Ank | Progressive ankylosis homolog (mouse) |
| BI395810 | Npy1r | neuropeptide Y receptor Y1 |
| NM_053714 | Ank | progressive ankylosis homolog (mouse) |
| NM_053843 | Fcgr3 | Fc receptor, IgG, low affinity III |

Lipid Metabolic Process

| | | |
|---|---|---|
| AA800240 | Hadha | hydroxyacyl-Coenzyme A dehydrogenase/3-ketoacyl-Coenzyme A |
| | | thiolase/enoyl-Coenzyme A hydratase (trifunctional protein), |
| | | alpha subunit |
| AA848820 | Hpgd | hydroxyprostaglandin dehydrogenase 15 (NAD) |
| AA892234 | Mgst3_predicted | microsomal glutathione S-transferase 3 (predicted) |
| AF022247 | Cubn | cubilin (intrinsic factor-cobalamin receptor) |
| AI013044 | — | Transcribed locus |
| AI136525 | Degs2 | degenerative spermatocyte homolog 2 (Drosophila), lipid desaturase |
| AI236455 | Anxa1 | annexin A1 |
| AI548897 | Soat1 | Sterol O-acyltransferase 1 |
| AJ245707 | Phyh2 | phytanoyl-CoA 2-hydroxylase 2 |
| AW530812 | RGD1311224_predicted | similar to fatty acid desaturase 2; linoleoyl-CoA desaturase (delta-6- |
| | | desaturase)-like 2; delta-6 fatty acid desaturase (predicted) |
| BE113958 | Nr2f2 | Nuclear receptor subfamily 2, group F, member 2 |
| BE116152 | Elovl6 | ELOVL family member 6, elongation of long chain fatty acids (yeast) |
| BF283070 | Cyp7b1 | cytochrome P450, family 7, subfamily b, polypeptide 1 |
| BF396857 | Elovl6 | ELOVL family member 6, elongation of long chain fatty acids (yeast) |
| BF417479 | Dhcr24 | 24-dehydrocholesterol reductase |
| BG671686 | — | — |
| BI278687 | Pltp_predicted | phospholipid transfer protein (predicted) |
| BM383809 | Mlstd2 | male sterility domain containing 2 |
| BM390399 | Hmgcr | 3-hydroxy-3-methylglutaryl-Coenzyme A reductase |
| BM390571 | Sult2b1_predicted | sulfotransferase family, cytosolic, 2B, member 1 (predicted) |
| BM390774 | Peci /// | peroxisomal delta3, delta2-enoyl-Coenzyme A isomerase /// similar to |
| | RGD1310224 | RIKEN cDNA 1810022C23 |
| J02585 | Scd1 | stearoyl-Coenzyme A desaturase 1 |
| NM_012703 | Thrsp | thyroid hormone responsive protein |
| NM_012777 | Apod | apolipoprotein D |
| NM_012824 | Apoc1 | apolipoprotein C-I |
| NM_013134 | Hmgcr | 3-hydroxy-3-methylglutaryl-Coenzyme A reductase |
| NM_013187 | Plcg1 | phospholipase C, gamma 1 |
| NM_013200 | Cpt1b | carnitine palmitoyltransferase 1b, muscle |
| NM_016986 | Acadm | acetyl-Coenzyme A dehydrogenase, medium chain |
| NM_016987 | Acly | ATP citrate lyase |
| NM_017268 | Hmgcs1 | 3-hydroxy-3-methylglutaryl-Coenzyme A synthase 1 |
| NM_022392 | Insig1 | insulin induced gene 1 |
| NM_024390 | Hpgd | hydroxyprostaglandin dehydrogenase 15 (NAD) |
| NM_031559 | Cpt1a | carnitine palmitoyltransferase 1a, liver |
| NM_031841 | Scd2 | stearoyl-Coenzyme A desaturase 2 |
| NM_053339 | Acox3 | acyl-Coenzyme A oxidase 3, pristanoyl |
| NM_053623 | Acsl4 | acyl-CoA synthetase long-chain family member 4 |
| NM_053674 | Phyh | phytanoyl-CoA hydroxylase |
| NM_130433 | Acaa2 | acetyl-Coenzyme A acyltransferase 2 (mitochondrial 3-oxoacyl-Coenzyme A |
| | | thiolase) |
| NM_134382 | Elovl5 | ELOVL family member 5, elongation of long chain fatty acids (yeast) |
| U13253 | Fabp5 | fatty acid binding protein 5, epidermal |

Lipid Biosynthetic Process

| | | |
|---|---|---|
| AI179334 | Fasn | fatty acid synthase |
| AW530769 | Fdft1 | farnesyl diphosphate farnesyl transferase 1 |
| BE105855 | Cyb5r2 | cytochrome b5 reductase 2 |
| BE116152 | Elovl6 | ELOVL family member 6, elongation of long chain fatty acids (yeast) |
| BF396857 | Elovl6 | ELOVL family member 6, elongation of long chain fatty acids (yeast) |
| BF417479 | Dhcr24 | 24-dehydrocholesterol reductase |
| BF419134 | Acbd3 | acyl-Coenzyme A binding domain containing 3 |
| BG664123 | Cyp51 | cytochrome P450, subfamily 51 |
| BM383809 | Mlstd2 | male sterility domain containing 2 |
| BM390399 | Hmgcr | 3-hydroxy-3-methylglutaryl-Coenzyme A reductase |
| BM390574 | Lss | Lanosterol synthase |
| J02585 | Scd1 | stearoyl-Coenzyme A desaturase 1 |
| J03867 | Cyb5r3 | cytochrome b5 reductase 3 |
| NM_012744 | Pc | Pyruvate carboxylase |
| NM_012851 | Hsd17b1 | hydroxysteroid (17-beta) dehydrogenase 1 |
| NM_012941 | Cyp51 | cytochrome P450, subfamily 51 |
| NM_013134 | Hmgcr | 3-hydroxy-3-methylglutaryl-Coenzyme A reductase |
| NM_016987 | Acly | ATP citrate lyase |
| NM_017268 | Hmgcs1 | 3-hydroxy-3-methylglutaryl-Coenzyme A synthase 1 |
| NM_017332 | Fasn | fatty acid synthase |
| NM_019238 | Fdft1 | farnesyl diphosphate farnesyl transferase 1 |
| NM_022193 | Acaca | acetyl-coenzyme A carboxylase alpha |
| NM_022389 | Dhcr7 | 7-dehydrocholesterol reductase |
| NM_022705 | Thedc1 | thioesterase domain containing 1 |
| NM_031840 | Fdps | farnesyl diphosphate synthase |
| NM_031841 | Scd2 | stearoyl-Coenzyme A desaturase 2 |
| NM_053539 | Idi1 | isopentenyl-diphosphate delta isomerase |
| NM_134382 | Elovl5 | ELOVL family member 5, elongation of long chain fatty acids (yeast) |

Steroid Biosynthetic Process

| | | |
|---|---|---|
| AI407163 | — | Transcribed locus |
| AW530769 | Fdft1 | farnesyl diphosphate farnesyl transferase 1 |
| BE105855 | Cyb5r2 | cytochrome b5 reductase 2 |
| BF417479 | Dhcr24 | 24-dehydrocholesterol reductase |
| BF419134 | Acbd3 | acyl-Coenzyme A binding domain containing 3 |
| BG664123 | Cyp51 | cytochrome P450, subfamily 51 |
| BM390399 | Hmgcr | 3-hydroxy-3-methylglutaryl-Coenzyme A reductase |
| BM390574 | Lss | Lanosterol synthase |
| J03867 | Cyb5r3 | cytochrome b5 reductase 3 |
| L17138 | Hsd3b6 | hydroxy-delta-5-steroid dehydrogenase, 3 beta- and steroid delta- |
| | | isomerase 6 |
| M57668 | Prlr | prolactin receptor |
| NM_012851 | Hsd17b1 | hydroxysteroid (17-beta) dehydrogenase 1 |
| NM_012941 | Cyp51 | cytochrome P450, subfamily 51 |
| NM_013134 | Hmgcr | 3-hydroxy-3-methylglutaryl-Coenzyme A reductase |
| NM_017268 | Hmgcs1 | 3-hydroxy-3-methylglutaryl-Coenzyme A synthase 1 |
| NM_019238 | Fdft1 | farnesyl diphosphate farnesyl transferase 1 |
| NM_022389 | Dhcr7 | 7-dehydrocholesterol reductase |
| NM_031840 | Fdps | farnesyl diphosphate synthase |
| NM_053539 | Idi1 | isopentenyl-diphosphate delta isomerase |

Cholesterol Metabolic Process

| | | |
|---|---|---|
| AF022247 | Cubn | cubilin (intrinsic factor-cobalamin receptor) |
| AI412322 | Acat2 | acetyl-Coenzyme A acetyltransferase 2 |
| AI502114 | Abca1 | ATP-binding cassette, sub-family A (ABC1), member 1 |
| AI548897 | Soat1 | Sterol O-acyltransferase 1 |
| AW918387 | Abca1 | ATP-binding cassette, sub-family A (ABC1), member 1 |
| BF283070 | Cyp7b1 | cytochrome P450, family 7, subfamily b, polypeptide 1 |
| BF284523 | Abca1 | ATP-binding cassette, sub-family A (ABC1), member 1 |
| BF417479 | Dhcr24 | 24-dehydrocholesterol reductase |
| NM_017225 | Pctp | phosphatidylcholine transfer protein |
| NM_022392 | Insig1 | insulin induced gene 1 |

Steroid Metabolic Process

| | | |
|---|---|---|
| AF022247 | Cubn | cubilin (intrinsic factor-cobalamin receptor) |
| AI548897 | Soat1 | Sterol O-acyltransferase 1 |
| BF283070 | Cyp7b1 | cytochrome P450, family 7, subfamily b, polypeptide 1 |
| BF417479 | Dhcr24 | 24-dehydrocholesterol reductase |
| BM390571 | Sult2b1_predicted | sulfotransferase family, cytosolic, 2B, member 1 (predicted) |
| NM_022392 | Insig1 | insulin induced gene 1 |

Glycolysis

| | | |
|---|---|---|
| AI411413 | LOC685778 /// | pyruvate dehydrogenase E1 alpha 1 /// pyruvate dehydrogenase E1 alpha 1 |
| | Pdha1 | pseudogene |
| AI411413 | LOC685778 /// | pyruvate dehydrogenase E1 alpha 1 /// pyruvate dehydrogenase E1 alpha 1 |
| | Pdha1 | pseudogene |
| BF561717 | Pdha1 | pyruvate dehydrogenase E1 alpha 1 |
| BI279760 | Pgk1 | phosphoglycerate kinase 1 |
| BI291434 | Pfkm | phosphofructokinase, muscle |
| BI295900 | Dlat | dihydrolipoamide S-acetyltransferase (E2 component of pyruvate |
| | | dehydrogenase complex) |
| BM389223 | Pdhb | pyruvate dehydrogenase (lipoamide) beta |
| NM_012497 | Aldoc | aldolase C |
| NM_012554 | Eno1 | enolase 1, alpha |
| NM_012949 | Eno3 | enolase 3, beta |
| NM_017328 | Pgam2 | phosphoglycerate mutase 2 |
| NM_031715 | Pfkm | phosphofructokinase, muscle |

Glucose Metabolic Process

| | | |
|---|---|---|
| AI411413 | LOC685778 /// | pyruvate dehydrogenase E1 alpha 1 /// pyruvate dehydrogenase E1 alpha 1 |
| | Pdha1 | pseudogene |
| BE108587 | Nisch | nischarin |
| BI295900 | Dlat | dihydrolipoamide S-acetyltransferase (E2 component of pyruvate |
| | | dehydrogenase complex) |
| BI395810 | Npy1r | neuropeptide Y receptor Y1 |
| NM_017006 | G6pdx | glucose-6-phosphate dehydrogenase X-linked |
| NM_031020 | Mapk14 | mitogen activated protein kinase 14 |
| NM_031559 | Cpt1a | carnitine palmitoyltransferase 1a, liver |
| NM_033230 | Akt1 | thymoma viral proto-oncogene 1 |
| NM_053826 | Pdk1 | pyruvate dehydrogenase kinase, isoenzyme 1 |
| U13253 | Fabp5 | fatty acid binding protein 5, epidermal |

Organ Development

| | | |
|---|---|---|
| AA800004 | Sept4 | septin 4 |
| AI713966 | Igfbp3 | insulin-like growth factor binding protein 3 |
| AW144216 | Enpep | glutamyl aminopeptidase |
| BF417479 | Dhcr24 | 24-dehydrocholesterol reductase |
| BG662519 | Ank | Progressive ankylosis homolog (mouse) |
| BG663483 | Pcdha1 /// | protocadherin alpha 4 /// |
| | Pcdha10 /// | protocadherin alpha 13 /// |
| | Pcdha11 /// | protocadherin alpha 10 /// |
| | Pcdha12 /// | protocadherin alpha 12 /// |
| | Pcdha13 /// | protocadherin alpha 3 /// |
| | Pcdha2 /// | protocadherin alpha 8 /// |
| | Pcdha3 /// | protocadherin alpha 1 /// |
| | Pcdha4 /// | protocadherin alpha 2 /// |
| | Pcdha5 /// | protocadherin alpha 5 /// |
| | Pcdha6 /// | protocadherin alpha 6 /// |
| | Pcdha7 /// | protocadherin alpha 7 /// |
| | Pcdha8 /// | protocadherin alpha 9 /// |
| | Pcdha9 /// | protocadherin alpha subfamily C, 1 /// |
| | Pcdhac1 /// | protocadherin alpha subfamily C, 2 /// |
| | Pcdhac2 | protocadherin alpha 11 |
| NM_012755 | Fyn | fyn proto-oncogene |
| NM_017240 | Myh6 /// Myh7 | myosin, heavy polypeptide 6, cardiac muscle, alpha /// myosin, heavy |
| | | polypeptide 7, cardiac muscle, beta |
| NM_019161 | RGD1566401_predicted | similar to GTL2, imprinted maternally expressed untranslated (predicted) |
| NM_031031 | Gatm | glycine amidinotransferase (L-arginine:glycine amidinotransferase) |
| NM_053714 | Ank | progressive ankylosis homolog (mouse) |
| NM_133303 | Bhlhb3 | basic helix-loop-helix domain containing, class B3 |
| U23056 | Ceacam1 /// | CEA-related cell adhesion molecule 1 /// CEA-related cell adhesion |
| | Ceacam10 | molecule 10 |
| U23407 | Crabp2 | cellular retinoic acid binding protein 2 |

Muscle Development

| | | |
|---|---|---|
| AA963276 | Etv1_predicted | ets variant gene 1 (predicted) |
| AA964492 | — | Transcribed locus |
| AI059914 | Etv1_predicted | ets variant gene 1 (predicted) |
| AI713966 | Igfbp3 | insulin-like growth factor binding protein 3 |
| BI277545 | Myh1 /// Myh2 | myosin, heavy polypeptide 1, skeletal muscle, adult /// myosin, heavy |
| | | polypeptide 2, skeletal muscle, adult |
| M15481 | Igf1 | insulin-like growth factor 1 |
| NM_012893 | Actg2 | actin, gamma 2 |
| NM_013172 | Myf6 | myogenic factor 6 |
| NM_017240 | Myh6 /// Myh7 | myosin, heavy polypeptide 6, cardiac muscle, alpha /// myosin, heavy |
| | | polypeptide 7, cardiac muscle, beta |
| NM_019212 | Acta1 | actin, alpha 1, skeletal muscle |
| U22520 | Cxcl10 | chemokine (C-X-C motif) ligand 10 |
| U44948 | Csrp2 | cysteine and glycine-rich protein 2 |
| X74293 | Itga7 | integrin alpha 7 |
| X74294 | Itga7 | integrin alpha 7 |

Positive Regulation of Cell Proliferation

| | | |
|---|---|---|
| AF062594 | Nap1l1 | nucleosome assembly protein 1-like 1 |
| AI144948 | — | Transcribed locus |
| BE108905 | Nap1l1 | Nucleosome assembly protein 1-like 1 |
| BE117736 | Tgfb2 | Transforming growth factor, beta 2 |
| BF553172 | — | Transcribed locus |
| BG670310 | Tgfa | transforming growth factor alpha |
| BM392321 | Hipk2_predicted | homeodomain interacting protein kinase 2 (predicted) |
| L14782 | Lyn | Yamaguchi sarcoma viral (v-yes-1) oncogene homolog |
| M15481 | Igf1 | insulin-like growth factor 1 |
| NM_012559 | Fgg | fibrinogen, gamma polypeptide |
| NM_012888 | Tshr | thyroid stimulating hormone receptor |
| NM_013060 | Id2 | inhibitor of DNA binding 2 |
| NM_017066 | Ptn | pleiotrophin |
| NM_053819 | Timp1 | tissue inhibitor of metallopeptidase 1 |
| U22520 | Cxcl10 | chemokine (C-X-C motif) ligand 10 |

Angiogenesis

| | | |
|---|---|---|
| AA818262 | Angptl4 | angiopoietin-like 4 |
| AI146037 | Map3k7_predicted | Mitogen activated protein kinase kinase kinase 7 (predicted) |
| AW144216 | Enpep | glutamyl aminopeptidase |
| BE117736 | Tgfb2 | Transforming growth factor, beta 2 |
| BF553172 | — | Transcribed locus |
| BF564217 | Ang1 | angiogenin, ribonuclease A family, member 1 |
| BG670310 | Tgfa | transforming growth factor alpha |
| BI285064 | — | Transcribed locus |
| NM_013085 | Plau | plasminogen activator, urokinase |
| NM_013194 | Myh9 | myosin, heavy polypeptide 9, non-muscle |
| NM_031020 | Mapk14 | mitogen activated protein kinase 14 |
| U23056 | Ceacam1 /// | CEA-related cell adhesion molecule 1 /// CEA-related cell adhesion |
| | Ceacam10 | molecule 10 |
| X83537 | Mmp14 | matrix metallopeptidase 14 (membrane-inserted) |

Blood Vessel Morphogenesis

| | | |
|---|---|---|
| AI454911 | Scye1 | small inducible cytokine subfamily E, member 1 |
| AW144216 | Enpep | glutamyl aminopeptidase |
| BE113958 | Nr2f2 | Nuclear receptor subfamily 2, group F, member 2 |
| NM_013085 | Plau | plasminogen activator, urokinase |
| U23056 | Ceacam1 /// | CEA-related cell adhesion molecule 1 /// CEA-related cell adhesion |
| | Ceacam10 | molecule 10 |
| X74293 | Itga7 | integrin alpha 7 |
| X74294 | Itga7 | integrin alpha 7 |

Anti-apoptosis

| | | |
|---|---|---|
| AA819804 | Zfp91 | zinc finger protein 91 |
| AA946430 | Syvn1 | synovial apoptosis inhibitor 1, synoviolin |
| AI236455 | Anxa1 | annexin A1 |
| AI407163 | — | Transcribed locus |
| AW143154 | Mkl1_predicted | megakaryoblastic leukemia (translocation) 1 (predicted) |
| BE111631 | Zfp91 | zinc finger protein 91 |
| BE112093 | Zfp91 | zinc finger protein 91 |
| BE112895 | Pea15 | phosphoprotein enriched in astrocytes 15 |
| BF399517 | — | Transcribed locus |
| BF417479 | Dhcr24 | 24-dehydrocholesterol reductase |
| BG662875 | Pea15 | phosphoprotein enriched in astrocytes 15 |
| BG670310 | Tgfa | transforming growth factor alpha |
| BI282281 | LOC500372 | similar to Stress-70 protein, mitochondrial precursor (75 kDa glucose |
| | | regulated protein) (GRP 75) (Peptide-binding protein 74) (PBP74) |
| | | (MTHSP70) (Mortalin) |
| BM391471 | Atf5 | activating transcription factor 5 |
| M15481 | Igf1 | insulin-like growth factor 1 |
| M57668 | Prlr | prolactin receptor |
| NM_012555 | Ets1 | v-ets erythroblastosis virus E26 oncogene homolog 1 (avian) |
| NM_012660 | Eef1a2 | eukaryotic translation elongation factor 1 alpha 2 |
| NM_012925 | Cd59 | CD59 antigen |
| NM_023987 | Birc3 | baculoviral IAP repeat-containing 3 |
| NM_031530 | Ccl2 | chemokine (C-C motif) ligand 2 |
| NM_031971 | Hspa1a /// | heat shock 70 kD protein 1A /// heat shock 70 kD protein 1B (mapped) |
| | Hspa1b | |
| NM_033230 | Akt1 | thymoma viral proto-oncogene 1 |
| NM_133416 | Bcl2a1 | B-cell leukemia/lymphoma 2 related protein A1 |
| S75280 | Hspa9a_predicted | heat shock 70 kDa protein 9A (predicted) |

Muscle Contraction

| | | |
|---|---|---|
| AA875132 | — | Transcribed locus |
| AF220558 | Trdn | triadin |
| AF370889 | Tpm1 | Tropomyosin 1, alpha |
| AF372216 | Tpm1 | tropomyosin 1, alpha |
| AJ243304 | Trdn | triadin |
| AW533848 | Mybpc2_predicted | myosin binding protein C, fast-type (predicted) |
| BF284889 | Actn2_predicted | actinin alpha 2 (predicted) |
| BF521859 | Tnnt3 | troponin T3, skeletal, fast |
| BF555973 | — | Transcribed locus |
| BG378588 | Mybpc2_predicted | myosin binding protein C, fast-type (predicted) |
| BI277545 | Myh1 /// Myh2 | myosin, heavy polypeptide 1, skeletal muscle, adult /// myosin, heavy |
| | | polypeptide 2, skeletal muscle, adult |
| BI289527 | Tmod4_predicted | tropomodulin 4 (predicted) |
| BM391169 | Myh4 | myosin, heavy polypeptide 4 |
| BM392106 | Cald1 | caldesmon 1 |
| L81169 | Oxtr | oxytocin receptor |
| NM_012605 | Mylpf | myosin light chain, phosphorylatable, fast skeletal muscle |
| NM_012606 | Myl3 | myosin, light polypeptide 3 |
| NM_017240 | Myh6 /// Myh7 | myosin, heavy polypeptide 6, cardiac muscle, alpha /// myosin, heavy |
| | | polypeptide 7, cardiac muscle, beta |
| NM_019131 | Tpm1 | tropomyosin 1, alpha |
| NM_019212 | Acta1 | actin, alpha 1, skeletal muscle |
| NM_133424 | Actn3 | actinin alpha 3 |

Phosphate Transport

| | | |
|---|---|---|
| AA958001 | Cthrc1 | collagen triple helix repeat containing 1 |
| AB013454 | Slc34a1 | solute carrier family 34 (sodium phosphate), member 1 |
| AI030021 | — | Transcribed locus |
| AI176393 | Col4a1 | procollagen, type IV, alpha 1 |
| AI501709 | C1qtnf7_predicted | C1q and tumor necrosis factor related protein 7 (predicted) |
| AJ245707 | Phyh2 | phytanoyl-CoA 2-hydroxylase 2 |
| BF418957 | C1qa | complement component 1, q subcomponent, alpha polypeptide |
| BG662519 | Ank | Progressive ankylosis homolog (mouse) |
| BM389001 | Col9a3_predicted | procollagen, type IX, alpha 3 (predicted) |
| NM_021760 | Col5a3 | procollagen, type V, alpha 3 |
| NM_053714 | Ank | progressive ankylosis homolog (mouse) |
| NM_133418 | Slc25a10 | solute carrier family 25 (mitochondrial carrier; dicarboxylate |
| | | transporter), member 10 |

Protein Complex Assembly

| | | |
|---|---|---|
| AA945909 | Cd3e_predicted | CD3 antigen, epsilon polypeptide (predicted) |
| AI044631 | Cd3g | CD3 antigen, gamma polypeptide |
| AI227800 | Kifap3_predicted | kinesin-associated protein 3 (predicted) |
| AW915948 | Cd3e_predicted | CD3 antigen, epsilon polypeptide (predicted) |
| BF282632 | Tspan4 | tetraspanin 4 |
| BF410366 | — | Transcribed locus |
| BI277545 | Myh1 /// Myh2 | myosin, heavy polypeptide 1, skeletal muscle, adult /// myosin, heavy |
| | | polypeptide 2, skeletal muscle, adult |
| NM_013169 | Cd3d | CD3 antigen delta polypeptide |
| NM_022952 | Ap2s1 | adaptor-related protein complex 2, sigma 1 subunit |
| NM_030989 | Tp53 | tumor protein p53 |
| NM_031827 | Vamp8 | vesicle-associated membrane protein 8 |

Calcium-mediated Signaling

| | | |
|---|---|---|
| AB070350 | Chp /// | calcium binding protein p22 /// similar to calcium binding protein P22 |
| | RGD1564956_predicted /// | (predicted) |
| | RGD1565588_predicted | |
| AI170193 | Dscr1 | Down syndrome critical region homolog 1 (human) |
| BF282228 | Ltb | lymphotoxin B |
| BM383464 | — | Transcribed locus |

Regulation of GTPase Activity

| | | |
|---|---|---|
| AI234095 | Smap1l | stromal membrane-associated protein 1-like |
| AI408598 | Git2 | G protein-coupled receptor kinase-interactor 2 |
| BG378709 | — | — |
| BI275873 | Centb1_predicted | centaurin, beta 1 (predicted) |
| U28830 | Ralbp1 | ralA binding protein 1 |

Protein Amino Acid Glycosylation

| | | |
|---|---|---|
| AA799400 | B3galt3 | UDP-Gal:betaGlcNAc beta 1,3-galactosyltransferase, polypeptide 3 |
| AI112158 | B3gnt1_predicted | UDP-GlcNAc:betaGal beta-1,3-N-acetylglucosaminyltransferase 1 |
| | | (predicted) |
| AI237192 | Fut4 | fucosyltransferase 4 |
| BG373352 | B3gnt1_predicted | UDP-GlcNAc:betaGal beta-1,3-N-acetylglucosaminyltransferase 1 |
| | | (predicted) |
| NM_031337 | St3gal5 | ST3 beta-galactoside alpha-2,3-sialyltransferase 5 |

Regulation of Cell Shape

| | | |
|---|---|---|
| BE110369 | Arhgef18_predicted | rho/rac guanine nucleotide exchange factor (GEF) 18 (predicted) |
| BM389644 | Rhoj | ras homolog gene family, member J |
| NM_012755 | Fyn | fyn proto-oncogene |
| NM_013194 | Myh9 | myosin, heavy polypeptide 9, non-muscle |
| X74293 | Itga7 | integrin alpha 7 |
| X74294 | Itga7 | integrin alpha 7 |

Rn B Cell Receptor NetPath 12

| | | |
|---|---|---|
| AA900477 | Vav2_predicted | Vav2 oncogene (predicted) |
| AA925583 | Blnk | B-cell linker |
| AA998516 | Ccna2 | cyclin A2 |
| AI101388 | GAlNAc4S6ST | N-acetylgalactosamine 4-sulfate 6-O-sulfotransferase |
| AI103918 | Hdac7a | histone deacetylase 7A |
| AI137137 | Lck | lymphocyte protein tyrosine kinase |
| AI146037 | Map3k7_predicted | Mitogen activated protein kinase kinase kinase 7 (predicted) |
| AI171093 | Prkcq | protein kinase C, theta |
| AI177373 | Cr2_predicted | Complement receptor 2 (predicted) |
| AI385291 | Blk | B lymphoid kinase |
| AI408289 | Cd22_predicted | CD22 antigen (predicted) |
| AI556056 | Cr2_predicted | complement receptor 2 (predicted) |
| AW920039 | RGD1311584 | similar to stem cell adaptor protein STAP-1 |
| AW920881 | Actr2 /// | ARP2 actin-related protein 2 homolog (yeast) /// similar to ARP2 |
| | LOC301861 | actin-related protein 2 homolog (yeast) (predicted) |
| BF282471 | Lcp2 | lymphocyte cytosolic protein 2 |
| BF288130 | Ptprc | protein tyrosine phosphatase, receptor type, C |
| BF290113 | RGD1311584 | similar to stem cell adaptor protein STAP-1 |
| BF401586 | — | Transcribed locus |
| BF545268 | Cr2_predicted | complement receptor 2 (predicted) |
| BF555968 | Nedd9 | neural precursor cell expressed, developmentally down-regulated |
| | | gene 9 |
| BI288619 | Jun | Jun oncogene |
| BI301465 | — | Transcribed locus |
| BM387946 | Zap70 | zeta-chain (TCR) associated protein kinase 70 |
| L14782 | Lyn | Yamaguchi sarcoma viral (v-yes-1) oncogene homolog |
| M10072 | Ptprc | protein tyrosine phosphatase, receptor type, C |
| M13706 | Prkcb1 | protein kinase C, beta 1 |
| NM_012755 | Fyn | fyn proto-oncogene |
| NM_013187 | Plcg1 | phospholipase C, gamma 1 |
| NM_019295 | Cd5 | CD5 antigen |
| NM_019311 | Inpp5d | inositol polyphosphate-5-phosphatase D |
| NM_031020 | Mapk14 | mitogen activated protein kinase 14 |
| NM_033230 | Akt1 | thymoma viral proto-oncogene 1 |
| NM_053355 | Ikbkb | inhibitor of kappaB kinase beta |
| NM_053669 | Aps | adaptor protein with pleckstrin homology and src homology 2 |
| | | domains |
| NM_053883 | Dusp6 | dual specificity phosphatase 6 |
| NM_130421 | Lcp2 | lymphocyte cytosolic protein 2 |
| NM_133533 | Cd79b | CD79B antigen |
| X04440 | Prkcb1 | protein kinase C, beta 1 |

Rn T-Cell Receptor NetPath 11

| | | |
|---|---|---|
| AA900477 | Vav2_predicted | Vav2 oncogene (predicted) |
| AA945909 | Cd3e_predicted | CD3 antigen, epsilon polypeptide (predicted) |
| AI009944 | Txk | TXK tyrosine kinase |
| AI010476 | Rac2 | RAS-related C3 botulinum substrate 2 |
| AI044622 | MGC125004 | similar to T cell receptor interacting molecule |
| AI044631 | Cd3g | CD3 antigen, gamma polypeptide |
| AI103918 | Hdac7a | histone deacetylase 7A |
| AI137137 | Lck | lymphocyte protein tyrosine kinase |
| AI171093 | Prkcq | protein kinase C, theta |
| AI176755 | Ptpn22_predicted | protein tyrosine phosphatase, non-receptor type 22 (lymphoid) |
| | | (predicted) |
| AI408598 | Git2 | G protein-coupled receptor kinase-interactor 2 |
| AI500952 | Sla | Src-like adaptor |
| AW532114 | Rasgrp2_predicted | RAS guanyl releasing protein 2 (calcium and DAG-regulated) (predicted) |
| AW532179 | LOC500449 | similar to SHP2-interacting transmembrane adaptor protein |
| AW915948 | Cd3e_predicted | CD3 antigen, epsilon polypeptide (predicted) |
| BE117044 | Cd8a | CD8 antigen, alpha chain |
| BF282471 | Lcp2 | lymphocyte cytosolic protein 2 |
| BF288130 | Ptprc | protein tyrosine phosphatase, receptor type, C |
| BF418025 | Grap | GRB2-related adaptor protein |
| BF555968 | Nedd9 | neural precursor cell expressed, developmentally down-regulated gene 9 |
| BI274326 | Muc1 | mucin 1, transmembrane |
| BI288619 | Jun | Jun oncogene |
| BI290909 | Scap1 | src family associated phosphoprotein 1 |
| BM387946 | Zap70 | zeta-chain (TCR) associated protein kinase 70 |
| D13555 | Cd3z | CD3 antigen, zeta polypeptide |
| L08447 | Cd3z | CD3 antigen, zeta polypeptide |
| L14782 | Lyn | Yamaguchi sarcoma viral (v-yes-1) oncogene homolog |
| M10072 | Ptprc | protein tyrosine phosphatase, receptor type, C |
| NM_012755 | Fyn | fyn proto-oncogene |
| NM_012855 | Jak3 | Janus kinase 3 |
| NM_013169 | Cd3d | CD3 antigen delta polypeptide |
| NM_013187 | Plcg1 | phospholipase C, gamma 1 |
| NM_019295 | Cd5 | CD5 antigen |
| NM_024147 | Evl | Ena-vasodilator stimulated phosphoprotein |
| NM_030853 | Lat | linker for activation of T cells |
| NM_031538 | Cd8a | CD8 antigen, alpha chain |
| NM_033230 | Akt1 | thymoma viral proto-oncogene 1 |
| NM_130421 | Lcp2 | lymphocyte cytosolic protein 2 |

Rn IL-4 NetPath 16

| | | |
|---|---|---|
| AA945737 | Cxcr4 | chemokine (C-X-C motif) receptor 4 |
| AB020615 | Prkci | protein kinase C, iota |
| AI137137 | Lck | lymphocyte protein tyrosine kinase |
| AI178808 | Il2rg | interleukin 2 receptor, gamma (severe combined immunodeficiency) |
| BE109926 | Pik3cd_predicted | phosphatidylinositol 3-kinase catalytic delta polypeptide (predicted) |
| BG378885 | Hmga1 | high mobility group AT-hook 1 |
| BG663208 | Jak1 | Janus kinase 1 |
| NM_012555 | Ets1 | v-ets erythroblastosis virus E26 oncogene homolog 1 (avian) |
| NM_012755 | Fyn | fyn proto-oncogene |
| NM_012855 | Jak3 | Janus kinase 3 |
| NM_013187 | Plcg1 | phospholipase C, gamma 1 |
| NM_019311 | Inpp5d | inositol polyphosphate-5-phosphatase D |
| NM_031020 | Mapk14 | mitogen activated protein kinase 14 |
| NM_033230 | Akt1 | thymoma viral proto-oncogene 1 |
| U54791 | Cxcr4 | chemokine (C-X-C motif) receptor 4 |

Rn IL-7 NetPath 19

| | | |
|---|---|---|
| AA998516 | Ccna2 | cyclin A2 |
| AI178808 | Il2rg | interleukin 2 receptor, gamma (severe combined immunodeficiency) |
| AI385291 | Blk | B lymphoid kinase |
| BF397766 | — | — |
| BG663208 | Jak1 | Janus kinase 1 |
| BI274326 | Muc1 | mucin 1, transmembrane |
| L14782 | Lyn | Yamaguchi sarcoma viral (v-yes-1) oncogene homolog |
| NM_012755 | Fyn | fyn proto-oncogene |
| NM_012855 | Jak3 | Janus kinase 3 |
| NM_033230 | Akt1 | thymoma viral proto-oncogene 1 |

Rn Eicosanoid Synthesis

| | | |
|---|---|---|
| AB048730 | Ptges | prostaglandin E synthase |
| L07316 | Dpep1 | dipeptidase 1 (renal) |
| NM_017260 | Alox5ap | arachidonate 5-lipoxygenase activating protein |
| NM_031598 | Pla2g2a | phospholipase A2, group IIA (platelets, synovial fluid) |
| NM_053591 | Dpep1 | dipeptidase 1 (renal) |
| NM_053639 | Ltc4s | leukotriene C4 synthase |

Rn Insulin Signaling

| | | |
|---|---|---|
| AA799981 | Prkch | protein kinase C, eta |
| AA957585 | Ehd3 | EH-domain containing 3 |
| AI010476 | Rac2 | RAS-related C3 botulinum substrate 2 |
| AI146037 | Map3k7_predicted | Mitogen activated protein kinase kinase kinase 7 (predicted) |
| AI171093 | Prkcq | protein kinase C, theta |
| BE109926 | Pik3cd_predicted | phosphatidylinositol 3-kinase catalytic delta polypeptide (predicted) |
| BF401586 | — | Transcribed locus |
| BG378166 | Map4k2_predicted | mitogen activated protein kinase kinase kinase kinase 2 (predicted) |
| BI279786 | Myo1d | myosin 1D |
| BI288619 | Jun | Jun oncogene |
| BI291434 | Pfkm | phosphofructokinase, muscle |
| BM389644 | Rhoj | ras homolog gene family, member J |
| M13706 | Prkcb1 | protein kinase C, beta 1 |
| NM_012663 | Vamp2 | vesicle-associated membrane protein 2 |
| NM_031020 | Mapk14 | mitogen activated protein kinase 14 |
| NM_031085 | Prkch | protein kinase C, eta |
| NM_031715 | Pfkm | phosphofructokinase, muscle |
| NM_033230 | Akt1 | thymoma viral proto-oncogene 1 |
| NM_053355 | Ikbkb | inhibitor of kappaB kinase beta |
| NM_053535 | Enpp1 | ectonucleotide pyrophosphatase/phosphodiesterase 1 |
| NM_053669 | Aps | adaptor protein with pleckstrin homology and src homology 2 domains |
| NM_053847 | Map3k8 | mitogen-activated protein kinase kinase kinase 8 |
| NM_053887 | Map3k1 | mitogen activated protein kinase kinase kinase 1 |
| U24150 | Tsc2 | tuberous sclerosis 2 |
| X04440 | Prkcb1 | protein kinase C, beta 1 |

Rn Cholesterol Biosynthesis

| | | |
|---|---|---|
| AW530769 | Fdft1 | farnesyl diphosphate farnesyl transferase 1 |
| BG664123 | Cyp51 | cytochrome P450, subfamily 51 |
| BM390399 | Hmgcr | 3-hydroxy-3-methylglutaryl-Coenzyme A reductase |
| BM390574 | Lss | Lanosterol synthase |
| NM_012941 | Cyp51 | cytochrome P450, subfamily 51 |
| NM_013134 | Hmgcr | 3-hydroxy-3-methylglutaryl-Coenzyme A reductase |
| NM_017268 | Hmgcs1 | 3-hydroxy-3-methylglutaryl-Coenzyme A synthase 1 |
| NM_019238 | Fdft1 | farnesyl diphosphate farnesyl transferase 1 |
| NM_022389 | Dhcr7 | 7-dehydrocholesterol reductase |
| NM_031840 | Fdps | farnesyl diphosphate synthase |
| NM_053539 | Idi1 | isopentenyl-diphosphate delta isomerase |

Rn Fatty Acid Synthesis BiGCaT

| | | |
|---|---|---|
| AI179334 | Fasn | fatty acid synthase |
| AW915152 | Echdc1 | enoyl Coenzyme A hydratase domain containing 1 |
| BI296153 | Acaca | acetyl-coenzyme A carboxylase alpha |
| J02585 | Scd1 | stearoyl-Coenzyme A desaturase 1 |
| NM_012744 | Pc | Pyruvate carboxylase |
| NM_016987 | Acly | ATP citrate lyase |
| NM_017332 | Fasn | fatty acid synthase |
| NM_022193 | Acaca | acetyl-coenzyme A carboxylase alpha |
| NM_053623 | Acsl4 | acyl-CoA synthetase long-chain family member 4 |
| NM_130433 | Acaa2 | acetyl-Coenzyme A acyltransferase 2 (mitochondrial 3-oxoacyl- |
| | | Coenzyme A thiolase) |

Rn Krebs-TCA Cycle

| | | |
|---|---|---|
| AI009657 | Cs | Citrate synthase |
| AI411413 | LOC685778 /// | pyruvate dehydrogenase E1 alpha 1 /// pyruvate dehydrogenase E1 alpha |
| | Pdha1 | 1 pseudogene |
| BF561717 | Pdha1 | pyruvate dehydrogenase E1 alpha 1 |
| BI295900 | Dlat | dihydrolipoamide S-acetyltransferase (E2 component of pyruvate |
| | | dehydrogenase complex) |
| NM_012744 | Pc | Pyruvate carboxylase |
| NM_024398 | Aco2 | aconitase 2, mitochondrial |
| NM_130755 | Cs | citrate synthase |

Rn Mitochondrial Fatty Acid Betaoxidation

| | | |
|---|---|---|
| NM_013200 | Cpt1b | carnitine palmitoyltransferase 1b, muscle |
| NM_016986 | Acadm | acetyl-Coenzyme A dehydrogenase, medium chain |
| NM_031559 | Cpt1a | carnitine palmitoyltransferase 1a, liver |
| NM_053623 | Acsl4 | acyl-CoA synthetase long-chain family member 4 |
| NM_130433 | Acaa2 | acetyl-Coenzyme A acyltransferase 2 (mitochondrial 3-oxoacyl- |
| | | Coenzyme A thiolase) |

Rn Striated Muscle Contraction

| | | |
|---|---|---|
| AA875132 | — | Transcribed locus |
| AF370889 | Tpm1 | Tropomyosin 1, alpha |
| AF372216 | Tpm1 | tropomyosin 1, alpha |
| AF399874 | Tnnt1 | troponin T1, skeletal, slow |
| AI104533 | Ttn | titin |
| AI104913 | Tmod1 | tropomodulin 1 |
| AI710682 | Tnnc1 | troponin C type 1 (slow) |
| AW533848 | Mybpc2_predicted | myosin binding protein C, fast-type (predicted) |
| BF284889 | Actn2_predicted | actinin alpha 2 (predicted) |
| BF521859 | Tnnt3 | troponin T3, skeletal, fast |
| BG378588 | Mybpc2_predicted | myosin binding protein C, fast-type (predicted) |
| NM_012605 | Mylpf | myosin light chain, phosphorylatable, fast skeletal muscle |
| NM_012606 | Myl3 | myosin, light polypeptide 3 |
| NM_017185 | Tnni2 | troponin I type 2 (skeletal, fast) |
| NM_017240 | Myh6 /// Myh7 | myosin, heavy polypeptide 6, cardiac muscle, alpha /// myosin, heavy |
| | | polypeptide 7, cardiac muscle, beta |
| NM_019131 | Tpm1 | tropomyosin 1, alpha |
| NM_019212 | Acta1 | actin, alpha 1, skeletal muscle |
| NM_057208 | Tpm3 | tropomyosin 3, gamma |
| NM_133424 | Actn3 | actinin alpha 3 |

Leukocyte Transendothelial Migration

| | | |
|---|---|---|
| AA900477 | Vav2_predicted | Vav2 oncogene (predicted) |
| AA945737 | Cxcr4 | chemokine (C-X-C motif) receptor 4 |
| AF002251 | Rassf5 | Ras association (RalGDS/AF-6) domain family 5 |
| AI009944 | Txk | TXK tyrosine kinase |
| AI010476 | Rac2 | RAS-related C3 botulinum substrate 2 |
| AI012081 | Rhoh | ras homolog gene family, member H |
| AI137640 | Cldn1 | claudin 1 |
| AI169104 | Cxcl4 | chemokine (C-X-C motif) ligand 4 |
| AI599794 | — | Transcribed locus, moderately similar to XP_580071.1 PREDICTED: |
| | | hypothetical protein XP_580071 [Rattus norvegicus] |
| AI638971 | Ocln | occludin |
| BE328951 | Cldn4 | claudin 4 |
| BF284889 | Actn2_predicted | actinin alpha 2 (predicted) |
| BF386742 | — | Transcribed locus |
| BF401586 | — | Transcribed locus |
| BI275966 | Sipa1 | signal-induced proliferation-associated gene 1 |
| BI281680 | Cldn5 | claudin 5 |
| BI292090 | Cldn8 | claudin 8 |
| BM384008 | Arhgap5 | Rho GTPase activating protein 5 |
| M13706 | Prkcb1 | protein kinase C, beta 1 |
| NM_013187 | Plcg1 | phospholipase C, gamma 1 |
| NM_030863 | Msn | moesin |
| NM_031020 | Mapk14 | mitogen activated protein kinase 14 |
| NM_031144 | Actb | actin, beta |
| NM_031699 | Cldn1 | claudin 1 |
| NM_031700 | Cldn3 | claudin 3 |
| NM_133424 | Actn3 | actinin alpha 3 |
| U54791 | Cxcr4 | chemokine (C-X-C motif) receptor 4 |
| X04440 | Prkcb1 | protein kinase C, beta 1 |

T Cell Receptor Signaling Pathway

| | | |
|---|---|---|
| AA900477 | Vav2_predicted | Vav2 oncogene (predicted) |
| AA945727 | Card11_predicted | caspase recruitment domain family, member 11 (predicted) |
| AA945909 | Cd3e_predicted | CD3 antigen, epsilon polypeptide (predicted) |
| AB070350 | Chp /// | calcium binding protein p22 /// similar to calcium binding protein P22 |
| | RGD1564956_predicted /// | (predicted) |
| | RGD1565588_predicted | |
| AF081196 | Rasgrp1 | RAS guanyl releasing protein 1 |
| AI044631 | Cd3g | CD3 antigen, gamma polypeptide |
| AI137137 | Lck | lymphocyte protein tyrosine kinase |
| AI170362 | Nfkb2 | nuclear factor of kappa light polypeptide gene enhancer in B-cells 2, |
| | | p49/p100 |
| AW915948 | Cd3e_predicted | CD3 antigen, epsilon polypeptide (predicted) |
| BE099038 | — | — |
| BE117044 | Cd8a | CD8 antigen, alpha chain |
| BF282471 | Lcp2 | lymphocyte cytosolic protein 2 |
| BI288619 | Jun | Jun oncogene |
| BM387946 | Zap70 | zeta-chain (TCR) associated protein kinase 70 |
| D13555 | Cd3z | CD3 antigen, zeta polypeptide |
| L08447 | Cd3z | CD3 antigen, zeta polypeptide |
| NM_012755 | Fyn | fyn proto-oncogene |
| NM_013169 | Cd3d | CD3 antigen delta polypeptide |
| NM_013187 | Plcg1 | phospholipase C, gamma 1 |
| NM_022610 | Icos | inducible T-cell co-stimulator |
| NM_030853 | Lat | linker for activation of T cells |
| NM_031538 | Cd8a | CD8 antigen, alpha chain |
| NM_033230 | Akt1 | thymoma viral proto-oncogene 1 |
| NM_053355 | Ikbkb | inhibitor of kappaB kinase beta |
| NM_053826 | Pdk1 | pyruvate dehydrogenase kinase, isoenzyme 1 |
| NM_053847 | Map3k8 | mitogen-activated protein kinase kinase kinase 8 |
| NM_130421 | Lcp2 | lymphocyte cytosolic protein 2 |

Tight Junction

| | | |
|---|---|---|
| AA799981 | Prkch | protein kinase C, eta |
| AB020615 | Prkci | protein kinase C, iota |
| AF255614 | Magi3 | membrane associated guanylate kinase, WW and PDZ domain |
| | | containing 3 |
| AI137640 | Cldn1 | claudin 1 |
| AI171093 | Prkcq | protein kinase C, theta |
| AI599794 | — | Transcribed locus, moderately similar to XP_580071.1 PREDICTED: |
| | | hypothetical protein XP_580071 [Rattus norvegicus] |
| AI638971 | Ocln | Occluding |
| AI717081 | — | Transcribed locus |
| BE328951 | Cldn4 | claudin 4 |
| BF284889 | Actn2_predicted | actinin alpha 2 (predicted) |
| BF288177 | Csnk2a1 | casein kinase II, alpha 1 polypeptide |
| BF386742 | — | Transcribed locus |
| BF401586 | — | Transcribed locus |
| BF411017 | LOC304000 | cell adhesion molecule JCAM |
| BI281680 | Cldn5 | claudin 5 |
| BI292090 | Cldn8 | claudin 8 |
| BI301465 | — | Transcribed locus |
| M13706 | Prkcb1 | protein kinase C, beta 1 |
| NM_013194 | Myh9 | myosin, heavy polypeptide 9, non-muscle |
| NM_031085 | Prkch | protein kinase C, eta |
| NM_031144 | Actb | actin, beta |
| NM_031699 | Cldn1 | claudin 1 |
| NM_031700 | Cldn3 | claudin 3 |
| NM_033230 | Akt1 | thymoma viral proto-oncogene 1 |
| NM_053999 | Ppp2r2a | protein phosphatase 2 (formerly 2A), regulatory subunit B (PR 52), |
| | | alpha isoform |
| NM_133424 | Actn3 | actinin alpha 3 |
| X04440 | Prkcb1 | protein kinase C, beta 1 |

B Cell Receptor Signaling Pathway

| | | |
|---|---|---|
| AA900477 | Vav2_predicted | Vav2 oncogene (predicted) |
| AA925583 | Blnk | B-cell linker |
| AA945727 | Card11_predicted | caspase recruitment domain family, member 11 (predicted) |
| AB070350 | Chp /// | calcium binding protein p22 /// similar to calcium binding protein P22 |
| | RGD1564956_predicted /// | (predicted) |
| | RGD1565588_predicted | |
| AI010476 | Rac2 | RAS-related C3 botulinum substrate 2 |
| AI170362 | Nfkb2 | nuclear factor of kappa light polypeptide gene enhancer in B-cells 2, |
| | | p49/p100 |
| AI177373 | Cr2_predicted | Complement receptor 2 (predicted) |
| AI408289 | Cd22_predicted | CD22 antigen (predicted) |
| AI556056 | Cr2_predicted | complement receptor 2 (predicted) |
| BF401586 | — | Transcribed locus |
| BF545268 | Cr2_predicted | complement receptor 2 (predicted) |
| BI288619 | Jun | Jun oncogene |
| L14782 | Lyn | Yamaguchi sarcoma viral (v-yes-1) oncogene homolog |
| M13706 | Prkcb1 | protein kinase C, beta 1 |
| NM_019311 | Inpp5d | inositol polyphosphate-5-phosphatase D |
| NM_033230 | Akt1 | thymoma viral proto-oncogene 1 |
| NM_053355 | Ikbkb | inhibitor of kappaB kinase beta |
| NM_133533 | Cd79b | CD79B antigen |
| X04440 | Prkcb1 | protein kinase C, beta 1 |

Complement and Coagulation Cascades

| | | |
|---|---|---|
| AA818521 | Thbd | Thrombomodulin |
| AI177373 | Cr2_predicted | Complement receptor 2 (predicted) |
| AI556056 | Cr2_predicted | complement receptor 2 (predicted) |
| AI639117 | Cfb | complement factor B |
| AI716125 | C2 | complement component 2 |
| BF545268 | Cr2_predicted | complement receptor 2 (predicted) |
| BG666306 | Thbd | Thrombomodulin |
| BM388525 | F13a1 | coagulation factor XIII, A1 subunit |
| NM_012488 | A2m | alpha-2-macroglobulin |
| NM_012516 | C4bpa | complement component 4 binding protein, alpha |
| NM_012559 | Fgg | fibrinogen, gamma polypeptide |
| NM_012925 | Cd59 | CD59 antigen |
| NM_013085 | Plau | plasminogen activator, urokinase |
| NM_017200 | Tfpi | tissue factor pathway inhibitor |
| NM_021698 | F13a1 | coagulation factor XIII, A1 subunit |
| NM_031771 | Thbd | Thrombomodulin |
| NM_130409 | Cfh | complement component factor H |

Fc epsilon RI Signaling Pathway

| | | |
|---|---|---|
| AA900477 | Vav2_predicted | Vav2 oncogene (predicted) |
| AI010476 | Rac2 | RAS-related C3 botulinum substrate 2 |
| BF282471 | Lcp2 | lymphocyte cytosolic protein 2 |
| BI301465 | — | Transcribed locus |
| L14782 | Lyn | Yamaguchi sarcoma viral (v-yes-1) oncogene homolog |
| M17153 | Fcer1a | Fc receptor, IgE, high affinity I, alpha polypeptide |
| NM_012755 | Fyn | fyn proto-oncogene |
| NM_013187 | Plcg1 | phospholipase C, gamma 1 |
| NM_017174 | Pla2g5 | phospholipase A2, group V |
| NM_019311 | Inpp5d | inositol polyphosphate-5-phosphatase D |
| NM_030853 | Lat | linker for activation of T cells |
| NM_031020 | Mapk14 | mitogen activated protein kinase 14 |
| NM_031598 | Pla2g2a | phospholipase A2, group IIA (platelets, synovial fluid) |
| NM_033230 | Akt1 | thymoma viral proto-oncogene 1 |
| NM_053826 | Pdk1 | pyruvate dehydrogenase kinase, isoenzyme 1 |
| NM_130421 | Lcp2 | lymphocyte cytosolic protein 2 |
| NM_133551 | Pla2g4a | phospholipase A2, group IVA (cytosolic, calcium-dependent) |

Toll-like Receptor Signaling Pathway

| | | |
|---|---|---|
| AA893384 | Irf3 | interferon regulatory factor 3 |
| AF057025 | Tlr4 | toll-like receptor 4 |
| AI170362 | Nfkb2 | nuclear factor of kappa light polypeptide gene enhancer in B-cells 2, |
| | | p49/p100 |
| AW526982 | Tlr2 | toll-like receptor 2 |
| BF281987 | Cxcl11 | chemokine (C-X-C motif) ligand 11 |
| BF289368 | Lbp | lipopolysaccharide binding protein |
| BF396350 | Irak1_predicted | interleukin-1 receptor-associated kinase 1 (predicted) |
| BI288619 | Jun | Jun oncogene |
| NM_021744 | Cd14 | CD14 antigen |
| NM_031020 | Mapk14 | mitogen activated protein kinase 14 |
| NM_031116 | Ccl5 | chemokine (C-C motif) ligand 5 |
| NM_033230 | Akt1 | thymoma viral proto-oncogene 1 |
| NM_053355 | Ikbkb | inhibitor of kappaB kinase beta |
| U22520 | Cxcl10 | chemokine (C-X-C motif) ligand 10 |

PPAR Signaling Pathway

| | | |
|---|---|---|
| AA818262 | Angptl4 | angiopoietin-like 4 |
| AI013044 | — | Transcribed locus |
| BI274189 | Rxrb | retinoid X receptor beta |
| BI278687 | Pltp_predicted | phospholipid transfer protein (predicted) |
| J02585 | Scd1 | stearoyl-Coenzyme A desaturase 1 |
| M30596 | Me1 | malic enzyme 1 |
| NM_012600 | Me1 | malic enzyme 1 |
| NM_013200 | Cpt1b | carnitine palmitoyltransferase 1b, muscle |
| NM_016986 | Acadm | acetyl-Coenzyme A dehydrogenase, medium chain |
| NM_024162 | Fabp3 | fatty acid binding protein 3 |
| NM_031559 | Cpt1a | carnitine palmitoyltransferase 1a, liver |
| NM_031841 | Scd2 | stearoyl-Coenzyme A desaturase 2 |
| NM_053339 | Acox3 | acyl-Coenzyme A oxidase 3, pristanoyl |
| NM_053623 | Acsl4 | acyl-CoA synthetase long-chain family member 4 |
| U13253 | Fabp5 | fatty acid binding protein 5, epidermal |

Biosynthesis of Steroids

| | | |
|---|---|---|
| AB052846 | Sc5d | sterol-C5-desaturase (fungal ERG3, delta-5-desaturase) homolog |
| | | (S. cerevisae) |
| AI710051 | Sc5d | sterol-C5-desaturase (fungal ERG3, delta-5-desaturase) homolog |
| | | (S. cerevisae) |
| AW530769 | Fdft1 | farnesyl diphosphate farnesyl transferase 1 |
| BF417479 | Dhcr24 | 24-dehydrocholesterol reductase |
| BG664123 | Cyp51 | cytochrome P450, subfamily 51 |
| BM390399 | Hmgcr | 3-hydroxy-3-methylglutaryl-Coenzyme A reductase |
| BM390574 | Lss | Lanosterol synthase |
| NM_012941 | Cyp51 | cytochrome P450, subfamily 51 |
| NM_013134 | Hmgcr | 3-hydroxy-3-methylglutaryl-Coenzyme A reductase |
| NM_019238 | Fdft1 | farnesyl diphosphate farnesyl transferase 1 |
| NM_022389 | Dhcr7 | 7-dehydrocholesterol reductase |
| NM_031840 | Fdps | farnesyl diphosphate synthase |
| NM_053539 | Idi1 | isopentenyl-diphosphate delta isomerase |

Glycolysis/Gluconeogenesis

| | | |
|---|---|---|
| BI279760 | Pgk1 | phosphoglycerate kinase 1 |
| BI291434 | Pfkm | phosphofructokinase, muscle |
| BI295900 | Dlat | dihydrolipoamide S-acetyltransferase (E2 component of pyruvate |
| | | dehydrogenase complex) |
| BM389223 | Pdhb | pyruvate dehydrogenase (lipoamide) beta |
| NM_012497 | Aldoc | aldolase C |
| NM_012554 | Eno1 | enolase 1, alpha |
| NM_012949 | Eno3 | enolase 3, beta |
| NM_017328 | Pgam2 | phosphoglycerate mutase 2 |
| NM_031715 | Pfkm | phosphofructokinase, muscle |

Arachidonic Acid Metabolism

| | | |
|---|---|---|
| AA800587 | Gpx2 | glutathione peroxidase 2 |
| AB048730 | Ptges | prostaglandin E synthase |
| NM_017156 | Cyp2b15 | cytochrome P450, family 2, subfamily b, polypeptide 15 |
| NM_017174 | Pla2g5 | phospholipase A2, group V |
| NM_023025 | Cyp2j3 /// | cytochrome P450, family 2, subfamily J, polypeptide 4 /// cytochrome |
| | Cyp2j4 | P450, family 2, subfamily j, polypeptide 3 |
| NM_031598 | Pla2g2a | phospholipase A2, group IIA (platelets, synovial fluid) |
| NM_053639 | Ltc4s | leukotriene C4 synthase |
| NM_133551 | Pla2g4a | phospholipase A2, group IVA (cytosolic, calcium-dependent) |

Pyruvate Metabolism

| | | |
|---|---|---|
| BI295900 | Dlat | dihydrolipoamide S-acetyltransferase (E2 component of pyruvate |
| | | dehydrogenase complex) |
| BI296153 | Acaca | acetyl-coenzyme A carboxylase alpha |
| BM389223 | Pdhb | pyruvate dehydrogenase (lipoamide) beta |
| M30596 | Me1 | malic enzyme 1 |
| NM_012600 | Me1 | malic enzyme 1 |
| NM_012744 | Pc | Pyruvate carboxylase |
| NM_022193 | Acaca | acetyl-coenzyme A carboxylase alpha |

Riboflavin Metabolism

| | | |
|---|---|---|
| NM_019370 | Enpp3 | ectonucleotide pyrophosphatase/phosphodiesterase 3 |
| NM_020072 | Acpp | acid phosphatase, prostate |
| NM_053535 | Enpp1 | ectonucleotide pyrophosphatase/phosphodiesterase 1 |

TABLE 12
Biochemical Pathways Associated With Genes Diffeientially Expressed in Liver Tissue as a Result of High
Protein Diet

| | | |
|---|---|---|
| Public ID | Gene Symbol | Gene Title |

Lipid Metabolic Process

| | | |
|---|---|---|
| AA817761 | Adh1 | alcohol dehydrogenase 1 (class I) |
| AA891362 | Hadhsc | L-3-hydroxyacyl-Coenzyme A dehydrogenase, short chain |
| AA899721 | Mte1 | mitochondrial acyl—CoA thioesterase 1 |
| | Cd36///LOC685953/// | cd36 antigen///similar to fatty acid translocase/CD36 |
| AF072411 | RGD1562323_predicted | (predicted)///similar to CD36 antigen |
| AW435376 | Lrp5_predicted | low density lipoprotein receptor-related protein 5 (predicted) |
| BE116152 | Elovl6 | ELOVL family member 6, elongation of long chain fatty |
| | | acids (yeast) |
| BF283070 | Cyp7b1 | cytochrome P450, family 7, subfamily b, polypeptide 1 |
| BF396857 | Elovl6 | ELOVL family member 6, elongation of long chain fatty |
| | | acids (yeast) |
| BF555448 | Mte1 | Mitochondrial acyl—CoA thioesterase 1 |
| BI295569 | RGD1564089_predicted | similar to peroxisomal long chain acyl—CoA thioesterase Ia |
| | | (predicted) |
| BM390571 | Sult2b1_predicted | sulfotransferase family, cytosolic, 2B, member 1 (predicted) |
| J02585 | Scd1 | stearoyl-Coenzyme A desaturase 1 |
| NM_012695 | Smp2a | rat senescence marker protein 2A gene, exons 1 and 2 |
| NM_012737 | Apoa4 | apolipoprotein A-IV |
| NM_012907 | Apobec1 | apolipoprotein B editing complex 1 |
| NM_013084 | Acadsb | acyl-Coenzyme A dehydrogenase, short/branched chain |
| NM_016987 | Acly | ATP citrate lyase |
| NM_031559 | Cpt1a | carnitine palmitoyltransferase 1a, liver |
| NM_031641 | Sult4a1 | sulfotransferase family 4A, member 1 |
| NM_031841 | Scd2 | stearoyl-Coenzyme A desaturase 2 |
| | | acetyl-Coenzyme A acyltransferase 2 (mitochondrial 3- |
| NM_130433 | Acaa2 | oxoacyl-Coenzyme A thiolase) |
| U88294 | Cpt1a | carnitine palmitoyltransferase 1a, liver |
| Y09333 | Mte1 | mitochondrial acyl—CoA thioesterase 1 |

Fatty Acid Metabolic Process

| | | |
|---|---|---|
| AA891362 | Hadhsc | L-3-hydroxyacyl-Coenzyme A dehydrogenase, short chain |
| AF072411 | Cd36/// | cd36 antigen///similar to fatty acid translocase/CD36 (predicted)/// |
| | LOC685953/// | similar to CD36 antigen |
| | RGD1562323_ | |
| | predicted | |
| BE116152 | Elovl6 | ELOVL family member 6, elongation of long chain fatty acids (yeast) |
| BF396857 | Elovl6 | ELOVL family member 6, elongation of long chain fatty acids (yeast) |
| NM_013068 | Fabp2 | fatty acid binding protein 2, intestinal |
| NM_013084 | Acadsb | acyl-Coenzyme A dehydrogenase, short/branched chain |
| NM_031559 | Cpt1a | carnitine palmitoyltransferase 1a, liver |
| NM_031561 | RGD1562323_ | similar to fatty acid translocase/CD36 (predicted) |
| | predicted | |
| NM_031605 | Cyp4a8 | cytochrome P450, family 4, subfamily a, polypeptide 8 |
| NM_130433 | Acaa2 | acetyl-Coenzyme A acyltransferase 2 (mitochondrial 3-oxoacyl- |
| | | Coenzyme A thiolase) |
| U88294 | Cpt1a | carnitine palmitoyltransferase 1a, liver |

Lipid Biosynthetic Process

| | | |
|---|---|---|
| AI179334 | Fasn | fatty acid synthase |
| BE116152 | Elovl6 | ELOVL family member 6, elongation of long chain fatty acids (yeast) |
| BF396857 | Elovl6 | ELOVL family member 6, elongation of long chain fatty acids (yeast) |
| J02585 | Scd1 | stearoyl-Coenzyme A desaturase 1 |
| NM_012744 | Pc | Pyruvate carboxylase |
| NM_016987 | Ac [symbol] y | ATP citrate lyase |
| NM_ 017235 | Hsd17b7 | hydroxysteroid (17-beta) dehydrogenase 7 |
| NM_ 017332 | Fasn | fatty acid synthase |
| NM_031841 | Scd2 | stearoyl-Coenzyme A desaturase 2 |

Fatty Acid Biosynthetic Process

| | | |
|---|---|---|
| AI179334 | Fasn | fatty acid synthase |
| BE116152 | Elovl6 | ELOVL family member 6, elongation of long chain fatty acids (yeast) |
| BF396857 | Elovl6 | ELOVL family member 6, elongation of long chain fatty acids (yeast) |
| BI282211 | Acsm3 | acyl-CoA synthetase medium-chain family member 3 |
| BI296153 | Acaca | acetyl-coenzyme A carboxylase alpha |
| J02585 | Scd1 | stearoyl-Coenzyme A desaturase 1 |
| NM_017332 | Fasn | fatty acid synthase |
| NM_031841 | Scd2 | stearoyl-Coenzyme A desaturase 2 |

Steroid Metabolic Process

| | | |
|---|---|---|
| AI072107 | Akr1c6 | aldo-keto reductase family 1, member C6 |
| BF283070 | Cyp7b1 | cytochrome P450, family 7, subfamily b, polypeptide 1 |
| BF403483 | — | Transcribed locus, moderately similar to NP_997413.2 oxysterol |
| | | binding protein-like 1 [Mus musculus] |
| BM390571 | Sult2b1_predicted | sulfotransferase family, cytosolic, 2B, member 1 (predicted) |
| NM_012695 | Smp2a | rat senescence marker protein 2A gene, exons 1 and 2 |
| NM_031641 | Sult4a1 | sulfotransferase family 4A, member 1 |

Proteolysis

| | | |
|---|---|---|
| AI104238 | Gzma | granzyme A |
| AI639241 | LOC684318/// | ubiquitin specific protease 12 (predicted)///similar to ubiquitin specific |
| | RGD1561481_ | protease 12 (predicted)///similar to ubiquitin specific protease 12 |
| | predicted/// | |
| | Usp12_predicted | |
| BF395791 | — | Transcribed locus |
| BI283159 | Scpep1 | serine carboxypeptidase 1 |
| BM383209 | RGD1561153_ | similar to myosin-V [symbol] b (predicted) |
| | predicted | |
| NM_012552 | Ela1 | elastase 1, pancreatic |
| NM_019323 | Mcpt10/// | mast cell protease 8///mast cell protease 10///mast cell protease 9/// |
| | Mcpt8/// | mast cell protease 8-like 2 |
| | Mcpt8 [symbol] 2/// | |
| | Mcpt9 | |
| NM_053299 | Ubd | ubiquitin D |
| NM_130422 | Casp12 | caspase 12 |
| U36476 | Mmp9 | matrix metallopeptidase 9 |

Carbohydrate Metabolic Process

| | | |
|---|---|---|
| AA892845 | Hexb | hexosaminidase B |
| AA997683 | Aldh1b1 | aldehyde dehydrogenase 1 family, member B1 |
| AB052294 | Abcc8 | ATP-binding cassette, sub-family C (CFTR/MRP), member 8 |
| AW530361 | Ppp1r3c | protein phosphatase 1, regulatory (inhibitor) subunit 3C |
| BE115496 | — | Transcribed locus |
| BI279202 | RGD1565709_ | similar to ovostatin-2 (predicted) |
| | predicted | |
| BI283882 | Gpi | glucose phosphate isomerase |
| M17685 | Pklr | pyruvate kinase, liver and red blood cell |
| M30596 | Me1 | malic enzyme 1 |
| NM_012600 | Me1 | malic enzyme 1 |
| NM_012624 | Pklr | pyruvate kinase, liver and red blood cell |
| NM_013144 | Igfbp1 | insulin-like growth factor binding protein 1 |
| NM_017025 | Ldha | lactate dehydrogenase A |
| NM_ 024381 | Gyk | glycerol kinase |
| NM_031741 | Slc2a5 | solute carrier family 2, member 5 |

Glucose Metabolic Process

| | | |
|---|---|---|
| NM_012603 | Myc | myelocytomatosis viral oncogene homolog (avian) |
| NM_013144 | Igfbp1 | insulin-like growth factor binding protein 1 |
| NM_017025 | Ldha | lactate dehydrogenase A |
| NM_031559 | Cpt1a | carnitine palmitoyltransferase 1a, liver |
| U88294 | Cpt1a | carnitine palmitoyltransferase 1a, liver |

Gluconeogenesis

| | | |
|---|---|---|
| BI283882 | Gpi | glucose phosphate isomerase |
| M18340 | Tat | tyrosine aminotransferase |
| NM_012744 | Pc | Pyruvate carboxylase |
| NM_031039 | Gpt1 | glutamic pyruvic transaminase 1, soluble |
| NM_053962 | Sds | serine dehydratase |

Amino Acid Metabolic Process

| | | |
|---|---|---|
| AA799700 | Sephs2 | selenophosphate synthetase 2 |
| AI411227 | RGD1563485_ | similar to mKIAA0534 protein (predicted) |
| | predicted | |
| AI411345 | LOC680409/// | similar to Proline oxidase, mitochondrial precursor (Proline |
| | LOC682565 | dehydrogenase) |
| D00252 | Got1 | glutamate oxaloacetate transaminase 1 |
| M18340 | Tat | tyrosine aminotransferase |
| NM_012522 | Cbs | cystathionine beta synthase |
| NM_017206 | Slc6a6 | solute carrier family 6 (neurotransmitter transporter, taurine), member 6 |
| NM_022521 | Oat | ornithine aminotransferase |
| NM_031330 | Asl | argininosuccinate lyase |
| NM_053962 | Sds | serine dehydratase |

Amine Metabolic Process

| | | |
|---|---|---|
| AA799700 | Sephs2 | selenophosphate synthetase 2 |
| AI168953 | Sult1c2a | sulfotransferase family, cytosolic, 1C, member 2a |
| AI411345 | LOC680409/// | similar to Proline oxidase, mitochondrial precursor (Proline |
| | LOC682565 | dehydrogenase) |
| BI300997 | Sult1c2/// | sulfotransferase family, cytosolic, 1C, member 2///sulfotransferase |
| | Sult1c2a | family, cytosolic, 1C, member 2a |
| NM_031330 | Asl | argininosuccinate lyase |
| NM_133547 | Sult1c2/// | sulfotransferase family, cytosolic, 1C, member 2///sulfotransferase |
| | Sult1c2a | family, cytosolic, 1C, member 2a |

Nitrogen Compound Metabolic Process

| | | |
|---|---|---|
| AA799700 | Sephs2 | selenophosphate synthetase 2 |
| AI411345 | LOC580409//// | similar to Praline oxidase, mitochondrial precursor (Proline |
| | LOC682565 | dehydrogenase) |
| BI289085 | Vnn1 | vanin 1 |
| NM_031039 | Gpt1 | glutamic pyruvic transaminase 1, soluble |
| NM_031330 | Asl | argininosuccinate lyase |

One-carbon Compound Metabolic Process

| | | |
|---|---|---|
| AB030829 | Ca3 | carbonic anhydrase 3 |
| AI408948 | Ca2 | carbonic anhydrase 2 |
| BI301687 | — | Transcribed locus |
| NM_019291 | Ca2 | carbonic anhydrase 2 |
| NM_019292 | Ca3 | carbonic anhydrase 3 |

Xenobiotic Metabolic Process

| | | |
|---|---|---|
| AI072107 | Akr1c6 | aldo-keto reductase family 1, member C6 |
| AI169331 | Gstm2 | glutathione S-transferase, mu 2 |
| K02422 | Cyp1a2 | cytochrome P450, family 1, subfamily a, polypeptide 2 |
| NM_031543 | Cyp2e1 | cytochrome P450, family 2, subfamily e, polypeptide 1 |
| U46118 | Cyp3a9 | cytochrome P450, family 3, subfamily a, polypeptide 9 |

Sodium Ion Transport

| | | |
|---|---|---|
| AB013454 | Slc34a1 | solute carrier family 34 (sodium phosphate), member 1 |
| AI232036 | Atp1b1 | ATPase, Na+/K+ transporting, beta 1 polypeptide |
| BG381311 | Slc13a5 | solute carrier family 13 (sodium-dependent citrate transporter), member |
| | | 5 |
| M14137 | Atp1b1 | ATPase, Na+/K+ transporting, beta 1 polypeptide |
| NM_031548 | Scnn1a | sodium channel, nonvoltage-gated 1 alpha |
| NM_053380 | Slc34a2 | solute carrier family 34 (sodium phosphate), member 2 |

Multicellular organismal development

| | | |
|---|---|---|
| AA818262 | Angptl4 | angiopoietin-like 4 |
| AA893169 | Timp3 | tissue inhibitor of metalloproteinase 3 (Sorsby fundus dystrophy, |
| | | pseudoinflammatory) |
| AI045767 | Ank | progressive ankylosis homolog (mouse) |
| AI180398 | — | Transcribed locus |
| AI411227 | RGD1563485_ | similar to mKIAA0534 protein (predicted) |
| | predicted | |
| AI599423 | Gadd45g | growth arrest and DNA-damage-inducible 45 gamma |
| AY082609 | Abcb1a/// | ATP-binding cassette, sub-family B (MDR/TAP), member 1B///ATP- |
| | Abcb1b | binding cassette, sub-family B (MDR/TAP), member 1A |
| BE104552 | Elavl1_predicted | ELAV (embryonic lethal, abnormal vision, Drosophila)-like 1 (Hu |
| | | antigen R) (predicted) |
| BE121383 | Ncam2 | neural cell adhesion molecule 2 |
| BF391129 | — | Transcribed locus |
| BF391308 | — | Transcribed locus |
| BG662519 | Ank | Progressive ankylosis homolog (mouse) |
| BM986536 | Hist1h4b | histone cluster 1, H4b |
| NM_013144 | Igfbp1 | insulin-like growth factor binding protein 1 |
| NM_017025 | Ldha | lactate dehydrogenase A |
| NM_017149 | Meox2 | mesenchyme homeobox 2 |
| NM_022542 | Rhob | ras homolog gene family, member B |
| NM_031330 | Asl | argininosuccinate lyase |
| NM_053714 | Ank | progressive ankylosis homolog (mouse) |

Regulation of Cell Growth

| | | |
|---|---|---|
| AF065161 | Cish | cytokine inducible SH2-containing protein |
| AI600057 | Crim1_predicted | cysteine-rich motor neuron 1 (predicted) |
| AI703807 | Crim1_predicted | cysteine-rich motor neuron 1 (predicted) |
| BE104060 | Igfbp5 | insulin-like growth factor binding protein 5 |
| BF386770 | — | Transcribed locus |
| BI289620 | Crim1_predicted | cysteine-rich motor neuron 1 (predicted) |
| BI291842 | — | Transcribed locus |
| NM_013144 | Igfbp1 | insulin-like growth factor binding protein 1 |

Myelination

| | | |
|---|---|---|
| AA892845 | Hexb | hexosaminidase B |
| AI137640 | Cldn1 | claudin 1 |
| NM_012798 | Mal | myelin and lymphocyte protein, T-cell differentiation protein |
| NM_031073 | Ntf3 | neurotrophin 3 |
| NM_031841 | Scd2 | stearoyl-Coenzyme A desaturase 2 |

Regulation of progression through cell cycle

| | | |
|---|---|---|
| AB019693 | Keg1 | kidney expressed gene 1 |
| AI599423 | Gadd45g | growth arrest and DNA-damage-inducible 45 gamma |
| AW143798 | Ccnd1 | Cyclin D1 |
| BE [symbol] 18653 | — | Transcribed locus |
| NM_017019 | Il1a | interleukin 1 alpha |
| NM_020072 | Acpp | acid phosphatase, prostate |
| NM_024127 | Gadd45a | growth arrest and DNA-damage-inducible 45 alpha |

Antigen Processing and Presentation

| | | |
|---|---|---|
| AI715202 | RT I-Bb | RT1 class II, locus Bb |
| AJ243338 | RT1-CE5 | RT1 class I, CE5 |
| AJ249701 | RT1-A2/// | RT1 class Ib, locus Aw2///RT1 class Ia, locus A2///RTI class I, A3 |
| | RT1-A3/// | |
| | RT1-A-2 | |
| BI292055 | — | Transcribed locus |
| BM387813 | — | Transcribed locus |
| M10094 | RT1-Aw2 | RT1 class Ib, locus Aw2 |
| M24024 | RT1-Aw2 | RT1 class Ib, locus Aw2 |
| NM_012645 | RT1-Aw2 | RT1 class Ib, locus Aw2 |
| NM_053299 | Ubd | ubiquitin D |

Rn Adipogenesis

| | | |
|---|---|---|
| AA874941 | Adfp | Adipose differentiation related protein |
| AB020967 | Trib3 | tribbles homolog 3 (Drosophila) |
| AI072892 | Frzb | frizzled-related protein |
| BE106199 | Rora_predicted | RAR-related orphan receptor alpha (predicted) |
| BF394158 | — | Transcribed locus |
| BI285616 | Adfp | adipose differentiation related protein |
| BI289506 | — | Transcribed locus |
| J02585 | Scd1 | stearoyl-Coenzyme A desaturase 1 |
| L48060 | Prlr | prolactin receptor |
| M57668 | Prlr | prolactin receptor |
| NM_012524 | Cebpa | CCAAT/enhancer binding protein (C/EBP), alpha |
| NM_013095 | Smad3 | MAD homolog 3 (Drosophila) |
| NM_024127 | Gadd45a | growth arrest and DNA-damage-inducible 45 alpha |

Rn Fatty Acid Synthesis BiGCaT

| | | |
|---|---|---|
| AA891362 | Hadhsc | L-3-hydroxyacyl-Coenzyme A dehydrogenase, short chain |
| AI179334 | Fasn | fatty acid synthase |
| BI296153 | Acaca | acetyl-coenzyme A carboxylase alpha |
| J02585 | Scd1 | stearoyl-Coenzyme A desaturase 1 |
| NM_012744 | Pc | Pyruvate carboxylase |
| NM_016987 | Acly | ATP citrate lyase |
| NM_017332 | Fasn | fatty acid synthase |
| NM_130433 | Acaa2 | acetyl-Coenzyme A acyltransferase 2 (mitochondrial 3-oxoacyl- |
| | | Coenzyme A thiolase) |

Rn Glycolysis and Gluconeogenesis

| | | |
|---|---|---|
| BI283882 | Gpi | glucose phosphate isomerase |
| D00252 | Got1 | glutamate oxaloacetate transaminase 1 |
| M17685 | Pklr | pyruvate kinase, liver and red blood cell |
| NM_012624 | Pklr | pyruvate kinase, liver and red blood cell |
| NM_012744 | Pc | Pyruvate carboxylase |
| NM_017025 | Ldha | lactate dehydrogenase A |

Rn Nuclear Receptors in Lipid Metabolism and Toxicity

| | | |
|---|---|---|
| AY082609 | Abcb1a/// | ATP-binding cassette, sub-family B (MDR/TAP), member 1B///ATP- |
| | Abcb1b | binding cassette, sub-family B (MDR/TAP), member 1A |
| K02422 | Cyp1a2 | cytochrome P450, family 1, subfamily a, polypeptide 2 |
| NM_031543 | Cyp2e1 | cytochrome P450, family 2, subfamily e, polypeptide 1 |
| NM_053754 | Abcg5 | ATP-binding cassette, sub-family G (WHITE), member 5 |
| U46118 | Cyp3a9 | cytochrome P450, family 3, subfamily a, polypeptide 9 |

Rn Fatty Acid Beta Oxidation 1 BiGCaT

| | | |
|---|---|---|
| AA891362 | Hadhsc | L-3-hydroxyacyl-Coenzyme A dehydrogenase, short chain |
| NM_024381 | Gyk | glycerol kinase |
| NM_031559 | Cpt1a | carnitine palmitoyltransferase 1a, liver |
| U88294 | Cpt1a | carnitine palmitoyltransferase 1a, liver |

Rn Fatty Acid Omega Oxidation BiGCaT

| | | |
|---|---|---|
| AA817761 | Adh1 | alcohol dehydrogenase 1 (class I) |
| K02422 | Cyp1a2 | cytochrome P450, family 1, subfamily a, polypeptide 2 |
| NM_022407 | Aldh1a1 | aldehyde dehydrogenase family 1, member A1 |
| NM_031543 | Cyp2e1 | cytochrome P450, family 2, subfamily e, polypeptide 1 |

PPAR Signaling Pathway

| | | |
|---|---|---|
| AA818262 | Angptl4 | angiopoietin-like 4 |
| AF072411 | Cd36/// | cd36 antigen///similar to fatty acid translocase/CD36 (predicted)/// |
| | LOC685953/// | similar to CD36 antigen |
| | RGD1562323_ | |
| | predicted | |
| BF398558 | — | Transcribed locus |
| J02585 | Scd1 | stearoyl-Coenzyme A desaturase 1 |
| M30596 | Me1 | malic enzyme 1 |
| NM_012600 | Me1 | malic enzyme 1 |
| NM_013068 | Fabp2 | fatty acid binding protein 2, intestinal |
| NM_024381 | Gyk | glycerol kinase |
| NM_031559 | Cpt1a | carnitine palmitoyltransferase 1a, liver |
| NM_031605 | Cyp4a8 | cytochrome P450, family 4, subfamily a, polypeptide 8 |
| NM_031841 | Scd2 | stearoyl-Coenzyme A desaturase 2 |
| U88294 | Cpt1a | carnitine palmitoyltransferase 1a, liver |

Metabolism of Xenobiotics by Cytochrome P450

| | | |
|---|---|---|
| AA817761 | Adh1 | alcohol dehydrogenase 1 (class I) |
| AA858993 | Ugt2b | UDP glycosyltransferase 2 family, polypeptide B |
| AI169331 | Gstm2 | glutathione S-transferase, mu 2 |
| K02422 | Cyp1a2 | cytochrome P450, family 1, subfamily a, polypeptide 2 |
| M13506 | Udpgtr2 | liver UDP-glucuronosyltransferase, phenobarbital-inducible form |
| NM_020540 | Gstm4 | glutathione S-transferase M4 |
| NM_031154 | Gstm3 | glutathione S-transferase, mu type 3 |
| NM_031533 | Ugt2b | UDP glycosyltransferase 2 family, polypeptide B |
| NM_031543 | Cyp2e1 | cytochrome P450, family 2, subfamily e, polypeptide 1 |
| U46118 | Cyp3a9 | cytochrome P450, family 3, subfamily a, polypeptide 9 |

Fatty Acid Metabolism

| | | |
|---|---|---|
| AA817761 | Adh1 | alcohol dehydrogenase 1 (class I) |
| AA891362 | Hadhsc | L-3-hydroxyacyl-Coenzyme A dehydrogenase, short chain |
| NM_013084 | Acadsb | acyl-Coenzyme A dehydrogenase, short/branched chain |
| NM_031559 | Cpt1a | carnitine palmitoyltransferase 1a, liver |
| NM_031605 | Cyp4a8 | cytochrome P450, family 4, subfamily a, polypeptide 8 |
| NM_130433 | Acaa2 | acetyl-Coenzyme A acyltransferase 2 (mitochondrial 3-oxoacyl- |
| | | Coenzyme A thiolase) |
| U88294 | Cpt1a | carnitine palmitoyltransferase 1a, liver |

Alanine and Aspartate Metabolism

| | | |
|---|---|---|
| BF416465 | — | Transcribed locus |
| D00252 | Got1 | glutamate oxaloacetate transaminase 1 |
| NM_012744 | Pc | Pyruvate carboxylase |
| NM_021577 | Asl | argininosuccinate lyase |
| NM_031039 | Gpt1 | glutamic pyruvic transaminase 1, soluble |
| NM_031330 | Asl | argininosuccinate lyase |

Arginine and Praline Metabolism

| | | |
|---|---|---|
| AI234919 | Eprs | glutamyl-prolyl-tRNA synthetase |
| AI411345 | LOC680409/// | similar to Praline oxidase, mitochondrial precursor (Praline |
| | LOC682565 | dehydrogenase) |
| D00252 | Got1 | glutamate oxaloacetate transaminase 1 |
| NM_021577 | Asl | argininosuccinate lyase |
| NM_022521 | Oat | ornithine aminotransferase |
| NM_031330 | Asl | argininosuccinate lyase |

Pyruvate Metabolism

| | | |
|---|---|---|
| BI296153 | Acaca | acetyl-coenzyme A carboxylase alpha |
| M17685 | Pklr | pyruvate kinase, liver and red blood cell |
| M30596 | Me1 | malic enzyme 1 |
| NM_012600 | Me1 | malic enzyme 1 |
| NM_012624 | Pklr | pyruvate kinase, liver and red blood cell |
| NM_012744 | Pc | Pyruvate carboxylase |
| NM_017025 | Ldha | lactate dehydrogenase A |

Glutamate Metabolism

| | | |
|---|---|---|
| AI234919 | Eprs | glutamyl-prolyl-tRNA synthetase |
| BF397926 | Gmps | guanine monphosphate synthetase |
| BF416465 | — | Transcribed locus |
| D00252 | Got1 | glutamate oxaloacetate transaminase 1 |
| J05499 | Gls2 | glutaminase 2 (liver, mitochondrial) |
| NM_031039 | Gpt1 | glutamic pyruvic transaminase 1, soluble |

Nitrogen Metabolism

| | | |
|---|---|---|
| AB030829 | Ca3 | carbonic anhydrase 3 |
| AI408948 | Ca2 | carbonic anhydrase 2 |
| J05499 | Gls2 | glutaminase 2 (liver, mitochondrial) |
| NM_017159 | Hal | histidine ammonia lyase |
| NM_019291 | Ca2 | carbonic anhydrase 2 |
| NM_019292 | Ca3 | carbonic anhydrase 3 |

Cysteine Metabolism

| | | |
|---|---|---|
| BI300997 | Sult1c2/// | sulfotransferase family, cytosolic, 1C, member 2///sulfotransferase |
| | Sult1c2a | family, cytosolic, 1C, member 2a |
| D00252 | Got1 | glutamate oxaloacetate transaminase 1 |
| NM_017025 | Ldha | lactate dehydrogenase A |
| NM_031641 | Sult4a1 | sulfotransferase family 4A, member 1 |
| NM_053962 | Sds | serine dehydratase |
| NM_133547 | Sult1c2/// | sulfotransferase family, cytosolic, 1C, member 2///sulfotransferase |
| | Sult1c2a | family, cytosolic, 1C, member 2a |

Tyrosine Metabolism

| | | |
|---|---|---|
| AA817761 | Adh1 | alcohol dehydrogenase 1 (class [symbol] ) |
| AI232328 | Hgd | homogentisate 1,2-dioxygenase |
| D00252 | Got1 | glutamate oxaloacetate transaminase 1 |
| M18340 | Tat | tyrosine aminotransferase |
| NM_012531 | Comt | catechol-O-methyltransferase |

[symbol]

indicates data missing or illegible when filed

TABLE 13
Biochemical Pathways Associated with Genes Differentially Expressed in Muscle Tissue as a Result of
High Protein Diet

| | | |
|---|---|---|
| Public ID | Gene Symbol | Gene Title |

Immune Response

| | | |
|---|---|---|
| AA892854 | LOC498335 | similar to Small inducible cytokine B13 precursor (CXCL13) (B |
| | | lymphocyte chemoattractant) (CXC chemokine BLC) |
| AI502757 | Cst7_predicted | cystatin F (leukocystatin) (predicted) |
| AI715202 | RT1-Bb | RT1 class II, locus Bb |
| AJ249701 | RT1-A2/// | RT1 class Ib, locus Aw2///RT1 class Ia, locus A2///RT1 class I, A3 |
| | RT1-A3/// | |
| | RT1-Aw2 | |
| BF290088 | Nkx2- | NK2 transcription factor related, locus 3 (Drosophila) (predicted) |
| | 3_predicted | |
| BI282920 | Ccl21b | chemokine (C-C motif) ligand 21b (serine) |
| BM389513 | LOC688090/// | RT1 class II, locus Bb///similar to RT1 class II histocompatibility |
| | RT1-Bb | antigen, B-1 beta chain precursor (RT1.B-beta(1)) |
| M24024 | RT1-Aw2 | RT1 class Ib, locus Aw2 |
| NM_019205 | Ccl11 | chemokine (C-C motif) ligand 11 |
| NM_053299 | Ubd | ubiquitin D |

Defense response

| | | |
|---|---|---|
| AI407339 | Irgm | immunity-related GTPase family, M |
| AJ249701 | RT1-A2///RT1-A3/// | RT1 class Ib, locus Aw2///RT1 class 1a, locus A2///RT1 class I, A3 |
| | RT1-Aw2 | |
| L81174 | Ankrd1 | ankyrin repeat domain 1 (cardiac muscle) |
| NM_012594 | Lalba | lactalbumin, alpha |
| NM_013220 | Ankrd1 | ankyrin repeat domain 1 (cardiac muscle) |
| NM_031971 | Hspa1a///Hspa1b | heat shock 70 kD protein 1A///heat shock 70 kD protein 1B (mapped) |

Inflammatory Response

| | | |
|---|---|---|
| AB001382 | Spp1 | secreted phosphoprotein 1 |
| AJ249701 | RT1-A2///RT1-A3/// | RT1 class Ib, locus Aw2///RT1 class Ia, locus A2///RT1 class I, |
| | RT1-Aw2 | A3 |
| BI282920 | Ccl21b | chemokine (C-C motif) ligand 21b (serine) |
| NM_019205 | Ccl11 | chemokine (C-C motif) ligand 11 |
| NM_024131 | Ddt | D-dopachrome tautomerase |

Rn Circadian Exercise

| | | |
|---|---|---|
| AI406939 | G0s2 | G0/G1 switch gene 2 |
| AW530361 | Ppp1r3c | protein phosphatase 1, regulatory (inhibitor) subunit 3C |
| BI274605 | Ucp3 | uncoupling protein 3 (mitochondrial, proton carrier) |
| U86635 | Gstm5 | glutathione S-transferase, mu 5 |
| U92069 | Ucp3 | uncoupling protein 3 (mitochondrial, proton carrier) |

The specification has disclosed typical preferred embodiments of the invention. Although specific terms are employed, they are used in a generic and descriptive sense only and not for purposes of limitation, the scope of the invention being set forth in the claims. Clearly, many modifications and variations of the invention are possible in light of the above teachings. It is therefore to be understood that within the scope of the appended claims the invention may be practiced otherwise than as specifically described.

What is claimed is:

1. A method for detecting differential expression of one or more genes differentially expressed in animals exhibiting a lean phenotype resulting from one or more lean phenotype-promoting treatments comprising (1) administration of CLA, (2) consumption of a high protein diet, and (3) increased exercise, as compared with normal animals, the method comprising:
a) providing probes comprising (i) polynucleotides that specifically hybridize to two or more gene encoding proteins selected from the group consisting of gene encoding proteins differentially expressed in adipose tissue; gene encoding proteins differentially expressed in liver tissue; gene encoding proteins differentially expressed in quadripcep tissues; gene encoding proteins differentially expressed in adipose and liver tissues; gene encoding proteins differentially expressed in adipose and quadriceps tissues; gene encoding proteins differentially expressed in liver and quadriceps tissues; and gene encoding proteins differentially expressed in adipose, liver and quadriceps tissues; wherein the polynucleotides comprise at least 10 contiguous nucleotides of the genes; or (ii) polypeptide binding agents that specifically bind to polypeptides comprising the two or more gene encoding proteins;
b) adding the probes to a sample comprising mRNA or proteins from an animal exhibiting the lean phenotype, in a manner enabling hybridization or binding of the probes to the mRNA or proteins in the sample, thereby forming hybridization or binding complexes in the sample;
c) optionally, adding the probes to another sample comprising mRNA or proteins from a normal animal, in a manner enabling hybridization or binding of the probes to the mRNA or proteins in the second sample, thereby forming hybridization or binding complexes in the other sample;
d) detecting the hybridization complexes in the sample or samples; and
e) comparing the hybridization or binding complexes from the first sample with the hybridization or binding complexes from a standard or, optionally, from the other sample, wherein at least one difference between the amount of hybridization or binding in the sample as compared with the standard or the optional other sample indicates differential expression of the one or more genes differentially expressed in animals exhibiting the lean phenotype.

2. The method of claim 1, wherein the genes encoding proteins are selected from the group consisting of: M24024, BI282114, AI555855, AF411318, BE329244, NM_{—}053999, J00710, BF524010, NM_{—}012594, AW251280, BE116152, NM_{—}012646, NM_{—}012600, AW533569, NM_{—}012703, NM_{—}024147, AA901290, AI137640, BE102861, AA955771, AI715202, NM_{—}012531, 302585, NM_{—}023025, AA799328, AI237143, AI179886, NM_{—}017332, NM_{—}030994, BI282114, AI232414, BI278180, AW522341, AI406386, NM_{—}031971, and AI599463.

3. The method of claim 1 wherein the probes specifically hybridize to, or specifically bind to, genes encoding proteins are selected from the group consisting of: M24024, BI282114, AI555855, BE329244, NM_{—}012646, AF411318, NM_{—}053999, J00710, BF524010, NM_{—}012594, AW251280, AI715202, NM_{—}012531, J02585, NM_{—}023025, AA799328, AI179886, NM_{—}017332, and NM_{—}030994.

4. The method of claim 1, wherein the probes specifically hybridize to gene encoding proteins selected from the group consisting of: M24024, BI282114, AI555855, BE329244, NM_{—}012646, AF411318, NM_{—}053999, J00710, BF524010, NM_{—}012594, AW251280, AI715202, NM_{—}012531, J02585, and NM_{—}023025.

5. The method of claim 1, wherein the probes specifically hybridize to, or specifically bind to, gene encoding proteins selected from the group consisting of: M24024, BI282114, AI555855, BE329244, NM_{—}012646, AF411318, NM_{—}053999, J00710, BF524010, and NM_{—}012594.

6. The method of claim 1, wherein the probes specifically hybridize or bind to canine or feline polynucleotides or polypeptides.

7. The method of claim 1, wherein the probes are bound to a substrate.

8. The method of claim 7, wherein the probes are in an array.

9. The method of claim 1, wherein the detecting is performed at intervals and used to monitor an animal's progress when attempting to promote the lean phenotype in the animal.

10. A method of determining if a test substance is likely to be useful in promoting a lean phenotype when administered to an animal, the method comprising:
a) determining a first gene expression profile by measuring the transcription or translation products of two or more polynucleotides selected from genes encoding proteins selected from the group consisting of gene encoding proteins differentially expressed in adipose tissue; gene encoding proteins differentially expressed in liver tissue; gene encoding proteins differentially expressed in quadripcep tissues; gene encoding proteins differentially expressed in adipose and liver tissues; gene encoding proteins differentially expressed in adipose and quadriceps tissues; gene encoding proteins differentially expressed in liver and quadriceps tissues; and gene encoding proteins differentially expressed in adipose, liver and quadriceps tissues; wherein the polynucleotides comprise at least 10 contiguous nucleotides of the genes, in a test system in the absence of the test substance;
b) determining a second gene expression profile by measuring the transcription or translation products of two or more polynucleotides selected from genes encoding proteins selected from the group consisting of gene encoding proteins differentially expressed in adipose tissue; gene encoding proteins differentially expressed in liver tissue; gene encoding proteins differentially expressed in quadripcep tissues; gene encoding proteins differentially expressed in adipose and liver tissues; gene encoding proteins differentially expressed in adipose and quadriceps tissues; gene encoding proteins differentially expressed in liver and quadriceps tissues; and gene encoding proteins differentially expressed in adipose, liver and quadriceps tissues; wherein the polynucleotides comprise at least 10 contiguous nucleotides of the genes, in a test system in the presence of the test substance; and
c) comparing the first gene expression profile with the second gene expression profile, wherein a change in the second gene expression profile as compared with the first gene expression profile indicates that the test substance is likely to be useful in promoting a lean phenotype when administered to an animal.

11. The method of claim 10, further comprising comparing at least the second gene expression profile with a reference gene expression profile obtained by measuring the transcription or translation products of two or more polynucleotides selected from genes encoding proteins selected from the group consisting of gene encoding proteins differentially expressed in adipose tissue; gene encoding proteins differentially expressed in liver tissue; gene encoding proteins differentially expressed in quadripcep tissues; gene encoding proteins differentially expressed in adipose and liver tissues; gene encoding proteins differentially expressed in adipose and quadriceps tissues; gene encoding proteins differentially expressed in liver and quadriceps tissues; and gene encoding proteins differentially expressed in adipose, liver and quadriceps tissues; wherein the polynucleotides comprise at least 10 contiguous nucleotides of the genes, in a test system in the presence of a reference substance known to promote a lean phenotype when administered to animals.

12. The method of claim 10, wherein the test system comprises a population of cultured cells.

13. The method of claim 10, wherein the test system comprises animals.

14. The method of claim 10, wherein the probes specifically hybridize to genes encoding proteins wherein the genes encoding proteins are selected from the group consisting of: M24024, BI282114, AI555855, AF411318, BE329244, NM_{—}053999, J00710, BF524010, NM_{—}012594, AW251280, BE116152, NM_{—}012646, NM_{—}012600, AW533569, NM_{—}012703, NM_{—}024147, AA901290, AI137640, BE102861, AA955771, AI715202, NM_{—}012531, J02585, NM_{—}023025, AA799328, AI237143, AI179886, NM_{—}017332, NM_{—}030994, BI282114, AI232414, BI278180, AW522341, AI406386, NM_{—}031971, and AI599463.

15. The method of claim 10, wherein the probes specifically hybridize to, or specifically bind to, genes encoding proteins are selected from the group consisting of: M24024, BI282114, AI555855, BE329244, NM_{—}012646, AF411318, NM_{—}053999, J00710, BF524010, NM_{—}012594, AW251280, AI715202, NM_{—}012531, J02585, NM_{—}023025, AA799328, AI179886, NM_{—}017332, and NM_{—}030994.

16. The method of claim 10, wherein the probes specifically hybridize to gene encoding proteins selected from the group consisting of: M24024, BI282114, AI555855, BE329244, NM_{—}012646, AF411318, NM_{—}053999, J00710, BF524010, NM_{—}012594, AW251280, AI715202, NM_{—}012531, J02585, and NM_{—}023025.

17. The method of claim 10, wherein the probes specifically hybridize to, or specifically bind to, gene encoding proteins selected from the group consisting of M24024, BI282114, AI555855, BE329244, NM_{—}012646, AF411318, NM_{—}053999, J00710, BF524010, and NM_{—}012594.

18. The method of claim 10, wherein the probes are bound to a substrate.

19. The method of claim 18, wherein the probes are in an array.

20. The method of claim 10, wherein the sample contains mRNA or proteins from a canine or feline.
